(19) United States
(12) Patent Application Publication
(10) Pub. No.: US 2024/0371462 A1
(43) Pub. Date: Nov. 7, 2024

(54) SYSTEMS AND METHODS FOR GENERATIVE DESIGN OF CUSTOM BIOLOGICS

(71) Applicant: Pythia Labs, Inc., Los Angeles, CA (US)

(72) Inventors: Thibault Marie Duplay, Los Angeles, CA (US); Lucas Zanini, Los Angeles, CA (US); Mohamed El Hibouri, Los Angeles, CA (US); Ramin Ansari, Los Angeles, CA (US); Julien Jorda, Los Angeles, CA (US); Lisa Juliette Madeleine Barel, Los Angeles, CA (US); Matthias Maria Alessandro Malago, Santa Monica, CA (US); Joshua Laniado, Los Angeles, CA (US); Wesley Michael Botello-Smith, Norwalk, CA (US); Tim-Henrik Buelles, Santa Monica, CA (US); Mohit Yadav, Los Angeles, CA (US)

(21) Appl. No.: 18/634,254

(22) Filed: Apr. 12, 2024

Related U.S. Application Data
(60) Provisional application No. 63/596,216, filed on Nov. 3, 2023.
(60) Provisional application No. 63/460,985, filed on Apr. 21, 2023.
(60) Provisional application No. 63/459,124, filed on Apr. 13, 2023.

(51) Int. Cl. G16B 15/30; G06F 30/27; G16B 40/20

(52) U.S. Cl. CPC G16B 15/30; G06F 30/27; G16B 40/20

(57) ABSTRACT

Presented herein are systems and methods for generative design of custom biologics. In particular, in certain embodiments, generative biologic design technologies of the present disclosure utilize a machine learning models to create custom (e.g., de-novo) peptide backbones that, among other things, can be tailored to exhibit desired properties and/or bind to specified target molecules, such as other proteins (e.g., receptors). Generative machine learning models described herein may be trained on, and accordingly leverage, a vast landscape of existing protein and peptide structures. Once trained, however, these generative models may create wholly new (de-novo) custom peptide backbones that are expressly tailored to particular targets. These generated custom peptide backbones can, e.g., subsequently, be populated with amino acid sequences to generate final custom biologics providing enhanced performance for binding to desired targets.

### CROSS-REFERENCE TO RELATED APPLICATIONS

This application claims priority to U.S. Provisional Application No. 63/596,216, filed Nov. 3, 2023, U.S. Provisional Application No. 63/460,985, filed Apr. 21, 2023, and U.S. Provisional Application No. 63/459,124, filed Apr. 13, 2023, the contents of each of which are incorporated herein by reference in their entirety.

### BACKGROUND

An increasing number of important drugs and vaccines are complex biomolecules referred to as biologics. For example, seven of the top ten best selling drugs as of early 2020 were biologics, including the monoclonal antibody adalimumab (Humira®). Biologics have much more complex structure than traditional small molecule drugs. The process of drug discovery, drug development, and clinical trials requires an enormous amount of capital and time. Typically, new drug candidates undergo in vitro testing, in vivo testing, then clinical trials prior to approval.

Software tools for in-silico design and testing of new drug candidates can cut the cost and time of the preclinical pipeline. However, biologics often have hard-to-predict properties and molecular behavior. To date, despite recent interest in software and computational tools (including artificial intelligence (AI) and machine learning) relating to biological molecules, the extraordinary complexity of biologics continues to challenge computational tools aiming to produce accurate predictions for biologics and advances are needed.

### SUMMARY

Presented herein are systems and methods for generative design of custom biologics. In particular, in certain embodiments, generative biologic design technologies of the present disclosure utilize a machine learning models to create custom (e.g., de-novo) peptide backbones that, among other things, can be tailored to exhibit desired properties and/or bind to specified target molecules, such as other proteins (e.g., receptors). Generative machine learning models described herein may be trained on, and accordingly leverage, a vast landscape of existing protein and peptide structures. Once trained, however, these generative models may create wholly new (de-novo) custom peptide backbones that are expressly tailored to particular targets. These generated custom peptide backbones can, e.g., subsequently, be populated with amino acid sequences to generate final custom biologics providing enhanced performance for binding to desired targets.

In one aspect, the invention is directed to a method for computer generation of a de-novo peptide backbone of a custom biologic, the method comprising: (a) receiving and/or generating, by a processor of a computing device a seed set comprising a plurality of feature vectors, each feature vector corresponding to a particular backbone site and comprising position and/or orientation components representing a position and/or orientation of the particular backbone site [e.g., wherein values of the position and/or orientation components of each feature vector are set to initial starting values (e.g., selected at random from an initial probability (e.g., uniform) distribution)]; (b) determining, by the processor, using a machine learning model, one or more (e.g., a plurality of) velocity fields and, beginning with the seed set, updating (e.g., iteratively), by the processor, values of the position and/or orientation components of the plurality of feature vectors according to the one or more velocity fields, thereby evolving the values of the position and/or orientation components of the plurality of feature vectors from a set of initial starting values into a set of final values representing positions and/or orientations of each backbone site of a generated peptide backbone; and (c) creating, by the processor, using, the set of final values, a generated scaffold model representing the de-novo peptide backbone [e.g., using the set of final values as the generated scaffold model representing the de-novo peptide backbone; e.g., determining, based on the set of final values, positions of backbone atoms (e.g., at each backbone site, a position of one or more backbone atoms (e.g., an N, alpha-Carbon, C, and O)) to be represented via the generated scaffold model]; and (d) storing and/or providing (e.g., for display and/or further processing), the generated scaffold model.

In certain embodiments, for each particular feature vector of the plurality of feature vectors, the position and/or orientation components of the particular feature vector represent a relative translation and/or rotation, respectively, of the corresponding backbone site relative to another, neighbor backbone site corresponding to another feature vector in the seed set.

In certain embodiments, for each particular feature vector of the plurality of feature vectors, the position and/or orientation components of the particular feature vector represent a global translation and/or rotation, respectively, with respect to a single common reference frame (e.g., origin) [e.g., and wherein the machine learning model is equivariant with respect to three-dimensional (3D) translations and/or rotations].

In certain embodiments, values of the position and/or orientation components of each feature vector are set to initial starting values selected at random from an initial probability (e.g., uniform) distribution.

In certain embodiments, the method comprises generating, by the processor, using a preliminary machine learning model to determine a plurality of sets of initial starting values, each set of initial starting values comprising an initial position and/or orientation and, at step (a), for each feature vector of the plurality of feature vectors selecting and using a set of initial starting values as values for the position and/or orientation components of the feature vector.

In certain embodiments, the seed set comprises 50 or more feature vectors {e.g., wherein the seed set comprises 100 or more feature vectors [e.g., wherein the seed set comprises 200 or more feature vectors (e.g., wherein the seed set comprises 500 or more feature vectors)]}.

In certain embodiments, step (b) comprises determining the one or more velocity fields and updating the values of the position and/or orientation components of the plurality of feature vectors in an iterative fashion [e.g., wherein each of the one or more velocity fields is an initial velocity field associated with and determined at a first iteration and/or a current velocity field associated with and determined at a particular subsequent iteration].

In certain embodiments, determining the one or more velocity fields and updating the values of the position and/or orientation components of the plurality of feature vectors in an iterative fashion comprises: determining initial starting values for the position and/or orientation components of each of the plurality of feature vectors; beginning with the initial starting values of the position and/or orientation components of the plurality of feature vectors, at a first iteration: determining, by the processor, using the machine learning model, an initial velocity field based on the initial starting values of the position and/or orientation components of the plurality of feature vectors; and updating values of the position and/or orientation components of the plurality of feature vectors according to the initial velocity field; and at subsequent iterations: using the updated values from a prior iteration as current values of the position and/or orientation components of the feature vectors; determining, using the machine learning model, a current velocity field based on the current values of the position and/or orientation components of the feature vectors; and updating values of the position and/or orientation components of the feature vectors according to the current velocity field; and upon reaching a final iteration, using the updated values of the position and/or orientation components of the feature vectors as the final set of values for creating the generated scaffold model representing the de-novo peptide backbone.

In certain embodiments, initial starting values of the position and/or orientation components of each feature vector are selected at random from an initial probability (e.g., uniform) distribution.

In certain embodiments, the method comprises determining, by the processor, the set of initial starting values using a preliminary machine learning model (e.g., a variational autoencoder).

In certain embodiments, each iteration corresponds to one of a plurality of time-points [e.g., separated according to a (e.g., pre-defined) time-step], and (i) the initial velocity field is determined based (e.g., further) at least in part on an initial time-point corresponding to the first iteration; and/or (ii) each current velocity field associated with and determined at a particular subsequent iteration is determined based (e.g., further) at least in part on a particular subsequent time-point corresponding to the subsequent iteration.

In certain embodiments, at each particular iteration, the machine learning model receives, as input, (i) the current values of the feature vectors and (ii) the time point corresponding to the particular iteration, and generates, as output, the current velocity field.

In certain embodiments, at each particular iteration, the machine learning model receives, as input, (i) the current values of the feature vectors and (ii) the time point corresponding to the particular iteration, and generates, as output, a set of prospective final values of the position and/or orientation components of the feature vectors (e.g., representing a current estimate of positions and/or orientations of each backbone site of a generated peptide backbone) and the current velocity field is determined based on the set of prospective final values and the time point corresponding to the current iteration (e.g., generating a current estimate of x₁ as output of the machine learning model and computing the current velocity field based on x₁ and the current time point).

In certain embodiments, the current time point is used, within the machine learning model, as a conditioning value.

In certain embodiments, at step (b), the machine learning model generates at least a portion (e.g., each) of the one or more velocity fields as output.

In certain embodiments, at step (b), the machine learning model generates a set of prospective final values of the position and/or orientation components of the feature vectors (e.g., representing a current estimate of positions and/or orientations of each backbone site of a generated peptide backbone) as output and the current velocity field is determined based on the set of prospective final values and a current time point [e.g., corresponding to a current iteration (e.g., generating a current estimate of x₁ as output of the machine learning model and computing the current velocity field based on x₁ and the current time point)].

In certain embodiments, the set of prospective final values comprises, for each particular feature vector: (i) a prospective final value of the position component represents a translation relative to a position component of the (e.g., same) particular feature vector at the current time (e.g., thereby expressing a final position of the backbone site represented by the feature vector as a translation relative to its position at an earlier, e.g., the current, time point); and/or (ii) a prospective final value of the orientation component represents a rotation relative to an orientation component of the (e.g., same) particular feature vector at the current time (e.g., thereby expressing a final orientation of the backbone site represented by the feature vector as a rotation relative to its orientation at an earlier, e.g., the current, time point).

In certain embodiments, the machine learning model is or comprises a transformer-based model.

In certain embodiments, the machine learning model receives and/or operates on an input graph representation of a peptide backbone, the input graph representation comprising a plurality of nodes and edges, each node corresponding to and representing a particular backbone (e.g., amino acid) site, and each edge associated with and relating two nodes [e.g., thereby representing (i) an interaction between and/or (ii) a relative position and/or orientation between two particular backbone (e.g., amino acid) sites], and wherein the machine learning model comprises at least one transformer-based edge retrieval layer that: (A) comprises one or more self-attention head(s), each of which determines a corresponding set of attention weights based at least in part on values of the feature vectors and/or node feature values determined therefrom, such that the edge retrieval layer determines one or more sets of attention weights; and (B) uses the one or more sets of attention weights to determine values of retrieved edge feature vectors.

In certain embodiments, the machine learning model is or comprises a graph neural network (GNN).

In certain embodiments, the machine learning model is or comprises an auto-regressive neural network.

In certain embodiments, the machine learning model receives, as input and/or determines, values of one or more edge features, each determined based on values of the position and/or orientation components of two or more feature vectors representing two or more different backbone sites.

In certain embodiments, the method comprises conditioning generation of the one or more velocity fields according to a set of one or more desired peptide backbone features.

In certain embodiments, the machine learning model receives, as input, and conditions generation of the one or more velocity fields on values one or more global property variables, each global property variable representing a desired property of a protein or peptide (e.g., having the generated peptide backbone).

In certain embodiments, the one or more global property variables comprise one or more of the following: (A) a protein family variable whose value identifies a particular one of a set of protein family types (e.g., a categorical variable that can take on one of a finite set of values, each identifying a particular protein family; e.g., finite length vector that represents a particular protein family via a one-hot encoding); (B) a thermostability variable whose value categorizes and/or measures (e.g., quantitatively) protein thermostability [e.g., a binary variable classifying a protein as thermophilic or not (e.g., based on PDB classification); e.g., a continuous real-number measuring melting temperature]; (C) an immunology variable whose value classifies and/or measures a propensity and/or likelihood of provoking an immune response [e.g., in a particular host organism (e.g., a human)]; (D) a function variable whose value classifies protein function; (E) a solubility variable whose value classifies and/or measures a protein solubility; and (F) a pH sensitivity variable whose value classifies and/or measures protein pH sensitivity.

In certain embodiments, the machine learning model receives, as input and conditions generation of the one or more velocity fields on, values of one or more node property variables, each node property variable associated with an representing a particular property of a particular amino acid site.

In certain embodiments, the one or more node property tokens comprise one or more of the following: (A) a side chain type variable that identifies a particular type of amino acid side chain; (B) an amino acid polarity variable that identifies a polarity and/or charge of an amino acid site; (C) a buriedness variable that classifies and/or measures an extent to which a particular amino acid site is buried and/or surface-accessible (e.g., a binary core/surface value); (D) a contact hotspot variable classifying a particular amino acid site according to a desired and/or threshold distance from one or more portions of another (e.g., target) molecule; and (E) a secondary structure variable whose value classifies and/or measures a secondary structure motif at the particular amino acid site.

In certain embodiments, the machine learning model receives, as input, values of one or more edge property variables (e.g., a contact hotspot token classifying the edge as associated with one or more particular amino acid sites having a desired and/or within a threshold distance from one or more portions of another (e.g., target) molecule).

In certain embodiments, the method comprises conditioning generation of the one or more velocity fields according to a representation of at least a portion of a target molecule (e.g., protein and/or peptide) and/or one or more particular sub-regions (e.g., prospective binding sites) thereof, thereby creating a de-novo peptide backbone suitable for binding to the target molecule (e.g., at a location within or comprising at least one of the one or more prospective binding sites).

In certain embodiments, the method comprises conditioning generation of the one or more velocity fields according to a representation of at least a portion of a desired final protein and/or peptide (e.g., a scaffold model representing at least a portion of a desired peptide backbone; e.g., an amino acid sequence of at least a portion of a desired final protein and/or peptide).

In certain embodiments, the method comprises: using the generated scaffold model as input to an interface designer module (e.g., comprising a machine learning model) for generating an amino acid interface for binding to a target molecule; and populating, by the processor, at least a portion of the generated scaffold model with a plurality of amino acids selected, by the processor (e.g., using a machine learning model) based at least in part on (i) the generated scaffold model and (ii) a target model representing at least a portion of the target molecule.

In certain embodiments, the method comprises creating, based at least in part on the generated scaffold model, a custom biologic [e.g., a therapeutic biologic; e.g., a diagnostic biologic; e.g., a theranostic biologic; e.g., an enzyme (e.g., for use in manufacturing or other industrial processes, e.g., disposal, clean up, etc.); e.g., a biologic that acts as a structural element].

In certain embodiments, the method comprises determining an amino acid sequence based at least in part on (e.g., that will and/or is predicted to fold into a three-dimensional structure corresponding to) the generated scaffold model.

In certain embodiments, the method comprises providing a polynucleotide encoding a sequence (e.g., an amino acid sequence) determined based on the generated scaffold model (e.g., the polynucleotide having a nucleic acid sequence that encodes a corresponding amino acid sequence determined using the generated scaffold model) and delivering the polynucleotide, for transcription and translation thereof, to a plurality of host cells, thereby producing the custom biologic.

In certain embodiments, the method comprises determining (e.g., at step (b); e.g., subsequent to step (b), e.g., via repeated use of the machine learning model), using the machine learning model, one or more sequence velocity fields and using the one or more sequence velocity fields to generate predicted sequence data representing an amino acid sequence of a protein and/or peptide having the de-novo peptide backbone.

In certain embodiments, the method comprises determining (e.g., at step (b); e.g., subsequent to step (b), via a repeated use of the machine learning model), using the machine learning model, one or more side chain geometry velocity fields and using the one or more side chain velocity fields to generate a prediction of a three-dimensional side chain geometry for an amino acid side chain (e.g., attached) at each of at least a portion of amino acid sites of the de-novo peptide backbone [e.g., wherein the three-dimensional side chain geometry at the portion of amino acid sites is represented via a set of side chain feature vectors, each corresponding to a particular side chain/amino acid site and having values representing a particular three-dimensional geometry of a side chain at that amino acid site (e.g., having four elements, each corresponding to one of four possible torsion angles), and the method comprises updating values of the feature vectors (e.g., repeatedly) according to the one or more determined side chain velocity fields].

In certain embodiments, the method comprises conditioning generation of the one or more velocity fields according to one or more protein fold representation(s) [e.g., wherein the machine learning model receives, as input, and conditions output on, the one or more protein fold representation(s)].

In certain embodiments, the one or more protein fold representation(s) are or comprise a set of secondary structure element (SSE) values (e.g., a SSE feature vector), each SSE value associated with a particular position (e.g., amino acid site) within a polypeptide chain of the custom biologic and having a value encoding a particular type of secondary structure [e.g., each value representing one of a plurality of possible secondary structure motifs/categories (e.g., helix, strand, loop, etc.)] at the particular position.

In certain embodiments, the one or more protein fold representation(s) are or comprise a block adjacency matrix [e.g., representing a spatial and orientational (e.g., angle) relationship between SSE's at pairs of amino acid sides], said block adjacency matrix comprising a plurality of elements, each element of the block adjacency matrix associated with a particular pair of positions (e.g., a pair of amino acid sites) within a polypeptide chain of the custom biologic and having a value representing a level (e.g., one of two or more discrete levels) of interaction between the particular pair of positions (e.g., that would occur in a desired 3D folded shape of the protein) (e.g., based on a proximity between the amino acid sites and/or a particular SSE value at each of the amino acid sites) [e.g., wherein each value of the block adjacency matrix is one of two possible binary values, specifying whether a pair of amino acid sites would interact (e.g., be within a particular cutoff distance) in a 3D structure according to the desired fold family].

In certain embodiments, the one or more protein fold representation(s) are or comprise a block adjacency matrix [e.g., representing a spatial and orientational (e.g., angle) relationship between SSE's at pairs of amino acid sides], said block adjacency matrix comprising a plurality of elements, each element of the block adjacency matrix associated with a particular pair of positions (e.g., a pair of amino acid sites) within a polypeptide chain of the custom biologic and having one or more values representing a relative position and/or orientation secondary structural elements (SSEs) at the particular pair of positions [e.g., the topological classification value selected from one of a set of categories (e.g., parallel, antiparallel, vertical; e.g., each element having a plurality of values (e.g., different channels), each associated with a particular topological classification category and having a binary value representing whether or not (the 3D folds at) the pair of positions has/is oriented in accordance with the particular topological classification category; e.g., a (e.g., continuous numerical value) representing a relative distance and/or angle between SSEs].

In another aspect the invention is directed to a method for computer-aided generative design of de-novo polypeptides and/or complexes thereof, the method comprising: (a) receiving and/or generating, by a processor of a computing device, a seed representation of one or more peptide backbones and/or amino acid sequences, the seed representation comprising a plurality of feature values representing (i) (e.g., locations and/or orientations of) amino acid sites and/or (ii) individual backbone atoms and/or side chain types thereof; (b) determining, by the processor, using one or more machine learning models, one or more (e.g., a plurality of) velocity fields and, beginning with the seed set, updating (iteratively), by the processor, the plurality of feature values according to the one or more velocity fields, thereby evolving the representation of one or more peptide backbones and/or amino acid sequences from initial feature values of the seed representation to a set of final feature values representing amino acid sites, and/or individual backbone atoms and/or side chain types, of one or more generated peptide backbones and/or amino acid sequences of the one or more de-novo polypeptide and/or a complex thereof; (c) creating, by the processor, using the set of final values, one or more generated scaffold model(s) and/or sequence string(s) representing the de-novo polypeptide(s) and/or complexes thereof; and (d) storing and/or providing (e.g., for display and/or further processing) the generated scaffold models and/or sequence strings.

In certain embodiments, the plurality of feature values comprise values representing locations (e.g., in Cartesian space) of one or more individual backbone atoms (e.g., N, C, alpha-Carbon, and O) for each of a plurality of amino acid sites of the de-novo polypeptide to-be and/or being generated.

In certain embodiments, the plurality of feature values comprise values representing positions and/or orientations of amino acid sites of the de-novo polypeptide to-be and/or being generated.

In certain embodiments, the plurality of feature values comprise sequence data representing an amino acid sequence of the de-novo polypeptide to-be and/or being generated.

In certain embodiments, the method comprises one or more features described herein, e.g., in paragraphs above (e.g., at paragraphs [0006]-[0044]).

In certain embodiments, the method comprises obtaining (e.g., receiving and/or accessing and/or generating), by the processor, a template model representing a partial sequence and/or three-dimensional structure of at least a portion of a reference protein and/or peptide; at step (a), generating the seed representation comprising the plurality of feature values, wherein a first subset of the plurality of feature values correspond to pre-defined regions of the polypeptide(s) being designed and are populated with feature values representing (e.g., and having been determined based on) the partial sequence and/or three dimensional structure of the reference protein and/or peptide (e.g., using the template model) and wherein a second subset of the plurality of feature values correspond to custom, to-be-designed, regions of the polypeptide(s) and are populated with initial starting values; and at step (b), determining the one or more velocity fields based at least in part on the plurality of feature values, including the first subset (corresponding to pre-defined regions) and the second subset (corresponding to unknown region that are, e.g., iteratively, updated) [e.g., thereby conditioning generation of the one or more velocity fields at least in part on the first subset of feature values] and updating the second subset of feature values based on the one or more velocity fields, while retaining/holding fixed the first subset of feature values.

In certain embodiments, the method comprises: obtaining (e.g., receiving and/or accessing and/or generating), by the processor, a template model representing a partial sequence and/or three-dimensional structure of one or more base portion(s) of a reference protein and/or peptide; determining, by the processor, one or more protein fold representation(s) based on the template model, wherein each of the one or more protein fold representation(s) represent (i) a secondary structure type of, and/or (ii) a relative proximity and/or orientation between amino acid sites of, the one or more base portion(s) of the reference protein and/or peptide [e.g., but do not include or represent definite three-dimensional coordinates of backbone atoms of the reference protein and/or peptide]; at step (a), generating the seed representation comprising the plurality of feature values, said plurality of feature values representing (i) locations and/or orientations of, and/or (ii) individual backbone atoms and/or side chain types of, a plurality of amino acid sites within a de-novo polypeptide (e.g., being designed), and wherein: a first subset of the plurality of feature values represent [e.g., (i) locations and/or orientations, and/or (ii) individual backbone atoms and/or side chain types of] amino acid sites within a first portion of the de-novo polypeptide, said first portion corresponding to the one or more base portion(s) of the reference polypeptide; and a second subset of the plurality of feature values represent [e.g., (i) locations and/or orientations, and/or (ii) individual backbone atoms and/or side chain types of] amino acid sites within a second portion of the de-novo polypeptide, said second portion adjacent to (e.g., having one or more amino acid sites adjacent to sites within) the first portion; and at step (b), determining the one or more velocity fields based at least in part on the one or more protein fold representation(s), such that the one or more generated peptide backbones and/or amino acid sequences comprise a first portion having a three-dimensional fold based on the one or more protein fold representation(s) and a second, custom, portion.

In certain embodiments, the reference protein and/or peptide is an antibody [e.g., and wherein the one or more base portions are or comprise heave and/or light chain region(s) of the antibody, but exclude complementary determining regions (CDRs) of the antibody].

In another aspect, the invention is directed to a method for (e.g., memory efficient) generating attention-based deep-learning predictions from input graph representations comprising nodes and edges, the method comprising: (a) receiving and/or accessing, by a processor of a computing device, an initial graph representation comprising a plurality of node feature vectors, each node feature vector associated with a particular node of the initial graph representation and comprising one or more initial node feature values; (b) determining, by the processor, using a machine learning model, a predicted graph representation, wherein the machine learning model: (i) receives the initial graph representation as input; (ii) comprises at least one edge retrieval layer, wherein the at least one edge retrieval layer: (A) comprises one or more self-attention head(s), each of which determines a corresponding set of attention weights based at least in part on values of the feature vectors and/or node feature values determined therefrom, such that the edge retrieval layer determines one or more sets of attention weights; and (B) uses the one or more sets of attention weights to determine values of retrieved edge feature vectors; and (iii) generates, as output, based at least in part on the values of the retrieved edge feature vectors, a plurality of predicted node feature vectors and/or one or more velocity fields, and wherein the predicted graph representation is determined using the plurality of predicted node feature vectors and/or one or more velocity fields; and (c) storing and/or providing (e.g., for display and/or further processing), the predicted graph representation.

In another aspect, the invention is directed to a method for designing a custom biologic having a desired three-dimensional (e.g., fold) fold, the method comprising: (a) receiving and/or generating, by a processor of a computing device, one or more protein fold representations, each encoding desired three-dimensional structural features of the desired fold at/in at least a portion of locations within the custom biologic; (b) receiving and/or generating, by the processor, a seed set comprising a plurality of feature vectors, each feature vector corresponding to a particular (e.g., amino acid) site within the one or more variable region(s) of template model and comprising one or both of: (A) position and/or orientation components representing a position and/or orientation of (e.g., individual backbone atoms and/or a local frame representation of) the particular site; and (B) a side chain type component, representing likelihoods of one or more particular amino acid side chain types occupying the particular site [e.g., wherein values of (A) the position and/or orientation components and/or (B) side chain type components of each feature vector are set to initial starting values (e.g., selected at random from an initial probability (e.g., uniform) distribution)]; (c) determining, by the processor, using a machine learning model, one or more (e.g., a plurality of) velocity fields based at least in part on the one or more protein fold representations, and, beginning with the seed set, updating (e.g., iteratively), by the processor, values of one or both of (e.g., (A) and (B)) (A) the position and/or orientation components and (B) the side chain type components of the plurality of feature vectors according to the one or more velocity fields, thereby evolving them a set of initial starting values into a set of final values representing (i) positions and/or orientations of each site of generated peptide backbone structures of the one or more variable regions, and/or (ii) likelihoods of (e.g., amino acid) side chain types at locations within the one or more variable regions; (d) generating, by the processor, using the set of final values, one or both of: (A) a scaffold model representing a three-dimensional structure of a de-novo peptide backbone for the custom biologic, and (B) sequence data representing an amino acid sequence of the custom biologic; and (c) storing and/or providing, by the processor, the generated (A) scaffold model and/or (B) sequence data for display and/or further processing and/or use in designing the custom biologic.

In certain embodiments, the one or more protein fold representation(s) are or comprise a set of secondary structure element (SSE) values (e.g., a SSE feature vector), each SSE value associated with a particular position (e.g., amino acid site) within a polypeptide chain of the custom biologic and having a value encoding a particular type of secondary structure [e.g., each value representing one of a plurality of possible secondary structure motifs/categories (e.g., helix, strand, loop, etc.)] at the particular position.

In certain embodiments, the one or more protein fold representation(s) are or comprise a block adjacency matrix [e.g., representing a spatial and orientational (e.g., angle) relationship between SSE's at pairs of amino acid sides], said block adjacency matrix comprising a plurality of elements, each element of the block adjacency matrix associated with a particular pair of positions (e.g., a pair of amino acid sites) within a polypeptide chain of the custom biologic and having a value representing a level (e.g., one of two or more discrete levels) of interaction between the particular pair of positions (e.g., that would occur in a desired 3D folded shape of the protein) (e.g., based on a proximity between the amino acid sites and/or a particular SSE value at each of the amino acid sites) [e.g., wherein each value of the block adjacency matrix is one of two possible binary values, specifying whether a pair of amino acid sites would interact (e.g., be within a particular cutoff distance) in a 3D structure according to the desired fold family].

In certain embodiments, the one or more protein fold representation(s) are or comprise a block adjacency matrix [e.g., representing a spatial and orientational (e.g., angle) relationship between SSE's at pairs of amino acid sides], said block adjacency matrix comprising a plurality of elements, each element of the block adjacency matrix associated with a particular pair of positions (e.g., a pair of amino acid sites) within a polypeptide chain of the custom biologic and having one or more values representing a relative position and/or orientation secondary structural elements (SSEs) at the particular pair of positions [e.g., the topological classification value selected from one of a set of categories (e.g., parallel, antiparallel, vertical; e.g., each element having a plurality of values (e.g., different channels), each associated with a particular topological classification category and having a binary value representing whether or not (the 3D folds at) the pair of positions has/is oriented in accordance with the particular topological classification category; e.g., a (e.g., continuous numerical value) representing a relative distance and/or angle between SSEs].

In certain embodiments, e.g., step (c) comprises performing the method of any one of various aspects and/or embodiments described herein, e.g., in paragraphs above (e.g., at paragraphs [0005]-[0053]) [e.g., using flow-matching; e.g., using the machine learning model to update and evolve feature values representing (i) (e.g., locations and/or orientations of) amino acid sites and/or (ii) individual backbone atoms and/or side chain types thereof] to generate the sequence and/or the three-dimensional peptide backbone structure, and wherein} the machine learning model receives the one or more protein fold representations as input, thereby conditioning generation of the one or more velocity fields the desired fold.

In certain embodiments, the method comprises, at step (a) generating the one or more protein fold representations based on a portion of an antibody template representing at least a portion (e.g., a Fab region, a variable heavy chain, a variable light chain, substantially all, all, etc.) of a reference antibody structure.

In certain embodiments, the portion of the antibody template is a base portion, representing a region of the reference antibody about one or more complementary-determining regions (CDRs) but excluding the CDRs themselves.

In certain embodiments, steps (b) through (d) comprises generating one or both of (i) a custom sequence and (ii) a custom three-dimensional peptide backbone structure of the one or more CDRs.

In certain embodiments, the method comprises one or more features described herein, e.g., in paragraphs above (e.g., at paragraphs [0006]-[0044]).

In another aspect, the invention is directed to a method for designing a custom antibody for binding to a target [e.g., a target antigen or portion (e.g., epitope) thereof], the method comprising: (a) receiving and/or generating, by a processor of a computing device, an antibody template representing a sequence and/or three-dimensional structure of at least a portion of a reference antibody, the antibody template comprising (i) a base portion located about one or more CDRs of the reference antibody, but (the base portion) excluding at least a portion of the one or more CDRs themselves; (b) determining, by the processor, one or more protein fold representations, each encoding and representing three-dimensional structural features of at least the base portion of the antibody template; (c) receiving and/or generating, by the processor, a seed set comprising a plurality of feature vectors, each feature vector corresponding to a particular (e.g., amino acid) site within one or more variable region(s) corresponding to, and representing to-be-designed custom versions of, CDR regions of the reference antibody and comprising one or both of: (A) position and/or orientation components representing a position and/or orientation of (e.g., individual backbone atoms and/or a local frame representation of) the particular site and (B) a side chain type component, representing likelihoods of one or more particular amino acid side chain types occupying the particular site [e.g., wherein values of (A) the position and/or orientation components and/or (B) side chain type components of each feature vector are set to initial starting values (e.g., selected at random from an initial probability (e.g., uniform) distribution)]; (c) determining, by the processor, using a machine learning model, one or more (e.g., a plurality of) velocity fields based at least in part on the one or more protein fold representations, and, beginning with the seed set, updating (e.g., iteratively), by the processor, values of one or both of (e.g., (A) and (B)) (A) the position and/or orientation components and (B) the side chain type components of the plurality of feature vectors according to the one or more velocity fields, thereby evolving them a set of initial starting values into a set of final values representing (i) positions and/or orientations of each site of generated peptide backbone structures of the one or more variable regions, and/or (ii) likelihoods of (e.g., amino acid) side chain types at locations within the one or more variable regions; (d) generating, by the processor, using the set of final values, one or both of: (A) a scaffold model representing a three-dimensional structure of a de-novo peptide backbone for one or more CDR regions of the custom antibody; and (B) sequence data representing an amino acid sequence of one or more CDR regions of the custom antibody; (c) storing and/or providing, by the processor, the generated (A) scaffold model and/or (B) sequence data, for display and/or further processing and/or use in designing the custom biologic.

In certain embodiments, the method comprises receiving and/or generating a target model representing a sequence and/or three-dimensional structure of at least a portion of the target (e.g., an epitope) and wherein step (c) comprises generating one or both of (i) the sequence and (ii) the three-dimensional peptide backbone structure based further on the target model [e.g., wherein the machine learning model receives, as input, and conditions output on, at least a portion of the target model and/or one or more features values derived therefrom].

In certain embodiments, the method comprises one or more features described herein, e.g., in paragraphs above (e.g., at paragraphs [0006]-[0044]).

In another aspect, the invention is directed to a method for in-silico generation and/or prediction of three-dimensional (3D) side chain geometries of a polypeptide chain (e.g., a protein and/or peptide), the method comprising: (a) receiving and/or generating, by a processor of a computing device, (i) sequence data representing an amino acid sequence of at least a portion of the polypeptide chain and (ii) a representation of a 3D peptide backbone geometry, and/or folds thereof, for the portion of the polypeptide chain; (b) receiving and/or generating, by a processor of a computing device a seed set comprising a plurality of feature vectors, each feature vector corresponding to a particular amino acid site of the portion of the polypeptide chain and comprising side chain geometry components representing a (e.g., 3D) geometry [e.g., position and/or orientation (e.g., as determined via one or more torsion angles)] of a side chain at the particular amino acid site [e.g., wherein values of the one or more side chain geometry components of each feature vector are set to initial starting values (e.g., selected at random from an initial probability (e.g., uniform) distribution)]; (c) determining, by the processor, using a machine learning model, one or more (e.g., a plurality of) (side chain) velocity fields based at least in part on (i) the sequence data and (ii) the representation of the 3D peptide backbone geometry and/or folds thereof and, beginning with the seed set, updating (e.g., iteratively), by the processor, values of the side chain geometry components of the plurality of feature vectors according to the one or more velocity fields, thereby evolving the values of the side chain geometry components of the plurality of feature vectors from a set of initial starting values into a set of final values representing (e.g., final, determined) geometries of each amino acid site of the portion of the polypeptide chain; and (c) creating, by the processor, using, the set of final values, a 3D polypeptide model of the portion of the polypeptide chain representing the 3D geometries of amino acid side chains within the portion of the polypeptide chain [e.g., using the set of final values as the 3D model representing the polypeptide chain; e.g., determining, based on the set of final values, positions of side chain atoms at each amino acid site]; and (d) storing and/or providing (e.g., for display and/or further processing), the 3D polypeptide model.

In certain embodiments, the method comprises one or more features described herein, e.g., in paragraphs above (e.g., at paragraphs [0006]-[0044]).

In another aspect, the invention is method for in-silico generation and/or prediction of an amino acid sequence of a polypeptide chain (e.g., a protein and/or peptide), the method comprising: (a) receiving and/or generating, by a processor of a computing device a seed set comprising a plurality of feature vectors, each feature vector corresponding to a particular (e.g., backbone) site of the portion of the polypeptide chain and a side chain type component representing a likelihood of one or more possible types of amino acid side chains [e.g., a likelihood of each of one of N (e.g., twenty) possible types of amino acid(s) (e.g., each feature vector having a plurality of elements, each corresponding to a particular amino acid type (e.g., of the N possible types, such as the twenty canonical amino acids) and having a value (e.g., between 0 and 1) representing a likelihood of that particular amino acid type occupying the particular backbone site) (e.g., each feature vector representing a distribution over a (e.g., N−1 dimension) simplex)] at the particular site [e.g., wherein values of the side chain type component of each feature vector are set to initial starting values (e.g., selected at random from an initial probability (e.g., uniform) distribution)]; (b) determining, by the processor, using a machine learning model, one or more (e.g., a plurality of) (sequence) velocity fields and, beginning with the seed set, updating (e.g., iteratively), by the processor, values of the side chain type components of the plurality of feature vectors according to the one or more (sequence) velocity fields, thereby evolving the values of the side chain type components of the plurality of feature vectors from a set of initial starting values into a set of final values representing (e.g., final, determined) likelihoods of amino acid side chain types at each site of the portion of the polypeptide chain; and (c) determining, by the processor, using, the set of final values, sequence data representing an amino acid sequence of the portion of the polypeptide chain representing (e.g., optionally, projecting each final feature vector of the set of final values from a N−1 dimensional simplex to a N dimensional likelihood vector, and taking an argmax to determine a particular amin acidy side chain type at each location within the portion of the polypeptide chain); and (d) storing and/or providing (e.g., for display and/or further processing), the sequence data.

In certain embodiments, the method comprises: receiving and/or generating, by the processor, a representation of a 3D peptide backbone geometry, and/or folds thereof, for at least a portion of the polypeptide chain; and at step (b), determining the one or more velocity fields based at least in part on the representation of the 3D peptide backbone geometry and/or folds thereof.

In certain embodiments, the method comprises one or more features described herein, e.g., in paragraphs above (e.g., at paragraphs [0006]-[0044]).

In another aspect, the invention is directed to a method for generating in-silico docking predictions for a plurality (e.g., two or more) of polypeptide chains (e.g., proteins and/or peptides), the method comprising: (a) receiving and/or generating, by a processor of a computing device, a first scaffold model representation of a first 3D peptide backbone of at least a portion of a first polypeptide chain; (b) receiving and/or generating, by a processor of a computing device, a second scaffold model representation of a second 3D peptide backbone of at least a portion of a second polypeptide chain; (c) receiving and/or generating, by a processor of a computing device a seed set comprising a plurality of feature vectors, the plurality of feature vectors comprising (i) a first set of feature vectors corresponding to and representing positions and/or orientations of (e.g., individual backbone sites of) the first 3D peptide backbone and/or (ii) a second set of feature vectors corresponding to and representing positions and/or orientations of (e.g., individual backbone sites of) the second 3D peptide backbone [e.g., wherein values of the position and/or orientation components of each feature vector are set to initial starting values (e.g., selected at random from an initial probability (e.g., uniform) distribution)]; (d) determining, by the processor, using a machine learning model, based at least in part on the first scaffold model and the second scaffold model, one or more (e.g., a plurality of) velocity fields, by the processor, and, beginning with the seed set, updating (e.g., iteratively), by the processor, values of (i) the first set of feature vectors and/or (ii) the second set of feature vectors according to the one or more velocity fields, thereby evolving values of the first and/or second feature vector sets from a set of initial starting values into a set of final values representing final position(s) and/or orientation(s) of the first 3D peptide backbone and/or the second 3D peptide backbone docket (e.g., forming a polypeptide complex) with each other [e.g., wherein the method comprises using the machine learning model to (e.g., iteratively) determine, at each of a plurality of time points, xₜ a velocity field v(xₜ, t) using: (i) a current x(t) as input, for which the first and second polypeptide chains are treated together—e.g., edge features relating sites within a single chain and relating sites in between chains are computed, and (ii) a conditioning input that is an “x1” for each chain—representing locations and orientations of each backbone site in the known structure of each chain as represented by the first and second scaffold models e.g., in which each chain is treated independently—e.g., only edge features for relationships within a same chain are computed and used as conditioning input]; (c) creating, by the processor, using, the set of final values, a generated polypeptide complex model representing first and second 3D peptide backbones docked to form a polypeptide complex; and (f) storing and/or providing (e.g., for display and/or further processing), the generated polypeptide complex model.

In certain embodiments, the method comprises one or more features described herein, e.g., in paragraphs above (e.g., at paragraphs [0006]-[0044]).

In another aspect, the invention is directed to a method for designing a custom biologic for binding to a target [e.g., a target antigen or portion (e.g., epitope) thereof], the method comprising: (a) receiving and/or generating, by the processor, a target model representing at least a portion of the target (e.g., comprising a desired epitope region, to target for binding); (b) receiving and/or generating, by a processor of a computing device, a template model representing a sequence and/or three-dimensional structure of at least a portion of a reference biologic (e.g., one or more polypeptide chains; e.g., a protein and/or peptide), the template model comprising a base portion representing a portion of the reference biologic located about (e.g., in proximity to, in regards to linear sequence and/or 3D folded structure of the reference biologic) one or more (e.g., predetermined) variable regions of the reference biologic designated (e.g., a-priori known, have been determined, or otherwise identified as regions that do, will, or are intended/desired to interact with and mediate binding to the target) as modifiable for binding to the target; (c) receiving and/or generating, by the processor, a seed set comprising a plurality of feature vectors, each feature vector corresponding to a particular (e.g., amino acid) site within the one or more variable region(s) of template model and comprising one or both of (i) position and/or orientation components representing a position and/or orientation of (e.g., individual backbone atoms and/or a local frame representation of) the particular site and (ii) a side chain type component, representing likelihoods of one or more particular amino acid side chain types occupying the particular site [e.g., wherein values of (i) the position and/or orientation components and/or (ii) side chain type components of each feature vector are set to initial starting values (e.g., selected at random from an initial probability (e.g., uniform) distribution)]; (d) determining, by the processor, using a machine learning model, one or more (e.g., a plurality of) velocity fields based at least in part on (i) the target model [e.g., including an identification of a desired target epitope thereof to bind with, e.g., via a categorical variable used as node feature input to the machine learning model] and (ii) the base portion of the template model and, beginning with the seed set, updating (e.g., iteratively), by the processor, values of one or both of (i) the position and/or orientation and (ii) the side chain type components of the plurality of feature vectors according to the one or more velocity fields, thereby evolving them a set of initial starting values into a set of final values representing (i) positions and/or orientations of each site of generated peptide backbone structures of the one or more variable regions, and/or (ii) likelihoods of (e.g., amino acid) side chain types at locations within the one or more variable regions; (e) creating, by the processor, using, the set of final values, one or both of: (i) a generated scaffold model representing a de-novo peptide backbone for the one or more variable regions of the custom biologic [e.g., using the set of final values as the generated scaffold model representing the de-novo peptide backbone; e.g., determining, based on the set of final values, positions of backbone atoms (e.g., at each backbone site, a position of one or more backbone atoms (e.g., an N, alpha-Carbon, C, and O)) to be represented via the generated scaffold model] and (ii) sequence data representing a generated amino acid sequence of the one or more variable region(s) (e.g., optionally, projecting each final feature vector of the set of final values from a N−1 dimensional simplex to a N dimensional likelihood vector, and taking an argmax to determine a particular amin acidy side chain type at each location within the portion of the polypeptide chain); and (f) storing and/or providing, by the processor, the peptide backbone structure and/or the generated sequence for display and/or further processing and/or use in designing the custom biologic.

In certain embodiments, the method comprises one or more features described herein, e.g., in paragraphs above (e.g., at paragraphs [0006]-[0044]).

In another aspect, the invention is directed to a system for computer generation of a de-novo peptide backbone of a custom biologic, the system comprising: a processor of a computing device; and memory having instructions stored thereon, wherein the instructions, when executed by the processor, cause the processor to: (a) receive and/or generate a seed set comprising a plurality of feature vectors, each feature vector corresponding to a particular backbone site and comprising position and/or orientation components representing a position and/or orientation of the particular backbone site; (b) determine, using a machine learning model, one or more velocity fields and, beginning with the seed set, updating, by the processor, values of the position and/or orientation components of the plurality of feature vectors according to the one or more velocity fields, thereby evolving the values of the position and/or orientation components of the plurality of feature vectors from a set of initial starting values into a set of final values representing positions and/or orientations of each backbone site of a generated peptide backbone; and (c) create, using, the set of final values, a generated scaffold model representing the de-novo peptide backbone; and (d) store and/or provide the generated scaffold model.

In certain embodiments, the system comprises one or more features described herein, e.g., in paragraphs above (e.g., at paragraphs [0006]-[0044]).

In another aspect, the invention is directed to a system for computer-aided generative design of de-novo polypeptides and/or complexes thereof, the system comprising: a processor of a computing device; and memory having instructions stored thereon, wherein the instructions, when executed by the processor, cause the processor to: (a) receive and/or generate a seed representation of one or more peptide backbones and/or amino acid sequences, the seed representation comprising a plurality of feature values representing (i) amino acid sites and/or (ii) individual backbone atoms and/or side chain types thereof; (b) determine, using one or more machine learning models, one or more velocity fields and, beginning with the seed set, updating, by the processor, the plurality of feature values according to the one or more velocity fields, thereby evolving the representation of one or more peptide backbones and/or amino acid sequences from initial feature values of the seed representation to a set of final feature values representing amino acid sites, and/or individual backbone atoms and/or side chain types, of one or more generated peptide backbones and/or amino acid sequences of the one or more de-novo polypeptide and/or a complex thereof; (c) create, using the set of final values, one or more generated scaffold model(s) and/or sequence string(s) representing the de-novo polypeptide(s) and/or complexes thereof; and (d) store and/or provide the generated scaffold models and/or sequence strings.

In certain embodiments, the system comprises one or more features described herein, e.g., in paragraphs above (e.g., at paragraphs [0006]-[0044]).

In another aspect, the invention is directed to a system for generating attention-based deep-learning predictions from input graph representations comprising nodes and edges, the system comprising: a processor of a computing device; and memory having instructions stored thereon, wherein the instructions, when executed by the processor, cause the processor to (a) receive and/or access an initial graph representation comprising a plurality of node feature vectors, each node feature vector associated with a particular node of the initial graph representation and comprising one or more initial node feature values; (b) determine, using a machine learning model, a predicted graph representation, wherein the machine learning model: (i) receives the initial graph representation as input; (ii) comprises at least one edge retrieval layer, wherein the at least one edge retrieval layer: (A) comprises one or more self-attention head(s), each of which determines a corresponding set of attention weights based at least in part on values of the feature vectors and/or node feature values determined therefrom, such that the edge retrieval layer determines one or more sets of attention weights; and (B) uses the one or more sets of attention weights to determine values of retrieved edge feature vectors; and (iii) generates, as output, based at least in part on the values of the retrieved edge feature vectors, a plurality of predicted node feature vectors and/or one or more velocity fields, and wherein the predicted graph representation is determined using the plurality of predicted node feature vectors and/or one or more velocity fields; and (c) store and/or provide, the predicted graph representation.

In another aspect, the invention is directed to a system for designing a custom biologic having a three-dimensional structure corresponding to a desired fold, the system comprising: a processor of a computing device; and memory having instructions stored thereon, wherein the instructions, when executed by the processor, cause the processor to: (a) receive and/or generate one or more protein fold representations, each encoding desired three-dimensional structural features of the desired fold at/in at least a portion of locations within the custom biologic; (b) receive and/or generate a seed set comprising a plurality of feature vectors, each feature vector corresponding to a particular site within the one or more variable region(s) of template model and comprising one or both of: (A) position and/or orientation components representing a position and/or orientation of the particular site and (B) a side chain type component, representing likelihoods of one or more particular amino acid side chain types occupying the particular site; (c) determine, using a machine learning model, one or more velocity fields based at least in part on the one or more protein fold representations, and, beginning with the seed set, update values of one or both of (A) the position and/or orientation components and (B) the side chain type components of the plurality of feature vectors according to the one or more velocity fields, thereby evolving them a set of initial starting values into a set of final values representing (i) positions and/or orientations of each site of generated peptide backbone structures of the one or more variable regions, and/or (ii) likelihoods of side chain types at locations within the one or more variable regions; (d) generate, using the set of final values, one or both of: (A) a scaffold model representing a three-dimensional structure of a de-novo peptide backbone for the custom biologic, and (B) sequence data representing an amino acid sequence of the custom biologic; and (c) store and/or provide the generated (A) scaffold model and/or (B) sequence data for display and/or further processing and/or use in designing the custom biologic.

In certain embodiments, the system comprises one or more features described herein, e.g., in paragraphs above (e.g., at paragraphs [0006]-[0044]).

In another aspect, the invention is directed to a system for designing a custom antibody for binding to a target, the system comprising: a processor of a computing device; and memory having instructions stored thereon, wherein the instructions, when executed by the processor, cause the processor to: (a) receive and/or generate an antibody template representing a sequence and/or three-dimensional structure of at least a portion of a reference antibody, the antibody template comprising (i) a base portion located about one or more CDRs of the reference antibody, but excluding at least a portion of the one or more CDRs themselves; (b) determine one or more protein fold representations, each encoding and representing three-dimensional structural features of at least the base portion of the antibody template; (c) receive and/or generate a seed set comprising a plurality of feature vectors, each feature vector corresponding to a particular site within one or more variable region(s) corresponding to, and representing to-be-designed custom versions of, CDR regions of the reference antibody and comprising one or both of: (A) position and/or orientation components representing a position and/or orientation of (e.g., individual backbone atoms and/or a local frame representation of) the particular site and (B) a side chain type component, representing likelihoods of one or more particular amino acid side chain types occupying the particular site; (c) determine, using a machine learning model, one or more velocity fields based at least in part on the one or more protein fold representations, and, beginning with the seed set, update values of one or both of (A) the position and/or orientation components and (B) the side chain type components of the plurality of feature vectors according to the one or more velocity fields, thereby evolving them a set of initial starting values into a set of final values representing (i) positions and/or orientations of each site of generated peptide backbone structures of the one or more variable regions, and/or (ii) likelihoods of side chain types at locations within the one or more variable regions; (d) generate, using the set of final values, one or both of: (A) a scaffold model representing a three-dimensional structure of a de-novo peptide backbone for one or more CDR regions of the custom antibody; and (B) sequence data representing an amino acid sequence of one or more CDR regions of the custom antibody; and (c) store and/or provide the generated (A) scaffold model and/or (B) sequence data, for display and/or further processing and/or use in designing the custom biologic.

In certain embodiments, the system comprises one or more features described herein, e.g., in paragraphs above (e.g., at paragraphs [0006]-[0044]).

In another aspect, the invention is directed to a system for in-silico generation and/or prediction of three-dimensional (3D) side chain geometries of a polypeptide chain, the system comprising: a processor of a computing device; and memory having instructions stored thereon, wherein the instructions, when executed by the processor, cause the processor to: (a) receive and/or generate (i) sequence data representing an amino acid sequence of at least a portion of the polypeptide chain and (ii) a representation of a 3D peptide backbone geometry, and/or folds thereof, for the portion of the polypeptide chain; (b) receive and/or generate a seed set comprising a plurality of feature vectors, each feature vector corresponding to a particular amino acid site of the portion of the polypeptide chain and comprising side chain geometry components representing a geometry of a side chain at the particular amino acid site; (c) determine, using a machine learning model, one or more velocity fields based at least in part on (i) the sequence data and (ii) the representation of the 3D peptide backbone geometry and/or folds thereof and, beginning with the seed set, update values of the side chain geometry components of the plurality of feature vectors according to the one or more velocity fields, thereby evolving the values of the side chain geometry components of the plurality of feature vectors from a set of initial starting values into a set of final values representing geometries of each amino acid site of the portion of the polypeptide chain; and (c) create, using, the set of final values, a 3D polypeptide model of the portion of the polypeptide chain representing the 3D geometries of amino acid side chains within the portion of the polypeptide chain; and (d) store and/or provide the 3D polypeptide model.

In certain embodiments, the system comprises one or more features described herein, e.g., in paragraphs above (e.g., at paragraphs [0006]-[0044]).

In another aspect, the invention is directed to a system for in-silico generation and/or prediction of an amino acid sequence of a polypeptide chain, the system comprising: a processor of a computing device; and memory having instructions stored thereon, wherein the instructions, when executed by the processor, cause the processor to: (a) receive and/or generate a seed set comprising a plurality of feature vectors, each feature vector corresponding to a particular site of the portion of the polypeptide chain and a side chain type component representing a likelihood of one or more possible types of amino acid side chains at the particular site; (b) determine, using a machine learning model, one or more velocity fields and, beginning with the seed set, update values of the side chain type components of the plurality of feature vectors according to the one or more velocity fields, thereby evolving the values of the side chain type components of the plurality of feature vectors from a set of initial starting values into a set of final values representing likelihoods of amino acid side chain types at each site of the portion of the polypeptide chain; and (c) determine, using, the set of final values, sequence data representing an amino acid sequence of the portion of the polypeptide chain; and (d) store and/or provide the sequence data.

In certain embodiments, the instructions cause the processor to: receive and/or generate a representation of a 3D peptide backbone geometry, and/or folds thereof, for at least a portion of the polypeptide chain and at step (b), determine the one or more velocity fields based at least in part on the representation of the 3D peptide backbone geometry and/or folds thereof.

In certain embodiments, the system comprises one or more features described herein, e.g., in paragraphs above (e.g., at paragraphs [0006]-[0044]).

In another aspect, the invention is directed to a system for generating in-silico docking predictions for a plurality (e.g., two or more) of polypeptide chains, the system comprising: a processor of a computing device; and memory having instructions stored thereon, wherein the instructions, when executed by the processor, cause the processor to: (a) receive and/or generate a first scaffold model representation of a first 3D peptide backbone of at least a portion of a first polypeptide chain; (b) receive and/or generate a second scaffold model representation of a second 3D peptide backbone of at least a portion of a second polypeptide chain; (c) receive and/or generate a seed set comprising a plurality of feature vectors, the plurality of feature vectors comprising (i) a first set of feature vectors corresponding to and representing positions and/or orientations of the first 3D peptide backbone and/or (ii) a second set of feature vectors corresponding to and representing positions and/or orientations of the second 3D peptide backbone; (d) determine, using a machine learning model, based at least in part on the first scaffold model and the second scaffold model, one or more velocity fields and, beginning with the seed set, update values of (i) the first set of feature vectors and/or (ii) the second set of feature vectors according to the one or more velocity fields, thereby evolving values of the first and/or second feature vector sets from a set of initial starting values into a set of final values representing final position(s) and/or orientation(s) of the first 3D peptide backbone and/or the second 3D peptide backbone docket with each other; (e) create, using, the set of final values, a generated polypeptide complex model representing first and second 3D peptide backbones docked to form a polypeptide complex; and (f) store and/or provide the generated polypeptide complex model

In certain embodiments, the system comprises one or more features described herein, e.g., in paragraphs above (e.g., at paragraphs [0006]-[0044]).

In another aspect, the invention is directed to a system for designing a custom biologic for binding to a target, the system comprising: a processor of a computing device; and memory having instructions stored thereon, wherein the instructions, when executed by the processor, cause the processor to: (a) receive and/or generate a target model representing at least a portion of the target; (b) receive and/or generate a template model representing a sequence and/or three-dimensional structure of at least a portion of a reference biologic, the template model comprising a base portion representing a portion of the reference biologic located about one or more variable regions of the reference biologic designated as modifiable for binding to the target; (c) receive and/or generate a seed set comprising a plurality of feature vectors, each feature vector corresponding to a particular site within the one or more variable region(s) of template model and comprising one or both of (i) position and/or orientation components representing a position and/or orientation of the particular site and (ii) a side chain type component, representing likelihoods of one or more particular amino acid side chain types occupying the particular site; (d) determine, using a machine learning model, one or more velocity fields based at least in part on (i) the target model and (ii) the base portion of the template model and, beginning with the seed set, update values of one or both of (i) the position and/or orientation and (ii) the side chain type components of the plurality of feature vectors according to the one or more velocity fields, thereby evolving them a set of initial starting values into a set of final values representing (i) positions and/or orientations of each site of generated peptide backbone structures of the one or more variable regions, and/or (ii) likelihoods of side chain types at locations within the one or more variable regions; (e) create, using, the set of final values, one or both of: (i) a generated scaffold model representing a de-novo peptide backbone for the one or more variable regions of the custom biologic; and (ii) sequence data representing a generated amino acid sequence of the one or more variable region(s); and (f) store and/or provide, by the processor, the peptide backbone structure and/or the generated sequence for display and/or further processing and/or use in designing the custom biologic.

In certain embodiments, the system comprises one or more features described herein, e.g., in paragraphs above (e.g., at paragraphs [0006]-[0044]).

Features of embodiments described with respect to one aspect of the invention may be applied with respect to another aspect of the invention.

### BRIEF DESCRIPTION OF THE DRAWING

The foregoing and other objects, aspects, features, and advantages of the present disclosure will become more apparent and better understood by referring to the following description taken in conjunction with the accompanying drawings, in which:

FIG. 1A shows three sets of graphs (3D plots) illustrating use of a flow matching approach to push an initial distribution to a final target distribution, at t=0, according to an illustrative embodiment.

FIG. 1B shows three sets of graphs (3D plots) illustrating use of a flow matching approach to push an initial distribution to a final target distribution, corresponding to 33% of completion, according to an illustrative embodiment.

FIG. 1C shows three sets of graphs (3D plots) illustrating use of a flow matching approach to push an initial distribution to a final target distribution, corresponding to 66% of completion, according to an illustrative embodiment.

FIG. 1D shows three sets of graphs (3D plots) illustrating use of a flow matching approach to push an initial distribution to a final target distribution, corresponding to 100% of completion, according to an illustrative embodiment.

FIG. 2A is a block flow diagram of an example machine learning model training process, according to an illustrative embodiment.

FIG. 2B is a schematic illustrating a path from an initial starting point to a final point, according to an illustrative embodiment.

FIG. 2C is a block flow diagram of an example process for using a machine learning model to generate de-novo peptide structures, according to an illustrative embodiment.

FIG. 3A is a schematic illustrating an approach for parameterizing protein backbones, according to an illustrative embodiment.

FIG. 3B is a schematic illustrating an approach for parameterizing protein backbones, according to an illustrative embodiment.

FIG. 4 is an illustrative schematic illustrating how positions and orientations of reference frames representing protein backbone sites can be iteratively updated via flow matching methods described herein to generate a de-novo peptide backbone structure, according to an illustrative embodiment.

FIG. 5 is a set of five graphs illustrating a use of flow matching approach on a 2-dimensional simplex path to push an initial uniform distribution to a final target distribution, according to an illustrative embodiment.

FIG. 6 is a box plot showing accuracy of flow matching approach over time, according to an illustrative embodiment.

FIG. 7A is a block flow diagram of an example process for generating attention-based deep-learning predictions from input graph representations, according to an illustrative embodiment.

FIG. 7B is a schematic illustrating an example transformer-based machine learning model architecture, used in certain embodiments.

FIG. 7C is a schematic illustrating an example transformer-based machine learning model architecture, used in certain embodiments.

FIG. 7D is a block flow diagram of an example process for designing a custom biologic having a three-dimensional structure belonging to a desired fold family, according to an illustrative embodiment.

FIG. 8 is a ribbon diagram illustrating TIM barrel (PDB: 8TIM) secondary structure.

FIG. 9A is a heatmap showing Block Adjacency Matrix of TIM barrel, according to an illustrative embodiment.

FIG. 9B is a heatmap showing multi-channel Block Adjacency Matrix of TIM barrel, according to an illustrative embodiment.

FIG. 9C is a block flow diagram of an example process for designing a custom antibody for binding to a target, according to an illustrative embodiment.

FIG. 10 is a block diagram of an exemplary cloud computing environment, used in certain embodiments.

FIG. 11 is a block diagram of an example computing device and an example mobile computing device, used in certain embodiments.

FIG. 12A is graph showing an example loss curve during training of a machine learning model used to estimate velocity fields in a flow-matching framework for generating de-novo peptide backbones, according to an illustrative embodiment.

FIG. 12B is graph showing an example loss curve showing validation of a machine learning model used to estimate velocity fields in a flow-matching framework for generating de-novo peptide backbones, according to an illustrative embodiment.

FIG. 12C is a series of snapshots showing computed atom positions at various time points as they are iteratively adjusted to create a generated scaffold model representing a generated de-novo peptide backbone, according to an illustrative embodiment.

FIG. 13 is a schematic showing an example neural network architecture, used in certain embodiments.

FIG. 14A is a schematic showing an example neural network architecture and approach for determining input features, according to an illustrative embodiment.

FIG. 14B is a schematic showing an example neural network architecture and approach for determining edge features, according to an illustrative embodiment.

FIG. 14C is an illustration of computed edge features, used in certain embodiments.

FIG. 14D is a schematic showing an example neural network architecture and approach for determining edge features, according to an illustrative embodiment.

FIG. 14E is a schematic showing an example architecture and approach for determining velocity predictions, according to an illustrative embodiment.

FIG. 15A is a schematic illustrating various metrics for evaluating model performance and/or training, according to an illustrative embodiment.

FIG. 15B is a schematic illustrating a metric for evaluating model performance and/or training, according to an illustrative embodiment.

FIG. 15C is a graph showing a metric for evaluating model performance and/or training, according to an illustrative embodiment.

FIG. 15D is a graph showing a metric for evaluating model performance and/or training, according to an illustrative embodiment.

FIG. 15E is a schematic illustrating a metric for evaluating model performance and/or training, according to an illustrative embodiment.

FIG. 16 shows an example batch creation procedure, used in certain embodiments.

FIG. 17 is a schematic showing an example neural network architecture, used in certain embodiments.

FIG. 18A is a schematic showing an example neural network architecture, used in certain embodiments.

FIG. 18B is a schematic showing an example neural network architecture, used in certain embodiments.

FIG. 19A is a graph showing a position loss function for a training set with increasing training epoch.

FIG. 19B is a graph showing an orientation loss function for a training set with increasing training epoch.

FIG. 19C is a graph showing a total loss function for a training set with increasing training epoch.

FIG. 19D is a graph showing a position loss function for a validation set with increasing training epoch.

FIG. 19E is a graph showing an orientation loss function for a validation set with increasing training epoch.

FIG. 19F is a graph showing a total loss function for a validation set with increasing training epoch.

FIG. 20 is a bar chart showing values of inter-residue clash scores (“between_clash_score”) and values of intra-residue clash scores (“within_clash_score”).

FIG. 21 is a bar chart showing values for bond angle scores determined for N—Cα-C, Cα-C—N, and C—N—Cα bonds.

FIG. 22 is a bar chart showing values for distance scores, in particular a consecutive residue Cα distance score (Cα-Cα distance score) and a C—N bond length score.

FIG. 23 is a bar chart showing values for number of unsatisfied bonds, in particular a number of peptide bond lengths and Cα-Cα distances.

FIG. 24 is a bar chart showing values for number of atoms clashing, in particular where inter-residue and intra-residue clash restrictions were.

FIG. 25A is a box plot showing computed RMSD score distributions for generated structures.

FIG. 25B is a box plot showing computed TM score distributions for generated structures.

FIG. 26A is a histogram showing computed RMSD score distributions for generated structures.

FIG. 26B is a histogram showing computed TM score distributions for generated structures.

FIG. 27 is a box plot showing values of a secondary structure run (“SS run”) metric.

FIG. 28 is a box plot showing values of a secondary structure composition (“SS composition”) metric.

FIG. 29 is a box plot showing values of a secondary structure contact (“SS contact”) metric.

FIG. 30A is a bar chart showing backbone dihedral (phi and psi angles) distribution metric.

FIG. 30B is a heatmap showing aggregate residue phi-psi distributions for generated designable structures.

FIG. 30C is a heatmap showing aggregate residue phi-psi distributions for a reference dataset.

FIG. 31A is a ribbon diagram showing an example of a generated protein backbone structures with 100 amino acids.

FIG. 31B is a ribbon diagram showing an example of a generated protein backbone structures with 150 amino acids.

FIG. 31C is a ribbon diagram showing an example of a generated protein backbone structures with 200 amino acids.

FIG. 31D is a ribbon diagram showing an example of a generated protein backbone structures with 250 amino acids.

FIG. 32A is a ribbon diagram showing an example of a generated protein backbone structures with 300 amino acids.

FIG. 32B is a ribbon diagram showing an example of a generated protein backbone structures with 350 amino acids.

FIG. 32C is a ribbon diagram showing an example of a generated protein backbone structures with 400 amino acids.

FIG. 32D is a ribbon diagram showing an example of a generated protein backbone structures with 450 amino acids.

FIG. 33 is a ribbon diagram showing an example of a generated protein backbone structures with 500 amino acids.

FIG. 34 shows ribbon diagrams illustrating predictions performed on PDB structure PDBID 1U2H with 23 residues masked and inpainted by the machine learning model.

FIG. 35 shows ribbon diagrams illustrating predictions performed on PDB structure PDBID 6MFW with 48 residues masked and inpainted by the machine learning model.

FIG. 36 shows ribbon diagrams illustrating predictions performed on PDB structure PDBID 4RUW with 50 residues masked and inpainted by the machine learning model.

FIG. 37 shows ribbon diagrams illustrating predictions performed on PDB structure PDBID 4ES6 with 50 residues masked and inpainted by the machine learning model.

FIG. 38 displays ribbon diagrams showing results for several PDB with the prediction shown alone along the top row and overlaid on ground truth.

FIG. 39 shows ribbon diagrams of binders generated using inpainting approaches.

FIG. 40A shows a ribbon diagram of a generated binder, where hotspot information was unknown, using example protein PDB ID 3nau.

FIG. 40B shows a ribbon diagram of a generated binder, where hotspot information was unknown, using example protein PDB ID 2vkl.

FIG. 40C shows a ribbon diagram of a generated binder, where hotspot information was unknown, using example protein PDB ID 4pwy.

FIG. 40D shows a ribbon diagram of a generated binder, where hotspot information was unknown, using example protein PDB ID 5rdv.

FIG. 41A shows a ribbon diagram of a generated binders, where hotspot information was known and provided, using example protein with PDB ID 4wja.

FIG. 41B shows a ribbon diagram of a generated binders, where hotspot information was known and provided, using example protein with PDB ID 5rdv.

FIG. 41C shows a ribbon diagram of a generated binders, where hotspot information was known and provided, using example protein with PDB ID 6ahp.

FIG. 41D shows a ribbon diagram of a generated binders, where hotspot information was known and provided, using example protein with PDB ID 5xta.

FIG. 42A is a ribbon diagram showing an example of a generated protein structures with 76 amino acids.

FIG. 42B is a ribbon diagram showing an example of a generated protein structures with 107 amino acids.

FIG. 42C is a ribbon diagram showing an example of a generated protein structures with 250 amino acids.

FIG. 42D is a ribbon diagram showing an example of a generated protein structures with 365 amino acids.

FIG. 43 is a ribbon diagram showing an example of a generated protein structures with 425 amino acids.

FIG. 44 is a bar chart showing values of inter-residue clash scores (“between_clash_score”) and values of intra-residue clash scores (“within_clash_score”).

FIG. 45 is a bar chart showing values for bond angle scores determined for N—Cα-C, Cα-C—N, and C—N—Cα bonds.

FIG. 46 is a bar chart showing values for distance scores, in particular a consecutive residue Ca distance score (Cα-Cα distance score) and a C—N bond length score.

FIG. 47 is a bar chart showing values for number of unsatisfied bonds, in particular a number of peptide bond lengths and Cα-Cα distances.

FIG. 48 is a bar chart showing values for number of atoms clashing, in particular where inter-residue and intra-residue clash restrictions were.

FIG. 49 is box plot showing statistics of per protein accuracy results for different tasks.

FIG. 50 is box plot showing statistics of per protein similarity results for different tasks.

FIG. 51 is box plot showing statistics of per protein accuracy results for various temperatures in binder design.

FIG. 52 is box plot showing statistics of per protein similarity results for various temperatures in binder design

FIG. 53 is box plot showing statistics of per protein accuracy on core and surface.

FIG. 54 is a heatmap showing how amino acid probabilities vary in a 19-dimensional simplex at a particular site over the course of multiple time steps.

FIG. 55 is a bar chart showing torsion angle accuracy for monomer side chain generation by percentage of surface residues.

FIG. 56 is a bar chart showing torsion angle accuracy for monomer side chain generation by number of residues.

FIG. 57 is a ribbon diagram showing side chain generation of PDB 4kdw using flow matching generative model overlaid with ground truth.

FIG. 58 is a ribbon diagram showing side chain generation of PDB 3icw (chain B) using flow matching generative model overlaid with ground truth backbone and side chains.

FIG. 59 is a box plot showing model distribution of violation values.

FIG. 60 is a box plot showing PDB distribution of violation values.

FIG. 61 is a ribbon diagram showing side chain generation of PDB 5nr7 (chain A) in orange as well as structure of backbone and side chains (chain B) in blue.

FIG. 62 is a plot showing example trajectories of vector fields approximated by flow matching generative model on the complex plane to generate protein side chain torsion angles.

FIG. 63 is a plot showing time step evolution of violation values of generated side chain conformation for various amino acid types.

FIG. 64 is a sunburst plot showing selected hierarchy of CATH database used for fold conditioning.

FIG. 65 is a box plot showing TM-scores for two models for fold conditioning.

FIG. 66 is a box plot showing the highest TM-scores selected from ten generation for each fold family for two models used for fold conditioning.

FIG. 67 is a box plot showing SSE accuracy for two models used for fold conditioning.

FIG. 68 shows a collection of ribbon diagrams for various CATH proteins on the left and three different samples for each protein on the right.

Features and advantages of the present disclosure will become more apparent from the detailed description of certain embodiments that is set forth below, particularly when taken in conjunction with the figures, in which like reference characters identify corresponding elements throughout. In the figures, like reference numbers generally indicate identical, functionally similar, and/or structurally similar elements.

### Certain Definitions

In order for the present disclosure to be more readily understood, certain terms are first defined below. Additional definitions for the following terms and other terms are set forth throughout the specification.

Comprising: A device, composition, system, or method described herein as “comprising” one or more named elements or steps is open-ended, meaning that the named elements or steps are essential, but other elements or steps may be added within the scope of the composition or method. To avoid prolixity, it is also understood that any device, composition, or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any device, composition, or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

A, an: As used herein, “a” or “an” with reference to a claim feature means “one or more,” or “at least one.”

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with which two or more binding partners associate with one another. Those skilled in the art are aware of a variety of assays that can be used to assess affinity, and will furthermore be aware of appropriate controls for such assays. In some embodiments, affinity is assessed in a quantitative assay. In some embodiments, affinity is assessed over a plurality of concentrations (e.g., of one binding partner at a time). In some embodiments, affinity is assessed in the presence of one or more potential competitor entities (e.g., that might be present in a relevant—e.g., physiological—setting). In some embodiments, affinity is assessed relative to a reference (e.g., that has a known affinity above a particular threshold [a “positive control” reference] or that has a known affinity below a particular threshold [a “negative control” reference”]). In some embodiments, affinity may be assessed relative to a contemporaneous reference; in some embodiments, affinity may be assessed relative to a historical reference. Typically, when affinity is assessed relative to a reference, it is assessed under comparable conditions.

Amino acid: The term “amino acid,” in its broadest sense, as used herein, refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acctylation, pegylation, glycosylation, phosphorylation, and/or substitution (e.g., of the amino group, the carboxylic acid group, one or more protons, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Antibody, Antibody polypeptide: As used herein, the terms “antibody polypeptide” or “antibody”, or “antigen-binding fragment thereof”, which may be used interchangeably, refer to polypeptide(s) capable of binding to an epitope. In some embodiments, an antibody polypeptide is a full-length antibody, and in some embodiments, is less than full length but includes at least one binding site (comprising at least one, and preferably at least two sequences with structure of antibody “variable regions”). In some embodiments, the term “antibody polypeptide” encompasses any protein having a binding domain which is homologous or largely homologous to an immunoglobulin-binding domain. In particular embodiments, “antibody polypeptides” encompasses polypeptides having a binding domain that shows at least 99% identity with an immunoglobulin binding domain. In some embodiments, “antibody polypeptide” is any protein having a binding domain that shows at least 70%, 80%, 85%, 90%, or 95% identity with an immuglobulin binding domain, for example a reference immunoglobulin binding domain. An included “antibody polypeptide” may have an amino acid sequence identical to that of an antibody that is found in a natural source. Antibody polypeptides in accordance with the present invention may be prepared by any available means including, for example, isolation from a natural source or antibody library, recombinant production in or with a host system, chemical synthesis, etc., or combinations thereof. An antibody polypeptide may be monoclonal or polyclonal. An antibody polypeptide may be a member of any immunoglobulin class, including any of the human classes: IgG, IgM, IgA, IgD, and IgE. In certain embodiments, an antibody may be a member of the IgG immunoglobulin class. As used herein, the terms “antibody polypeptide” or “characteristic portion of an antibody” are used interchangeably and refer to any derivative of an antibody that possesses the ability to bind to an epitope of interest. In certain embodiments, the “antibody polypeptide” is an antibody fragment that retains at least a significant portion of the full-length antibody's specific binding ability. Examples of antibody fragments include, but are not limited to, Fab, Fab′, F(ab′)2, scFv, Fv, dsFv diabody, and Fd fragments. Alternatively or additionally, an antibody fragment may comprise multiple chains that are linked together, for example, by disulfide linkages. In some embodiments, an antibody polypeptide may be a human antibody. In some embodiments, the antibody polypeptides may be a humanized. Humanized antibody polypeptides include may be chimeric immunoglobulins, immunoglobulin chains or antibody polypeptides (such as Fv, Fab, Fab′, F(ab′)2 or other antigen-binding subsequences of antibodies) that contain minimal sequence derived from non-human immunoglobulin. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Backbone, peptide backbone: As used herein, the term “backbone,” for example, as in a backbone or a peptide or polypeptide, refers to the portion of the peptide or polypeptide chain that comprises the links between amino acid of the chain but excludes side chains. In other words, a backbone refers to the part of a peptide or polypeptide that would remain if side chains were removed. In certain embodiments, the backbone is a chain comprising a carboxyl group of one amino acid bound via a peptide bond to an amino group of a next amino acid, and so on. Backbone may also be referred to as “peptide backbone”. It should be understood that, where the term “peptide backbone” is used, it is used for clarity, and is not intended to limit a length of a particular backbone. That is, the term “peptide backbone” may be used to describe a peptide backbone of a peptide and/or a protein.

Biologic: As used herein, the term “biologic” refers to a composition that is or may be produced by recombinant DNA technologies, peptide synthesis, or purified from natural sources and that has a desired biological activity. A biologic can be, for example, a protein, peptide, glycoprotein, polysaccharide, a mixture of proteins or peptides, a mixture of glycoproteins, a mixture of polysaccharides, a mixture of one or more of a protein, peptide, glycoprotein or polysaccharide, or a derivatized form of any of the foregoing entities. Molecular weight of biologics can vary widely, from about 1000 Da for small peptides such as peptide hormones to one thousand kDa or more for complex polysaccharides, mucins, and other heavily glycosylated proteins. In certain embodiments, a biologic is a drug used for treatment of diseases and/or medical conditions. Examples of biologic drugs include, without limitation, native or engineered antibodies or antigen binding fragments thereof, and antibody-drug conjugates, which comprise an antibody or antigen binding fragments thereof conjugated directly or indirectly (e.g., via a linker) to a drug of interest, such as a cytotoxic drug or toxin. In certain embodiments, a biologic is a diagnostic, used to diagnose diseases and/or medical conditions. For example, allergen patch tests utilize biologics (e.g., biologics manufactured from natural substances) that are known to cause contact dermatitis. Diagnostic biologics may also include medical imaging agents, such as proteins that are labelled with agents that provide a detectable signal that facilitates imaging such as fluorescent markers, dyes, radionuclides, and the like. In certain embodiments, biologics may, but need not necessarily be, used for medical applications. For example, biologics may include enzymes useful in e.g., industrial processes such as manufacturing, waste disposal, etc. In certain embodiments, a biologic may be a structural protein or peptide (e.g., based on and/or analogues to collagen, keratin, etc.), which may be designed for medical, cosmetic, industrial, research, or other purposes.

In vitro: The term “in vitro” as used herein refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Native, wild-type (WT): As used herein, the terms “native” and “wild-type” are used interchangeably to refer to biological structures and/or computer representations thereof that have been identified and demonstrated to exist in the physical, real world (e.g., as opposed to in computer abstractions). The terms, native and wild-type may refer to structures including naturally occurring biological structures, but do not necessarily require that a particular structure be naturally occurring. For example, the terms native and wild-type may also refer to structures including engineered structures that are man-made, and do not occur in nature, but have nonetheless been created and (e.g., experimentally) demonstrated to exist. In certain embodiments, the terms native and wild-type refer to structures that have been characterized experimentally, and for which an experimental determination of molecular structure (e.g., via x-ray crystallography) has been made.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Pose: As used herein, the term “pose” refers to a relative three-dimensional rotation and/or translation of one object, such as a polypeptide chain (e.g., protein) or peptide backbone thereof (e.g., represented by a scaffold model), with respect to another, such as a target molecule, e.g., a target protein. Accordingly, as used herein, for example in the context of molecular binding, where a particular protein and/or peptide backbone (e.g., a candidate peptide backbone) is referred to and/or described as being oriented at or according to a particular pose with respect to another protein (e.g., a target protein), it should be understood that the particular protein and/or peptide backbone may be rotated and/or translated in three dimensions relative to the other protein (e.g., target protein). A variety of manners and approaches may be used for representing poses, which may, for example, include representing rotations and/or transformations relative to a reference, such as the target and/or an initial pose of a scaffold representation, using one or more fixed coordinate systems, etc. For example, in certain embodiments, a particular pose may be represented as a combination one or more rotations and/or translations in three-dimensional space, relative to a particular, reference object (e.g., such as a target protein) or coordinate (e.g., a location at or within a target protein representation), such as values of three rotational angles and/or three distances/angles defining a 3D translation (e.g., defined in a particular coordinate system, such as rectangular, cylindrical, spherical). In certain embodiments, a target and a scaffold model may be represented in a particular coordinate system, with the scaffold model oriented with respect to the target at a particular initial pose. Additional poses may then be represented relative to the initial pose, for example as relative translations and/or rotations (in three dimensions) with respect to the initial pose.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Target: As used herein, the terms “target,” and “receptor” are used interchangeably and refer to one or more molecules or portions thereof to which a binding agent—e.g., a custom biologic, such as a protein or peptide, to be designed-binds. In certain embodiments, the target is or comprises a protein and/or peptide. In certain embodiments, the target is a molecule, such as an individual protein or peptide (e.g., a protein or peptide monomer), or portion thereof. In certain embodiments, the target is a complex, such as a complex of two or more proteins or peptides, for example, a macromolecular complex formed by two or more protein or peptide monomers. For example, a target may be a protein or peptide dimer, trimer, tetramer, etc. or other oligomeric complex. In certain embodiments, the target is a drug target, e.g., a molecule in the body, usually a protein, that is intrinsically associated with a particular disease process and that could be addressed by a drug to produce a desired therapeutic effect. In certain embodiments, a custom biologic is engineered to bind to a particular target. While the structure of the target remains fixed, structural features of the custom biologic may be varied to allow it to bind (e.g., at high specificity) to the target.

Treat: As used herein, the term “treat” (also “treatment” or “treating”) refers to any administration of a therapeutic agent (also “therapy”) that partially or completely alleviates, ameliorates, eliminates, reverses, relieves, inhibits, delays onset of, reduces severity of, and/or reduces incidence of one or more symptoms, features, and/or causes of a particular disease, disorder, and/or condition. In some embodiments, such treatment may be of a patient who does not exhibit signs of the relevant disease, disorder and/or condition and/or of a patient who exhibits only early signs of the disease, disorder, and/or condition. Alternatively, or additionally, such treatment may be of a patient who exhibits one or more established signs of the relevant disease, disorder and/or condition. In some embodiments, treatment may be of a patient who has been diagnosed as suffering from the relevant disease, disorder, and/or condition. In some embodiments, treatment may be of a patient known to have one or more susceptibility factors that are statistically correlated with increased risk of development of a given disease, disorder, and/or condition. In some embodiments the patient may be a human.

Machine learning module, machine learning model: As used herein, the terms “machine learning module” and “machine learning model” are used interchangeably and refer to a computer implemented process (e.g., a software function) that implements one or more particular machine learning algorithms, such as an artificial neural networks (ANN), convolutional neural networks (CNNs), random forest, decision trees, support vector machines, and the like, in order to determine, for a given input, one or more output values. In some embodiments, machine learning modules implementing machine learning techniques are trained, for example using curated and/or manually annotated datasets. Such training may be used to determine various parameters of machine learning algorithms implemented by a machine learning module, such as weights associated with layers in neural networks. In some embodiments, once a machine learning module is trained, e.g., to accomplish a specific task such as determining scoring metrics as described herein, values of determined parameters are fixed and the (e.g., unchanging, static) machine learning module is used to process new data (e.g., different from the training data) and accomplish its trained task without further updates to its parameters (e.g., the machine learning module does not receive feedback and/or updates). In some embodiments, machine learning modules may receive feedback, e.g., based on user review of accuracy, and such feedback may be used as additional training data, for example to dynamically update the machine learning module. In some embodiments, a trained machine learning module is a classification algorithm with adjustable and/or fixed (e.g., locked) parameters, e.g., a random forest classifier. In some embodiments, two or more machine learning modules may be combined and implemented as a single module and/or a single software application. In some embodiments, two or more machine learning modules may also be implemented separately, e.g., as separate software applications. A machine learning module may be software and/or hardware. For example, a machine learning module may be implemented entirely as software, or certain functions of a ANN module may be carried out via specialized hardware (e.g., via an application specific integrated circuit (ASIC), field programmable gate arrays (FPGAs), and the like).

Substantially: As used herein, the term “substantially” refers to the qualitative condition of exhibiting total or near-total extent or degree of a characteristic or property of interest.

Scaffold Model: As used herein, the term “scaffold model” refers to a computer representation of at least a portion of a peptide backbone of a particular protein and/or peptide. In certain embodiments, a scaffold model represents a peptide backbone of a protein and/or peptide and omits detailed information about amino acid side chains. Such scaffold models, may, nevertheless, include various mechanisms for representing sites (e.g., locations along a peptide backbone) that may be occupied by prospective amino acid side chains. In certain embodiments, a particular scaffold models may represent such sites in a manner that allows determining regions in space that may be occupied by prospective amino acid side chains and/or approximate proximity to representations of other amino acids, sites, portions of the peptide backbone, and other molecules that may interact with (e.g., bind, so as to form a complex with) a biologic having the peptide backbone represented by the particular scaffold model. For example, in certain embodiments, a scaffold model may include a representation of a first side chain atom, such as a representation of a beta-carbon, which can be used to identify sites and/approximate locations of amino acid side chains. For example, a scaffold model can be populated with amino acid side chains (e.g., to create a ligand model that represents at least a portion of protein and/or peptide) by creating full representations of various amino acids about beta-carbon atoms of the scaffold model (e.g., the beta-carbon atoms acting as ‘anchors’ or ‘placeholders’ for amino acid side chains). In certain embodiments, locations of sites and/or approximate regions (e.g., volumes) that may be occupied by amino acid side chains may be identified and/or determined via other manners of representation for example based on locations of an alpha-carbons, hydrogen atoms, etc. In certain embodiments, scaffold models may be created from structural representations of existing proteins and/or peptides, for example by stripping amino acid side chains. In certain embodiments, scaffold models created in this manner may retain a first atom of stripped side chains, such as a beta-carbon atom, which is common to all side chains apart from Glycine. As described herein, retained beta-carbon atoms may be used, e.g., as a placeholder for identification of sites that can be occupied by amino acid side chains. In certain embodiments, where an initially existing side chain was Glycine, the first atom of glycine, which is hydrogen, can be used in place of a beta-carbon and/or, in certain embodiments, a beta carbon (e.g., though not naturally occurring in the full protein used to create a scaffold model) may be added to the representation (e.g., artificially). In certain embodiments, for example where hydrogen atoms are not included in a scaffold model, a site initially occupied by a Glycine may be identified based on an alpha-carbon. In certain embodiments, scaffold models may be computer generated (e.g., and not based on an existing protein and/or peptide). In certain embodiments, computer generate scaffold models may also include first side chain atoms, e.g., beta carbons, e.g., as placeholders of potential side chains to be added.

### DETAILED DESCRIPTION

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Documents are incorporated herein by reference as noted. Where there is any discrepancy in the meaning of a particular term, the meaning provided in the Definition section above is controlling.

Headers are provided for the convenience of the reader—the presence and/or placement of a header is not intended to limit the scope of the subject matter described herein.

Described herein are methods, systems, and architectures for Artificial-Intelligence (AI)-based design of custom biologics using one or more generative machine learning models (also referred to as “generative networks”). In particular, as described in further detail herein, in certain embodiments, technologies described herein apply a flow-matching technique to peptide backbone design, whereby machine learning models are trained and used to determine adjustments to positions and/or orientations of a collection of seed backbone sites and applied repeatedly to an initial starting distribution (e.g., an initial ‘guess’) to produce a final arrangement of backbone sites representing a newly generated de-novo peptide backbone.

Among other things, the present disclosure encompasses the insight that flow-matching, previously described for image generation in Y. Lipman et al., “Flow matching for generative learning,” the content of which is hereby incorporated by reference in its entirety, can be applied to protein structure design. Beginning with existing flow-matching framework, that have previously been limited to image generation, to create approaches suitable for protein design is non-trivial, and involved, among other things, creation of new featurization approaches to encode protein backbone structures as well as processes for training machine learning models to approximate velocity fields that are used to compute positional and/or orientation adjustments. These approaches are described in further detail herein.

### A. FLOW MATCHING

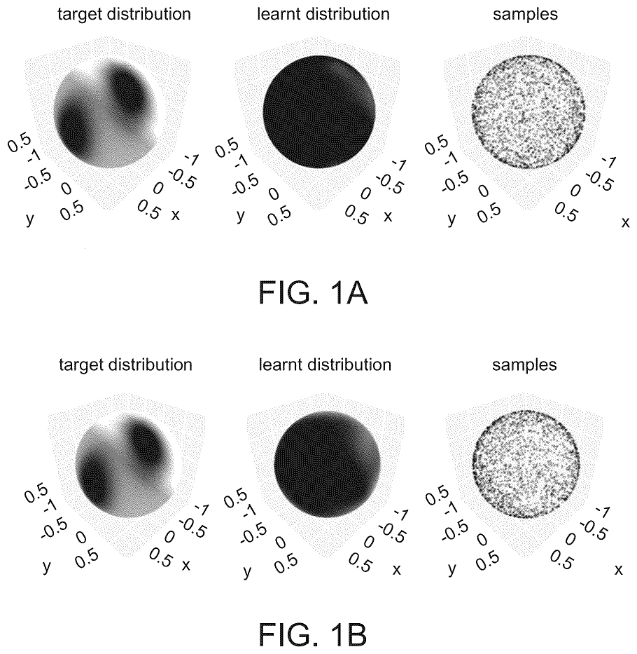

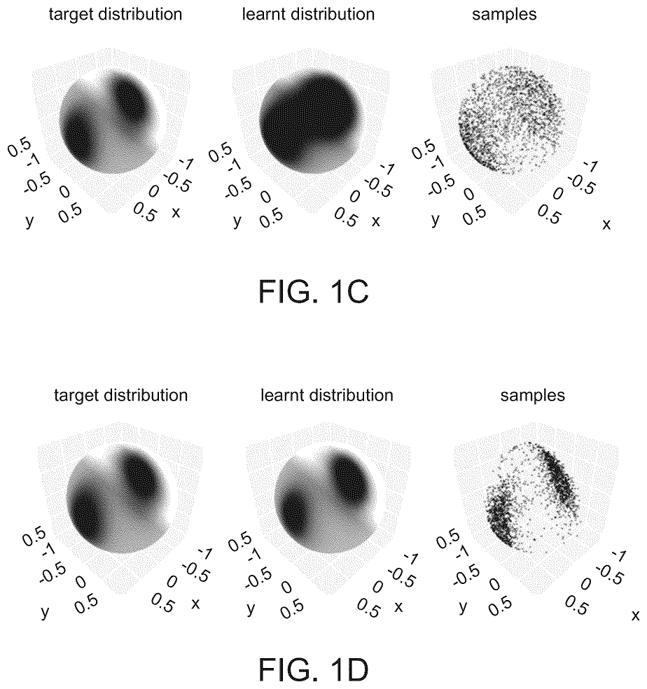

Without wishing to be bound to any particular theory, turning to FIGS. 1A-1D, given a dataset of samples, flow-matching techniques are based on the assumption that the samples of the dataset are or were generated according to (e.g., selected from) an underlying target probability distribution, p₁. Accordingly, in certain embodiments, flow matching approaches seek to generate new samples, like those in the dataset, according to the underlying target probability distribution, p₁, such that they have desired properties and/or are realistic, as embodied by the original examples of the dataset.

Flow matching methods address this challenge by beginning with an initial starting distribution, p₀, and computing a target velocity field v(x, t) that, when applied to a sample x₀ selected from p₀, over a period of time (e.g., from t=0 to t=1), will transform (e.g., “push”) it to a sample x₁ selected from target distribution p₁. In particular, in certain embodiments, flow matching approaches train a machine learning model (e.g., a deep neural network) v_{θ}(x, t) to approximate the target velocity field, v(x, t) (the subscript θ indicating/representing learnable parameters of the machine learning model).

FIGS. 1A-1D are snapshots of graphs showing, at each of four time-steps, an example target distribution p₁ (left plot, red coloration), a current distribution (middle plot, blue coloration), and individual example points xₜ (right plot, blue dots). FIG. 1A is a snapshot at t=0 and, accordingly, shows an initial starting distribution, p₀, which was selected to be a uniform distribution. FIGS. 1B, 1C, and 1D show snapshots at subsequent time-steps, corresponding to 33%, 66%, and 100% of completion, illustrating how a current distribution and sample points evolve to match target distribution, p₁.

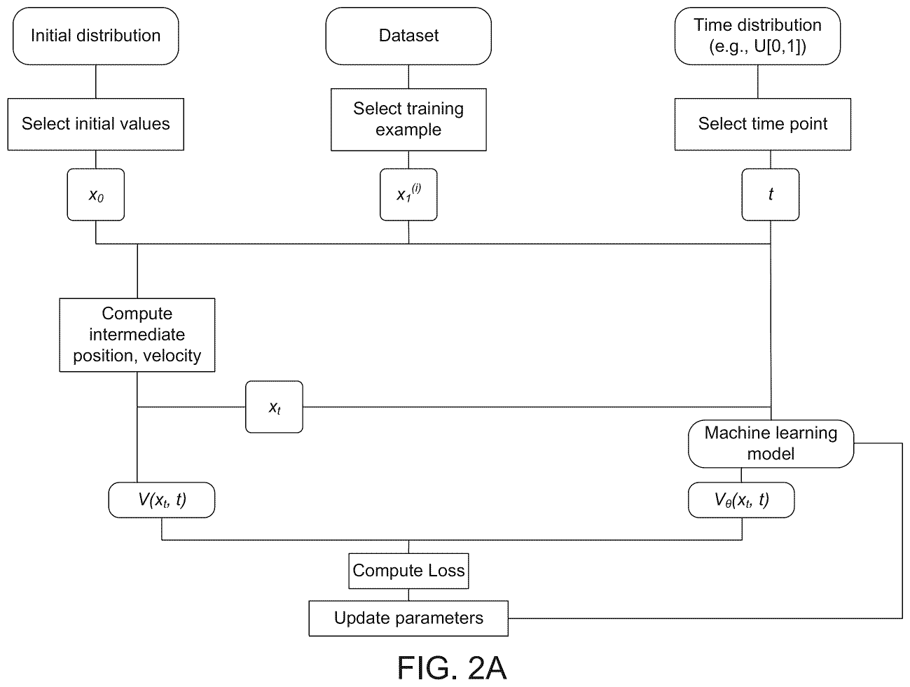

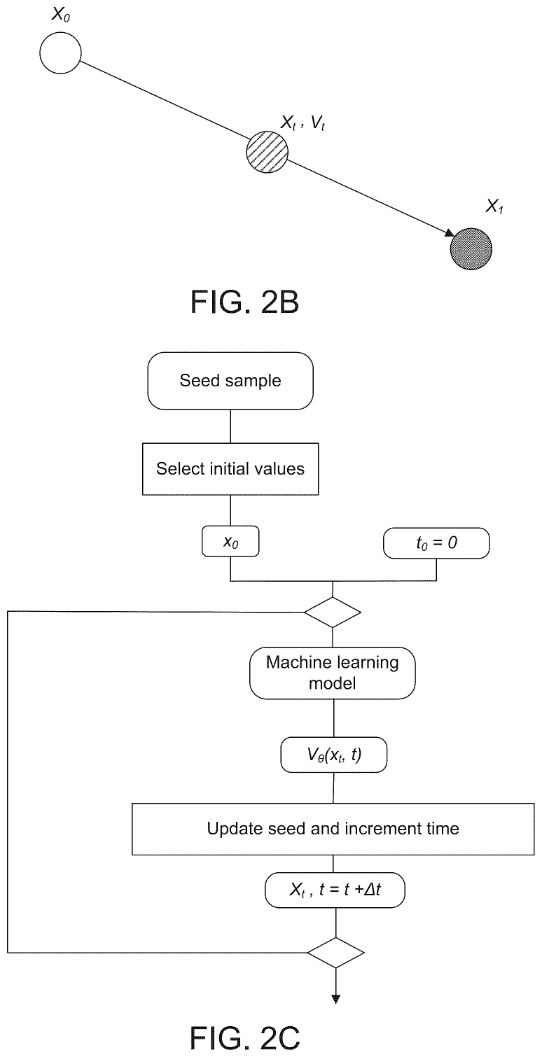

Turning to FIG. 2A, in certain embodiments, a machine learning model v_{θ}(x, t) is trained to approximate a target velocity field using a training dataset, comprising a plurality of training examples, x₁⁽ⁱ⁾, and having a distribution p_{data}. At a particular training step, an initial seed sample, x₀, is selected from initial starting distribution p₀, a training example, x₁⁽ⁱ⁾ is selected from p_{data}, and a particular time point, t, selected at random, for example from a uniform distribution of times ranging from zero to one (e.g., U([0, 1])). In certain embodiments, seed sample x₀, training example x₁⁽ⁱ⁾, and selected time t can be used to compute a ground truth intermediate position, x₁⁽ⁱ⁾, and velocity, v(xₜ⁽ⁱ⁾, t), at the selected time point, along a path from x₀ to x₁⁽ⁱ⁾. For example, in certain embodiments, a target position and velocity are related via an ordinary differential equation (ODE), as shown below:

$$\frac{d x}{d t} = v ( x , t )$$

with x₀ and x₁ (i) being known values of x(t) at t=0 and t=1, respectively.

In certain embodiments, a velocity field v(x, t) can be used to update values of feature vectors using a numerical approximation technique, e.g., for ODE solution, such as Euler's method. In certain embodiments, one or more constraints may be placed on a path from x₀ to x₁⁽ⁱ⁾ and/or a target velocity field, for example to facilitate and/or obtain a particular form of v(x, t). For example, as shown in FIG. 2B, in certain embodiments, a straight-line path—illustrated in 2D space, but, in certain embodiments, corresponding to a geodesic path along a 3D surface—may be used as a constraint (e.g., a hyperparameter) to facilitate evaluation of intermediate position xₜ⁽ⁱ⁾ and velocity, v(x, t). In certain embodiments, velocity field may be assumed (e.g., constrained) to be constant with respect to time.

In certain embodiments, a machine learning model is used to compute, based on an intermediate position, xᵢ⁽ⁱ⁾ and time, t, an estimated velocity, v_{θ}(xₜ⁽ⁱ⁾, t). This estimated velocity (as computed by the machine learning model) can then be compared to the directly computed (as described above), ground truth, intermediate velocity and used to evaluate an error of the machine learning model's estimated velocity, e.g., a loss function. For example, in certain embodiments, a flow matching loss function may be determined according to the below equation:

$$Loss = {‖ {v}_{θ} ( {x}_{t} , t ) - {v}_{t} ‖}^{2}$$

In certain embodiments, other loss functions, e.g., as described herein, may be used.

In certain embodiments, training parameters may be updated, and the above described process may be repeated for a plurality of training iterations, updating parameters of a machine learning model at each iteration (e.g., based on the loss function) to train the machine learning model to generate increasingly accurate estimates of target velocity field.

In certain embodiments, once trained, a machine learning model v_{θ}(x, t) can be used for inference, to generate new data points that are not included within the original dataset, but fit within the target distribution, p₁.

For example, turning to FIG. 2C, in certain embodiments, at inference, a seed sample, having initial values selected, e.g., from a starting distribution (e.g., a uniform distribution) can be used as a starting point, x₀˜p₀. In inference process, rather than select an x₁ from a set of training examples, p_{data}, machine learning model is used to generate a new, not previously seen, x₁ by repeatedly computing velocity field estimates, which are then used to update feature values of seed sample, thereby evolving initial seed sample x₀ into a new, generated point x₁.

### B. PEPTIDE BACKBONE ENCODING AND GENERATION

Among other things, the present disclosure includes systems and methods for a flow-matching based framework that can be used to generate de-novo custom peptide backbones. In particular, approaches described herein include methods for encoding peptide backbone structures in feature vectors and for evolving these feature vectors via machine learning model deterred adjustments to create new custom peptide backbones that nonetheless follow rules and structural motifs of existing native peptides and, accordingly, are likely to be viable.

### B.i Peptide Backbone Encoding

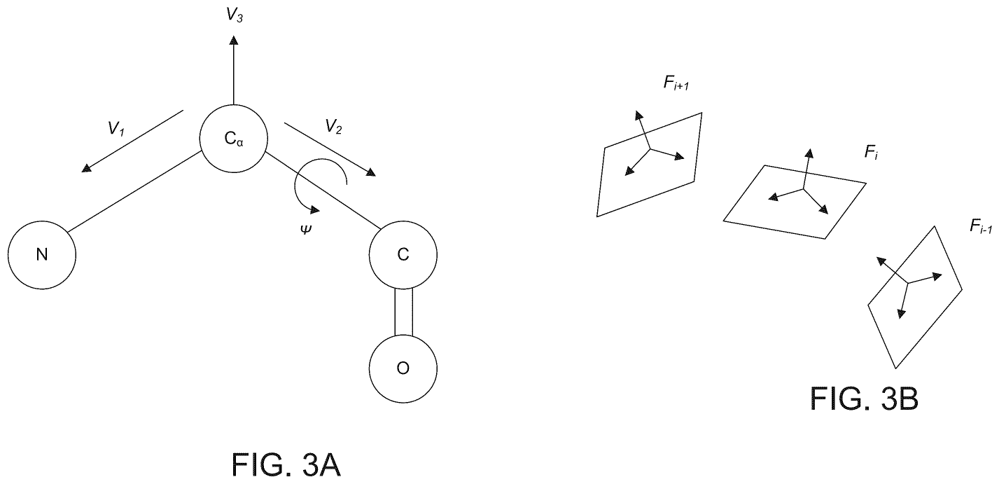

Turning to FIGS. 3A and 3B, in certain embodiments, systems and methods of the present disclosure include approaches for representing peptide backbone sites. For example, as shown in FIG. 3A, a peptide backbone site includes atoms that form a backbone portion of a peptide chain at each amino acid site (e.g., atoms that would remain if side chains were removed from each amino acid site). As shown in FIG. 3A, a backbone site may include a nitrogen (N), an alpha-carbon (Ca), another carbon (C), and an oxygen (O), e.g., bound to each other as illustrated in FIG. 3A.

### (a) Local Frame Representations

In certain embodiments, approaches described herein encode backbone sites using a plurality of feature vectors, each corresponding a particular backbone site and comprising a position and/or orientation component representing a position and/or orientation, respectively, of the corresponding backbone site. In this manner, in certain embodiments, a peptide backbone can be representing using plurality of feature vectors.

In certain embodiments, each feature vector comprises a position component, representing three-dimensional position of a backbone site. In certain embodiments, each feature vector comprises an orientation component, representing a 3D orientation of a corresponding backbone site. In certain embodiments, each feature vector comprises a position and an orientation component. For example, in certain embodiments, each feature vector has a form [T, R], where T is a position component, representing a position of a corresponding backbone site and R is an orientation component, representing a 3D orientation of the corresponding backbone site.

In certain embodiments, a local frame, Fᵢ, can be determined for, and used to represent, each backbone site. In certain embodiments, local frames can be determined based on a three-dimensional [e.g., Cartesian, (x, y, z), coordinate] location of a C_{α} atom and orientations based on C_{α}—N and C_{α}—C axes. For example, in certain embodiments, a local frame can be determined as a frame having an origin coincident with C_{α} of a particular backbone site. For example, in certain embodiments, an orthonormal basis—a set of three vectors—can be used to define a local frame for each backbone site. In certain embodiments, an orthonormal basis can be determined using positions of atoms in (e.g., represented via a structural model of a) backbone site as illustrated in FIG. 3A. A first basis vector, v₁, can be determined using positions of a C_{α} and N, as a normalized vector pointing from C_{α} to N. A second basis vector, v₂, can be determined as a vector is orthogonal to v₁ and in a plane comprising v₁ and a C_{α}—C bond (and normalizing the result). A third basis vector, v₃, can be determined by computing a cross product between v₁ and v₂ (and normalizing the result). In this manner, an orthonormal basis (v₁, v₂, v₃) representing a local frame can be determined for each backbone site of a peptide chain. Additionally or alternatively, each local frame can be used to compute (e.g., approximate) positions of atoms of a corresponding backbone site, for example using ideal bond lengths and angles.

A peptide chain can, accordingly, be represented as a set of local frames, each representing a particular backbone site of the peptide chain and having a position and orientation. In certain embodiments, positions and orientations of each local frame Fᵢ are represented in a relative fashion, from a point of view of each of one or more other local frames. For example, in certain embodiments, each local frame may be assigned an index (e.g., according to a sequence order of a corresponding backbone site that it represents), and its position and orientation represented as a relative translation and rotation from a point of view of a preceding local frame, Fᵢ₋₁. That is, each feature vector encodes a position and orientation of a local frame according to Fᵢ=[Tᵢ, Rᵢ], where Tᵢ and Rᵢ are a translation and rotation of Fᵢ relative to Fᵢ₋₁. A translation may be determined using relative positions of the two origins of the two local frames, and a rotation may be determined using the two sets of orthonormal basis vectors.

For example, as illustrated in FIG. 3B, local frame Fᵢ₊₁ can be represented via a translation and rotation relative to local frame Fᵢ, local frame Fᵢ can be represented via a translation and rotation relative to local frame Fᵢ₋₁, and so on. In this manner, backbone sites of a peptide chain can be represented in an entirely relative fashion that is invariant to global translation and/or rotation of the entire peptide chain. In certain embodiments, this invariance/equivariance with respect to global translations and/or rotations is advantageous, for example allowing use of machine learning (e.g., neural network) architectures that are not inherently equivariant.

In certain embodiments, each local frame is represented relative to multiple other frames. In this manner, multiple relative representations of each local frame can be fed into machine learning models described herein and used to generate multiple predictions of positions and/or orientations of backbone sites and/or adjustments thereto, allowing for a consensus or average of the predictions to be used/selected.

Additionally or alternatively, in certain embodiments, positions and/or orientations of local frames may be represented relative to their own positions and/or orientations at other time points/iterations. For example, a position and/or orientation of a particular local frame at a particular time point or iteration may be represented as a translation and/or rotation, respectively, relative to that particular local frame's position and/or orientation and one or more previous time points or iterations.

In certain embodiments, each local frame's position and orientation can be represented in a global fashion, relative to a single common global reference point/axis, such as a common origin and set of three unit vectors (e.g., Cartesian unit vectors (nₓ, n_{y}, n_{z})).

Accordingly, in certain embodiments, a peptide chain can be parameterized using a set or list of feature vectors, each representing a translation and rotation of a particular local frame that represents a particular backbone site. Each feature vector may, e.g., be or be viewed as a row in a matrix, and take the form x₍ᵢ₎=Fᵢ=[Tᵢ, Rᵢ], for an i^{th} row/feature vector representing an i^{th} backbone site, where T and R are the translation and rotation of the local frame. A three-dimensional translation, T, may be represented via three values, each representing a shift along a particular coordinate, such as T=(Tₓ, T_{y}, T_{z}). A rotation R may be represented via a quaternion (e.g., a set of four values representing a rotation about an axis u by an angle theta), such as R=(q₁, q₂, q₃, q₄). Accordingly, in certain embodiments, each feature vector may be or comprise a seven-element vector, and a peptide chain having length k represented by k feature vectors, e.g., a 7 by k matrix.

### (b) Individual Backbone Atom Representations

In certain embodiments, (e.g., additionally or alternatively to using a local frame-based representation as described herein) atoms of backbone sites—i.e., N, C_{α}, C, and O atoms—can be represented directly, via their location in three-dimensional space, for example via a Cartesian (x, y, z) coordinate for each atom. In this manner, each backbone site may be represented via a twelve-element coordinate vector, such as x₍ᵢ₎=[Nᵢ, C_{αi}, Cᵢ, Oᵢ], where, for example, Nᵢ, C_{αi}, Cᵢ, and O; are (x, y, z) coordinates of the N, Ca, C, and O atoms of the i^{th} amino acid site. Accordingly, in certain embodiments, each feature vector may be or comprise a twelve-element vector representing a position of each of the four backbone atoms, and a peptide chain having length k represented by k feature vectors, e.g., a twelve by k matrix.

### (c) Characteristics and Benefits of Local Frame and Individual Backbone Atom Representations

In certain embodiments, an individual (four) backbone atom representation can be advantageous, since it represents backbone sites via a list of coordinates in 3D space. Each of these coordinate values thus lie in R³ (real coordinate space), which is relatively easy to manipulate and is linear, thereby avoiding more complex mathematical formulations which, for example rely on logarithmic and/or exponential mapping tools. Moreover, in certain embodiments, by representing each atom of each backbone site directly, an individual backbone atom representation approach does not use assumptions, such as perfect bond length and angles. Individual backbone atom representation approaches, however, do not inherently enforce a fixed relationship between relative positions of individual backbone sites, and, accordingly, increase a number of dimensions to be learned by machine learning models described herein. For example, in certain embodiments, learned velocities via flow matching techniques described herein use twelve (12) coordinates per amino acid site when an individual backbone representation is used, as opposed to e.g., six, when a local frame representation is used.

Accordingly, in certain embodiments, local frame representations can be advantageous in that they simplify the problem in terms of the number dimensions to use. Additionally or alternatively, in certain embodiments, local frame representations can facilitate creation of equivariant machine learning architectures from invariant features, for example, as described herein with respect to self-attention based SO(3) transformer versus applying frame rotation.

In certain embodiments, approaches described herein address challenges associated with use of a local frame representation. Among other things, in certain embodiments, where local frame representations are used, machine learning models described herein utilize a logarithmic mapping technique, which can become undefined when an angle of rotation approaches π. This can, in turn, cause velocities to be undefined, leading to numerical instabilities. Additionally, or alternatively, machine learning approaches described herein account for a two-to-one (2-to-1) mapping where quaternion representations are used (e.g., since q and −q represent a same rotation). As described herein, local frame representation approaches also may include techniques to recover individual atom positions, for example based on assumptions of (e.g., ideal) bond lengths and angles, from local frame Ca coordinates and rotations.

### B.ii Training and Inference

In certain embodiments, flow matching technologies for generating de-novo peptide backbones of the present disclosure include systems and methods for training machine learning models, such as neural networks (e.g., deep neural networks), to model velocity fields that can be used to adjust positions and/orientations of feature vectors representing peptide backbone sites to create new backbone structures.

### (a) Training

In certain embodiments, a machine learning model is trained to model a velocity field using 3D protein backbone structures, for example, selected as examples from data sources such as the protein data bank (PDB). For example, a training dataset may be created from 3D structures of proteins by converting each structure into a list of feature vectors matching a desired parameterization, such as the local frame representation described above.

In certain embodiments, at a particular training step, an example representation of a backbone structure, x₁⁽ⁱ⁾, is selected from a training dataset. As described herein, in certain embodiments, x₁⁽ⁱ⁾ is or comprises a plurality of feature vectors, each representing a corresponding backbone site via a local frame. In certain embodiments, e.g., as described herein with respect to FIG. 2A, a time point may be selected, for example at random from a uniform distribution.

In certain embodiments, a seed set comprising a plurality of feature vectors is used as an initial starting point, x₀. Values of position and/or orientation components of each feature vector of seed set may be set to initial starting values. Initial starting values for each feature vector may be selected at random, for example from an initial probability distribution, such as a uniform distribution of position and/or orientations. In certain embodiments, initial starting values may be determined, for example, using a preliminary machine learning model (different from a machine learning model used to model velocity field v_{θ}(x, t)). A preliminary machine learning model may be, for example, be trained to generate a prior distribution that more closely matches a target distribution p₁ than, e.g., random selection from a uniform distribution, thereby providing an improved ‘initial guess’ as a set of initial starting values.

In certain embodiments, based on the initial starting point x₀⁽ⁱ⁾, the selected time point t, and training example x₁⁽ⁱ⁾, an intermediate feature set x₁⁽ⁱ⁾ and velocity, v(xₜ⁽ⁱ⁾, t), can be calculated (e.g., using x⁽ⁱ⁾(t=0)=x₀⁽ⁱ⁾ to x⁽ⁱ⁾(t=1)=x₁⁽ⁱ⁾), for example using numerical ODE solution approaches such as Euler's method and, additionally or alternatively, various constraints, such as particular forms of paths from x₀ to x₁⁽ⁱ⁾, functional forms of velocity field v(x, t), etc. In certain embodiments, a (e.g., analytical) derivative of position

$$v ( {x}_{t}^{( i )} , t ) = \frac{d {x}_{t}^{( i )}}{dt}$$

can be used if a closed form exists. Additionally or alternatively, derivatives can be approximated, for example via

$$v ( {x}_{t}^{( i )} , t ) \approx \frac{{x}_{t + Δ t}^{( i )} - {x}_{t}^{( i )}}{Δ t} .$$

Intermediate feature set xₜ⁽ⁱ⁾ comprises updated values for position and/or orientation components of the plurality of feature vectors in the initial seed set, and, accordingly, represents intermediate positions and/or orientations of each backbone site of a peptide backbone being generated, at a position (in feature space) along a path from the initial starting values to the final positions and orientations of the backbone sites as they are in the selected training example, x₁⁽ⁱ⁾—e.g., a real peptide chain. Determined velocity, v(xₜ⁽ⁱ⁾, t), represents an adjustment to each feature vector—i.e., to a position and orientation of each backbone site, in a direction of their final positions and orientations.

In certain embodiments, intermediate feature set xₜ⁽ⁱ⁾ and selected time t can be used as input to a machine learning model that determines, as output, an estimated velocity v_{θ}(xₜ⁽ⁱ⁾, t) associated with intermediate feature set xₜ⁽ⁱ⁾ and time t. This estimated velocity, as determined by the machine learning model (at a particular training iteration), can then be compared with directly computed velocity v(xₜ⁽ⁱ⁾, t), to evaluate a loss or error function at the particular training step. This process can be repeated, for a number of training steps, as parameters of the machine learning model are adjusted to obtain increasingly accurate estimations of velocity, until a desired number of evaluations and/or a desired accuracy is reached. In this manner, a machine learning model can be trained to determine, for example, for a particular feature set (e.g., a position and orientation of a plurality of local frames representing peptide backbone sites) and time, a velocity that can be used to update the particular feature set and “push” its values towards those that mirror those of the training examples—i.e., real physical peptide chains.

In certain embodiments, training does not necessarily proceed one example at a time. For example, in certain embodiments, a batch of a plurality of training examples and a plurality of sets of initial starting values are selected. Each set of initial starting values may then be matched with a particular training example that it is nearest to, e.g., using a Sinkhorn algorithm, to improve efficiency of training.

### (b) Inference

Accordingly, in certain embodiments, a trained machine learning model can be used to compute velocities that can be used to update position and/or orientation component values of feature vectors so as to progressively ‘push’ them from initial values representing, e.g., a random distribution of backbone sites or an initial guess, to a set of final values that represent positions and orientations that are indicative/representative of real, physical peptide chains. In this manner, peptide backbones created via generative techniques described herein may be new, not previously seen, yet nonetheless have a high likelihood of being viable, functioning (e.g., properly folded/stable) peptide backbones.

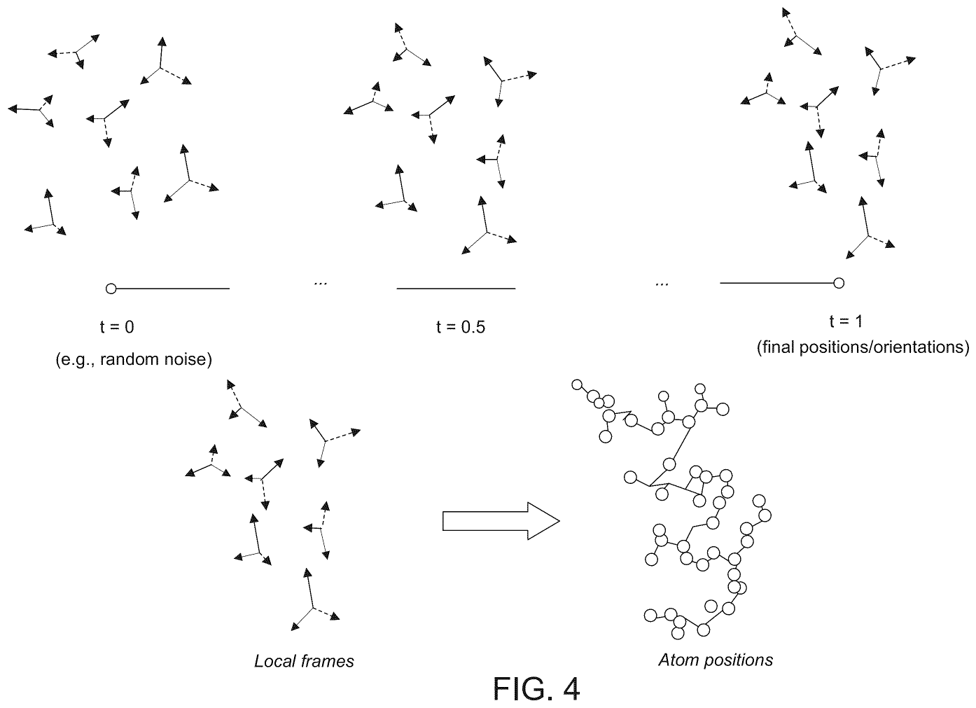

For example, as shown in FIG. 4, to generate a new, custom peptide backbone, approaches described herein may begin with a seed set comprising a plurality of feature vectors, each having position and/or orientation components representing positions and/or orientations, respectively, of a corresponding peptide backbone site. Initial values of position and/or orientation components of each feature vector may be determined in a variety of manners, as described herein, such as at random, from an initial distribution. Accordingly, for example, seed set may begin, e.g., at time t=0, by representing a plurality of peptide backbone sites having random positions and/or orientations—e.g., random noise. In certain embodiments, beginning with the initial starting values of the position and/or orientation components of the plurality of feature vectors, at a first iteration, a machine learning model trained as described herein to estimate a velocity field may be used to compute an initial velocity filed, based on the initial values of the position and orientation (e.g., as well as the selected initial time point, t=0). The initial velocity field may then be used to update values of the position and/or orientation components of the plurality of feature vectors. These updated values may be used as current values of position and/or orientation components of the feature vectors at a subsequent iteration, which, in turn, can be used (e.g., together with an incremented, current time point) to determine a current velocity field. The current values of the position and/or orientation components can then be updated according to the current velocity field, and the process repeated in an iterative fashion, until a final iteration (e.g., time step) is reached.

Final values of the feature vectors can then be used to generate a scaffold model representing a final, generated, custom peptide backbone, for example by computing backbone atom positions from each local frame representation.

### (c) Approaches for Velocity Field Estimation

In certain embodiments, one or more (e.g., different) approaches may be used for determining velocity fields using a machine learning model. In certain embodiments, a machine learning model is used to directly estimate a velocity field at each iteration. For example, a machine learning model may receive, as input, a current set of feature vector values and a current time point and generate, as output, a current velocity field that can be used to update feature vector values, e.g., for use in subsequent iterations.

Additionally or alternatively, in certain embodiments, a machine learning model may not necessarily directly output an estimated velocity field, but, rather, may output a set of prospective final feature vector values—e.g., a current estimate of x₁, representing a current estimate of positions and/or orientations of each backbone site of a generated peptide backbone, based on current feature vector values at a current time point. A current velocity field may then be determined based on the set of prospective final feature vector values and a current time point [e.g., corresponding to a current iteration (e.g., generating a current estimate of x₁ as output of the machine learning model and computing the current velocity Field based on x₁ and the current time point)]. This approach may proceed iteratively, with the machine learning model repeatedly generating refined calculations of prospective final feature vector values, which are then used to compute a current velocity field and adjust position and/or orientation components of the feature vectors.

In certain embodiments, approaches wherein a machine learning model generates a prospective/current prediction of x₁ as output may use a single type of local frame representation and/or may combine multiple representations. For example, in certain embodiments, initial starting values of feature vector position and/or orientation components may represent position and/or orientations of local frames in a (e.g., purely) spatial fashion—e.g., relative to one or more neighboring frames and/or to a common global reference. In certain embodiments, a machine learning model may generate output predictions of x₁ using a same representation as the initial starting values, e.g., also in a spatial fashion. In certain embodiments, a machine learning model may generate output predictions of x₁ using a different representation as the initial starting values, for example by computing, for each frame i, a translation and/or rotation of that frame at time t=1 (a final time point) relative to its position and/or orientation at a current time point.

### C. AMINO ACID SEQUENCE ENCODING AND GENERATION

Additionally or alternatively, generative, flow-matching-based technologies of the present disclosure may be used to generate (e.g., de-novo) amino acid sequences for custom proteins and/or peptides. In certain embodiments, flow-matching methods and systems for generation of amino acid sequences may be implemented and/or used as dedicated (e.g., separate) systems and methods, for example via dedicated machine learning models, distinct from those used for design of other polypeptide features, such as backbone design and/or side-chain packing, described herein. In certain embodiments, as described in further detail herein, flow-matching technologies for generative design of amino acid sequences may be combined with other flow-matching prediction technologies, such as peptide backbone design and/or side chain packing techniques, via a single (e.g., unified) multi-input and/or multi-task model. In this manner, sequence design technologies of the present disclosure can be used alone and/or in connection with other design modules and/or techniques to provide a range of capabilities in the context of in-silico design of custom biologics.

### C.i Sequence Encoding

In certain embodiments, sequence design technologies of the present disclosure leverage particular representations of polypeptide sequences to address a key challenge in flow-matching-based generative design of amino acid sequences-namely, the categorical, discrete nature of amino acid sequences. That is, for example, proteins are large biomolecules composed of one or more lengthy polypeptide chains, each comprising a plurality of amino acid residues linked together via peptide bonds. Accordingly, an amino acid sequence of a polypeptide chain and/or an overall protein represents order and type of the multiple amino acid residues that the one or more chains of a protein and/or peptide molecule comprises. An amino acid sequence of a polypeptide chain can, accordingly, be represented as an ordered list of characters, numbers, or other variables, each having a particular value selected from a discrete set representing possible amino acid types. A set of possible amino acid types may be or comprise the twenty standard amino acids and non-standard amino acids.

For example, an amino acid sequence may be represented as a list of three-character strings, each representing and identifying a particular one of the twenty standard amino acid, e.g., “Glu Ala Gln Ile Thr Gly . . . Thr Leu . . . .” Additionally or alternatively, amino acids may be represented via single characters, such that this same sequence may be represented via a string, “EAQITG . . . TL . . . .”. In certain embodiments, an amino acid sequence of a polypeptide chain can be represented as an ordered list of integers, each integer identifying a particular one of the twenty standard amino acids. For example, an amino acid sequence may be represented as shown below

$${\{ {r}_{i} \}}_{i = 1}^{n} , 0 s . t . , {r}_{i} ∈ \{ 1 , … , 20 \} ,$$

where n is a number of amino acid residues in the polypeptide chain and rᵢ is a categorical type of amino acid present at that position, constrained to the set {1, . . . , 20}, reflecting the 20 standard amino acids. This representation effectively maps the linear sequence of a protein to a numerical sequence, with each number corresponding to a distinct type of amino acid.

Applications of flow-matching techniques typically involve generating continuous variables, such as real numbers. Applying flow matching to discrete domains presents a complex challenge. This difficulty primarily arises because flow-matching is based on modeling continuous flow of probability paths and is facilitated by solution of differential equations in this, continuous, context. Setting up differential equations on discrete domains, such as polypeptide sequence representations, is non-trivial, since, among other things, derivatives are not straightforwardly defined on discrete domains. Accordingly, the continuous nature of generative modeling techniques based on flow-matching presents a fundamental challenge to their use where quantities to be modelled take on and/or are restricted to a finite number of discrete values.

Accordingly, in certain embodiments, technologies of the present disclosure for amino acid sequence generation include approaches for representing amino acid sequences on continuous domains. For example, in certain embodiments systems and methods of the present disclosure include steps of converting representations of amino acid residues from discrete to continuous values.

In particular, in certain embodiments, amino acid sequence generation technologies described herein utilize a relaxed continuous domain of simplices for modeling amino acid types, which facilitates use of flow matching-based machine learning models for generation of amino acid sequences of polypeptide chains. In certain embodiments, a domain of residue types is transformed from discrete values to a representation within a 19-dimensional simplex, thereby effectively treating each amino acid type as a point in a continuous probability distribution. This approach models rᵢ as an element in [symbol] ²⁰, subject to conditions that ∥rⁱ∥₁=1 and each component of the rᵢ vector lies (i.e., takes on a value) between 0 and 1. Among other things, this method establishes a one-to-one correspondence between 20-dimensional categorical probability distributions and points within the simplex, underscoring the suitability of the simplex domain for the sequence generation technologies of the present disclosure. In this manner, a polypeptide (e.g., protein and/or peptide) sequence may be represented via a set of twenty-element feature vectors, each corresponding to and representing a particular amino acid type at a particular location (e.g., site) within a protein and/or peptide. Each of the twenty amino acid types may be associated with a particular index or location within a feature vector, and each of the twenty elements of a particular feature vector may have a value between 0 and 1, representing a probability of a particular type of amino acid. Accordingly, a sequence of a protein having k amino acids may be represented via a set of k 20-element feature vectors, or a k×20 size matrix. Machine learning models may then be used to generate velocity fields for iteratively updating values of these, sequence, feature vectors, updating probabilities of various types of amino acids at sites across a polypeptide chain, until they, e.g., coalesce into final values peaked at a one or a few likely amino acid types. Feature vectors may then be converted into a final identification of a particular, single, amino acid type via, for example, an argmax function.

### C.ii Training and Inference

### (a) Training

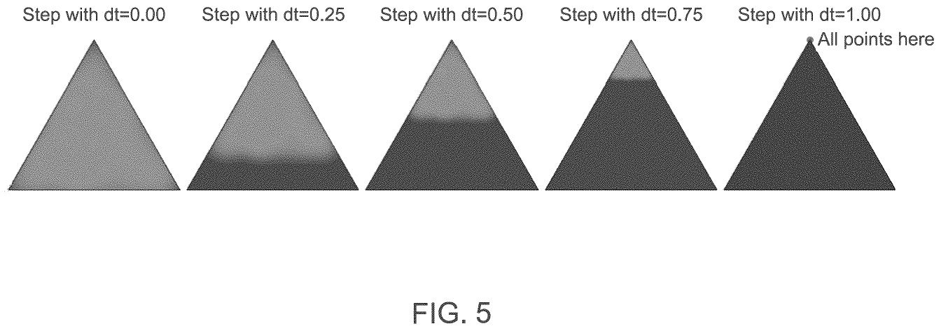

Turning to FIG. 5, sequence design approaches of the present disclosure, may, accordingly, utilize continuous representations of protein sequences, such as the simplex approach described herein, to leverage flow matching methods and systems described herein with regard to generation of peptide backbone structures. In particular, an interpolant or path constraint, ϕ, representing a path interpolating between an initial seed point, x₀, selected from an initial starting distribution p₀, and a final target, x1, selected from a dataset of training examples, p_{data}. may be used to map points from an initial starting distribution to a desired, target distribution represented and/or approximated by a dataset. See, e.g., [Chen, 2016, “Neural Ordinary Differential Equations”].

Adopting the linear continuous interpolation methodology (e.g., used for backbone C_{α} atoms, i.e. corresponding to Euclidean manifold) for sequence generation is facilitated via the simplex domain representation approach described above, which constitutes a convex set within the Euclidean space. In particular, in certain embodiments, a straight line, linear interpolation,

$$∀ t ∈ [ 0 , 1 ] , {ϕ}_{t} ( {x}_{0} \vert {x}_{1} ) = ( 1 - t ) \times {x}_{0} + t \times {x}_{1} ,$$

may be used. Without wishing to be bound to any particular theory, it is believed that the entire interpolation path remains within the simplex for all times t, since the interpolated point is a convex combination of points, which, by definition of convex sets, is always contained within the simplex, underscoring the suitability of simplex as the domain for this application. In certain embodiments, any path deviation from a simplex (e.g., due to a numerical error) may be corrected (e.g., adjusted) to be back on the simplex.
In certain embodiments, an initial starting distribution from which a seed points x₀ are selected is a uniform distribution on a simplex, for example, uniform distribution on the simplex: i.e., x₀˜p₀= [symbol] ir (α=1₂₀=(1, 1, . . . , 1)∈ [symbol] ²⁰ where [symbol] ir denotes the Dirichlet distribution. In certain embodiments, other initial starting distributions (e.g., not necessarily uniform; distributions that a priori introduce specific bias (e.g., used for conditioning); different Dirichlet distributions; different distributions across amino acid types; completely different distribution on the domain of simplex distribution) may be used.

FIG. 5 shows an example of a uniform distribution on a simplex evolving to a final value at x₁=[0, 0, 1].

Accordingly, by virtue the continuous representation of amino acid sequences described herein, flow-matching techniques for sequence generation can be trained in a similar manner to those described above with respect to peptide backbone generation approaches. That is, a particular training example x₁⁽ⁱ⁾ may be selected from a training dataset, having a distribution p_{data}. An initial seed point, x₀⁽ⁱ⁾, may be selected from an initial starting distribution, here a uniform distribution on a 19-dimensional simplex, and a (e.g., arbitrary/random) time point t selected. In the context of sequence generation, points x₁⁽ⁱ⁾ and x₀⁽ⁱ⁾ comprise sequence feature sets, representing an example amino acid sequence selected from the training dataset and a random initial starting point, where at each amino acid site probabilities across different amino acid types are random, based on a uniform distribution. From these selected points, and the interpolant form, ϕ, an intermediate feature set xₜ⁽ⁱ⁾ and intermediate velocity, v(x_{t, t}) (e.g., a sequence velocity) may be determined. As with the peptide backbone generation approaches described herein, the machine learning model may then be tasked with determining velocity estimates, v_{θ}(xₜ⁽ⁱ⁾, t), (e.g., sequence velocity estimates) and/or outputs from which they (sequence velocity estimates) can be determined, which can, in turn, be compared with the determined intermediate velocity, via a loss function.

In certain embodiments, for example when a simplex approach as described herein is used, an intermediate velocity v(xₜ, t)=uₜ=x₁−x₀ may be determined directly from x₁ and x₀.

A variety of approaches may be used to determine estimated velocity fields v_{θ}(xₜ⁽ⁱ⁾, t), from a machine learning model for sequence generation. In particular, a machine learning model, such as various invariant models described herein may generate an invariant representation, hₜ. In certain embodiments, representation hₜ is invariant by virtue of a particular, invariant, machine learning architecture. This invariant representation may itself be used as output, for example such that the machine learning model directly generates a predicted velocity, or one or more additional operators (e.g., softmax, sp, clipping, etc.) may be applied to generate various desired forms of output.

Four example approaches for generating estimated velocity fields via a machine learning model are listed below, without limitation.

1. Delta Prediction. In certain embodiments, an approach, referred to herein as delta prediction, is used whereby a machine learning model generates (e.g., as output) a prospective final sequence feature vector—i.e., an estimate of x₁⁽ⁱ⁾, denoted x̅₁. Predicted target values may be obtained from an invariant representation, hₜ, generated within a machine learning model via one or more differentiable projection operations that map hₜ to a 19-dimensional simplex, such as a softmax operation. Sequence velocity fields may then be generated from the prospective final sequence representation according to a desired path, for example, using the linear path in Euclidean space described herein, as

$$v ( {x}_{t} , t ) = \frac{{\overline{x}}_{1} - {x}_{t}}{1 - t} .$$

2. Double Prediction. In certain embodiments, an invariant representation, hₜ, may be divided into two separate vectors and projected onto a (e.g., continuous) representation of an amino acid sequence, such as a simplex. Without wishing to be bound to any particular theory, for example, it is believed that this double prediction approach mirrors the form of a velocity field in a linear approximation, where it is computed as a difference between two sequence feature vectors. For example, in certain embodiments hₜ is a 40-dimensional invariant representation, which may be projected onto a (e.g., 19 dimensional) simplex via a sp operator (e.g., any operator that transforms 20-dimensional vector into a simplex, e.g., such that, values of resultant vectors are between 0 and 1 and all sum to 1), as shown below:

$$v ( {x}_{t} , t ) = sp ( {h}_{t} [ 0 : 20 ] ) - s p ( {h}_{t} [ 20 : 40 ] ) .$$

3. Raw Prediction. In certain embodiments, an invariant representation, hₜ, is used directly as output, such that v_{θ}(xₜ⁽ⁱ⁾, t)=hₜ. In this manner, the machine learning model learns (e.g., via training) to generate, directly velocity field vectors.

4. Raw Prediction with max norm clipping. In certain embodiments, a raw prediction, e.g., v_{θ}(xₜ⁽ⁱ⁾, t)=hₜ, is clipped to predict vectors having a particular maximum norm, for example a maximum L2 norm of √2, to mirror and/or enforce a desired norm known to be true for a ground truth velocity field.

In certain embodiments, machine learning model-based predictions may be evaluated, e.g., during training, via a variety of loss functions, which, in turn, may be used to update model weights and refine predictions during a training procedure. Loss functions may be structured to determine based on velocity field estimates and/or directly based on prospective final sequence representations.

For example, in certain embodiments, where amino acid sequences are represented via simplexes, an expected velocity given a particular position and time takes on the below relationship,

$$\begin{matrix}V ( x , t ) = 𝔼 [ {X}_{1} - {X}_{0} \vert {X}_{t} = x ] \\ = 𝔼 [ \frac{{X}_{1} - {X}_{t}}{1 - t} \vert {X}_{t} = x ] \\ = \frac{E [ {X}_{1} \vert {X}_{t} = x ] - x}{1 - t} \\ = \frac{{[ p ( {x}_{1} = {e}_{i} \vert {X}_{t} = x ]}_{i = 1}^{20} - x}{1 - t} .\end{matrix}$$

Accordingly, estimating a velocity and/or final prospective sequence feature vector may be considered equivalent, and, accordingly, loss functions can be structured based on either approach.

For example, where a machine learning model is used to estimate a velocity field, the following two loss functions may be used:
1. MSE loss function:

$$L = {E}_{t ∼ [ 0 , 1 ] , {x}_{0} ∼ {p}_{0}} {‖ ( {x}_{1} - {x}_{0} ) - \tilde{v} ( {x}_{t} , t ) ‖}_{2}^{2} .$$

2. Cross entropy with forward prediction:

$$L = {E}_{t ∼ [ 0 , 1 ] , {x}_{0} ∼ {p}_{0}} CrossEntropy ( {x}_{1} , {x}_{t} + \tilde{v} ( {x}_{t} , t ) \times ( 1 - t ) ) .$$

In certain embodiments, where a machine learning model is used to estimate a prospective final sequence representation, \tilde{x}₁, one or more of the following loss functions may be used:
1. MSE loss function:

$${L}_{MSE} = {E}_{t ∼ [ 0 , 1 ] , {x}_{0} ∼ {p}_{0}} {‖ ( {x}_{1} - {x}_{0} ) - \frac{{\tilde{x}}_{1} ( {x}_{t} , t ) - {x}_{t}}{1 - t} ‖}_{2}^{2} .$$

2. Cross entropy with forward prediction:

$${L}_{CE} = {E}_{t ∼ [ 0 , 1 ] , {x}_{0} ∼ {p}_{0}} CrossEntropy ( {x}_{1} , {\tilde{x}}_{1} ( {x}_{t} , t ) ) .$$

3. Sum of MSE and Cross entropy:

$${L}_{tot} = α {L}_{MSE} + β {L}_{CE} .$$

4. Cross entropy with raw prediction: L=E_{t˜[0, 1],x}_{₀}_{˜p}_{₀} CrossEntropy(x₁, hₜ), where hₜ represents the outputted invariant representation.

In certain embodiments, a non-uniform time sampling is used during training, for example to account for the categorical distribution of amino acid sequences, particularly where times lie close to 1. In particular, here, at times close to 1 the argmax function effectively minimizes the loss function-accordingly, where these (close to 1) times are used, the machine learning model may be essentially trained replicate an argmax function.

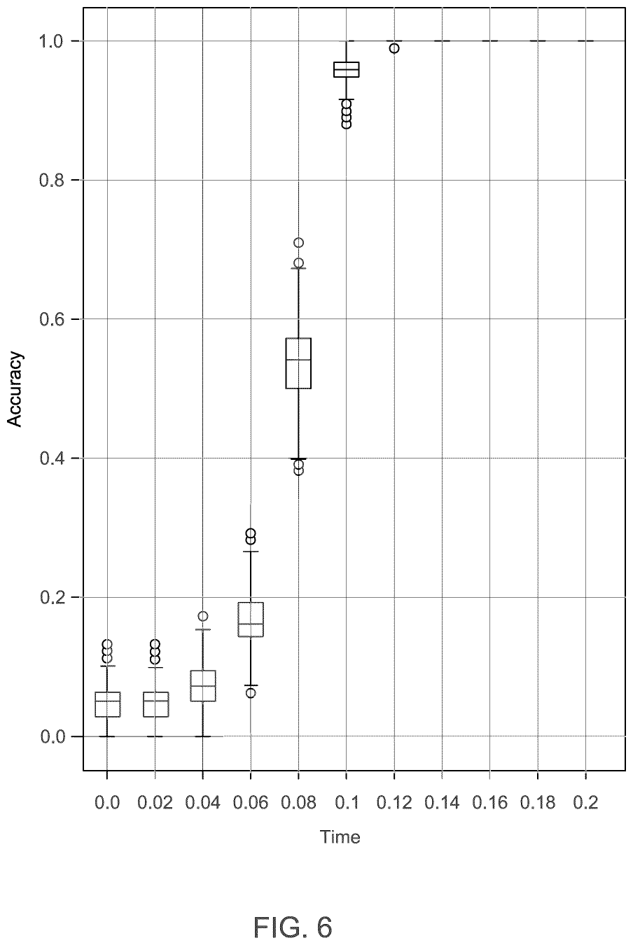

For example FIG. 6 shows a plot produced by sampling 100 points from a Dirichlet distribution (e.g., [symbol] ir (α=1₂₀=(1, 1, . . . , 1)∈ [symbol] ²⁰) and an arbitrary, p₁ distribution, taken to be uniform eᵢ∈{1, . . . , 20} to simulate a flow matching path pₜ. The graph plots accuracies determined as argmax(xₜ)=argmax(x₁) for any discretization over 50 time steps. As shown in FIG. 6, accuracy of the argmax function rapidly approaches 1 for times over about 0.12. Accordingly, sampling time uniformly between 0 and 1 can significantly slow down training, since the functional form that minimizes the loss at later times is captured by the relatively simple argmax function, while desired, more complex behavior at earlier times may be under-represented in the training examples. In certain embodiments, sequence prediction training approaches of the present disclosure, accordingly, sample time non-uniformly during training, to place a greater emphasis on values closer to t→0 and less on values approaching t→1. For example, in certain embodiments, a time value, t, may be sample from a particular distribution, such as a Beta distribution [e.g., B(α, β) where α<β], an exponential decay distribution [e.g., t=ƒ(x)=exp (−λx), where λ>1 and x˜U(0, α) for any α>100], and the like.

### (b) Inference

In certain embodiments, a machine learning model having been trained, for example as described herein, to determine predictions of velocity field and/or final sequence representations, may be used to generate polypeptide sequences via a flow-matching approach. In certain embodiments, generating (e.g., de-novo) polypeptide sequences comprises using a machine learning model to determine estimates of velocities and/or final sequence representations for use in an ordinary differential equation (ODE) solver, such as a Euler or Runge-Kutta method.

For example, in certain embodiments, beginning with an initial, starting, sequence representation, x₀, (e.g., a continuous representation of an amino acid sequence, such as a simplex representation as described herein), an ODE solution approach may be used to integrate velocity predictions obtained via a machine learning model to evolve x₀ to a final, generated sequence representation, x₁. In certain embodiments, determining a final sequence representation x1 comprises repeatedly generating velocity field predictions using a machine learning model and updating a sequence representation, e.g., from xₜ to xₜ+Δₜ.

In certain embodiments, a sequence representation xₜ is updated using a linear push forward function. In certain embodiments, where a machine learning model is used to determine and estimate of a velocity field, e.g., directly, sequence representations may be updated via a linear push forward function according to the following equation:

$${x}_{t + Δ t} = {x}_{t} + \tilde{v} ( x , t ) Δ t .$$

In certain embodiments, where a machine learning model estimates a prospective final sequence representation, the following equations may be used, e.g., to first determine a velocity field based on the prospective final sequence representation, and then used the determined velocity field to update the sequence representation:

$$\tilde{v} ( {x}_{t} , t ) = \frac{{\tilde{x}}_{1} - {x}_{t}}{1 - t}$$

$${x}_{t + Δ t} = {x}_{t} + \tilde{v} ( {x}_{t} , t ) Δ t .$$

In certain embodiments, linear push forward functions may be used in connection with machine learning models having been trained using loss functions such as, without limitation, a MSE for velocity fields and/or prospective final sequence representations, and a cross-entropy loss function for estimates of prospective final sequence representations.

In certain embodiments, a sequence representation xₜ is updated using a conditional path push forward function. As described, for example in Stark et al. (2024) “Dirichlet Flow Matching with Applications to DNA Sequence Design”, (which deals with simpler, nucleotide sequences), a conditional push forward function provides a parameterization of a velocity field as a weighted sum of conditional simplex velocities, where weights p(x₁=eᵢ|xₜ) are learned by a machine learning model (e.g., neural network).

In certain embodiments, where a machine learning model is used to predict p(x₁|xₜ), the following equations may be used for a conditional push forward approach:

$$\tilde{v} ( {x}_{t} , t ) = {\sum }_{i = 1}^{20} \tilde{p} ( {x}_{1} = {e}_{i} \vert {x}_{t} ) c ( {x}_{1} = {e}_{i} \vert {x}_{t} ) , where c ( {x}_{1} = {e}_{i} \vert {x}_{t} ) = {\sum }_{i = 1}^{20} \frac{i - {x}_{t} ( t : )}{1 - t} ,$$

$${x}_{t + Δ t} = {x}_{t} + \tilde{v} ( {x}_{t} , t ) Δ t ,$$

where the conditional probability of xₜ leading to the category variable i and eᵢ is a one hot vector the for i^{th} category. p(x₁=eᵢ|xₜ) is the probability of the xₜ belonging to the class i as given by the neural network prediction, which can be obtained e.g., using raw velocity prediction and apply operation to get an estimate of p(xₜ|x₁=eᵢ).

In certain embodiments, machine learning model generated predictions of v(x, t):

$$\tilde{p} ( {x}_{1} \vert {x}_{t} ) = {x}_{t} + ( 1 - t ) \tilde{v} ( x , t ) ,$$

$$\tilde{u} ( {x}_{t} , t ) = {\sum }_{i = 1}^{20} \tilde{p} ( {x}_{1} = {e}_{i} \vert {x}_{t} ) c ( {x}_{1} = {e}_{i} \vert {x}_{t} ) , where c ( {x}_{1} = {e}_{i} \vert {x}_{t} ) = {\sum }_{i = 1}^{20} \frac{{e}_{i} - {x}_{t} ( i )}{1 - t} ,$$

$${x}_{t + Δ t} = {x}_{t} + \tilde{u} ( {x}_{t} , t ) Δ t .$$

Accordingly, in the above, the probabilities may be determined based on outputs of the machine learning model, for example as values associated with each amino acid time based on a simplex representation. These probabilities can be viewed as magnitudes of vectors associated with each amino acid type, while the quantity c( . . . ) provides a vector direction.

In certain embodiments, applying a push forward function to update sequence representations may result in points outside the simplex. Accordingly, in certain embodiments, for example, to guard against such possibilities, updating a sequence representation comprises projecting an initially updated point back to, for example, a nearest point on the simplex. In this manner, an ODE solver always obtains the input on the simplex.

In certain embodiments, probability distributions may be adjusted to, for example, to be increasingly peaky and/or smoother, for example using temperature annealing. For example, where a machine learning model generates an output hₜ corresponding to an invariant representation within a simplex, a probability p(x₁=eᵢ|xₜ) may be determined using the below equation:

$${\tilde{p}}_{T} ( {x}_{1} = {e}_{i} \vert {x}_{t} ) = \frac{exp ( \frac{{h}_{i}}{T} )}{{\sum }_{k = 1}^{20} exp ( \frac{{h}_{k}}{T} )} .$$

In certain embodiments, an initial updated sequence representation is obtained, for example, using a linear push forward and/or conditional push forward function, as described herein, and adjusted based on its proximity to a nearest corner of a simplex. For example, sequence feature values may be updated according to a sequence velocity field generated by a machine learning model and each amino acid feature connected with a nearest corner on a simplex (e.g., a corner corresponding to its maximum value) and adjusted towards or away from the nearest corner.

In certain embodiments, a norm of a velocity determined by a machine learning model may be adjusted by a scaling factor. Since we always project the final point after the push forward function, the resultant point will still represent a probability distribution over all categorical variables.

Accordingly, as described herein, the present disclosure provides methods and systems that allow for flow matching-based generation of protein and/or peptide sequences. As described in further detail herein sequence generation may be performed unconditionally, to generate de-novo amino acid sequences for arbitrary new protein or peptide structure, or may be conditioned on various desired protein properties, such as particular backbone geometries, partial sequence information, and various categorical, global, protein properties.

Additionally or alternatively, sequence generation may be performed via a dedicated machine learning model (e.g., that does not and/or has not been trained to perform other tasks), or via a multi-task machine learning model, that has been trained not only to generate outputs that can be used to determine sequence velocity fields as described above, but also to generate outputs that are associated with and can be used to generate other velocity fields, such as those used for peptide backbone generation, side chain packing, and/or other tasks.

A detailed experimental example demonstrating flow matching-based sequence generation in various contexts described herein is provided herein in Example 10.

### D. SIDE CHAIN GEOMETRIES

In certain embodiments, flow-matching technologies of the present disclosure include systems and methods for determining side chain packing in a protein structure—that is, generating viable three-dimensional arrangements of side chains in the context of particular protein peptide backbone and/or amino acid sequences. In certain embodiments, flow-matching technologies for side chain generation mirror those described herein for peptide backbone generation and/or sequence design is associated. As described herein, overall flow matching techniques aim effectively learn a joint distribution of protein backbone, sequence, and side chain geometries. In certain embodiments, flow-matching techniques for side chain generation leverage machine learning models to learn a conditional distributions of protein backbone, sequence, and side chains (e.g., instead of the joint distribution).

As described herein, flow-matching-based machine learning techniques for determining side chain geometries utilize, and evolve feature values that represent 3D side chain geometries along, probability paths interpolating between a prior and a final, target distribution. A machine learning model may be trained and used (e.g., at inference) to determine velocity fields that are used to repeatedly update feature values, and thereby push them along these probability paths, for example by utilizing various solutions and/or numerical approaches for evaluating ODEs. As described herein, this machine learning models such as equivariant graph neural networks may be used in this context.

### D.i Side Chain Geometry Encoding

A protein structure comprises of a sequence of amino acids (residues) folded into a 3D geometry. This 3D geometry includes a peptide backbone geometry as well as 3D orientations of the individual amino acid side chains. That is, each residue has four backbone atoms N—Cα-C—O whose 3D geometry may be represented via a local frame representation, such as a two sets of values, (Cα, q), where Cα is the (centered) 3D coordinate of Cα and q∈SU(2) is a unit quaternion representing the frame (orthogonal matrix) of the residue under the map SU(2)→SO(3).

Protein side chains are attached to the Cα atoms and their three-dimensional geometries described by torsion angles, which represent rotations around the bonds within the side chain. These angles, often denoted as χ angles, define the orientation of each part of the side chain relative to the protein backbone and to each other. Each side chain, depending on its size and complexity, can have up to four torsion angles, typically denoted as χ angles (χ₁, χ₂, χ₃, χ₄). The χ₁ angle describes the rotation around the bond between the α-carbon (Cα) and a first carbon of the side chain (usually β-carbon, Cβ). Subsequent χ angles (χ₂, χ₃, etc.) describe rotations around bonds further along the side chain. The specific number and values of these torsion angles determine the three-dimensional conformation of the side chain. By altering these angles, the side chain can adopt various spatial arrangements, affecting the protein's overall structure and interaction capabilities. Precisely describing the side chains' conformations using these torsion angles facilitates understanding protein function and for computational modeling of protein structures.

In certain embodiments, torsion angles may be represented in radians between 0 and 2π. However, in certain cases, this (0 to 2π) representation may be undesirable because values close to 0 and values close to 2π may be treated very differently by a machine learning model, but in reality, should be considered close to each other. Among other things, this representation may produce unwanted discontinuities and resulting in unstable training and poor performance. Accordingly, additionally or alternatively, torsion angles may be represented as a pair of (cos(χ), sin(χ)), which represents an angle χ as a point on the unit circle:

$$S 1 = \{ z ∈ C \vert ‖ z ‖ = 1 \} .$$

This is the coordinate representation of the complex number exp(iχ) using Euler's formula, where i is the imaginary unit. In this manner, a 3D geometry of a side chain may be represented as a tuple of four complex numbers, one for each torsion angle. Since not all side chains require all four torsion angles (e.g., smaller side chains may be described using fewer angles), in certain embodiments, where a side chain less than the maximum, four, torsion angles remaining values may be set to (complex) 0. For example, the 3D geometry of Proline can be described via two torsion angles (χ₁, χ₂), which, in the four-value representation described above, may be represented as (exp(iχ₁), exp(iχ₂), 0, 0). Among other things, this approach may be advantageous as it places non-existing and/or missing angles in a center of the complex plane, equidistant from all data points.

In certain embodiments, a flow matching technique for side chain generation may be setup comprising Riemannian flow on S1. Initial, seed values for side chain geometry features may be uniformly at random on a unit circle, and updated according to velocity fields to travel along interpolating probability paths following geodesics, i.e. connecting an initial prior distribution and a data distribution along arcs. This approach, however, may face challenges and/or suffer from volatility, for example, in situations where a prior and data are approximately opposite to each other on a unit circle, since the corresponding angles differ by roughly π. Accordingly, in certain embodiments, flow matching technologies for side chain generation of the present disclosure leverage this insight, utilize Euclidean geometries on the complex plane. This approach is facilitated via the 4-element complex tuple representation, and may lead to better results than Riemannian flow, allowing for stabilized training and reliable inference.

### D.ii Training and Inference

### (a) Training

Accordingly, in certain embodiments, side chain geometry feature values are complex numbers within a unit disk centered at the origin.

$$D = \{ 𝓏 ∈ ℂ \vert ‖ 𝓏 ‖ \leq 1 \} .$$

Initial starting values, here complex numbers, z₀, may be selected from an initial starting distribution, p₀, which may, for example be a uniform distribution, p₀=U(D), for each residue and each torsion angle on the disk. Training examples may be selected from a training dataset to obtain examples, z₁⁽ⁱ⁾, and used, together with a particular form (e.g., interpolation) of probability path to determine an intermediate feature set at a selected time, t, and corresponding velocity field. For example, in certain embodiments, straight paths between z₀˜p₀ and data z₁˜p_{data} may be used, such that:

$${𝓏}_{t} = ( 1 - t ) {𝓏}_{0} + t {𝓏}_{1} , t ∈ [ 0 , 1 ] .$$

In certain embodiments, a constant velocity, z₁-z₀, such that velocities determined according to:

$${v}_{t} ( 𝓏 ) = 𝔼 [ {𝓏}_{1} - {𝓏}_{0} \vert {𝓏}_{t} = 𝓏 ] .$$

In certain embodiments, each torsion angle may be represented as a tuple, z. In certain embodiments, an initial seed, z₀˜p₀, is sampled independently for each torsion angle and each residue of a protein.

In certain embodiments, as described herein, a neural network is trained to approximate the velocity field vₜ(z), that is, to determine, for a particular current time, t, and set of side chain geometry feature values, zₜ, an estimated velocity field, \hat{v}ₜ(z). During training, this estimated velocity field is compared with the velocity determined via knowledge of a desired final target—i.e., a particular training example, together with a selected/desired path and velocity function. For example, where linear paths and constant velocities are used, as described above, the below loss function is used:

$${ℒ}_{t}^{FM} ( z ) = {𝔼}_{{z}_{0} , {z}_{1}} [ {‖ {\hat{v}}_{t} - ( {z}_{1} - {z}_{0} ) ‖}^{2} ] .$$

In certain embodiments, training may also utilize dynamic weights, to account for the fact that the empirical distribution of the 20 amino acids (AAs) in proteins is not uniform, i.e. different amino acids occur with varying frequencies. In certain embodiments, non-uniform distribution of AAs in a protein affects training a model on protein data by directing the model to learn the properties of the frequently occurring AAs more accurately, while downplaying the intricacies of less frequently occurring AAs. Accordingly, to address this (e.g., bias), in certain embodiments, training approaches incorporate dynamic weights that scale loss based on amino acid type, in relation to their (e.g., empirical) frequency. For example, in certain embodiments, dynamical weighting comprises computing, for each batch b of proteins an empirical distribution β(b) and multiply the flow matching loss by the value of the inverse 1/β(b) for each residue in the condition mask, according to its amino acid type.

In certain embodiments, additionally or alternatively, auxiliary loss terms may be used, for example to reflect, and penalize deviations from, known properties and/or requirements of physical protein structure. For example, an auxiliary loss may be used to enforce avoidance of steric clashes, since physical protein structures exhibit little to no steric clashes. In certain embodiments, an auxiliary loss function associated with steric clashes comprises an auxiliary loss γ(t) [symbol] ^{Aux}, which is a time dependent penalty for steric clashes. In certain embodiments, the auxiliary loss is associated only with sufficiently large time values. In certain embodiments, the auxiliary loss is associated with t>0.75. Such late cutoff is associated with the understanding/belief that a model should not be penalized at early stages of the probability paths when the model is still exploring various directions.

### (b) Inference

The model uses learned vector field \hat{v}ₜ(b, z) to generate torsion angles following the ordinary differential equation (ODE) dynamics

$$d \hat{z} = \hat{{v}_{t}} ( b , z ) dt .$$

The vector field determines the trajectory of the complex number 2 on its way from an initial seed point \hat{z}₀ to a final set of feature values \hat{z}₁ as time t evolves from 0 to 1. At inference time, an initial starting distribution may be chosen freely to accommodate for preferences regarding accuracy, novelty, or diversity of the generations. However, perturbing the starting distribution at inference time comes with the caveat of introducing a bias in the conditional distributions generated by following the learned vector field.

At each time step, a prediction of the torsion angle \hat{χ} as the phase of the complex number is obtained, which amounts to projecting \hat{z} onto the unit circle S¹ and computing its angle, i.e.

$$\frac{\hat{z}}{‖ \hat{z} ‖} = exp ( i χ ) .$$

For each \hat{z} along the trajectory the predicted torsion angles \hat{χ} was then used to generate the coordinates of all atoms in the protein structure. The amino acid type of each residue determines the number and type of the atoms in the side chain. Finally, the full 3D protein structure may be obtained by rotating the groups of atoms around their bonds, one group at a time, using the predicted torsion angles.

In certain embodiments, side chain geometries depend on a particular type of amino acid, and accordingly, approaches for predicting side chain geometries may first receive, as input, an amino acid sequence, e.g., thereby identifying a particular amino acid type at each location, whose detailed three-dimensional geometry may then be determined via the flow-matching technique described above. Amino acid sequences used in this manner may be determined in a variety of fashions, including, without limitation, via the sequence generation approaches described herein. For example, in certain embodiments, a single model may be trained to perform both sequence generation and side chain geometry determination, and used twice, e.g., in succession, once to generate an amino acid sequence and then to determine geometries for each side chain thereof. Additionally or alternatively, in certain embodiments, side chain geometries and sequence may be determined jointly, at the same time, by a single flow-matching model trained to perform both sequence generation and side chain geometry prediction, as described herein.

In certain embodiments, additionally or alternatively, the procedure described herein with regard to generation of x angles, can be applied to the generation of bond angles. By adapting this procedure, capture the nuanced dynamics of bond angles, instead of building atoms positions based on the ideal angel assumptions. This extension not only demonstrates the versatility of the original method but could enable more accurate modeling of molecular geometries.

### E. MACHINE LEARNING MODEL ARCHITECTURES

A variety of machine learning architectures may be utilized in connection with peptide backbone generation technologies described herein. Particular architectures may include, without limitation, transformers, graph neural networks (GNNs), auto-regressive networks, encoders, decoders, and combinations thereof.

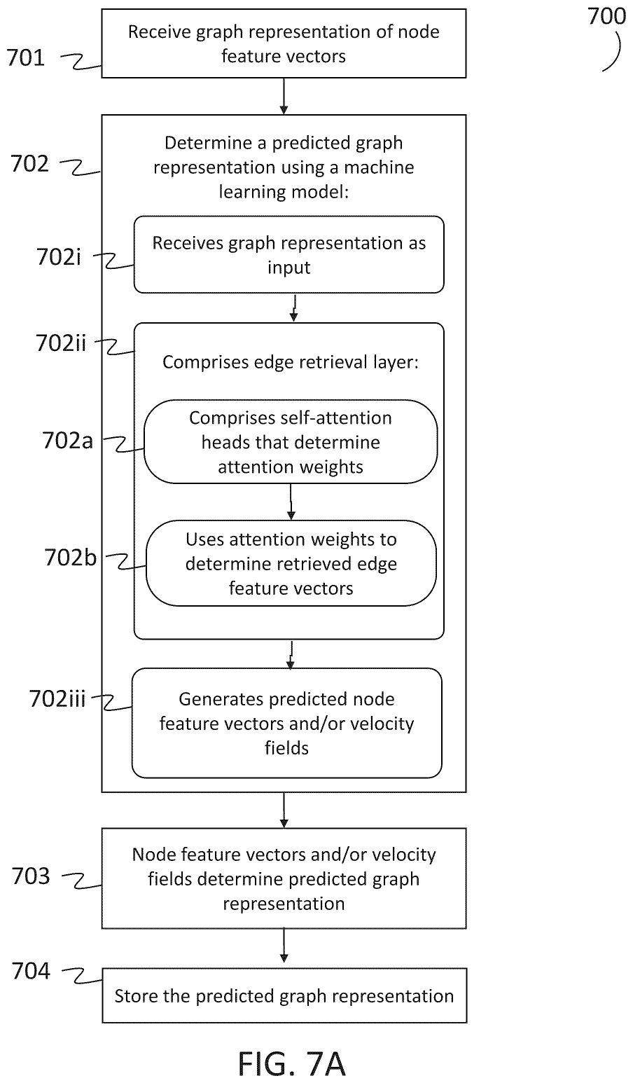

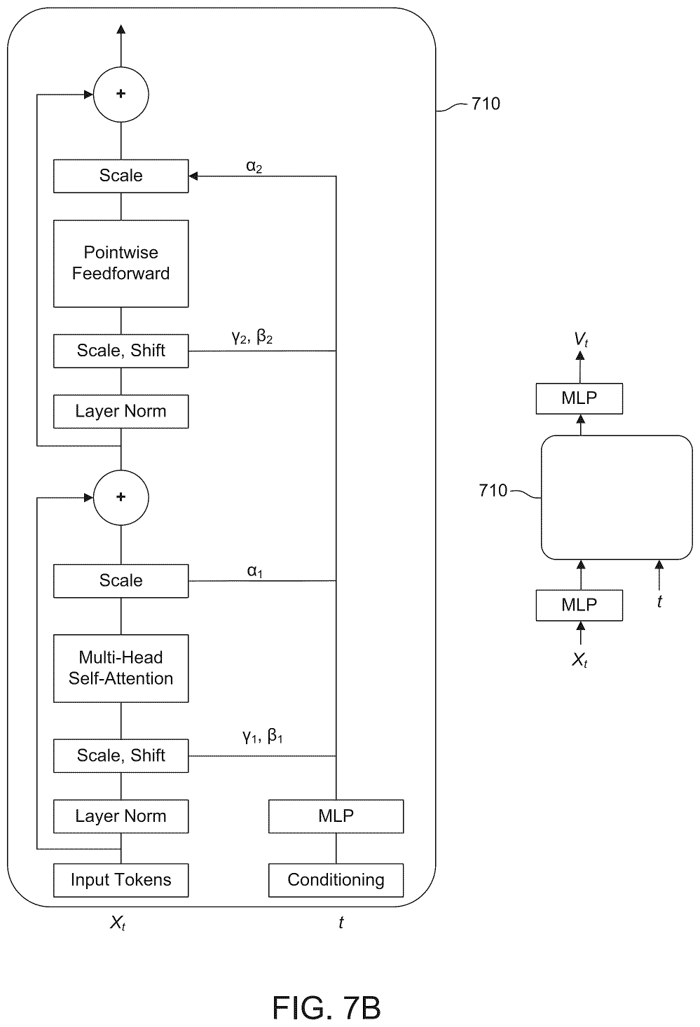

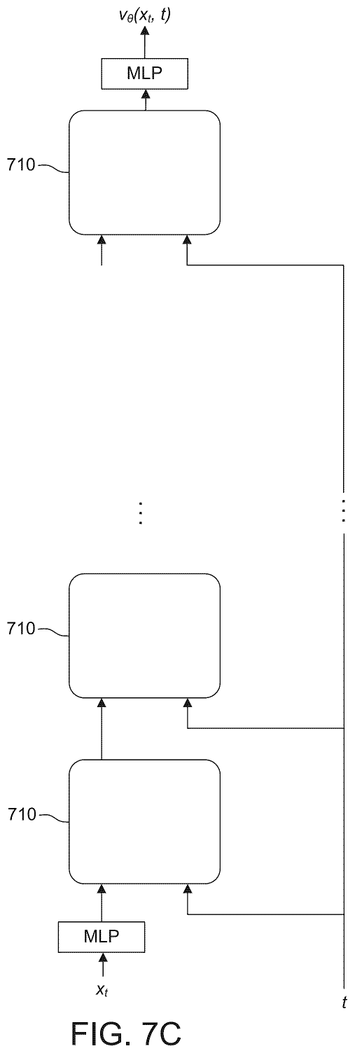

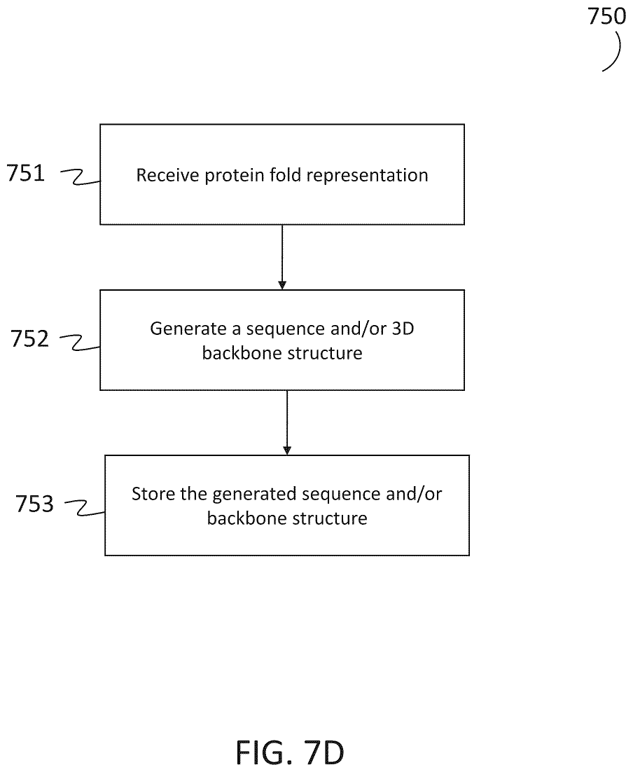

FIG. 7A shows an example process 700 for (e.g., memory efficient) generating attention-based deep-learning predictions from input graph representations. An initial graph representation may be received and/or accessed 701, for example retrieved from memory, either locally or on a PACS server, cloud, etc. A predicted graph representation may be determined 702 using a machine learning model. This determination may involve the following steps. The machine learning model may receive 702i the initial graph representation as input. The machine learning model may comprise 702ii edge retrieval layers. The edge retrieval layers may, in turn, comprise self-attention heads to determine 702a attention weights. The attention weights may be used to determine 702b values of retrieved edge feature vectors. The machine learning model may generate 702iii as output predicted node feature vectors and/or velocity fields based at least in part on the retrieved edge feature vectors. The predicted node feature vectors and/or velocity fields determine 703 the predicted graph representation. Once determined, the predicted graph representation may be stored, provided for display, and/or provided for further processing 704.

FIGS. 7B and 7C illustrate an example transformer-encoder architecture, used in certain embodiments. As shown in FIG. 7B, block 710 receives, as input, a feature set comprising relative positions and orientations of each backbone site and a (e.g., current) time value. Each backbone site is represented via a translation and rotation of its local frame relative to that of a preceding backbone site. As described herein, translations and rotations may be represented via sets of three and four values, respectively, such that input x, to block 710 can be viewed as a list of seven element vectors, each corresponding to and representing a position and orientation of a particular backbone site.

In certain embodiments, input xx is encoded via a Multi-Layer Perceptron (MLP), e.g., before being fed to transformer block 710, as illustrated in the right-hand side of FIG. 7B. In certain embodiments, velocity is generated as output from a transformer block 710, following a final MLP. In certain embodiments, a positional embedding is used to encode a position (e.g., location in a sequence) of each backbone site, for example using an approach as described in A. Vaswani et al., “Attention is all you need,” NIPS 2017 the content of which is hereby incorporated by reference in its entirety. In certain embodiments, a bi-directional positional encoding is used, such that for a particular backbone site i, of a peptide of length n, position i and position n−i are both encoded, so as to provide transformer block 710 information about how far a particular backbone site is from a start and end of a sequence. In certain embodiments, bidirectional positional encoding is used for edges. In certain embodiments, relative positional encoding is used for edges.

In certain embodiments, a current time value is received as input, e.g., as a feature. Additionally or alternatively, in certain embodiments, a current time value is not used as a feature and (e.g., instead) is inputted, separately, and used to condition transformer intermediate representations. For example, as shown in FIG. 7B, a time value is received as input, encoded by a multi-layer perceptron (MLP) and the resultant encoding used to modulate intermediate features that are learned by a transformer.

In certain embodiments, for example as shown in FIG. 7C, multiple (e.g., a plurality of) blocks 710 are stacked in sequence, with output from one block feeding into a next.

While FIGS. 7B and 7C show a particular machine learning structure, as described herein, other machine learning structures may be used in various embodiments.

### F. MULTI-INPUT AND MULTI-MODAL PEPTIDE/PROTEIN DESIGN

In certain embodiments, the present disclosure includes a multi-model polypeptide generation technology whereby scaffold model generation techniques described herein are adapted and extended to allow for generation of (i) scaffold models representing peptide backbones and/or (ii) sequence data, representing amino acid sequences. Moreover, in certain embodiments, scaffold and/or polypeptide generation approaches described herein can receive a variety of inputs on which to condition scaffold model and/or sequence data generation.

Generative scaffold design technologies described herein can, for example be used to create scaffold models based on a variety of initial conditions, input information, and for a variety of purposes.

### F.i Unconditional Generation

Scaffold models can, for example, be generated without an initial conditioning, for example to generate new scaffold models representing new peptide backbones of various sizes (e.g., a number of amino acid sites may be fixed). In certain embodiments, sequence data can also be generated, for example together with a scaffold model. A scaffold model and its sequence can, for example, be generated without conditioning on initial features or properties, so as to allow for creation of a de-novo protein and/or peptide including, not only a viable 3D structure, but also a corresponding sequence (e.g., predicted to fold into the 3D structure).

F.ii Conditioning on Backbone Structure and/or Sequence

In certain embodiments, scaffold generation can be conditioned on various initial scaffold structures and/or sequences. For example, in certain embodiments, scaffold models can be generated based on—that is, conditioned on—various initial partial scaffolds.

For example, scaffold generation can be conditioned on an input target scaffold model that represents a known and desired target, such that generated scaffold models represent custom candidate peptide backbones that are favorable for binding to the target.

Additionally, or alternatively, scaffold generation can be conditioned on a pre-existing partial scaffold that represents a partially known and/or desired backbone structure, such that generated scaffolds fill in (e.g., inpaint) unknown and/or variable regions.

In certain embodiments, scaffold generation techniques can be used to dock two known protein scaffolds.

In certain embodiments, scaffolds can be conditioned on oligomeric state.

In certain embodiments, sequence data may be received as input and/or generated as output. For example, in certain embodiments, generative technologies described herein can be used to generate a sequence, conditioned on a particular scaffold model. In certain embodiments, scaffold models can be generated conditioned on an amino acid sequence (folding problem). In certain embodiments, scaffold models can be repeatedly generated, conditioned on a particular sequence. For each new scaffold model, a different initial condition is sampled from a prior/initial distribution and, accordingly, this statistical approach can be used to generate a plurality of scaffold models for a particular sequence, thereby sampling the conformational landscape of potential folded backbones associated with a particular sequence. These possible conformations can be used to assess energy, stability, and other properties of a particular molecule, for a variety of scientific and practical applications.

In certain embodiments, generative technologies of the present disclosure can be used to dock two known proteins having a particular known monomeric structure and/or sequence.

### F.iii Conditioning on Global Properties

In certain embodiments, categorical variables can also be received as input, and used for conditioning. These may include, for example, protein family, thermophilly, immunology, function, solubility, pH sensitivity, etc.

### F.iv Conditioning on Node Properties

Additional node properties can also be used for conditioning. These may include, for example, amino acid type (e.g., via a one-hot encoding), a polarity categorical variable (e.g., having 5 possible polarities-polar, apolar, negatively charged, positively charged), burriedness (e.g., a binary variable indicating whether a node is at a surface or core), as well as secondary structure (e.g., a variable encoding whether a node is part of a helix or beta-sheet).

In certain embodiment, a binary variable can be used to identify and/or pre-define those nodes that represent hotspots—i.e., amino-acid sites that lie within a particular threshold distance of another molecule, such as a target and/or other chain. Hotspot identification approaches are described in further detail, e.g., in U.S. Pat. No. 11,450,407, issued Sep. 20, 2022, the content of which is hereby incorporated by reference in its entirety.

For example, in certain embodiments, sites referred to as “hotspots” may be identified on a ligand and/or receptor. For a ligand, hotspots refer to sites which, when occupied by an amino acid side chain, place at least a portion of the amino acid side chain in proximity to one or more side chains and/or atoms of the receptor. Likewise, for a receptor, hotspots are sites which, when occupied via an amino acid side chain, place at least a portion of the amino acid side chain in proximity to one or more side chains and/or atoms of the ligand.

In certain embodiments, for example since size, geometry, and orientation of various acid side chains may vary, hotspots may be identified based on distances between beta carbon (Cβ) atoms of a ligand and receptor of a complex. For example, a ligand hotspot may be identified as a particular site on the ligand that, when occupied by an amino acid side chain, will place a Cβ atom of the side chain located at the site within a threshold distance of a Cβ atom of the receptor. Receptor hotspots may be identified analogously. Since Cβ atoms are common to every amino acid side chain apart from Glycine, this approach provides a uniform criterion for identifying hotspots, independent of a particular amino acid that occupies a particular site. In certain embodiments, in the singular case where a Glycine residue occupies a particular site, Glycine's hydrogen atom may be used in place of a Cβ, but hotspots identified in an otherwise identical fashion. Additionally or alternatively, in certain embodiments, distances between alpha-carbons (Cα) associated with amino-acid sites may be determined, e.g., in a similar manner to which distances between Cβ atoms are determined. In this manner, Cα distances may be compared with various threshold values to identify hotspots.

Various threshold distances may be used for identification of hotspots. For example, in certain embodiments, a hotspot threshold distance of 8 Å (i.e., 8 Ångstroms) is used. In some embodiments, other thresholds may be used for defining a hotspot (such as less than 3 Å, less than 4 Å, less than 5 Å, less than 6 Å, less than 7 Å, less than 9 Å, less than 10 Å, less than 12 Å, less than 15 Å, less than 20 Å, as well as other suitable thresholds).

In certain embodiments, hotspots may be identified based on comparison of values computed by various functions—e.g., of one or both of a Cα and Cβ distance—with one or more threshold values. Such functions may take into account features such as bond angles, surface area, etc.

### F.v Conditioning on Protein Folds

Protein topological properties can also be used for conditioning. These may include properties of protein secondary structure, folding properties. In certain embodiments, protein topological properties may be represented by a block adjacency matrix.

A “fold family” refers to a group of proteins that share a similar three-dimensional structure or fold, despite potentially having different amino acid sequences. Proteins with similar folds often exhibit similar overall shapes and structural features, even if their primary sequences (the order of amino acids) differ. While the specific sequence of amino acids determines protein's unique structure and function, certain structural motifs, or folds, repeat themselves throughout different proteins. These folds can be highly conserved across evolution, and proteins with the same or similar folds often perform related functions.

Understanding fold families is crucial in protein design and engineering because it allows scientists to predict the structure and function of a newly designed protein based on the known characteristics of proteins with similar folds. This knowledge aids in designing proteins with desired functions, such as enzymes for specific biochemical reactions or proteins for therapeutic purposes. It also facilitates the exploration of structure-function relationships in proteins.

FIG. 7D shows an example process 750 for designing a custom biologic having a three-dimensional (e.g., fold) structure belonging to a desired fold family according to various embodiments described herein. Protein fold representation may be received and/or accessed 751, for example retrieved from memory, either locally or on a PACS server, cloud, etc. The protein fold representation may encode and represent desired three-dimensional structural features of the desired fold family and present within the custom biologic. A sequence and/or a three-dimensional peptide backbone structure may be generated 752, using a machine learning model, based on the protein fold representation. The generated sequence and/or 3D peptide backbone structure may be stored, provided for display, and/or provided for further processing 753.

The block adjacency matrix (BAM) represents the adjacency relationships between different secondary structure segments of a protein based on a specified distance cutoff. In certain embodiments, BAM is calculated based on protein's Secondary Structure Elements (SSE), backbone coordinates, and a cutoff distance. SSE may be represented by a (e.g., one-dimensional) tensor, where values of the tensor represent a numeric encoding of SSEs at each position in the protein sequence. The numeric encoding may be as follows: 0 for Helix, 1 for Strand, 2 for Loop, and 3 for Masked regions. The backbone coordinates may be represented by a tensor containing (e.g., Cartesian) coordinates of the backbone N, Ca, and C atoms for each residue in the protein. The cutoff value (e.g., 6.0 Angstroms) is used to determine whether two residues are considered adjacent. In certain embodiments, BAM calculations comprise inclusion criteria. In certain embodiments, the inclusion criteria comprise a flag to denote whether certain regions (e.g., loop regions) should be included as contacts in the block adjacency matrix.

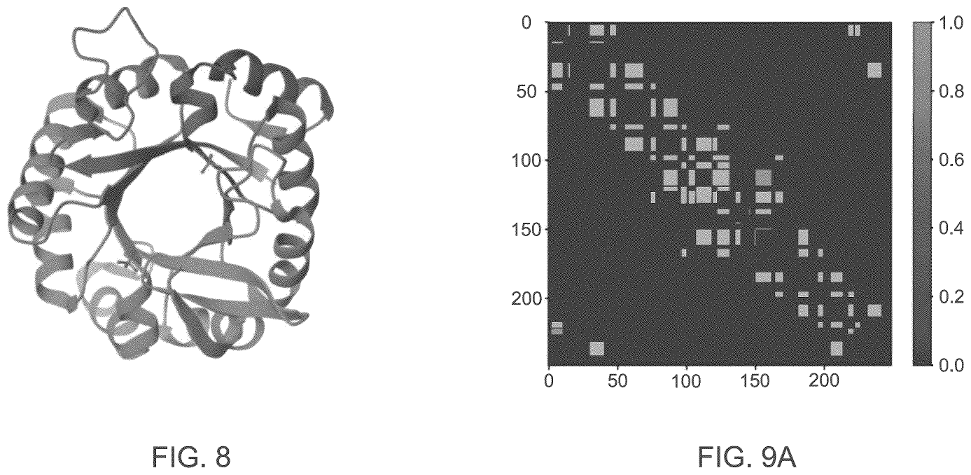

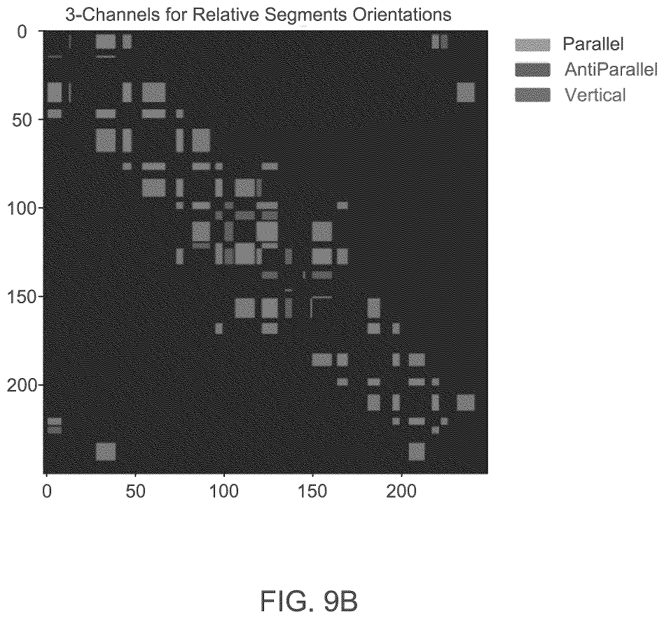

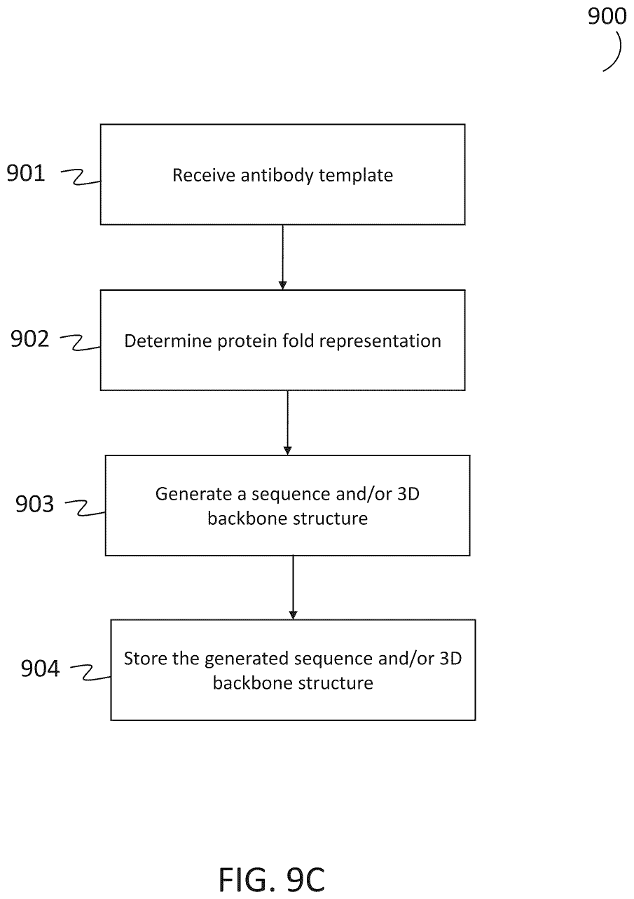

A block adjacency matrix may be represented by a (e.g., 2D binary) matrix (e.g., of size (L, L), where L is the length of the protein sequence). For example, each element in the matrix is set to 1 if the corresponding SSEs are considered adjacent based on the distance cutoff and the inclusion criteria. For example, FIG. 8 shows a TIM barrel secondary structure and FIG. 9A shows a block adjacency matrix calculated for a TIM barrel according to methods described herein. In certain embodiments, BAM calculations, additionally or alternatively, produce a segment identification and/or a block adjacency construction. The segment identification comprises values associated with identification of continuous segments of the same SSE type and values associated with start and end indices of these continuous segments. The block adjacency construction comprises values associated with a check if any pair of residues (e.g., one from each segment) falls within the specified distance cutoff (e.g., if a pair distance is below the cutoff distance, the corresponding block in the adjacency matrix is filled with ones, indicating adjacency).

In certain embodiments, additional information about the topology of secondary structure elements (SSEs) in protein structures may be incorporated into (e.g., concatenated with) BAM, providing a comprehensive representation of the protein's structural and topological features. For example, the topological relationships between SSEs (e.g., as parallel, antiparallel, or vertical) may be classified and included into BAM. BAM may be represented as a (e.g, binary) matrix (e.g., of size (L, L, 4), where L is the length of the protein sequence). FIG. 9B shows a multi-channel block adjacency matrix calculated for a TIM barrel according to methods described herein. As a result, BAM comprises (e.g., binary; binned; one hot vector) values associated with topology classification (e.g., classified as parallel, antiparallel, and vertical).

The axis of each secondary structure segment is calculated using the coordinates of the first five (e.g., three, ten) residues in the SSE and the SSE type (e.g., alpha or beta). The orientation between two secondary structure segments is determined by the angle between their axes. The classification criteria for topology classification may be as follows: (1) Parallel-if the angle between the axes is less than 60 (e.g., 30, 45) degrees; (2) Antiparallel-if the angle is greater than 120 (e.g., 150, 135, respectively) degrees; (3) Vertical-if the angle is between 60 (e.g., 30, 45) and 120 (e.g., 150, 135, respectively) degrees.

### G. CUSTOM BIOLOGIC DESIGN VIA FLOW-MATCHING

In certain embodiments, generative technologies described herein may also include conditioning approaches, so as to produce generated scaffold models that represent peptide backbones having desired features such as particular properties, structural motifs, etc. In certain embodiments, conditioning may be used in connection with a representation of one or more prospective binding sites of a target molecule (e.g., protein and/or peptide), thereby allowing for creation of a de-novo peptide backbone suitably for binding to the target molecule (e.g., at a location within or comprising at least one of the one or more prospective binding sites).

In certain embodiments, generated scaffold models representing de-novo peptide backbones may be used as input to an interface designer module (e.g., comprising a machine learning model) for generating an amino acid interface for binding to a target molecule. Interface designer approaches, that, for example, populate scaffold models with amino acid sequences in order to create custom interfaces for binding to particular targets are described in further detail, for example, in International publication WO 2023/004116 A1 of PCT/US22/38014, entitled “SYSTEMS AND METHODS FOR ARTIFICIAL INTELLIGENCE-GUIDED BIOMOLECULE DESIGN AND ASSESSMENT,” and filed Jul. 22, 2022, included herein as an Appendix and the content of which is hereby incorporated by reference in its entirety.

### G.i Application Examples for Sequence Generation

This section describes frameworks of using flow matching on protein sequences for various applications.

### (a) Unconditional Sequence Generation

In certain embodiments, generative technologies described herein may be used to produce a protein sequence from scratch (e.g., without specifying any explicit conditions or context to guide the generation process). For example, a model may be trained on sequence data only (e.g., no structural information), and use as input:
Nodes simplicial
Sequence position
Time
Any conditioning on node and/or global properties is masked

### (b) Conditional Sequence Generation

In certain embodiments, generative technologies described herein may be used to generate a protein sequence from scratch, by specifying explicitly conditions or context to guide the generation process. For example, a model may use as input:
Nodes simplicial representation (xₜ), i.e. a point in the 19-dimensional simplex as explained in the present disclosure.
Sequence position
Time
Any conditioning on node and/or global properties.

In certain embodiments, generative technologies described herein may be used to generate a protein sequence that is predicted to fold into a specific three-dimensional structure (e.g., backbone). For example, a model may use as input:
Nodes simplicial representation (xₜ), i.e. a point in the 19-dimensional simplex as explained in the present disclosure
Sequence position
Time
Position of amino acids (e.g., CA position)
Backbone orientation (e.g., Canocical frames)
Relatative information (e.g., edges, 6DOF)
Any conditioning on node and/or global properties.

### (c) Co-Design

In certain embodiments, generative technologies described herein may be used to generate both a protein backbone and a protein sequence. For example, a model may be trained on the sum of two loss objectives associated with backbone generation and sequence generation. The model may perform all tasks as related to protein generation and sequence generation as described in the present disclosure. The model may receive all conditioning associated with protein generation tasks and sequence generation tasks as described in the present disclosure. Additionally or alternatively, the model may replace one-hot encoding representation of amino acids as used, for example, for backbone generation with the corresponding values on the simplex as used, for example, for sequence generation.

In certain embodiments, generative technologies described herein may be used to generate both a protein side chain and a protein sequence conditioned on a protein backbone. For example, a model may be trained on the sum of two loss objectives associated with side chain generation and sequence generation. The model may perform all tasks as related to side chain generation and sequence generation as described in the present disclosure. The model may receive all conditioning associated with side chain generation tasks and sequence generation tasks as described in the present disclosure. Additionally or alternatively, the model may replace one-hot encoding representation of amino acids as used, for example, for side chain generation with the corresponding values on the simplex as used, for example, for sequence generation.

In certain embodiments, generative technologies described herein may be used to generate a protein backbone, a protein side chain, and a protein sequence unconditionally. For example, a model may be trained on the sum of three loss objectives associated with backbone generation, side chain geometry prediction, and sequence generation. In certain embodiments, for side chain geometry generation, for example, multiple predictions of side chain geometries may be generated assuming all 20 amino acids for each location and then combined using the probability of each amino acid type, at each location throughout the protein. In certain embodiments, loss objectives for three tasks may remain separated and/or be trained using different weights.

### G.ii Application Examples for Fold Conditioning

This section describes frameworks of using flow matching with fold conditioning for applications in designing de novo antibody/nanobody that binds antigens.

(a) Inpainting from a Known Pose

The determinant of the antigen, referred to as epitope, is the part of the antigen that is typically recognized by an antibody. Epitopes are typically linear (i.e. a continuous sequence segment) or conformational (i.e. discontinuous fragments in sequence space but adjacent in 3D space).

Given an antibody/epitope complex, the inpainting task refers to designing a complementary region of the antibody that can specifically and tightly bind to the epitope. The task involves manipulating the complementarity-determining regions (CDRs) of the antibody, which are the parts of the antibody that directly interact with the epitope. Any part that is masked is generated by the model.

CDRs design. In certain embodiments, generative technologies described herein may be used to design de novo CDR regions of an antibody that is interacting with a given target antigen with high specificity and affinity. For example, an input representation at t=0 may take form:
1. Antibody:
   a. Available: everything except CDRs
   b. Masked: CDR coordinates and sequence (optional)
2. Antigen:
   a. Available: everything
   b. Masked: nothing
      The coordinates and (optionally) the sequence of the CDRs are generated as an output.

Redesign of CDRs can potentially improve many physico-chemical and PK properties of the antibody. For example:
Increasing Affinity: Modifying CDRs to enhance the strength of the interaction between the antibody and the antigen
Improving Specificity: Ensuring the antibody binds only to the intended antigen and/or not to similar structures on other molecules, thereby reducing off-target effects.
Improving thermodynamic and thermal stability: Designing CDRs that maintain their structure and function under physiological conditions, improving the durability and shelf-life of antibodies.
Reducing Immunogenicity: Modifying or “humanizing” CDRs in therapeutic antibodies derived from non-human sources to prevent them from being recognized as foreign by the human immune system.
Identification of critical hotspots through mutations:
   Mutations: simulate how CDR's mutations affect binding regions
   Insertions/deletion: extend/shorten the length of CDR to affect the binding regions

Epitope relaxation. In certain embodiments, generative technologies described herein may be used to design de novo epitope interfaces of an antigen interacting with a given antibody with high specificity and affinity. This application opens new avenues in terms of targeting strategies of specific therapeutic targets by expanding the universe of possible epitopes.

For example, input representation at t=0 may take form:
1. Antibody:
   a. Available: everything
   b. Masked: nothing
2. Antigen:
   a. Available: everything excepts epitope coordinates
   b. Masked: epitope coordinates
      The coordinates of the epitope may be generated as an output.

Examples of epitope relaxation:
Increased Specificity/Immunogenicity: Modify the epitope to ensure that the immune response is highly specific to the target antigen, reducing the risk of cross-reactivity with non-target antigens.
Improved Stability: Enhance the physical and chemical stability of the epitope, ensuring that it maintains its structure and immunogenicity under a wide range of conditions.
Mutations: Simulate how epitopes' mutation affect the binding regions.

Epitope relaxation+CDRs design. In certain embodiments, generative technologies described herein may be applied in various ways to achieve both objectives of epitope relaxation and CDRs design as described herein. For example, iteratively repeating for any arbitrary number of times: (1) Epitope relaxation objective then CDRs design objective or (2) CDRs design objective then Epitope relaxation objective. In certain embodiments, epitope relaxation as well as the CDRs design may be performed at the same time. For example, both objectives are achieved simultaneously. For example, input representation at t=0 may take form:
1. Antibody:
   a. Available: everything except CDRs
   b. Masked: coordinates and sequence
2. Antigen:
   a. Available: everything except epitope coordinates
   b. Masked: epitope coordinates
      (b) Antibody Design from an Unknown Pose

Any information used from existing antibody (e.g., BAM, SSE) may be modified/fine-tuned to get full control on the conditioning. For example, the general fold of the antibody to design may be given (e.g., through BAM) or one may start with internal coordinates of the framework (i.e. non-CDRs regions) and simultaneously dock and design an antibody.

Moreover, the framework may be modified through insertion and/or deletion on different parts of the framework. It would then affect only the length of the framework. If insertion are used, it would just be considered as unknown for the model. If deletion are used, only the length will be affected but the information elsewhere will remain the same.

Any masked information sequence, position, and side chains for the antibody may be generated and (optionally) simultaneously position and side chains on the epitope can also be generated. Hence, multiple levels of given input to condition on (on top of the target itself) may be used.

FIG. 9C shows an example process 900 for designing a custom antibody for binding to a target, according to various embodiments described herein. Antibody template representing a sequence and/or three-dimensional structure of a reference antibody may be received and/or accessed 901, for example retrieved from memory, either locally or on a PACS server, cloud, etc. The antibody template may comprise: (i) a base portion located about CDRs of the reference antibody, but (the base portion) excluding CDRs themselves, and (ii) CDRs portion, each CDR portion associated with and comprising a CDR of the reference antibody. Protein fold representation that encodes and represents three-dimensional structural features of the base portion of the antibody template may be determined 902. A sequence and/or a three-dimensional peptide backbone structure may be generated 903, using a machine learning model, based on the protein fold representation. The generated sequence and/or three-dimensional peptide backbone structure may include custom CDRs. The generated sequence and/or 3D peptide backbone structure may be stored, provided for display, and/or provided for further processing 904.

New antibody generation conditioned on fold. In certain embodiments, generative technologies described herein may be used to design de novo antibodies conditioned on specific fold properties to interact with a given antigen with high specificity and affinity. For example, by utilizing one or more existing antibodies, a BAM and/or SSE may be calculated to then inform the conditioning of the generation process. In doing this, de novo antibodies may be created through loose conditioning, with the expectation of discovering new antibody folds. The CDRs are not conditioned to give them full freedom at generation time. For example, input representation at t=0 may take form:

### 1. Antibody:

a. Available: partial BAM and SSE (e.g., masked CDRs) for heavy and/or light chains (e.g., structural elements of the antibody)
b. Masked: coordinates and sequence

### 2. Antigen:

a. Available: everything
b. Masked: nothing
   The coordinates as well as the sequence of the antibody are generated as an output.

New antibody generation conditioned on fold with partial sequence. In certain embodiments, generative technologies described herein may be used to design de novo antibodies conditioned on specific fold properties with partial sequence to interact with a given antigen with high specificity and affinity. The inclusion of partial sequence may enforce even more the conditioning on parts of the antibody. For example, the sequence of an existing antibody from the light and/or heavy chains is partially kept. For example, input representation at t=0 may take form:

### 1. Antibody:

a. Available: partial sequence, partial BAM and SSE (e.g. masked CDRs) for heavy and/or light chains
b. Masked: coordinates and partial sequence

### 2. Antigen:

a. Available: everything
b. Masked: nothing
   The coordinates as well as the masked sequence of the antibody are generated as an output.

New antibody generation conditioned on scaffold. In certain embodiments, generative technologies described herein may be used to design de novo antibodies conditioned on specific scaffold properties to interact with a given antigen with high specificity and affinity. For example, by utilizing one or more existing antibodies, six degrees of freedom (6DOF) may be calculated, which may further inform the conditioning of the generation process. In doing this, de novo antibodies may be created through stronger conditioning, with the expectation of discovering new antibody folds. The CDRs are not conditioned to give them full freedom at generation time. For example, input representation at t=0 may take form:

### 1. Antibody:

a. Available: partial 6 DOFs (e.g. masked CDRs) for heavy and/or light chains
b. Masked: coordinates and sequence

### 2. Antigen:

a. Available: everything
b. Masked: nothing
   The coordinates as well as the sequence of the antibody are generated as an output.

New antibody generation conditioned on scaffold with partial sequence. In certain embodiments, generative technologies described herein may be used to design de novo antibodies conditioned on specific scaffold properties with partial sequence to interact with a given antigen with high specificity and affinity. For example, partially keeping the sequence of an existing antibody from the light and/or heavy chains may enforce the conditioning on parts of the antibody. For example, input representation at t=0 may take form:

### 1. Antibody:

a. Available: partial sequence, partial 6 DOFs (e.g. masked CDRs) for heavy and/or light chains
b. Masked: coordinates and partial sequence

### 2. Antigen:

a. Available: everything
b. Masked: nothing
   The coordinates as well as the masked sequence of the antibody are generated as an output.

### (c) Docking

In certain embodiments, generative technologies described herein may be used to design a docking process (e.g., values associated with two polypeptides interacting with each other; e.g., optimal configuration of molecules that results in the highest binding affinity) for a given antibody and an antigen. For example, input representation at t=0 may take form:

### 1. Antibody:

a. Available: BAM (optional), SSE (optional), Sequence, 6 DOF for heavy and/or light chains
b. Masked: coordinates (optional)

### 2. Antigen:

a. Available: BAM (optional), SSE (optional), Sequence, 6 DOF for heavy and/or light chains
b. Masked: coordinates (optional)
   Generated output may comprise either: (i) the antibody coordinates (e.g., initially masked) and make the antigen coordinates available in input; (ii) the antigen coordinates (e.g., initially masked) and make the antibody coordinates available in input; or (iii) the antibody and antigen coordinates (e.g., initially masked).

### (d) Relaxation

In certain embodiments, generative technologies described herein for applications described herein may comprise (e.g., simultaneous) relaxation of an epitope of an antigen. For example, relaxation of the epitope may be performed by adapting the antigen representation as follows. For example, input representation at t=0 may take form:

### Antigen:

1. Available: everything except on epitope
2. Masked on antigen: coordinates of the epitope

Additionally or alternatively, relaxation may be performed first on an antibody as described in, for example, (Antibody design from an unknown pose) or (Docking) and then apply relaxation performed on an antigen as described in, for example (Epitope relaxation).

### G.iii Synthesis of Custom Biologics

In certain embodiments, custom proteins and/or peptides are synthesized according to in-silico designed—e.g., sequences—determined herein. For example, in certain embodiments, once an amino acid sequence of a particular custom biologic is designed, e.g., as described herein, polynucleotides may be designed that encode the desired amino acid sequence. Such custom sequences may be or comprise sequences of antibodies or antigen-binding fragments thereof, e.g., as provided herein. Accordingly, the present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein.

In some embodiments, the present disclosure provides polynucleotides which encode at least one CDR region, such as at least two, such as at least three CDR regions from the heavy or light chain of an antibody provided herein. In some embodiments, polynucleotides encode all or substantially all of the variable region sequence of the heavy chain and/or the light chain of an antibody.

Nucleic acid sequences can be produced by de novo solid-phase DNA synthesis or by PCR mutagenesis of an existing sequence (e.g., sequences as provided in the Examples below) encoding an antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a nucleic acid sequence by PCR can be performed as described in, e.g., PCR Technology: Principles and Applications for DNA Amplification, H. A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Various expression vectors can be employed to express polynucleotides encoding custom biologics, such as antibodies, or antigen-binding fragments thereof. Both viral-based and nonviral expression vectors can be used to produce antibodies or antigen-binding fragments thereof in a mammalian host cell. Nonviral vectors and systems include plasmids, episomal vectors, typically with an expression cassette for expressing a protein or RNA, and human artificial chromosomes (see, e.g., Harrington et al., Nat Genet 15:345, 1997). Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an antibody or antigen-binding fragment thereof. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of custom biologics, such as an antibody or antigen-binding fragment thereof.

Host cells for harboring and expressing various custom biologics generated in accordance with systems and methods of the present disclosure may be either prokaryotic or eukaryotic. E. coli is one prokaryotic host useful for cloning and expressing polynucleotides. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation.

In some embodiments, mammalian host cells may be used. These include any normal mortal or normal or abnormal immortal animal or human cell. For example, a number of suitable host cell lines capable of secreting intact immunoglobulins have been developed including the CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable.

Methods for introducing expression vectors containing the nucleic acid sequences of interest vary depending on the type of cellular host. For example, calcium chloride transfection is commonly utilized for prokaryotic cells, whereas calcium phosphate treatment or electroporation may be used for other cellular hosts. Other methods include, e.g., electroporation, calcium phosphate treatment, liposome-mediated transformation, injection and microinjection, ballistic methods, virosomes, immunoliposomes, polycation: nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

In certain embodiments, custom biologics of the present disclosure are or comprise monoclonal antibodies (mAbs), which can be produced by a variety of techniques, including conventional monoclonal antibody methodology e.g., the standard somatic cell hybridization technique of Kohler and Milstein, 1975 Nature 256:495. Many techniques for producing mAbs can be employed e.g., viral or oncogenic transformation of B lymphocytes.

### H. SYSTEM, AND NETWORK ENVIRONMENT

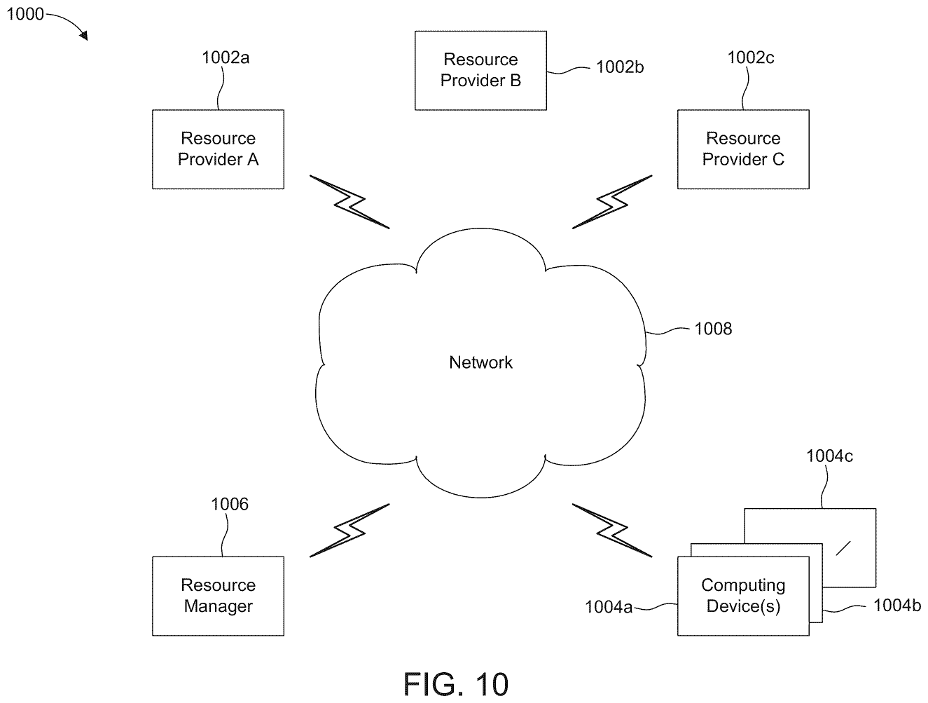

Turning to FIG. 10, an implementation of a network environment 1000 for use in providing systems, methods, and architectures as described herein is shown and described. In brief overview, referring now to FIG. 10, a block diagram of an exemplary cloud computing environment 1000 is shown and described. The cloud computing environment 1000 may include one or more resource providers 1002a, 1002b, 1002c (collectively, 1002). Each resource provider 1002 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 1002 may be connected to any other resource provider 1002 in the cloud computing environment 1000. In some implementations, the resource providers 1002 may be connected over a computer network 1008. Each resource provider 1002 may be connected to one or more computing device 1004a, 1004b, 1004c (collectively, 1004), over the computer network 1008.

The cloud computing environment 1000 may include a resource manager 1006. The resource manager 1006 may be connected to the resource providers 1002 and the computing devices 1004 over the computer network 1008. In some implementations, the resource manager 1006 may facilitate the provision of computing resources by one or more resource providers 1002 to one or more computing devices 1004. The resource manager 1006 may receive a request for a computing resource from a particular computing device 1004. The resource manager 1006 may identify one or more resource providers 1002 capable of providing the computing resource requested by the computing device 1004. The resource manager 1006 may select a resource provider 1002 to provide the computing resource. The resource manager 1006 may facilitate a connection between the resource provider 1002 and a particular computing device 1004. In some implementations, the resource manager 1006 may establish a connection between a particular resource provider 1002 and a particular computing device 1004. In some implementations, the resource manager 1006 may redirect a particular computing device 1004 to a particular resource provider 1002 with the requested computing resource.

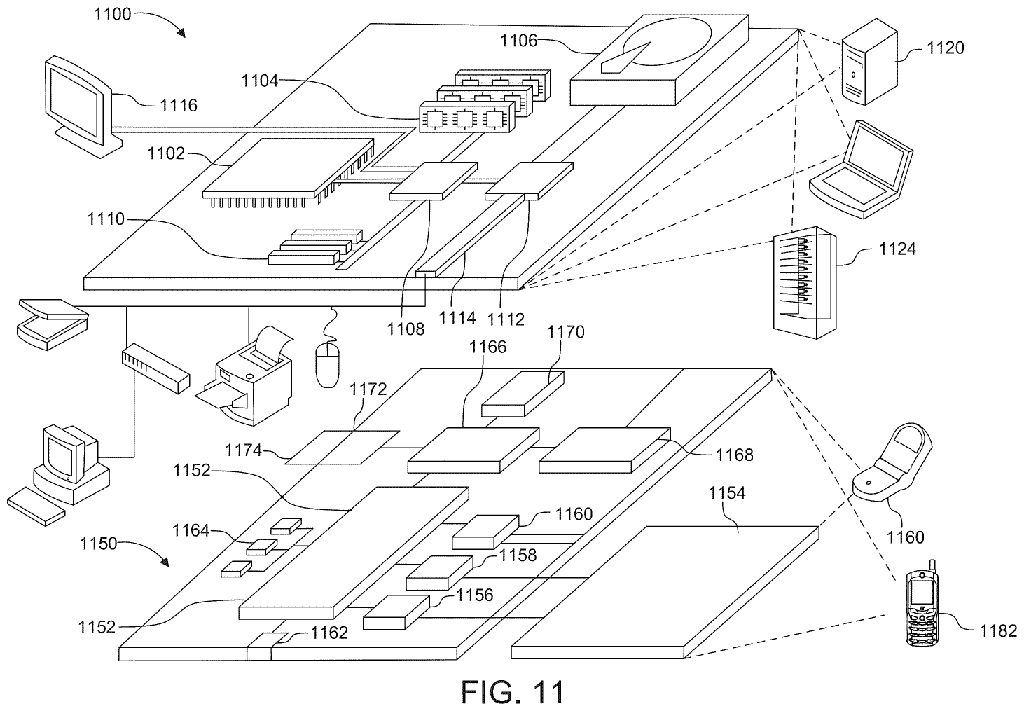

FIG. 11 shows an example of a computing device 1100 and a mobile computing device 1150 that can be used to implement the techniques described in this disclosure. The computing device 1100 is intended to represent various forms of digital computers, such as laptops, desktops, workstations, personal digital assistants, servers, blade servers, mainframes, and other appropriate computers. The mobile computing device 1150 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 1100 includes a processor 1102, a memory 1104, a storage device 1106, a high-speed interface 1108 connecting to the memory 1104 and multiple high-speed expansion ports 1110, and a low-speed interface 1112 connecting to a low-speed expansion port 1114 and the storage device 1106. Each of the processor 1102, the memory 1104, the storage device 1106, the high-speed interface 1108, the high-speed expansion ports 1110, and the low-speed interface 1112, are interconnected using various busses, and may be mounted on a common motherboard or in other manners as appropriate. The processor 1102 can process instructions for execution within the computing device 1100, including instructions stored in the memory 1104 or on the storage device 1106 to display graphical information for a GUI on an external input/output device, such as a display 1116 coupled to the high-speed interface 1108. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 1104 stores information within the computing device 1100. In some implementations, the memory 1104 is a volatile memory unit or units. In some implementations, the memory 1104 is a non-volatile memory unit or units. The memory 1104 may also be another form of computer-readable medium, such as a magnetic or optical disk.

The storage device 1106 is capable of providing mass storage for the computing device 1100. In some implementations, the storage device 1106 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 1102), perform one or more methods, such as those described above. The instructions can also be stored by one or more storage devices such as computer- or machine-readable mediums (for example, the memory 1104, the storage device 1106, or memory on the processor 1102).

The high-speed interface 1108 manages bandwidth-intensive operations for the computing device 1100, while the low-speed interface 1112 manages lower bandwidth-intensive operations. Such allocation of functions is an example only. In some implementations, the high-speed interface 1108 is coupled to the memory 1104, the display 1116 (e.g., through a graphics processor or accelerator), and to the high-speed expansion ports 1110, which may accept various expansion cards (not shown). In the implementation, the low-speed interface 1112 is coupled to the storage device 1106 and the low-speed expansion port 1114. The low-speed expansion port 1114, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 1100 may be implemented in a number of different forms, as shown in the figure. For example, it may be implemented as a standard server 1120, or multiple times in a group of such servers. In addition, it may be implemented in a personal computer such as a laptop computer 1122. It may also be implemented as part of a rack server system 1124. Alternatively, components from the computing device 1100 may be combined with other components in a mobile device (not shown), such as a mobile computing device 1150. Each of such devices may contain one or more of the computing device 1100 and the mobile computing device 1150, and an entire system may be made up of multiple computing devices communicating with each other.

The mobile computing device 1150 includes a processor 1152, a memory 1164, an input/output device such as a display 1154, a communication interface 1166, and a transceiver 1168, among other components. The mobile computing device 1150 may also be provided with a storage device, such as a micro-drive or other device, to provide additional storage. Each of the processor 1152, the memory 1164, the display 1154, the communication interface 1166, and the transceiver 1168, are interconnected using various buses, and several of the components may be mounted on a common motherboard or in other manners as appropriate.

The processor 1152 can execute instructions within the mobile computing device 1150, including instructions stored in the memory 1164. The processor 1152 may be implemented as a chipset of chips that include separate and multiple analog and digital processors. The processor 1152 may provide, for example, for coordination of the other components of the mobile computing device 1150, such as control of user interfaces, applications run by the mobile computing device 1150, and wireless communication by the mobile computing device 1150.

The processor 1152 may communicate with a user through a control interface 1158 and a display interface 1156 coupled to the display 1154. The display 1154 may be, for example, a TFT (Thin-Film-Transistor Liquid Crystal Display) display or an OLED (Organic Light Emitting Diode) display, or other appropriate display technology. The display interface 1156 may comprise appropriate circuitry for driving the display 1154 to present graphical and other information to a user. The control interface 1158 may receive commands from a user and convert them for submission to the processor 1152. In addition, an external interface 1162 may provide communication with the processor 1152, so as to enable near area communication of the mobile computing device 1150 with other devices. The external interface 1162 may provide, for example, for wired communication in some implementations, or for wireless communication in other implementations, and multiple interfaces may also be used.

The memory 1164 stores information within the mobile computing device 1150. The memory 1164 can be implemented as one or more of a computer-readable medium or media, a volatile memory unit or units, or a non-volatile memory unit or units. An expansion memory 1174 may also be provided and connected to the mobile computing device 1150 through an expansion interface 1172, which may include, for example, a SIMM (Single In Line Memory Module) card interface. The expansion memory 1174 may provide extra storage space for the mobile computing device 1150, or may also store applications or other information for the mobile computing device 1150. Specifically, the expansion memory 1174 may include instructions to carry out or supplement the processes described above, and may include secure information also. Thus, for example, the expansion memory 1174 may be provide as a security module for the mobile computing device 1150, and may be programmed with instructions that permit secure use of the mobile computing device 1150. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. The instructions, when executed by one or more processing devices (for example, processor 1152), perform one or more methods, such as those described above. The instructions can also be stored by one or more storage devices, such as one or more computer- or machine-readable mediums (for example, the memory 1164, the expansion memory 1174, or memory on the processor 1152). In some implementations, the instructions can be received in a propagated signal, for example, over the transceiver 1168 or the external interface 1162.

The mobile computing device 1150 may communicate wirelessly through the communication interface 1166, which may include digital signal processing circuitry where necessary. The communication interface 766 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 1168 using a radio-frequency. In addition, short-range communication may occur, such as using a Bluetooth®, Wi-Fi™, or other such transceiver (not shown). In addition, a GPS (Global Positioning System) receiver module 1170 may provide additional navigation- and location-related wireless data to the mobile computing device 1150, which may be used as appropriate by applications running on the mobile computing device 1150.

The mobile computing device 1150 may also communicate audibly using an audio codec 1160, which may receive spoken information from a user and convert it to usable digital information. The audio codec 1160 may likewise generate audible sound for a user, such as through a speaker, e.g., in a handset of the mobile computing device 1150. Such sound may include sound from voice telephone calls, may include recorded sound (e.g., voice messages, music files, etc.) and may also include sound generated by applications operating on the mobile computing device 1150.

The mobile computing device 1150 may be implemented in a number of different forms, as shown in the figure. For example, it may be implemented as a cellular telephone 1180. It may also be implemented as part of a smart-phone 1182, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Actions associated with implementing the systems may be performed by one or more programmable processors executing one or more computer programs. All or part of the systems may be implemented as special purpose logic circuitry, for example, a field programmable gate array (FPGA) or an application-specific integrated circuit (ASIC), or both. All or part of the systems may also be implemented as special purpose logic circuitry, for example, a specially designed (or configured) central processing unit (CPU), conventional central processing units (CPU) a graphics processing unit (GPU), and/or a tensor processing unit (TPU).

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, modules described herein can be separated, combined or incorporated into single or combined modules. The modules depicted in the figures are not intended to limit the systems described herein to the software architectures shown therein.

### I. EXAMPLES

### I.i Example 1: Example Training Curves and Generation of De-Novo Structures

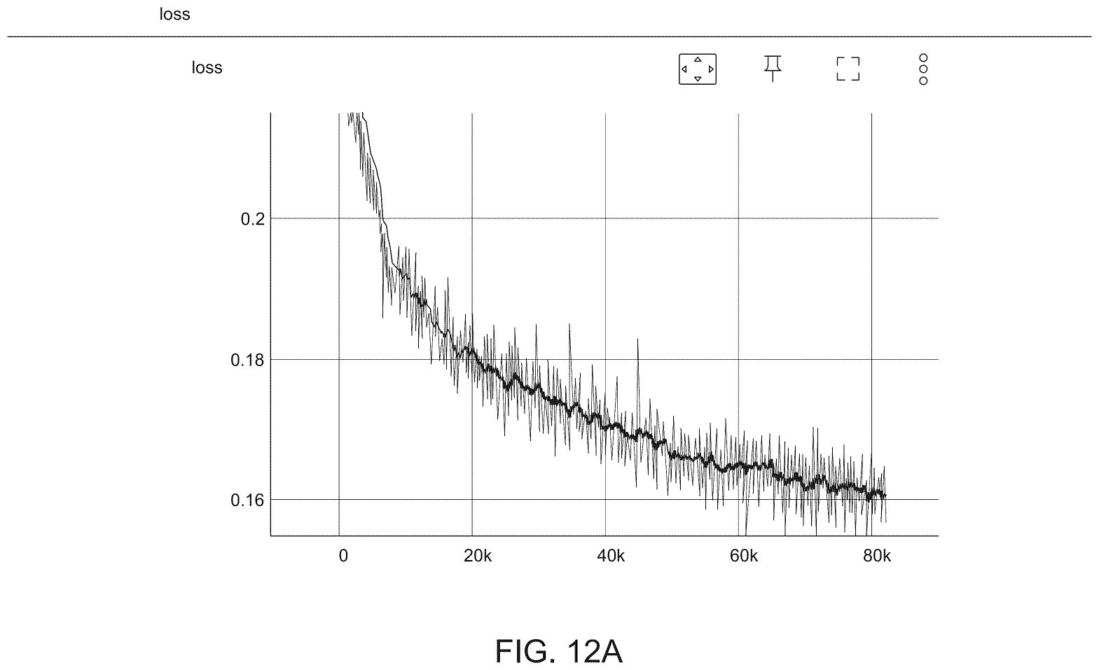

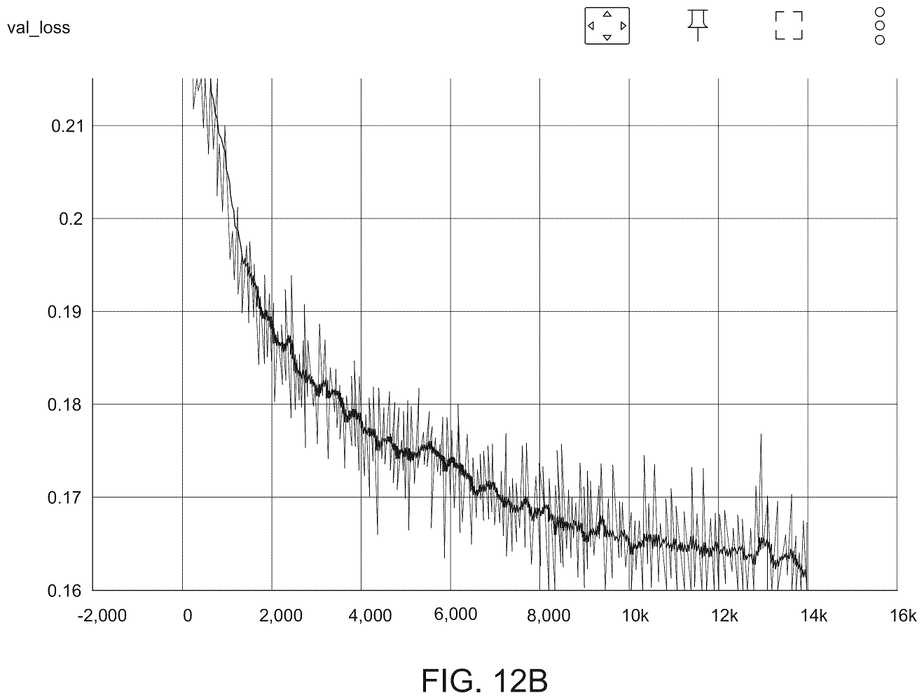

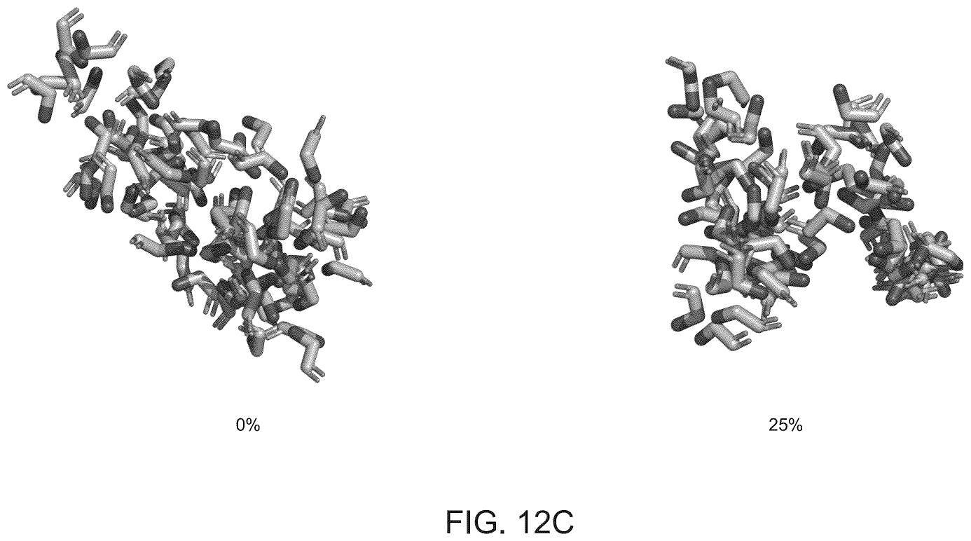

This example demonstrates training of a generative machine learning model according to various embodiments described herein and its use for generating a de-novo peptide backbone structure. FIG. 12A is graph showing an example loss curve during training of a machine learning model used to estimate velocity fields in a flow-matching framework for generating de-novo peptide backbones.

FIG. 12B is graph showing an example loss curve showing validation of a machine learning model used to estimate velocity fields in a flow-matching framework for generating de-novo peptide backbones, according to an illustrative embodiment.

FIG. 12C is a series of snapshots showing computed atom positions at various time points (0%, 25%, 50%, 75%, and 100%) as they are iteratively adjusted to create a generated scaffold model representing a generated de-novo peptide backbone. Overlays at a final, 100%, structure show generated helical motifs.

### I.ii Example 2: Example Metrics for Assessing Final Structure Quality

While FIGS. 12A and 12B evaluate loss, during training and validation, based on determined velocity field values, in certain embodiments, additional and/or alternative validation metrics may be determined based on/using a final generated scaffold model. For example, in certain embodiments, a self-consistency template modeling score (SC TM) may be determined using a scaffold model generated via various embodiments described herein. In certain embodiments, a SC TM score may be determined by populating a generated scaffold model with amino acids to create a generated amino acid sequence. The generated amino acid sequence can then be used, for example via one or more protein folding models (e.g., machine learning models) to create a predicted 3D folded protein structure. The backbone of the predicted 3D folded protein structure can then be compared to determine a level of consistency/similarity, via a metric such as a TM score (template modeling score).

### I.iii Example 3: Example Equivariant Model

This example describes an example model that is equivariant with respect to three-dimensional rotations and/or translations of its input. The model of the present example leverages multiple representations of local frames, shifting between them, and makes use of an approach whereby positions and/or orientations of each local frame can be represented relative to its position and/or orientation at another, e.g., earlier, time point.

In certain embodiments, approaches described herein may utilize machine learning models alone or in combination with various feature vector representations (of peptide backbone sites) to compute quantities such as velocity fields and/or feature vector values representing positions and/or rotations of backbone sites in a manner that is invariant and/or equivariant with respect to three-dimensional translation and/or rotation of a received input. An invariant function or model, as used herein, refers to a function or model whose output is invariant with respect to certain operations, such as rotation or translation, on its input. That is, a model that is invariant with respect to three-dimensional rotations and translations will, (i), for a particular input received, produce a particular corresponding output, and, (ii), if a rotated and/or translated version of that particular input is received, still produce that same particular corresponding output. An equivariant function or model, as used herein, refers to a function or model whose output is equivariant with respect to certain operations, such as rotation and/or translation, of its input. That is, a model that is equivariant with respect to three-dimensional rotations and translations will, (i), for a particular input received, produce a particular corresponding output, and, (ii), if a rotated and/or translated version of that particular input is received, produce a likewise rotated and/or translated version (i.e., having a same rotation and/or translation as the input) of the particular corresponding output. That is, in brief, an invariant model's output does not vary upon rotation and/or translation of its input, while an equivariant model's output rotates and/or translates with rotation and/or translation of its input.

Without wishing to be bound to any particular theory, as described in this example, in certain embodiments, an equivariant model facilitates certain formats of a flow-matching framework, and can be desirable/allow for improved accuracy, particularly with respect to modelling large proteins and/or their tertiary structure.

### (a) Equivariant Framework

In this example, several approaches for representing local frames that correspond to peptide backbone sites are used to allow for machine learning models to generate predictions of sets of prospective final feature vector values and, from them, compute velocity fields in an equivariant fashion with respect to rotations and/or translations of input.

In one representation, an orientation and position of a local frame, i, at a time t may be represented using a set of rotations and translations with reference to a common global frame as shown in Equation (1), below.

$$\{ \begin{matrix}{q}_{i} ( t ) ∈ {ℝ}^{4} , \\ {x}_{i} ( t ) ∈ {ℝ}^{3}\end{matrix} \qquad Eq . ( 1 )$$

In Equation (1), qᵢ(t) is a quaternion used to represent a rotation of frame i at time t and xᵢ(t) is a three-dimensional translation of frame i at time t. The representation shown in Equation (1) uses a global reference frame, such that rotations and/or translations of each frame i are defined with respect to a single common global coordinate system, and not relative to any other frame and/or time point.

In another representation, a local frame j at a time t may be represented from a point of view of another backbone site—i.e., another local frame, i, also at a same time t, as shown in Equation (2), below.

$$\{ \begin{matrix}{q}_{ij} ( t ) = {q}_{i} {( t )}^{- 1} {q}_{j} ( t ) , \\ {x}_{ij} ( t ) = {{q}_{i} ( t )}^{- 1} [ {x}_{j} ( t ) - {x}_{i} ( t ) ]\end{matrix} \qquad Eq . ( 2 )$$

In another representation, a local frame, i, at a time t′ may be represented from a point of view of that same local frame at another time, t, as shown in Equation (3), below.

$$\{ \begin{matrix}{q}_{i} ( t , {t}^{′} ) = {{q}_{i} ( t )}^{- 1} {q}_{i} ( {t}^{′} ) , \\ {x}_{i} ( t , {t}^{′} ) = {{q}_{i} ( t )}^{- 1} [ {x}_{i} ( {t}^{′} ) - {x}_{i} ( t ) ]\end{matrix} \qquad Eq . ( 3 )$$

The relations in Equations (1) to (3) can be used to shift between a global representation of local frames and various relative representations, e.g., representing a particular local frame relative to neighboring local frames and/or a position/orientation of that same local frame at another (e.g., earlier) time point.

As shown in the below, shifting between global and various relative local frame representations can be used to employ a machine learning model (e.g., a deep neural network) that generates predictions of a set of prospective final feature vectors representing final positions and/or orientations of each local frame.

In particular, a function ƒ that is invariant with respect to translations and/or rotations of its input may be determined as shown in Equation (4), below.

$$f : [ {x}_{i} ( t ) , {q}_{i} ( t ) ] \to [ {x}_{ij} ( t ) , {q}_{ij} ( t ) ] {\to }^{{f}_{θ}} [ ( t , 1 ) , ( t , 1 ) ] \qquad Eq . ( 4 )$$

In Equation (4), beginning with a global representation of local frame positions and orientations, a relative representation according to Equation (2) is created, and then used as input to a machine learning model (the subscript, θ, denoting learned parameters of the model), such as a deep neural network. Machine learning model ƒ_{θ} generates predictions of a set of prospective final feature vector values, [ [symbol] (t, 1), [symbol] (t, 1)], as output, where t=1 is a final time point (t ranging from [0,1]). Accordingly, function ƒ takes, as input, a representation of each local frame at time t according to a common, global reference, [xᵢ(t), qᵢ(t)] and generates, as output, a set of prospective (predicted) final feature vector values, [ [symbol] (t, 1), [symbol] (t, 1)], represented as a translation and rotation relative to that local frame's representation at time t (i.e., the input values).

In order to produce an equivariant output, that shifts and/or rotates in line with its input, an additional transformation can be used to shift back to a global reference, as shown in Equation (5), below, where ƒ is as defined in Equation (4):

$$g : [ {x}_{i} ( t ) , {q}_{i} ( t ) ] {\to }^{f} [ \hat{{x}_{i}} ( t , 1 ) , ( t , 1 ) ] \to [ {q}_{i} ( t ) \hat{{x}_{i}} ( t , 1 ) + {x}_{i} ( t ) , {q}_{i} ( t ) ( t , 1 ) ] \qquad Eq . ( 5 )$$

The final output is equal to [\hat{x}ₜ(t), \hat{q}ₜ(1)], via Equation (6), below, and, accordingly, is equivariant.

$$\{ \begin{matrix}{q}_{i} ( t ) ( t , 1 ) = ( 1 ) , \\ {q}_{i} ( t ) ( t , 1 ) + {x}_{i} ( t ) = ( t )\end{matrix} \qquad Eq . ( 6 )$$

Accordingly, by implementing a function, g, in accordance with Equation (5), predictions of a set of final prospective feature vector values that represent positions and/or orientations of local frames can be generated using a machine learning model.

### (b) Velocity Computations

An equivariant model for generating predictions of prospective final feature vector values can be used, in turn, to generate predictions of a velocity field at a particular time point, t. As described herein, predicted velocity fields can be used (i) during training, to compare with a ground truth predicted velocity and evaluate a loss function in order to train a machine learning model and (ii) during inference, to adjust position and/or orientation components of feature vectors at each time point.

As described herein, in certain embodiments, a machine learning model can be used to generate velocity field estimates directly, as output.

In this example, however, as described above, a machine learning model is used to generate predictions of a set of prospective final feature vector values, representing predicted positions and/or orientations of each backbone site's position and/or orientation in a final generated peptide. Accordingly, at each training step and/or time point, a set of prospective final feature vector values is generated and then used to compute a corresponding velocity field estimate that, in turn, can be used to compute loss (for training) or adjust position and/or orientation components of a current feature vector, e.g., to advance to a next iteration in a process of generating a final peptide structure.

In terms of training, at each step, velocity predictions and loss can be calculated as follows:
1. Machine learning model ƒ receives current feature vector values and a current time point as input and generates, as output, estimated final feature vector values (at time t=1):

$$f ( {x}_{t} , t ) =$$

2. A velocity field estimate, \hat{v}ₜ, at time t is computed based on the estimated final feature vector values, [symbol] , the current feature vector values, xₜ, and the current time point, t; and
3. A current loss is computed based on the estimated (\hat{v}ₜ) and ground truth (vₜ) velocity fields:

$$Loss ( \hat{{v}_{t}} , {v}_{t} ) ,$$

with x in this and the following sections of this example denoting a feature vector (e.g., inclusive of both position and/or orientation components and not necessarily solely the position components).

Where paths between x₀ and x₁ are geodesics, a ground truth velocity field and an estimated velocity field can be calculated using the relation in Equation (7a), below:

$$If {v}_{t} = v ( t , {x}_{0} , {x}_{1} ) , then {v}_{t} = v ( 0 , {x}_{t} , {x}_{1} ) / ( 1 - t ) . \qquad Eq . ( 7 a )$$

Equation (7a) can be used to compute estimated velocity fields from predicted final feature vector values generated by a machine learning model as well, for example as shown in Equation (7b), below:

$$\hat{{v}_{t}} = v ( 0 , {x}_{t} , {\hat{x}}_{1} ) / ( 1 - t ) . \qquad Eq . ( 7 b )$$

In a Euclidean space, Equations (7a) and (7b) follow from a constraint that paths between x₀ and x₁ are geodesics, as follows:

$$\begin{matrix}{x}_{t} = {x}_{0} + t ( {x}_{1} - {x}_{0} ) \\ = ( 1 - t ) {x}_{0} + {tx}_{1} ; \\ v = \frac{dx}{dt} = {x}_{1} - {x}_{0} ; and \\ \begin{matrix}v ( 0 , {x}_{t} , {x}_{1} ) = {x}_{1} - {x}_{t} \\ = {x}_{1} - {x}_{0} - t ( {x}_{1} - {x}_{0} ) \\ = ( 1 - t ) ( {x}_{1} - {x}_{0} ) = ( 1 - t ) {v}_{t}\end{matrix}\end{matrix}$$

On a spherical surface, a geodesic between x₀ and x1 is an arc between x₀ and x₁ of a circle resulting from an intersection of S³ and a plane made by x₀, x₁, and O, where O is an origin. This arc can be parameterized using a complex unit circle and its classical 1 to 1 mapping. In this representation, x₀ becomes exp(iθ₀), x₁ becomes exp(iθ₁), and xₜ becomes exp(iθₜ)=exp{i[tθ₁+(1−t)θ₀]}. Without a loss of generality, these three points can be represented according to their complex 1 to 1 mapping.

Accordingly,

$${x}_{t} = exp ( i {θ}_{t} ) = exp \{ i [ t {θ}_{1} + ( 1 - t ) {θ}_{0} ] \} \\ {v}_{t} = \frac{dx}{dt} = i ( {θ}_{1} - {θ}_{0} ) exp \{ i [ t {θ}_{1} + ( 1 - t ) {θ}_{0} ] \} \\ \begin{matrix}v ( 0 , {x}_{t} , {x}_{1} ) = i ( {θ}_{1} - {θ}_{0} ) exp ( i {θ}_{t} ) \\ = i ( {θ}_{1} - [ t {θ}_{1} + ( 1 - t ) {θ}_{0} ] ) exp ( i [ t {θ}_{1} + ( 1 - t ) {θ}_{0} ] ) \\ = i ( 1 - t ) ( {θ}_{1} - {θ}_{0} ) exp \{ i [ t {θ}_{1} + ( 1 - t ) {θ}_{0} ] \} = ( 1 - t ) {v}_{t}\end{matrix}$$

Euclidean equations shown in this example may be used for translations and their velocities, which are in a 3D Euclidean space (R³). Spherical equations shown in this example may be used for quaternions, which are on a 4D sphere (S³), and have speeds on Tangent® planes.

### (c) Example Training and Inference in Euclidean Space

Accordingly, in Euclidean space, the above described framework provides the below equations:

$$\begin{matrix}{v}_{t} = v ( t , {x}_{0} , {x}_{1} ) & Eq . ( 8 a ) \\ {v}_{t} = v ( 0 , {x}_{t} , {x}_{1} ) / ( 1 - t ) & Eq . ( 8 b ) \\ {x}_{t} = {tx}_{1} + ( 1 - t ) {x}_{0} & Eq . ( 8 c ) \\ {v}_{t} = {x}_{1} - {x}_{0} = v ( t , {x}_{0} , {x}_{1} ) & Eq . ( 8 d ) \\ {v}_{t} = v ( 0 , {x}_{t} , {x}_{1} ) / ( 1 - t ) = ( {x}_{1} - {x}_{t} ) / ( 1 - t ) & Eq . ( 8 e )\end{matrix}$$

These equations can be used, at training and inference steps, for example, as follows:

### 1. Example Training Steps:

1. Given: x₀˜p₀; t˜U[0, 1]; and x₁˜p_{data}
2. Compute ‘ground truth’/analytical:

$$\begin{matrix}{x}_{t} = {tx}_{1} + ( 1 - t ) {x}_{0} via \& & Eq . ( 8 c ) \\ {v}_{t} = ( {x}_{1} - {x}_{t} ) / ( 1 - t ) via & Eq . ( 8 e )\end{matrix}$$

[or, additionally or alternatively, Eq. (8d)—i.e., vₜ=x₁−x₀]
3. Use a machine learning model to generate predictions:

(xₜ,t)→ƒ_{θ}→x₁^{(est)} (superscript (est) indicating estimate)
4. Compute loss:

$${v}_{t}^{( est )} = ( {x}_{1}^{( est )} - {x}_{t} ) / ( 1 - t ) via \qquad Eq . ( 8 e )$$

[or, additionally or alternatively, Eq. (8d)—i.e., vₜ^{(est)}=x₁^{(est)}−x₀]

$$Loss = L ( {v}_{t}^{( est )} , {v}_{t} ) ( e . g . , {‖ {v}_{t}^{( est )} - {v}_{t} ‖}^{2} )$$

### 2. Example Inference Steps:

1. Given x₀˜p₀, N desired iterations, so some time interval dt=1/N
2. at iteration 0:

$$( {x}_{0} , 0 ) \to {f}_{θ} \to {x}_{1 ( 0 )}^{( est )} \\ {v}_{t ( 0 )}^{( est )} = ( {x}_{1 ( 0 )}^{( est )} - {x}_{0} ) / ( 1 - 0 ) via Eq . ( 8 e )$$

[at this step, identical to eq. (8d), i.e., vₜ=x₁−x₀]

$${x}_{t ( 1 )}^{( est )} = {x}_{0} + {v}_{t ( 0 )}^{( est )} dt$$

3. for iterations i=1 through N−1:

$$( {x}_{t ( i )} , {t}_{( i )} ) \to {f}_{θ} \to {x}_{1 ( i )}^{( est )} ( where {t}_{( i )} = N dt ) \\ {v}_{t ( i )}^{( est )} = ( {x}_{1 ( i )}^{( est )} - {x}_{t ( i )}^{( est )} ) / ( 1 - {t}_{( i )} ) via ( 5 )$$

[or, additionally or alternatively Eq. (8d)—i.e., vₜ₍ᵢ₎^{(est)}=x₁₍ᵢ₎^{(est)}−x₀]

$${x}_{t ( i + 1 )}^{( est )} = {x}_{t ( i )}^{( est )} + {v}_{t ( i )}^{( est )} dt$$

The equations above are shown for Euclidean space for illustrative purposes—analogous formulations can be used for other (e.g., spherical, cylindrical) formulations described herein.

Accordingly, this example describes a particular form of a flow matching approach in accordance with certain embodiments described herein that utilizes a machine learning model to generate predictions of final feature vector position and/or orientation components from which velocity estimates are computed. Without wishing to be bound to any particular theory, in certain embodiments, it is believed that this approach may provide advantages, particularly where large proteins are being generated and/or with respect to tertiary structure predictions. For example, during inference, velocities may be repeatedly calculated at each of a plurality of iterations and used to adjust positions and/or orientations of backbone sites to create a final generated backbone structure. Where velocities are directly computed by a machine learning model, even where errors in individual velocity predictions are small, these errors may compound, resulting in reduced accuracies in long range/high level structure of larger protein molecules, since several such predictions are used together to repeatedly adjust backbone site positions and orientations. By utilizing a machine learning model to output predictions of final backbone site positions and orientations, this compounding issue can be reduced, since the machine learning model predictions at each iteration are made with respect to a final structure, rather than a localized adjustment.

### I.iv Example 4: Example Framework for Flow Matching in Euclidean Space and SO(3) Manifolds for Protein Generation

This example describes a generalized framework for using flow matching techniques in Euclidean space and/or on manifolds to generate protein structures.

### (a) Paths on Manifolds

As described herein, flow matching-based protein generation techniques may utilize a local frame representation that includes rotational components that represent 3D orientations of local frames used to represent peptide backbone sites. Accordingly, among other things, training and inference procedures for models that use a local frame representation may be facilitated by approaches for generating straight paths in 3D rotational space, SO(3), which can be used to interpolate rotational velocity values during training and update rotational components via push-forward equations during inference and/or generation steps. See, e.g., [Lipman 2022] and [Chen and Lipman 2023]. In particular, exponential and logarithmic maps of Riemannian geometries can be used to generate convenient forms of equations representing linear interpolations between rotational frames, which, in turn, can be used to compute losses during training and push rotational components along a desired path during inference.

Equation (9a), below, describes a linear interpretation between two frame rotation components, R₀ and R₁, as a function of time, where time varies from 0 to 1,

$$R ( t ) = {R}_{0}^{1 - t} {R}^{t} , t ∈ [ 0 , 1 ] . \qquad Eq . ( 9 a )$$

Using the relation R^{a}=exp[a log(R)] and denoting an initial velocity, V₀=log(R₀⁻¹ R₁), equation (9b) describes another interpolation between two frame rotation components, which may, in certain embodiments, be more convenient to use in practice than Eq. (9a),

$$R ( t ) = {R}_{0} exp ( {tV}_{0} ) . \qquad Eq . ( 9 b )$$

Accordingly, by training a neural network to predict initial velocity, V₀, equation (9b) can be used to update (e.g., push) rotational components of feature vectors in a local frame representation along a path, R(t). See, e.g., [Bose 2023] and [Yim 2023]. An inverse of initial frame, R₀⁻¹, which appears in the relation for V₀ can be computed via equations (9c) and (9d) for rotations represented via 3D rotation matrices (i.e., as a matrix transpose) and unit quaternions (i.e., as a conjugate), respectively.

$$\begin{matrix}{R}_{0}^{- 1} = {R}_{0}^{T} for SO ( 3 ) . & Eq . ( 9 c ) \\ {R}_{0}^{- 1} = \overline{{R}_{0}} for unit quaternions . & Eq . ( 9 d )\end{matrix}$$

### (b) Invariance Properties

Physical proteins are rotationally and translationally invariant—that is, a rotated and translated (moved in 3D space) version of a particular protein is still that same particular protein (and not, e.g., a new, different, protein). Accordingly, flow matching approaches formulated for proteins can, in certain embodiments, enforce rotational and translational invariances.

For example, translational invariance can be accomplished by centering a position of each protein representation before processing.

Methods for ensuring rotational invariance include (i) randomly rotating protein representations at t=0 and t=1, before interpolating them, and/or (ii) aligning x₁ and x₀ before interpolating them. Alignment can be accomplished using approaches such as a Kabsch algorithm. By utilizing rotation and/or alignment approaches, neural networks can be trained to predict an equivariant velocity field, e.g., as shown below, and encode a desired invariance property.

$$v ( Rx , t ) = Rv ( x , t ) .$$

Example procedures for training a generative flow matching model using either method are described in the following, for both individual backbone atom representation and local frame representation approaches for encoding protein backbone geometries. In the below, x^{CA} refers to a position of a Ca atom, used in certain embodiments as a position of a local frame.

### 1. Example Training Steps-Individual Backbone Atom Representation

1. Given/sample: x₀˜p₀; t˜U(0, 1); and x₁˜p₁;
2. Center x₀ and x₁;
3. Align and/or rotate x₀ and x₁ (for rotational equivariance):
   Option 1: Align x₀ and x₁ (e.g., via Kabsch algorithm), or
   Option 2: Randomly rotate x₀ and x₁ (e.g., sample two 3D rotations from a uniform distribution on SO(3));
4. Determine a value of x(t) by interpolating along a geodesic from x₀ to x₁, e.g., according to:

$$x ( t ) = {x}_{0} + t ( {x}_{1} - {x}_{0} ) ;$$

and
5. Determine an example velocity at origin as v₀=(x₁−x₀).

### 2. Example Training Framework-Local Frame Representation

1. Sample x₀^{CA}˜p0, R₀˜Q₀, (x₁^{CA}, R₁)˜p₁ and t˜U(0, 1);
2. Center x₁^{CA} and x₀^{CA};
3. Align and/or rotate x₀^{CA} and x₁^{CA} (rotational-equivariance):
   Option 1: Align x₀^{CA} and x₁^{CA} (e.g., via Kabsch algorithm), or
   Option 2: Randomly rotate x₀^{CA} and x₁^{CA} (e.g., sample two 3D rotations from a uniform distribution on SO(3));
4. Determine a value of x(t)=(xₜ^{CA}, Rₜ) by interpolating along geodesics from (i) x₀^{CA} to x₁^{CA} and (ii) R₀ to R₁, e.g., according to:

$${x}_{t}^{CA} = ( 1 - t ) \times {x}_{0}^{CA} + t \times {x}_{1}^{CA} , and \\ {R}_{t} = {R}_{0} exp ( t log ( {R}_{0}^{- 1} {R}_{1} ) ) ;$$

5. Determine velocities at an origin via:

$${v}_{0}^{CA} = {x}_{1} - {x}_{0} , and \\ {V}_{0} = log ( {R}_{0}^{- 1} {R}_{1} ) .$$

Accordingly, steps 1 through 5, above, can be used, in connection with approaches that utilize an individual backbone atom representation and/or local frame representation of peptide backbones, to determine (1) velocity fields at an origin and (2) values of x(t) along a geodesic path from x₀ to x₁ at a sampled time point, e.g., t˜U(0, 1), based on values of x (1) at t=0 and t=1 sampled from an initial seed (e.g., prior) distribution and a dataset of example protein structures, respectively. At each training step, a machine learning model may then be provided inputs of x(t), and t—e.g., (xₜ, t) or (xₜ^{CA}, Rₜ, t), depending on whether an individual backbone atom or local frame representation is used—and generate, as output, predicted velocity fields at an origin. These predicted velocity fields can then be compared with directly computed velocity fields (e.g., according to step 5, above) and used to determine loss values (e.g., a mean-squared loss (MSE)) determined velocities determined and asked to predict velocities at the origin and compare with computed velocities at the origin based on the above. A (e.g., thus) determined loss may then be used to update machine learning model parameters.

### (c) Example Seed Distributions

As described herein, flow matching techniques sample feature vector components, such as individual backbone atom positions and/or alpha-Carbon and frame rotations (e.g., quaternions) from initial seed distributions during training as well as during inference, e.g., to obtain initial starting values. Flow matching techniques as described herein may work with a variety of seed distributions. Among other things, for example, machine learning models that use an individual backbone atom representation may sample locations of each of four backbone atoms (for each amino acid site)—N, C, O, C_{α}—from an isotropic Gaussian distribution. In certain embodiments, machine learning model that use local frame representations may sample alpha-Carbon positions from an isotropic Gaussian distribution and may sample rotational component values from a uniform distribution on SO(3). Other approaches for generating seeds include use of random walks (e.g., a random walk Cα seed), using an isotropic Gaussian for Ca positions in a local frame representation and then using a traveling salesman optimization approach to find a shortest path between nodes, etc.

### (d) Generation/Inference

In certain embodiments, in order to generate a backbone, an ordinary differential equation (ODE) solution approach, such as a Euler method, a Runge-Kutta method, etc., can be used to update feature vector values, e.g., in an iterative manner, based on predicted velocity fields as time increments are stepped through. In certain examples described herein, such as GNN and transformer implementations demonstrated in Examples 7 and 8, an Euler method was used and found to provide appropriate performance. In certain implementations and embodiments, other ODE solution methods may be utilized. As described herein, 3D positions of individual backbone atoms and/or alpha-Carbon atoms evolve to traverse paths in Euclidian space and, additionally or alternatively, 3D rotation components traverse geodesic paths along a SO(3) manifold. Example Euler ODE formulations in Euclidean space and on a SO(3) manifold are shown in equations (9e) and (9f), respectively, below. Equation (9e) may be used as a “push-forward” function for evolving positions of individual backbone atoms and/or local frames (e.g., alpha-Carbon atom positions) and equation (9f) may be used as a push-forward function for evolving rotations.

$$\begin{matrix}X ( t + Δ t ) = X ( t ) + Δ t \times V ( t ) . & Eq . ( 9 e ) \\ R ( t + Δ t ) = R ( t ) exp ( Δ {tV}_{0} ) . & Eq . ( 9 f )\end{matrix}$$

In equations (9e) and (9f), above, V(t) (in certain embodiments, V(t)=v0) and V₀ are predicted via a machine learning model, e.g., as described herein (e.g., above) and Δt is a time increment. In certain embodiments, while velocities shown in Equations (9e) and (9f) are constant values, they are predicted anew at each iteration by a machine learning model receiving updated input—accordingly, velocity predicted at each iteration may change significantly e.g., in norm and direction.

In certain embodiments, protein backbone generation techniques described herein may utilize a timestep scheduler, whereby a scheduling function changes a speed traveled along a geodesic. In particular, a path on a manifold can be written according to

$$R ( t ) = {R}_{0} exp ( {tV}_{0} ) .$$

In certain implementations, a scheduling function, α(t), can be used to adjust a speed along a geodesic. An adjusted path, incorporating a scheduling function, can, accordingly, be written as shown below.

$$R ( t ) = {R}_{0} exp ( α ( t ) {V}_{0} ) .$$

A corresponding, adjusted, push-forward equation used to determine a rotation at a subsequent time step may then be written as shown below, where α′ is a time derivative of α(t).

$$R ( t + Δ t ) = R ( t ) exp ( {α}^{′} ( t ) Δ t \times {V}_{0} ) .$$

Specifically, when Δt is small,

$$α ( t + Δ t ) \approx α ( t ) + {α}^{′} ( t ) Δ t ,$$

$$R ( t ) = {R}_{0} exp ( α ( t ) {V}_{0} )$$

$$R ( t + Δ t ) = {R}_{0} exp ( α ( t + Δ t ) {V}_{0} )$$

$$α ( t + Δ t ) \approx α ( t ) + Δ t * {α}^{′} ( t ) ,$$

Hence we can deduce:

$$R ( t + Δ t ) \approx {R}_{0} exp ( α ( t ) {V}_{0} + {α}^{′} ( t ) Δ {tV}_{0} ) ,$$

$$R ( t + Δ t ) \approx {R}_{0} exp ( α ( t ) {V}_{0} ) \times exp ( {α}^{′} ( t ) Δ {tV}_{0} ) ,$$

$$R ( t + Δ t ) = R ( t ) exp ( {α}^{′} ( t ) Δ t \times {V}_{0} ) .$$

In certain implementations, it was found that accelerating a path of frame rotations at earlier time steps during inference improved a quality of generated backbones, albeit, in certain cases, at a cost of reducing a diversity of generated backbones. A variety of functional forms can be used. An example scheduling function used in certain examples described herein can be defined in terms of its derivative,

$${α}^{′} ( t ) = k ( 1 - t )$$

and was found to generate high quality scaffolds for values of k such as 5, 7, and 10 (other values may be used). Other scheduler functions can also be used. For example, a scheduler with the derivative α′(t)=k was also evaluated and found to generate satisfying scaffolds for values of k from 2 to 5.

### (e) Example Use Cases

Generative approaches of the present disclosure can be used to create custom peptide backbone structures and/or amino acid sequences, in certain embodiments, from scratch (e.g., referred to as unconditioned generation), based on various partial input structures and/or amino acid sequences, and/or desired properties. Where technologies of the present disclosure receive particular input, such as desired (e.g., partial) structural and/or amino acid sequence features and/or other properties, and generate output scaffold models and/or sequences based therein, generation is referred to as “conditioned on” such particular input.

Several example use cases are listed below. Detailed examples describing approaches for training and then using machine learning models to generate output in accordance with various use cases (e.g., including, but not limited to, those listed below) are provided in Examples 5-8 below.

Unconditional generation. In certain implementations and/or uses, generative technologies of the present disclosure may be used to generate scaffold models representing de-novo protein backbones of a variety of sizes, without use of conditioning input. In certain implementations, additionally or alternatively, techniques of described herein may be used to generate amino acid sequences (e.g., together with a de-novo backbone, thereby providing a new backbone structure along with an amino acid sequence predicted to, e.g., fold into that backbone structure).

Generation conditioned on structural and/or sequence data. In certain implementations and/or uses, conditioning generation on various structural and/or sequence properties can be used to train model(s) that can perform (without limitation) one or more of the following tasks:
Design a binder to a desired (e.g., known, pre-defined, etc.) target (e.g., protein and/or peptide, such as a particular receptor). Binder designs may be conditioned based on known hotspots (e.g., locations on a protein determined to be within a threshold distance of a binding partner and, accordingly, participate in binding) (e.g., on the target), or without knowledge of binding hotspots;
Sample a conformations of particular protein(s) and/or peptide(s) (e.g., sampling conformations can be used to evaluate energy, stability, and other properties of molecules);
Generate a sequence conditioned on a particular scaffold model (e.g., representing a peptide backbone);
Inpaint one or more subsets/portions of a provided partial (e.g., desired, a-priori known, etc.) peptide backbone and/or protein complex;
Dock two or more particular protein and/or peptides based on their monomeric structures (e.g., conditioned on scaffold models and/or sequence data representing peptide backbones and/or amino acid sequences of the particular proteins and/or peptide monomers);
Generate scaffold models and/or sequences thereof representing peptide backbones and/or amino acid sequences conditioned on a particular oligomeric state (e.g., monomer, dimer, multimer, etc.);
Generate a scaffold model representing a peptide backbone conditioned on a particular amino acid sequence (e.g., protein folding);
Generation of side chains (e.g., rotamer structure) from a known backbone and sequence.

Generation conditioned on global and/or node variables. In certain embodiments, input variables may be used to represent particular (e.g., desired) global properties—for example, these may include, without limitation, one or more of the following:
a protein family variable whose value identifies a particular one of a set of protein family types (e.g., a categorical variable that can take on one of a finite set of values, each identifying a particular protein family; e.g., finite length vector that represents a particular protein family via a one-hot encoding);
a thermostability variable whose value categorizes and/or measures (e.g., quantitatively) protein thermostability [e.g., a binary variable classifying a protein as thermophilic or not (e.g., based on PDB classification); e.g., a continuous real-number measuring melting temperature];
an immunology/immunogenicity variable whose value classifies and/or measures a propensity and/or likelihood of provoking an immune response [e.g., in a particular host organism (e.g., a human)];
a function variable whose value classifies protein function;
solubility variable whose value classifies and/or measures a protein solubility; and
a pH sensitivity variable whose value classifies and/or measures protein pH sensitivity.

In certain implementations, additionally or alternatively, generation may be conditioned based on values of one or more node property variables, each node property variable associated with and representing a particular property of a particular amino acid site. Node property variables may include, without limitation, one or more of the following:
a side chain type variable that identifies a particular type of amino acid side chain (e.g., via a one-hot encoding scheme);
an amino acid polarity variable that identifies a polarity and/or charge of an amino acid site (e.g., a categorical variable having one of four values identifying a particular side chain as polar, apolar, negatively charged, positively charged or unknown (e.g., node to predict or modified/synthetic amino acids));
a buriedness variable that classifies and/or measures an extent to which a particular amino acid site is buried and/or surface-accessible (e.g., a binary variable classifying a particular node as representing an amino acid site at a surface or core of a protein and/or peptide);
a contact hotspot variable classifying a particular amino acid site according to a desired and/or threshold distance from one or more portions of another (e.g., target) molecule [in certain embodiments, similar and/or related edge property variables may be used, e.g., a contact hotspot variable classifying the edge as associated with one or more particular amino acid sites having a desired and/or within a threshold distance from one or more portions of another (e.g., target) molecule]; and
a secondary structure variable whose value classifies and/or measures a secondary structure motif (e.g., helical, beta-sheet, etc.) at the particular amino acid site.

### (f) Side Chain Design Via Flow Matching

In certain embodiments, once a structure and sequence have been designed (e.g., to create a binder for instance) side chains may be generated, for example to assess designability of the generated protein structure. For example, whether sufficient space is present between neighboring amino acids to accommodate particular side chains may not be apparent/feasible to determine from backbone geometry alone.

Accordingly, in certain embodiments, side chain structure can be designed using flow matching generative approaches. In certain embodiments, for example, a side chain design approach may be implemented as an additional step, e.g., following generation of a backbone and/or sequence. Side chain generation approaches described herein may also be implemented and/or used separately from structure design approaches, for example to predict side chain geometries based on a priori known and/or otherwise determined (e.g., via a variety of methods) sequence and backbone geometries.

Approaches for side chain generation of the present disclosure may use various techniques for representing side chains, for example by representing individual amino acid side chain atoms in 3D space and/or using side chain dihedral (e.g., χ) angles.

In certain embodiments, where side chains are represented via 3D coordinates of individual constituent atoms, flow matching techniques for Euclidean spaces described herein can be used to predict and evolve locations of individual side chain atoms, in a manner similar to approaches described herein for generation of peptide backbone structures using representations of individual backbone atoms. Fixed and/or known backbone and amino acid sequence information can be provided as conditioning input, e.g., via an edge retrieval layer and/or node embeddings, respectively, analogously to approaches described in examples below for performing inpainting tasks, using an equivariant model architecture. Side chain atom position seeds could be selected from, for example, a Gaussian distribution centered about the amino acid's Cα backbone atom (e.g., keeping side chain atom points near a Cα atom).

Additionally or alternatively, models using a dihedral angle representation of amino acid side chain structure may perform flow matching on a manifold geometry, representing each angle as an element of a 2D sphere. Appropriate machine learning model architectures include invariant models (e.g., such as an invariant encoder portion used herein with respect to described transformer models), since dihedral angles are invariant with respect to rotations of an entire protein. Fixed and/or known backbone and amino acid sequence information can be provided as conditioning input, e.g., via an edge retrieval layer and/or node embeddings, respectively, analogously to approaches described in examples below for performing inpainting tasks, using an equivariant model architecture. Seed angle values may be sampled uniformly over a 2D sphere. Training and generation approaches analogous to those described with respect to local frame representations that make use of quaternion representations can be adapted to apply to side chain geometry prediction based on dihedral angles, which also describe angles on a sphere (a 2D sphere, as opposed to a 4D sphere).

### I.v Example 5: Example Model Architectures

This example describes several example machine learning model architectures that can be used in connection with flow-matching technologies for protein backbone generation described herein.

### (a) Building Block Components

### 1. SO(3) Equivariant Model Components

As described herein, machine learning models in accordance with certain embodiments aim to learn to generate predictions of a vector field (i.e., velocities) that is equivariant to rotations. Accordingly, model architectures used must also be equivariant to rotations.

An SO(3)-equivariant model can be built by stacking various blocks, such as (i) an SO(3)-invariant node encoder, and (ii) a mix of SO(3)-invariant layers and SO(3)-equivariant layers, interleaved such that a final output is equivariant. Node encoders can be used to encode node-level information, such as an amino acid side chain type embedding, geometric features (e.g., individual backbone atom relationships) at and/or within a particular amino acid site, as well as various node property variables described herein, which, may, for example, be used as conditioning input.

A variety of types of invariant layers can be used, including, without limitation, graph neural network layers, transformer layers, and layers used to retrieve edge information. Layers for retrieving edge information, referred to hereinafter as “edge retriever layers” are described in further detail below.

Equivariant layers may include equivariant graph neural network (EGNN) layers, for example as described in [Satorras 2021], as well as particular transformer variants of the present disclosure, for example described in further detail in Section (b), below.

In certain implementations, where a local frame representation is used, invariant layer outputs can be transformed into equivariant output via equation (10), below,

$${v}_{i , equivariant}^{p r e d} ( t ) = {R}_{i} ( t ) {v}_{i , i n v ariant}^{p r e d} \qquad Eq . ( 10 )$$

### 2. Global Conditioning

Machine learning models described herein may also receive global input variables, such as time, protein size, as well as various categorical variables that can be used to condition scaffold generation on. Global input can be incorporated via various techniques. For example, certain embodiments of machine learning models in accordance with the present disclosure may use a conditional gating approach, whereby at various (e.g., each) layer(s), global information is input to a feed-forward network (FFN), whose output is, in turn, used to modulate layer outputs within the model architecture. Conditional gating is described in further detail, for example, in [Peebles 2022]. Another approach to including global input is to use global information nodes, whereby artificial nodes are added to an input, and used as registers to store and process global variables. See, e.g., [Darcet 2023].

### (b) Example Transformer Architecture

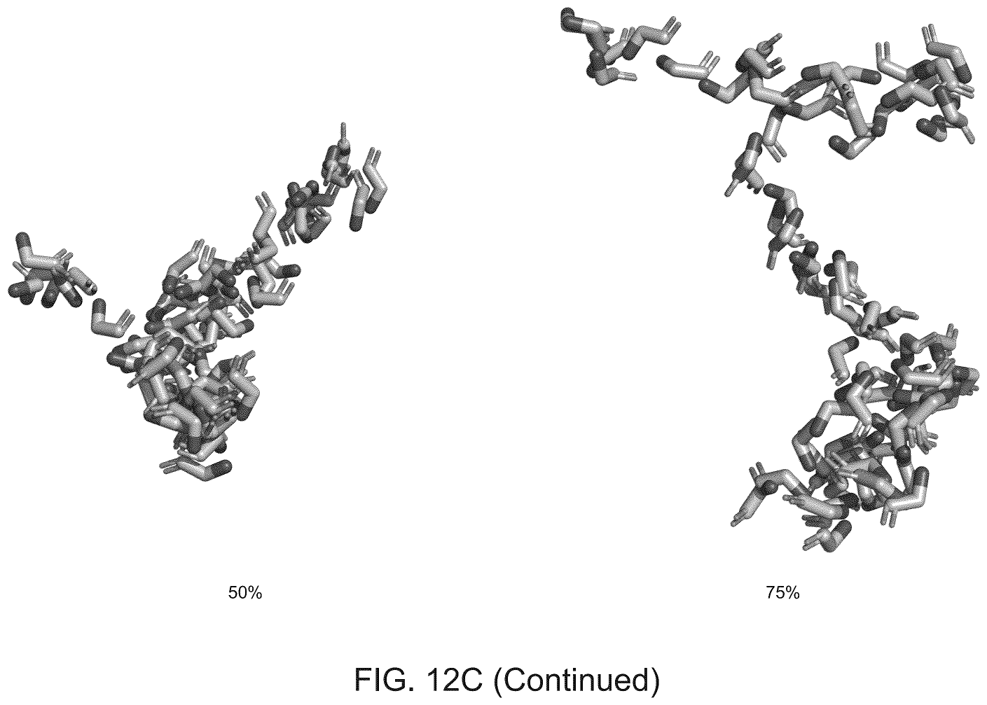

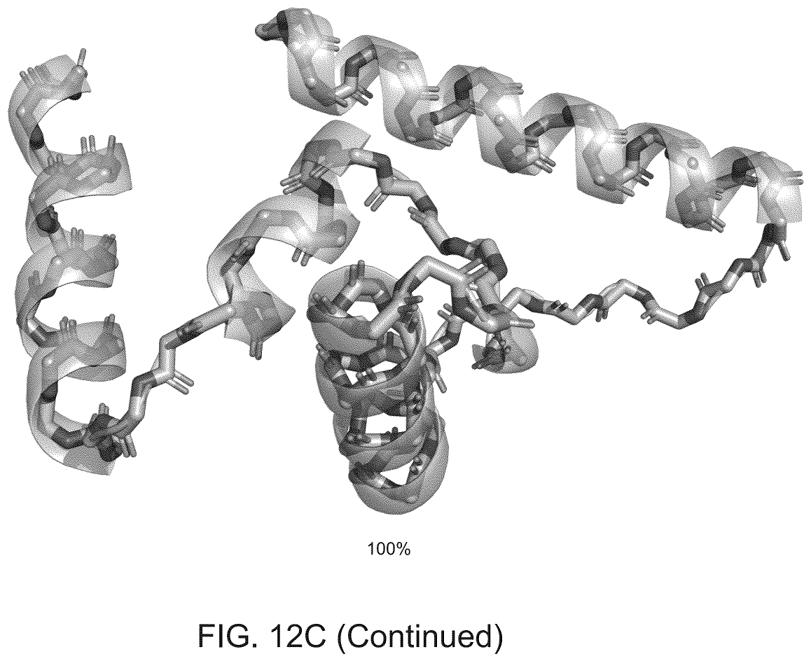

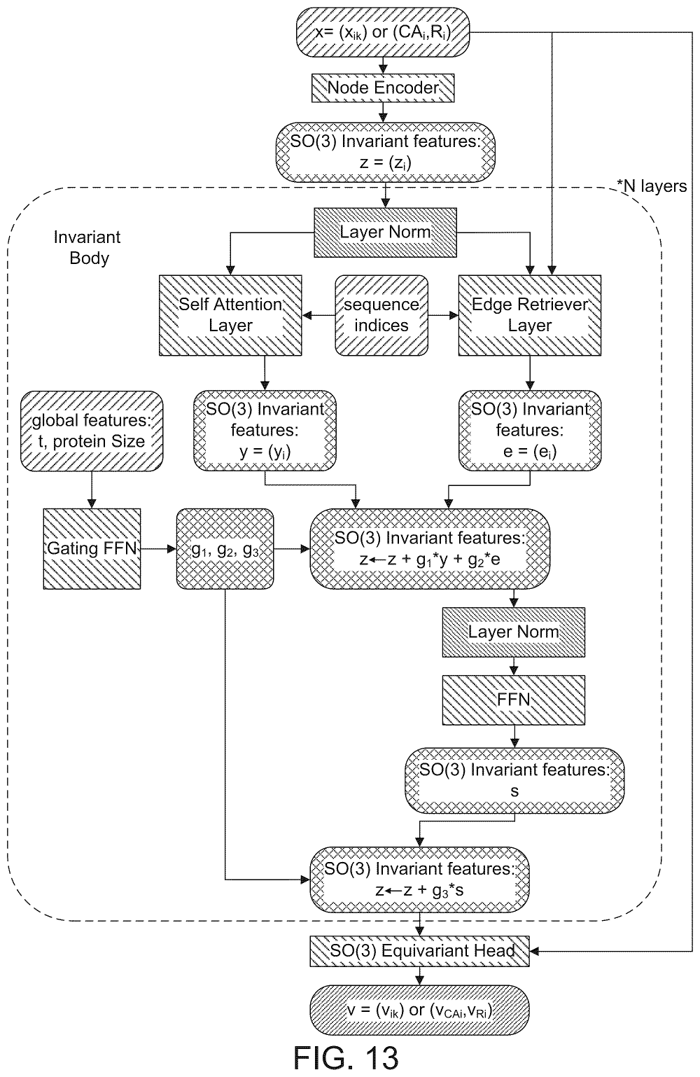

Turning to FIG. 13, among other things, the present disclosure provides an example transformer architecture. In FIG. 13, blocks with up left right diagonal course stripes are used to represent model inputs, blocks with up right left diagonal course stripes identify layers with learnable parameters, blocks with diagonal crosshatch identify intermediate representations used and/or determined within a model, and a block with up left right diagonal fine stripes shows final output (velocity fields). As shown in FIG. 13, provided example transformer architectures include a self-attention-based layer to retrieve edge information (described in further detail below), and use conditional gating approach to modulate output of (e.g., each) layer based on global variable values, which, among other things, may include a time variable, as well as a specified protein size (e.g., chain length). As shown in FIG. 13, a body of the example network is fully invariant. An equivariant layer is used as a final head. An approach for creating a self-attention based SO(3) equivariant head, which can, for example, be used in connection with an individual backbone atom representation is described in further detail below. Where a local frame representation is used, equation (10) may be implemented, e.g., to create a final equivariant output from an invariant output.

Example approaches described herein may also a use rotary embedding (described in further detail in [Su 2021] to take into account a relative position of amino acids in a sequence in self-attention layers and edge retriever layers. A rotary embedding may also, optionally, be used in a SO(3) equivariant head implemented in connection with models that utilize an individual backbone atom representation.

### 1. Node Encoder

In certain embodiments, transformer architectures as described herein use a graph representation to represent peptide backbone, amino acid sequence, and other (e.g., node properties) polypeptide information that is received as input, manipulated and/or updated during structure generation, and/or used as conditioning input. Graph representations of polypeptide structures may be determined based on, and used in connection with, local frame representations and/or individual backbone atom representations described herein.

For example, in certain embodiments, each node of an input graph representation is associated with, and represents, a particular amino acid site. Node feature vectors associated with a particular node may include values of feature vectors, for example used to represent the particular amino acid site.

That is, for example, using an individual backbone atom representation as described herein, each particular node i representing the i^{th} amino acid site is associated with a feature vector x=(xᵢₖ), where the subscript k indexes a particular backbone atom. Using a local frame representation as described here, each particular node is associated with a feature vector x=(xᵢ^{CA}, Rᵢ), having values identifying each local frame coordinate (e.g., alpha-Carbon coordinate) and rotation.

As shown in FIG. 13, a node encoder may be used to generate SO(3) invariant features, z=(zi), from input feature vectors. Node encoder shown in FIG. 13 may also account for additional node properties, such as amino acid side chain types and/or positional encoding (e.g., if a rotary embedding approach is not used). Node encoder may encode such node-level information by summing an amino acid side-chain type embedding and a positional encoding.

A node encoder used in connection with models that utilize an individual backbone atom representation may encode geometric information based on individual backbone atom positions at each amino acid site by passing a concatenation of norms, dot products and determinants computed using individual backbone atom positions within each site, according to the below. In one embodiment, norms, dot products, and determinants are calculated based on individual backbone atom positions, concatenated, and processed by a feed forward network (FFN) to produce an encoding. The concatenation of these three sets of features is SO(3) invariant. Example norm, dot product, and determinant quantities used herein include the below:
Norms:

$$‖ {x}_{i} ( N ) - {x}_{i} ( CA ) ‖$$

$$‖ {x}_{i} ( C ) - {x}_{i} ( CA ) ‖$$

$$‖ {x}_{i} ( O ) - {x}_{i} ( C ) ‖$$

Dot products

$$〈 {x}_{i} ( N ) - {x}_{i} ( CA ) , {x}_{i} ( C ) - {x}_{i} ( C A ) 〉$$

$$〈 {x}_{i} ( O ) - {x}_{i} ( C ) , {x}_{i} ( C A ) - {x}_{i} ( C ) 〉$$

Determinant

$$det [ {x}_{i} ( N ) - {x}_{i} ( C A ) , {x}_{i} ( C ) - {x}_{i} ( C A ) , {x}_{i} ( O ) - {x}_{i} ( C A ) ]$$

Where a machine learning model utilizes a local frame representation, node-level geometric information is not used, since individual backbone atoms of amino acid sites are not explicitly modeled (e.g., substructure of amino acid sites can be recovered, e.g., based on ideal bond lengths and bond angles).

### 2. Edge Retrieval Layers

FIG. 13 includes one or more edge retrieval layers, which were developed in the present approach to address challenges in utilizing transformer architectures to generate predictions from graph representation inputs. In particular, conventional transformer approaches only operate on node features, but not edge features. In conventional transformer approaches, while node representations can be updated through a self-attention layer and feed-forward layer, edge features are not taken into account.

Accordingly, the present example introduces an approach for retrieving relative edge information in a dynamic fashion, using self-attention, and then using retrieved edge information to update latent node representations. Among other things, approaches developed and described herein leverage edge feature structure, whereby an edge feature eᵢⱼ=ƒ(xᵢ, xⱼ), to avoid a need to explicitly compute them. Accordingly, edge retrieval layers introduced herein are as memory- and computationally-efficient as conventional transformer layers (but perform functions and open the door for functionality that conventional transformer layers do not). Among other things, for example, self-attention mechanisms that can be implemented using memory-efficient attention approaches (e.g., flash attention) can be used, which allow memory usage to scale in a linearly with a number of nodes in a graph representation.

In the following, xᵢ∈ [symbol] ^{p} are used to denote node features, zᵢ∈ [symbol] ^{d} refers to an invariant representation of node i, and eᵢⱼ=ƒ(xᵢ, xⱼ)∈ [symbol] ^{q} are edge features. In the present example, in a first step, attention weights are used to retrieve, for a particular node i, a neighbor, j, via query and key values as shown below,

$${q}_{i} = {M}^{( Q )} {z}_{i}$$

$${k}_{j} = {M}^{( K )} {z}_{j}$$

$${w}_{i j} = softmax ( \frac{{q}_{i}^{T} {k}_{j}}{d} )$$

In certain embodiments, computed attention weights wᵢⱼ can be used to query node features via

$${x}_{i}^{⋆} = {\sum }_{j = 1}^{n} {w}_{i j} {x}_{i}$$

Edge features may then be computed from retrieved node features, using the following,

$${e}_{i *} = f ( {x}_{i} , {x}_{i}^{⋆} )$$

### 3. Self-Attention-Based SO(3)-Equivariant Layer

In certain implementations, machine learning models such as the transformer architecture shown in FIG. 13, used in technologies described herein introduce memory efficient self-attention-based SO(3) equivariant layers. For example, weighted summations of positions have been shown to produce SO(3) equivariant layers, for example as described in [Satorras 2021], via the equation below,

$${x}_{i}^{( l + 1 )} = {\sum }_{j ∈ N ( i )} w ( {z}_{i} , {z}_{j} ) ( {x}_{j}^{( l )} - {x}_{i}^{( l )} )$$

where (xᵢ)ᵢ are points in ( [symbol] ³)ⁿ and (zᵢ)ᵢ∈( [symbol] ^{p})ⁿ are SO(3)-invariant encodings.

This weighted summation approach can be combined with self-attention to create memory efficient SO(3)-equivariant layers, with all to all interactions, by computing the weights, w(zᵢ, zⱼ), using self-attention. For example, for a set of key/query weights M^{(Q)}, M^{(K)}, weights can be computed as follows:

$${q}_{i} = {M}^{( Q )} {z}_{i}$$

$${k}_{j} = {M}^{( K )} {z}_{j}$$

$${w}_{i j} = softmax ( \frac{{q}_{i}^{T} {k}_{j}}{d} )$$

In certain implementations, additional components/steps are introduced to address two potential shortcomings: first, softmax weights typically concentrate on a single value, whereas approaches that average multiple directions may be desired and, second, since weights computed solely above can only be positive and sum to one, not all directions can be represented.

In certain implementations, approaches described herein use several weighted attention heads to address these two issues. Each attention head h∈{1, . . . , H} can be used to select a single neighbor through via a softmax function. Corresponding directions (xᵢ*−xᵢ) may then be weighted by a feed-forward net (FNN) based on invariant encodings,

$${( {α}_{i}^{h} )}_{h} = F F N ( {z}_{i} )$$

and summed to produce the final output, as shown below

$${x}_{i}^{( l + 1 )} = {Σ}_{h = 1}^{H} {α}_{i}^{h} {Σ}_{j = 1}^{n} {w}_{i j}^{h} ( {x}_{j}^{( l )} - {x}_{i}^{( l )} ) .$$

Accordingly, because weights αᵢ^{h} can be negative, all directions are accessible. In certain embodiments, this layer can be implemented using memory-efficient attention (such as flash attention).

### 4. Example Edge Features for Edge Retrieval Layers

As described herein, edge retrieval layers utilize self-attention mechanisms to retrieve edge features. Edge features used in edge retrieval layers may be distinct from those used (e.g., typically) to represent relationships between nodes in input graphs. For example, among others, edge features used herein may include the following:

### Individual Backbone Representation Edge Retrieval Layer Edge Features

Node Features (xᵢₖ∈ [symbol] ³)ᵢₖ, where i is Amino Acid Index and k is Atom Index.
Linear Projection of Concat (Distances, Relative Orientations, Relative Positions) with
   Distances ∥xⱼₖ−xᵢₖ∥ for all k (atom types)
   Relative Orientations: Aᵢ^{T}Aⱼ with: Aᵢ=[uᵢ, vᵢ, wᵢ].

$${u}_{i} = {x}_{i} ( N ) - {x}_{i} ( CA )$$

$${v}_{i} = {x}_{i} ( C ) - {x}_{i} ( C A )$$

$${w}_{i} = {u}_{i} \times {v}_{i} ( cross product )$$

Relative Positions: Aᵢ^{T}(xⱼₖ−xᵢₖ) for all k with Aᵢ defined as above

### Local Frame Representation Edge Retrieval Layer Edge Features

Node Features: (xᵢ(CA), Rᵢ)
Linear Projection of Concat (Distances, Relative Orientations, Relative Positions) with
   Distances ∥xⱼ(CA)−xᵢ(CA)∥
   Relative Orientations: Rᵢ^{T}Rⱼ
   Relative Positions: Rᵢ^{T}(xⱼ(CA)−xᵢ(CA))

### (c) Example GNN Architecture

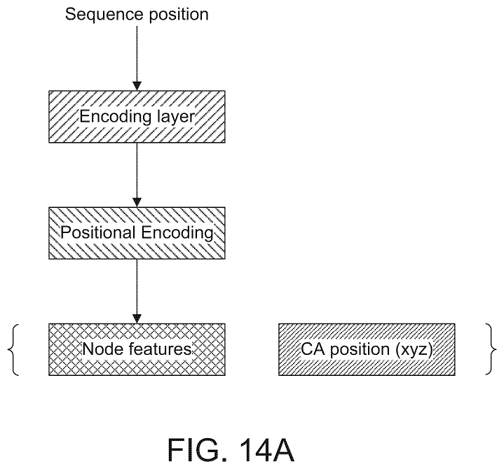

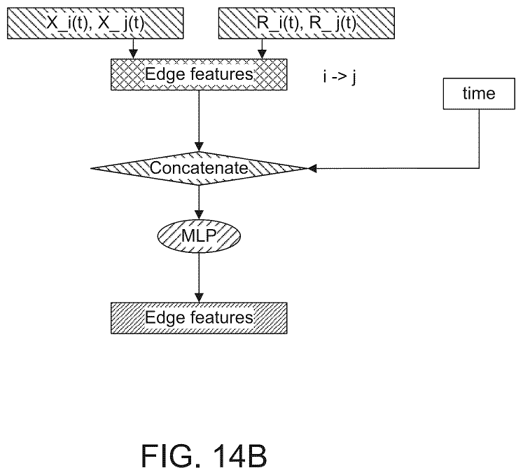

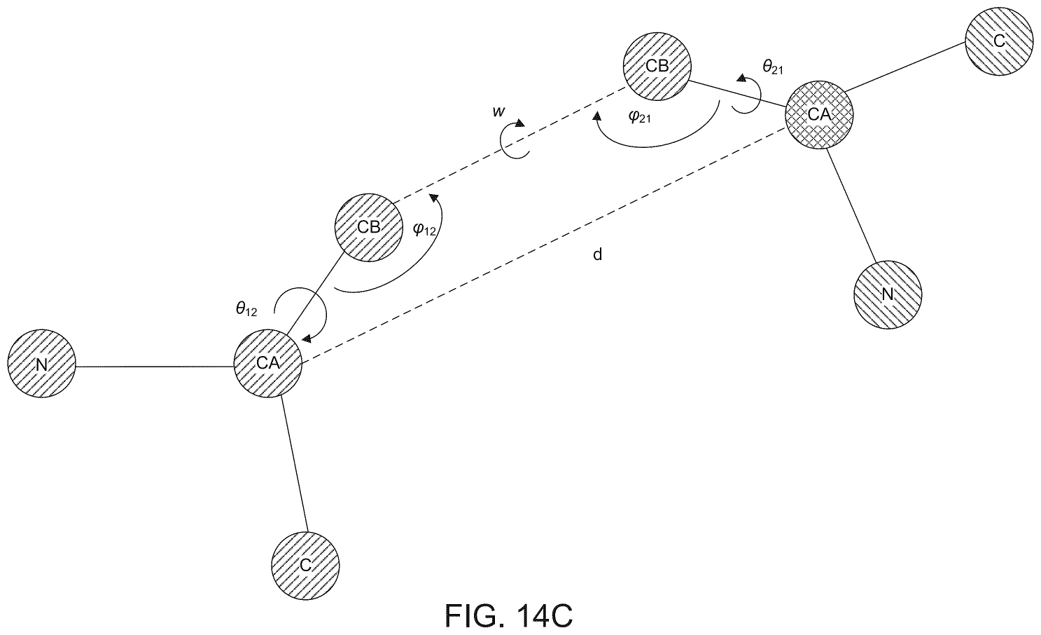

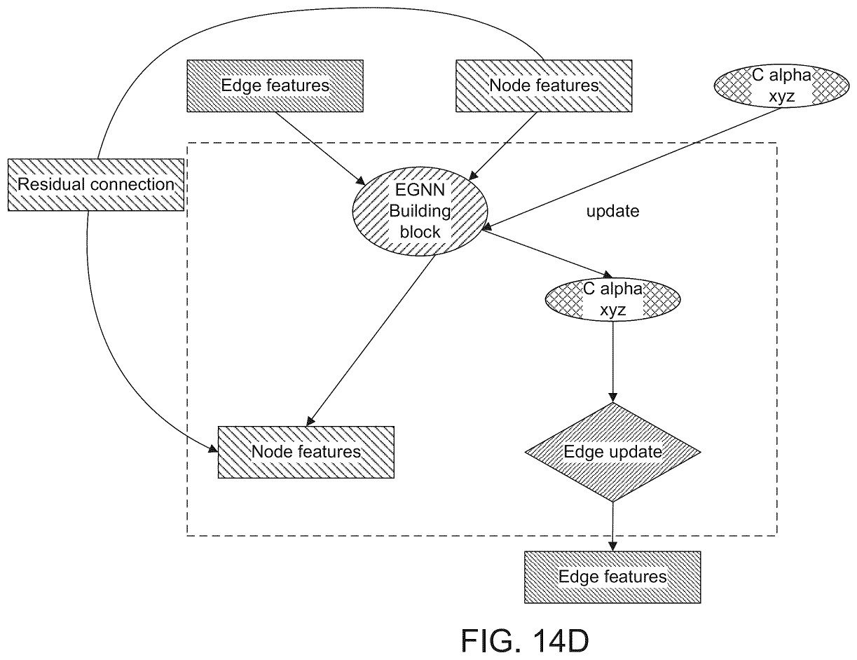

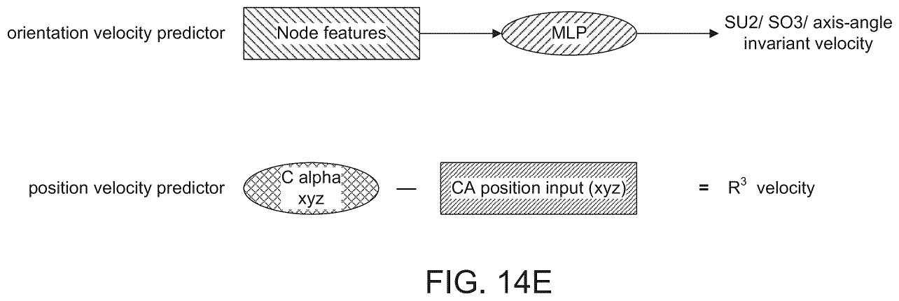

Turning to FIGS. 14A-14E, in certain embodiments, among other things, the present disclosure provides an example graph neural network architecture. The body of the network is using equivariant and invariant features.

### 1. Node Encoder

FIG. 14A shows a node encoder. Node encoder provides, among other things, a positional encoding which can be implemented via sine and cosine functions of varying frequencies:

$$P ( k , 2 i ) = sin ( \frac{k n 2 i}{d} )$$

$$P ( k , 2 i + 1 ) = cos ( \frac{k n 2 i}{d} )$$

where, k identifies a position of an object in an input sequence, 0≤k<L/2, d is a dimension of an output embedding space, P(k, j) is a position function for mapping a position k in an input sequence to an index (k, j) of a positional matrix, n is a user-defined scalar (e.g., set to 10,000, e.g., as used in [Vaswani 2017]), and the variable i is used for mapping to column indices 0≤i<d/2, where a single value of i maps to both sine and cosine functions.

### 2. Edge Encoder

FIG. 14B shows an example edge encoder. Edge encoder shown in FIG. 14B utilizes edge features computed using positions and orientation components, such as alpha carbon and frame rotation components from two amino acid sites (at a particular time, t), e.g., xᵢ(t) and Rᵢ(t) of frame i and xⱼ(f) and Rⱼ(t) of frame j.

In certain embodiments, e.g., rather than evaluate edges between every node in a graph, a k nearest neighbor approach may be used, whereby edges are evaluated and considered between the k nearest neighbors of each node. In certain embodiments, k nearest neighbors can be determined based on Euclidian space coordinates and/or sequence position. For example, in Euclidean space, a particular amino acid site (and accordingly, node)'s k nearest neighbors may be selected based alpha-Carbon (Cα-Cα) distances (e.g., excluding neighbors in sequence, in certain embodiments). A set of k nearest neighbors may also be determined based on position in sequence. In certain embodiments both approaches are used, such that sets of edges are determined with respect to a first k nearest neighbors in Euclidean space and a second k nearest neighbors in sequence position. Values of k may be, for example, selected during training, pre-defined etc. Values such as 2, 4, 8, 16, 24, 32, etc. may be chosen.

Turning to FIG. 14C, computed edge features may include (e.g., without limitation), (i) alpha-Carbon distances, dᵢⱼ=Cαᵢ−Cαⱼ (e.g., which is symmetric), (ii) three dihedral angles, ω (e.g., which is symmetric), θ₁₂, and θ₂₁ (e.g., which are asymmetric) and (iii) two planar angles, ϕ₁₂, ϕ₂₁ (e.g., which are asymmetric). Distance and angles measures are also described in [Yang 2019], albeit using beta-Carbon as opposed to alpha-Carbon distances.

These features may be used to (e.g., fully) represent a relative orientation of any two amino acid sites, i and j. In certain implementations, one or more (e.g., each) of the five angles (e.g., three dihedral and two planar) can be represented via a sine and cosine values. In certain implementations, each of the five angles shown in FIG. 14C are represented in this manner when input as edge features, resulting in eleven (11) edge features representing relative orientation between two residues (ten angles plus d). In certain embodiments, e.g., in addition to these geometry features, time input is also attached to the edge features, resulting in 12 total edge features.

In certain embodiments, computation of dihedral and planar angles used to represent relative orientations between amino acid sites, as shown in FIG. 14C, relies on locations of four atoms—namely, N, C, Cα, and Cβ. In certain embodiments, where a local frame representation is used, three pseudo atoms are generated for each node (e.g., based on idealized bond distances and angles), to represent locations of N, C, and Cβ (e.g., Cα being included in the local frame representation) and used to compute edge features as described above.

In certain embodiments, edge features may also include a binary variable that identifies whether two nodes represent neighboring amino acid sites connected by a peptide bond (e.g., having a value of 1 if two sites are connected via a peptide bond and 0 otherwise). This binary feature may be concatenated with other, e.g., geometric, edge features.

As shown in FIG. 14D, alpha carbon distances may be computed within a GNN block.

### 3. EGNN Layer

In certain embodiments, a EGNN layer is used to update x(t) in an equivariant manner, for example based on an adapted version of way.

This corresponds to an adapted version of the approach described in [Satorras 2021]. Equations for updating xᵢ(t), node embedding hᵢ, and edge features eᵢⱼ.

$${m}_{i j} = {ϕ}_{e} ( {h}_{i}^{l} , {h}_{i}^{l} , {‖ {x}_{i}^{l} ( t ) - {x}_{j}^{l} ( t ) ‖}^{2} , {e}_{i j}^{l} )$$

$${x}_{i}^{l + 1} ( t ) = {x}_{i}^{l + 1} ( t ) + \frac{1}{deg ( i )} {\sum }_{j \neq i} \frac{{x}_{i}^{l} ( t ) - {x}_{j}^{l} ( t )}{‖ {x}_{i}^{l} ( t ) - {x}_{j}^{l} ( t ) ‖} {ϕ}_{x} ( {m}_{i j} )$$

$${m}_{i} = {\sum }_{j \neq i} {m}_{i j}$$

$${h}_{i}^{l + 1} = {ϕ}_{h} ( {h}_{i}^{l} , {m}_{i} )$$

$${{A}_{i} ( t )}^{l + 1} = concat ( {R}_{i}^{T} ( t ) + {x}_{i}^{l + 1} ( t ) , {x}_{i}^{l + 1} ( t ) )$$

$${{e}_{i j} ( t )}^{l + 1} = f ( {A}_{i}^{l + 1} ( t ) , {A}_{j}^{l + 1} ( t ) )$$

Additionally or alternatively, an aggregation function for message, mᵢ can be computed as:

$${m}_{i} = \frac{1}{deg ( i )} {\sum }_{j \neq i} {m}_{i j}$$

In certain embodiments, an EGNN blocks as shown in FIG. 14D generates two outputs: updated node features and updated Cα positions. Updated Cα positions can, accordingly, be used to update edge features between each EGNN layer. In particular, edge features can be updated by building a set of three pseudo atoms, by computing

$${A}_{i}^{l + 1} ( t ) = concat ( {R}_{i}^{T} ( t ) + C {A}_{i}^{l + 1} ( t ) , {CA}_{i}^{l + 1} ( t ) )$$

Using the updated Cα position and three pseudo atoms, edge features can be computed and updated as described herein (e.g., five angles, with two values—sine and cosine—each, a binary variable identifying nodes representing sites connected via a peptide bond, time, and Cα distance).

Turning to FIG. 14E, node features and alpha-Carbon positions may be used to generate velocity predictions via a velocity prediction layer as shown in FIG. 14E and below

$${v}_{i} ( t ) = {x}_{i}^{l = n} ( t ) - {x}_{i}^{l = 0} ( t )$$

Additionally or alternatively, xᵢ^{l=n}(t) may be used as a predictor of x(t=1), in which case one can compute the velocity at t as:

$${v}_{i} ( t ) = \frac{{x}_{i}^{l = n} ( t ) - {x}_{i}^{l = 0} ( t )}{1 - t}$$

In certain embodiments, this approach can be used to generate equivariant and invariant predictions of velocities for Cα atom positions in Euclidean space and frame rotations on SO(3). In certain embodiments, any final invariant embedding can be used to create an invariant vector field, e.g., via equation (10) above, also shown below.

In certain implementations, any invariant to SO(3) layer may be used to operate on invariant features (e.g., transformer, MLP, ResNet, etc.). In certain implementations, relative positional encoding may also be used with edge representations. In certain embodiments, any invariant update function may be applied to Rᵢ^{T} at each layer before updating edge features.

### I.vi Example 6: Example Training and Model Evaluation Metrics and Dataset

This example describes several metrics and loss functions that can be used to (i) evaluate and refine (e.g., train) machine learning model performance during training and/or (ii) measure performance of machine learning models (e.g., once trained). This example also describes approaches for creating training datasets and selecting training examples therefrom.

### (a) Example Metrics

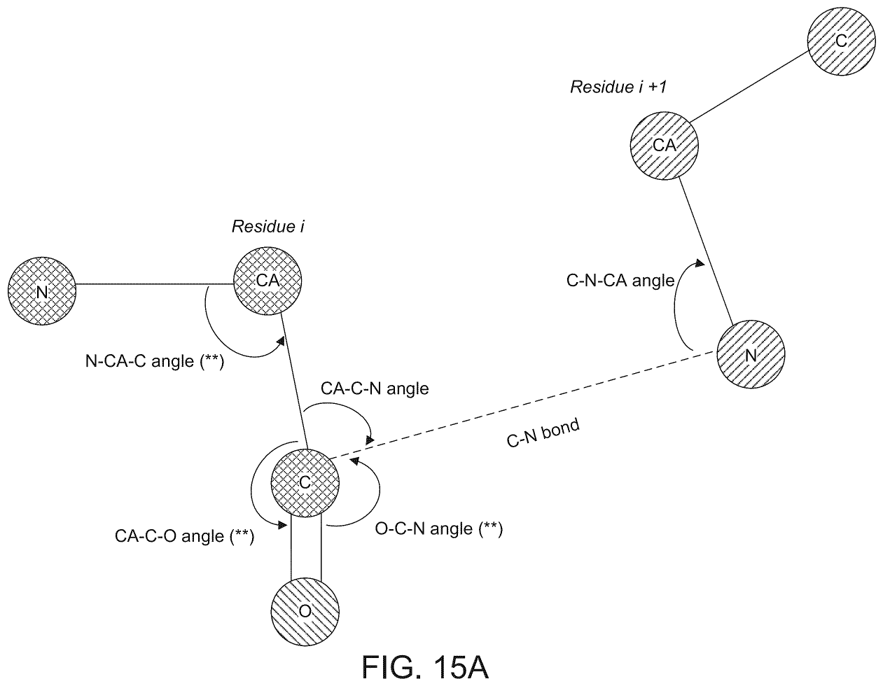

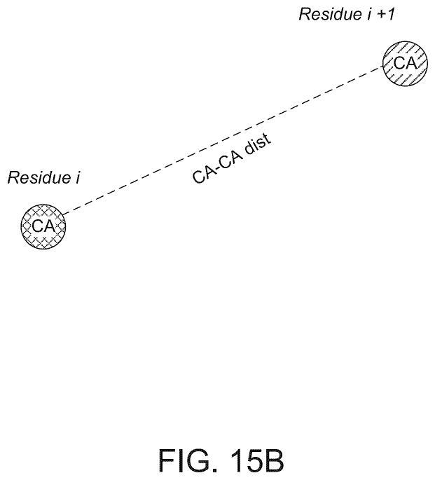

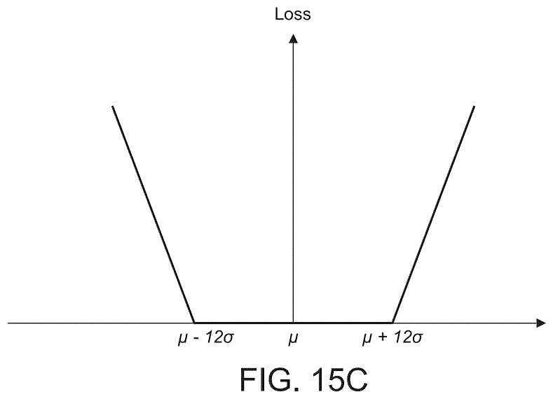

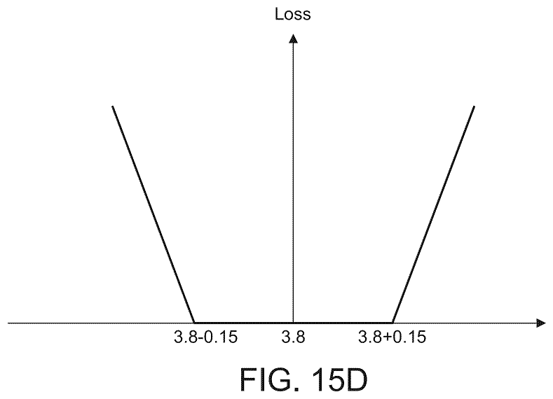

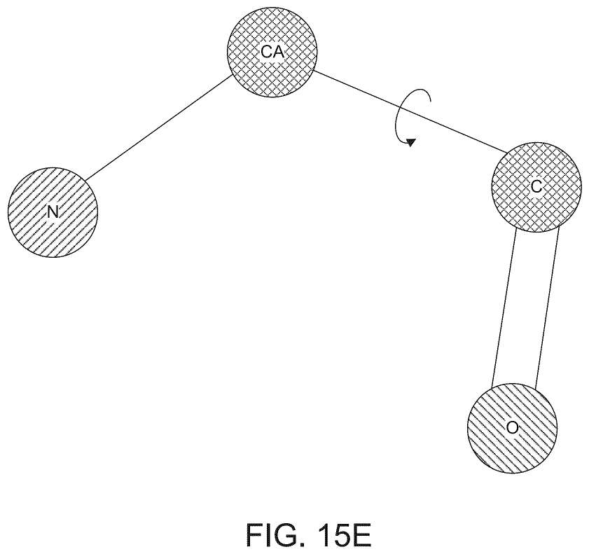

Turning to FIG. 15A, which shows portions of two consecutive amino acids (e.g., their backbone atoms), approaches described herein may use several different metrics to evaluate accuracy and/or quality of generated structures, for example, based on how closely they resemble real, physical protein and/or peptide structures.

### 1. Bond Length

In certain embodiments, metrics may be calculated as or evaluated using one or more measures of bond length, which refers to a distance between nuclei of two bonded atoms (e.g., in a molecule). As shown in FIG. 15A, a bond length of a C—N bond (e.g., a C—N bond length) may be measured, for example as a distance between a Carbon (e.g., just Carbon, not beta-carbon or alpha-Carbon) of an amino acid residue and a Nitrogen of a subsequent, next, amino acid residue in sequence (e.g., between a C atom of the i^{th} amino acid residue and the N atom of the (i+1)^{th} amino acid). In certain embodiments, a C—N bond length of a generated structure may be compared to one or more reference values, such as a mean and/or standard deviation of C—N bond lengths obtained from one or more reference protein and/or peptide structures (e.g., in a PDB), reference datasets, public resources, etc. Example reference values are a mean bond length of 1.329 Å and a standard deviation of 0.014 Å.

### 2. Bond Angle

In certain embodiments, metrics used may include and/or be determined based on one or more bond angles, measured as, for example, an angle formed between three atoms in a molecule, wherein two atoms are bonded to a central atom, e.g., as shown in FIG. 15A (in FIG. 15A, and elsewhere, CA is used to denote an alpha-Carbon atom, also labeled/identified herein via Ca). Bond angles, for example in protein backbone sites (i.e., formed between three peptide backbone atoms), that can be evaluated include, without limitation, a CA-C—N bond angle, a C—N—CA bond angle, an O—C—N bond angle, a N—CA-C bond angle, and a CA-C—O bond angle, as illustrated in FIG. 15A. In certain embodiments, approaches using local frame representations may utilize only CA-C—N and C—N—CA bond angles, while approaches using individual backbone atom representations may utilize CA-C—N, C—N—CA, O—C—N, N—CA-C, and CA-C—O bond angles.

In certain embodiments, bond angles may be compared to one or more reference values, such as mean and/or standard deviations of various (e.g., corresponding) bond angles obtained from one or more reference protein and/or peptide structures (e.g., in a PDB), reference datasets, public resources, etc. For example, reference values for various backbone atom bond angles may be found in Berkholz et al., “Conformation Dependence of Backbone Geometry in Proteins,” Structure, 17 (10), pgs. 1316-1325 (2009), see, for example, Table 1 thereof. FIG. 15B illustrates the definition of CA-CA distance between two neighboring residues.

TABLE 1
Mean (mu) and standard deviation (std) values for cosines of N—CA—C,
CA—C—O, CA—C—N, and C—N—CA bond angles.

| | Cos(N—CA—C) | Cos(CA—C—O) | Cos(CA—C—N) | Cos(O—C—N) | Cos(C—N—CA) |
|---|---|---|---|---|---|
| Mu | −0.358368 | −0.501511 | −0.457098 | −0.540240 | −0.358368 |
| Std | 0.048869 | 0.029671 | 0.034907 | 0.027925 | 0.052360 |

### 3. Consecutive Residue Alpha-Carbon Distance

Turning to FIG. 15C, in certain embodiments, metrics may be calculated as or evaluated using one or more measures of a distance between CA atoms of pairs of consecutive amino acids. In certain embodiments, a CA distance, e.g., of a generated structure, may be compared to one or more reference values, such as a mean and/or standard deviation of CA distances obtained from one or more reference protein and/or peptide structures (e.g., in a PDB), reference datasets, public resources, etc. Example reference values are a mean bond length of 3.80 Å and a standard deviation of 0.05 Å.

### 4. Dihedral Angle

In certain embodiments, metrics may be calculated as or evaluated using one or more measures of dihedral angles (also known and/or referred to as torsion angles and/or torsional angles), which measure a rotation about a chemical bond between two atoms in a molecule. In particular, in certain embodiments, a dihedral angle associated with a particular bond may be measured as an angle between two intersecting planes—a first plane determined based on locations of two atoms (e.g., the plane in which the two atoms are located) on one side of a particular bond and a second plane determined based on locations of two atoms (e.g., the plane in which the two atoms located) on another side of the particular bond.

### 5. Steric Clash

In certain embodiments, metrics may be calculated as and/or evaluated using one or more measures of steric clash (also known and/or referred to as steric hindrance). In certain embodiments, steric clash refers to, and provides a measure of, interference and/or repulsion between bulky groups or atoms within a molecule. This interference may occur when a spatial arrangement of these groups prevents them from adopting their ideal and/or most stable geometric positions. Steric clash can arise due to physical size of substituents and/or atoms, which can create unfavorable interactions. Measures and/or identifications of steric clash can, for example, be determined using values of Van der Waals Radii (e.g., from literature, public datasets, etc.) and a tolerance threshold.

In certain embodiments of generative technologies described herein, steric clash can be determined based on peptide backbone atoms, e.g., N, CA, C, O, of generated structures. Two types of steric clash can be determined and/or identified: (i) inter-residue steric clash, determined based on and/or measuring and/or identifying steric clash between atoms from different residues and (ii) intra-residue steric clash, determined based on and/or measuring and/or identifying steric clash between atoms of a same residue.

### (b) Loss Scores

Loss scores can be computed for various generated outputs of machine learning models described herein using and/or based on various (e.g., combinations of) above described metrics. Loss scores can be used to (i) assess model performance during generation and/or (ii) as part of an overall loss calculation (e.g., as auxiliary loss terms) during training.

For example, loss scores can be used to evaluate model performance (e.g., once trained) by calculating values of various metrics described herein and/or loss functions based on the metrics for both generated structures and physical protein and/or peptide structures obtained, e.g., from datasets (e.g., PDB, proprietary datasets, etc.). A comparison of loss scores and value distributions can, accordingly, be used to assess a model's capability to produce protein structures resembling those found in data.

Additionally or alternatively, loss scores can be used during training, for example as auxiliary loss terms. For example, during training, as described herein, model performance can be evaluated by comparing values of velocities predicted by the machine learning model in with those calculated from data, e.g., by determining a MSE between the two. This “flow matching loss” can be used to update model parameters, e.g., as described in Example 4, above. In certain embodiments, auxiliary loss terms can be used to enforce certain biology/chemistry-based guardrails (e.g., domain knowledge) on generated structures, e.g., to penalize generated structures that have structural and/or sequence properties that fall outside expected norms of what is or is expected based on biological and/or chemical considerations. Auxiliary loss terms can be applied throughout training, and/or for particular time points, for example at time points after a particular threshold time, such as time points after 0.5, 0.75, 0.8, etc. Auxiliary loss terms can be scaled, for example via a constant (e.g., pre-selected) scaling value, λₐᵤₓ.

An example equation for determining a total loss, Lₜₒₜₐₗ, incorporating flow matching loss, L_{flow matching}, and a scaled auxiliary loss at later time points (e.g., t>0.75) is shown below.

$${L}_{total} = {L}_{flow matching} + 1 [ t > 0.75 ] {λ}_{a u x} {L}_{a u x}$$

To use the above equation, for time points after 0.75, push-forward equations described herein can be used to predict the Euclidean/manifold quantities at t=1. Resultant protein backbone structure can then be determined (based on quantities at t=1) and used calculate various auxiliary loss terms described herein (e.g., below). These can then be combined, e.g., as a weighted sum, with flow matching loss as shown in the equation above.

Several example loss terms are described below. An auxiliary loss, Lₐᵤₓ, can be computed as a combination, such as a weighted sum, of various combinations, without limitation, of the below described individual loss terms.

### 1. Peptide Bond Loss

In certain embodiments, a peptide bond loss can be determined and used. In certain embodiments, peptide bond loss may be determined based on and/or as an adapted version of equations 44 and 45 in [Jumper 2021], Supplementary Section 1.9.11.

In certain embodiments, a peptide bond loss includes several sub-components, such as: (i) C—N bond length loss, (ii) CA-C—N angle loss, (iii) a C—N—CA angle loss, (iv) N—CA-C angle loss, (v) CA-C—O angle loss, and (vi) O—C—N angle loss. In certain embodiments, angle losses (ii) through (vi) are measured using cosines of the relevant angles (e.g., as opposed to the angles themselves). In certain embodiments, a combination of (i), (ii), and (iii) is used for models that represent peptide backbones via a local frame representation as described herein. In certain embodiments, a combination (e.g., all) of (i) through (vi) is used for models that represent peptide backbones via an individual backbone atom representation as described herein.

In certain embodiments, a peptide bond loss computed for a generated structure is compared with an average of peptide bond loss computed for reference structures and/or based on reference values. Such a comparison may include, e.g., an activation function whose value is determined based on a tolerance value (e.g., an integer multiple of standard deviations) and an reference average peptide bond loss. For example, FIG. 15C shows an example activation function computed using a tolerance value of 12 standard deviations (12σ) from an average ideal angle/length (μ) in proteins structures obtained from the PDB. As shown in FIG. 15C, using an activation function, peptide bond loss is zero where angle/length values are within (e.g., plus or minus) 12 standard deviations from an average value and then increases linearly for angle/length values beyond 12 standard deviations.

### 2. CA-CA Distance Loss

In certain embodiments, a CA-CA distance metric as described herein, measuring alpha Carbon distances between pairs of consecutive amino acids in a sequence can be used, for example to evaluate whether a model generates structures with reasonable CA-CA distances. CA-CA distances for generated structures can be compared with a reference value, e.g., from literature data, public datasets, etc. For various examples described herein, a reference value of an average acceptable CA-CA distance used was 3.80 Å, with a tolerance of 0.15 Å. A CA-CA loss function based on an activation function (e.g., CA-CA distances of 3.80 Å plus or minus 0.15 Å were assigned zero loss, and then a linearly increasing loss for distances that fell outside of the plus or minus 0.15 Å window), as shown in FIG. 15D.

### 3. Oxygen Dihedral Angle Loss

In certain embodiments, CAᵢ-Cᵢ—Oᵢ—Nⱼ—CAⱼ (j=i+1 in the sequence) are on a same plane. Accordingly, predictions of CAᵢ and CAⱼ positions can be used to infer a position of an Oxygen backbone atom, e.g., using idealized bond angles. In certain embodiments, to compensate the potential error of using ideal angles, a predicted dihedral angle between a plane that contains N—CA-C and CA-C—O, as illustrated in FIG. 15E, can be used to compensate error and avoid potential clashes with other atoms. In certain embodiments, where dihedral angle is predicted via a machine learning model, this dihedral angle can be included as an auxiliary loss term (e.g., during training). Additionally or alternatively, an oxygen dihedral angle loss can be used as an evaluation metric, e.g., to assess quality of generated structures from a trained model.

### 4. Intra-Residue Clash Loss

In certain embodiments, an intra-residue clash loss term can be calculated to penalize any steric violations and/or clashes between atoms (e.g., that are not chemically bonded with each other) within a residue. In certain embodiments, an intra-residue clash loss can be calculated based on and/or as an adapted version of equation 46 in [Jumper 2021], Suppl. Sec. 1.9.11.

In certain embodiments, an intra-residue clash loss term can be determined based on distances of/between non-bonded atoms in predicted structure and adapted to penalize models where these distances are larger (e.g., outside of a tolerance) a reference value, such as an accepted Van der Waals Radii. In certain implementations herein, Van der Waals Radii were taken from literature sources and a tolerance of 1.0 Å was used.

### 5. Inter-Residue Clash Loss

In certain embodiments, an inter-residue clash loss term can be calculated to penalize any steric violations and/or clashes between atoms (e.g., that are not chemically bonded with each other) within a residue. In certain embodiments, an inter-residue clash loss can be calculated based on and/or as an adapted version of equation 46 in Jumper et al. (2021) Suppl. Sec. 1.9.11.

In certain embodiments, calculating an inter-residue clash loss for all amino acid pairs in a protein and/or peptide structure is computationally expensive. Accordingly, in certain embodiments, a k nearest neighbor approach is used, wherein a particular reference amino acid site is selected, and distances between (e.g., all) atoms of the reference amino acid site and those of its k nearest neighbors are calculated and evaluated for inter-residue steric clashes. The number of nearest neighbors, k, may be an integer such as 1, 2, 5, 10, 15, 20, etc. In certain implementations described herein, k=5 was used. This process can be performed for each amino acid site in a generated structure, i.e., selecting each as a reference amino acid site, and then evaluating inter-residue clash losses with its k nearest neighbors.

In certain embodiments, an inter-residue clash loss term can be determined based on distances of/between non-bonded atoms in predicted structure and adapted to penalize models where these distances are larger (e.g., outside of a tolerance) a reference value, such as an accepted Van der Waals Radii. In certain implementations herein, Van der Waals Radii were taken from literature sources and a tolerance of 1.0 Å was used. In certain embodiments, clashes between C—N bonded atoms of two neighboring amino acid sites were excluded from consideration in inter-residue clash loss terms, since a peptide bond loss already accounts for/penalizes non-physical distances between these atoms.

### (c) Data-Set Creation

During training and evaluation of models described in examples herein, example protein and/or peptide structures were selected from the public PDB database. To create datasets used herein, only monomers of sizes between 32 and 512 amino acids were selected, to obtain a total of 111,715 example structures.

Example structures were clustered based on chain sequence similarity, using a 30% identity, resulting in 10,492 clusters. A roughly 85%/15% split was used to split example structures into training and test datasets. In particular, 8,833 clusters were selected and assigned to the training dataset (84.2% of the clusters) and 1,659 clusters were selected and assigned to the test dataset (15.8% of the clusters). This process resulted in the training dataset comprising a total of 98,376 arranged into 8,833 clusters and the test dataset comprising 13,339 monomers arranged into 1,659 clusters.

Training examples were also obtained for multimers, also selected from the PDB database. Multimers having sizes between 32 and 512 amino acids in a complex were selected, to obtain a total of 161,459 example structures.

Example multimer structures were clustered based on chain sequence similarity, using a 30% identity, resulting in 17,213 clusters. A roughly 85%/15% split was used to split example structures into training and test datasets. In particular, 14,550 clusters were selected and assigned to the training dataset (84.5% of the clusters) and 2,663 clusters were selected and assigned to the test dataset (15.5% of the clusters). This process resulted in the training dataset comprising a total of 142,627 entries arranged into 14,550 clusters of chains (8,635 of which were appropriate for multimer training settings) and the test dataset comprising 18,831 monomers arranged into 2,663 clusters of chains (1,410 of which were appropriate for multimer training settings).

Structures were refined to retain only the amino acids that had at least three atoms, namely, CA-C—N. Additionally or alternatively, various filters can be applied to datasets to further refine examples used to train and/or test models described herein. These (e.g., additional) filters include, for example and without limitation, filtering based on secondary structure motifs/content (e.g., filtering out backbones having a loop content of 30% or more), filtering based on data completeness [e.g., filtering out PDB structures with an excessive number of missing values (e.g., amino acids in sequence, atom coordinates, etc.)], filtering based on protein and/or peptide geometric size (e.g., diameters), etc.

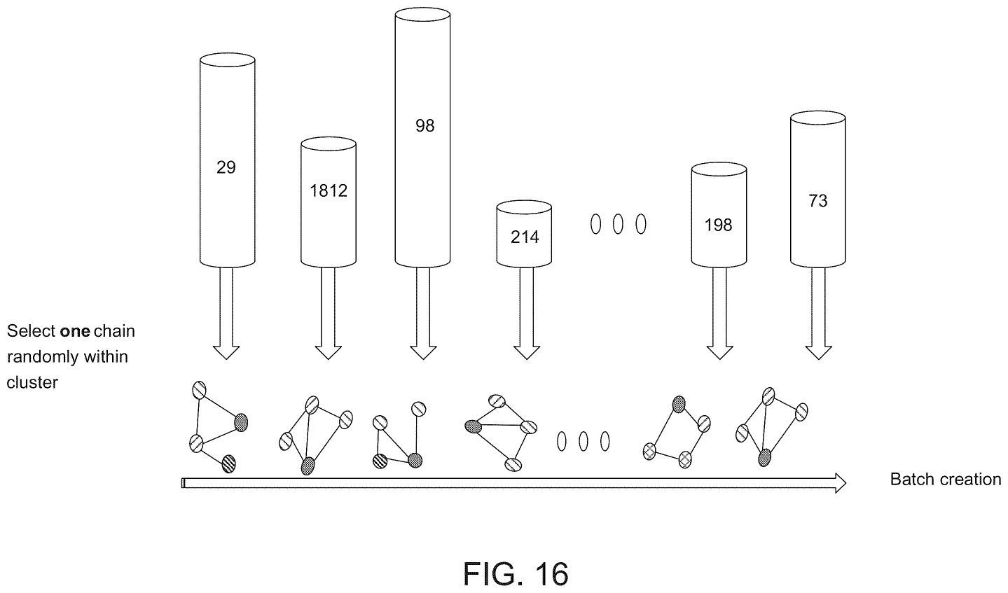

Turning to FIG. 16, during training, batches were created by shuffling cluster indices and selecting an example chain (e.g., monomer structure) from a cluster to create a batch.

### I.vii Example 7: A Multi-Modal and Multi-Task Global Framework for Generative Modeling of Protein and/or Peptide Structures

This example describes how generative technologies described herein can be used in connection with a joint training and conditioning approach to obtain a single generative machine learning model that can operate on multiple input modalities and accomplish a variety of tasks, predicting a variety of output types. Among other things, as explained and demonstrated in further detail herein, this multi-modal/multi-task approach allows a single generative model to learn task such as (i) unconditional backbone and/or sequence generation, (ii) inpainting subregions of a protein and/or peptide structure, (iii) generating a custom binder conditioned on a target, (iv) docking two proteins and/or peptides given their monomeric structures and/or sequences, (v) protein sequence design, and (vi) folding prediction.

As described and demonstrated herein, technologies of the present disclosure can be used to generate all structural and/or sequential components of a peptide and/or protein representation, including already known data on which generation is conditions (such as, for example, a backbone and/or sequence of a target to a custom protein that binds thereto is being designed). Conditioning input can include peptide backbones and/or sequence data representing portions of proteins and/or peptides being designed, targets for binders, other members of protein complexes, and the like. Conditioning input can be provided as invariant feature vectors, extracted using dedicated node encoding and/or edge retrieval layers, for example, analogous to those used to encode input representations as described, e.g., examples 4 and 5 above. In this manner, representations of conditioning data and input representations of structures being generates co-exist in a same residual stream processed and operated on by/within machine learning models described here.

### (a) Joint Training for Multiple Backbone Generation Tasks

In certain embodiments, training techniques can, for example, include a training approach that allows machine learning techniques described herein to perform multiple peptide backbone generation tasks via a single model. In an example training process, multimer structures are obtained from data, and amino acids of the example structures are partitioned into two sets. Each set may be masked, or not masked and provided as conditioning input to a model being trained. During training runs, invariant features from examples are extracted for sets that are not masked, with sets treated independently by node encoders and edge retrievers. Table 2, below shows certain example masking techniques, which can be used to create training examples that mirror particular tasks.

TABLE 2
Example Backbone Design Tasks and Input Mask Approaches.

| Task & Description | Set 1 | Set 2 |
|---|---|---|
| Unconditional generation. Generate a de-novo | Masked | Empty |
| peptide backbone | | |
| Inpainting. Given a partial protein and/or peptide | Not Masked | Masked |
| backbone, generate remaining portions | | |
| Target-Binder Backbone Design. Given a target | Not Masked | Masked |
| molecule (e.g., protein, peptide), generate a backbone | | |
| favorable for biding to it. | | |
| Docking (e.g., backbone). Given two monomers, | Not Masked | Not Masked |
| generate a viable complex comprising the two (e.g., | | |
| in a docked configuration). | | |

In a joint, multi-task, training process, a multimer from a dataset can be randomly sampled, one of a set of desired tasks, such as those shown in Table 1 above and Table 2 below, as well as others, and a mask generated based on the particular selected desired task and a masked example used as a conditioning input to a model being trained.

The above-described backbone generation tasks shown in Table 2 do not necessarily consider/include amino acid side chain types but can be adapted as explained herein to include amino acid side chain types and/or sequence data.

Amino acid side chain types can be incorporated into a model's learning procedure, with a model receiving, as described above, two sets of protein and/or peptide structures as input, with various masking layouts applied to each. With amino acid side chain type (e.g., sequence data) included, masks may obscure one or both of backbone geometry (e.g., locations amino acid residues) and side chain type. This same approach can be used to train models for all tasks described in Table 1 by masking all side chain types, and also used to provide amino acid side chain (sequence) information to a model as conditioning input, for example as shown in Table 3, below.

TABLE 3
Example Design Tasks Including Sequence and Input Mask Approaches.

| | Set 1 | Set 1 | Set 2 | Set 2 |
|---|---|---|---|---|
| | Backbone | Sequence | Backbone | Sequence |
| Task & Description | Geometry | Data | Geometry | Data |
| Sequence design. Generate a sequence | Not | Masked | Empty | Empty |
| for (e.g., that will fold into) a particular | Masked | | | |
| (e.g., given) backbone. | | | | |
| Folding. Given a sequence, generate a | Masked | Not | Empty | Empty |
| 3D structure (e.g., that it will fold into). | | Masked | | |
| Target-Binder (Backbone and | Not | Not | Masked | Masked |
| Sequence) Design. Given a target | Masked | Masked | | |
| molecule (e.g., protein, peptide), | | | | |
| generate protein and/or peptide | | | | |
| structure favorable for binding to it. | | | | |
| Docking (Backbone and Sequence). | Not | Not | Not | Not |
| Given two monomers, generate a viable | Masked | Masked | Masked | Masked |
| complex comprising the two (e.g., in a | | | | |
| docked configuration). | | | | |

Where sequence data is included, models may generate predictions of amino acid sequences, e.g., a side chain type at each amino acid site. In this approach, amino acid side chain types can be represented via a one-hot vector, for example a twenty-element vector populated with zeros and a single 1 to identify one of the twenty amino acid side chain types. For example, each side chain type can be assigned a label from 1 to 20, such that a side chain labeled 3 would be represented as [0, 0, 1, 0, . . . , 0] [symbol] ²⁰.

Although amino acid type is a discrete variable, a one-hot vector representing an amino acid type can be treated as continuous within a generative model using flow matching frameworks as described herein and a final predicted output converted to a discrete value to obtain a predicted side chain type. For example, flow matching frameworks can use a seed sampled from a uniform distribution in [0, 1]²⁰ and then utilize Euclidian space approaches described herein to evolve and generate a twenty-element vector-machine learning model output for this twenty-element vector may be continuous real numbers, which can be converted/used to determine a particular amino acid side chain type, for example via an arg max of the vector (e.g., to identify the element/index having a highest value).

### (b) Transformer Model Architecture and Implementation

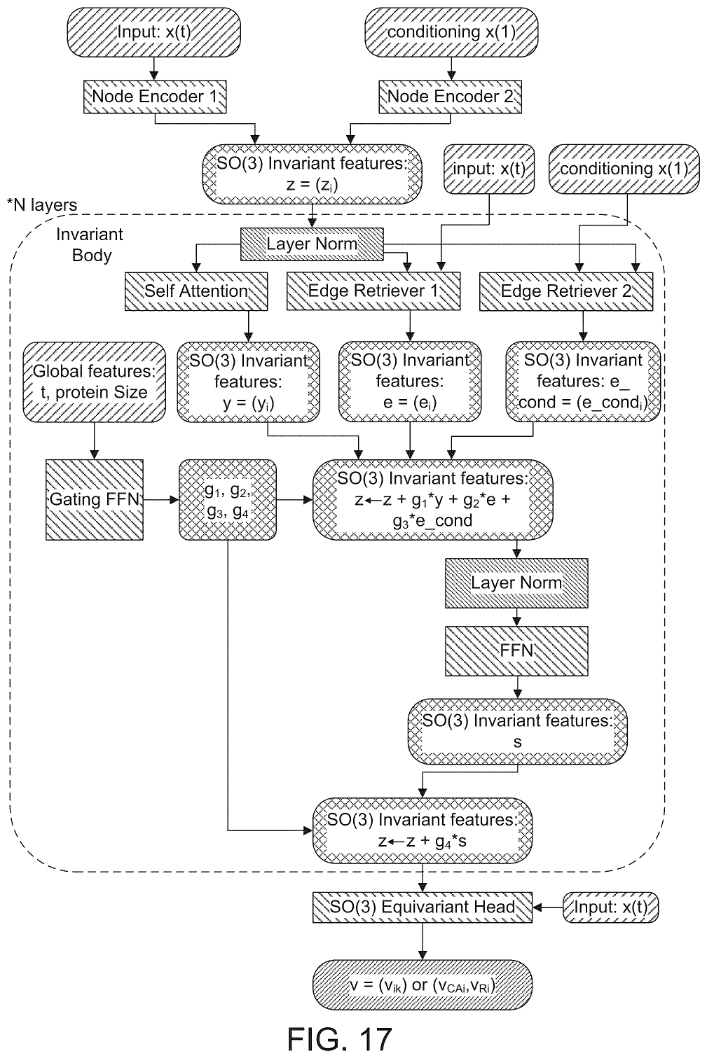

FIG. 17 provides an example transformer model architecture that can be used to include conditioning input, x(1), as described herein. An edge retrieval layer is included to operate on x(1). This edge retriever layer can function as described in Example 5, above. In certain embodiments, where conditioning input represents a monomer and/or multimer that is split into two sets as described herein, each set is processed independently from the other—that is, edge retriever operating on x(1) as shown in FIG. 17 may query edge features within each particular protein and/or peptide (set), but can be disallowed from querying edge features between two different sets, since, for example in docking and/or target binding applications, relative positions and/or orientations between constituent proteins and/or peptides are treated as unknown, to be learned and/or predicted by the machine learning model.

While described above with respect to two sets (or partial protein structures), approaches described herein can be extended to partition structures from training data into more than two sets, allowing for additional tasks to be trained on and learned. For example, using multiple, such as three, sets, a model could learn generate a custom ligand that would dock with given (e.g., known) protein structures that ordinarily would not interact, thereby allowing them to form a complex and/or bring them in proximity, in its presence. An example masking approach for training a machine learning model to accomplish this task would be to use two sets, set 1 and set 2, to represent the two given (e.g., target) proteins and a third set (set 3) to represent the ligand to be generated. Backbone geometries and/or sequences of sets 1 and 2 would not be masked, and, accordingly, used as conditioning input, while set 3, representing a ligand to be generated, would be masked (e.g., both its backbone geometry and sequence masked).

### (c) Graph Neural Network Architecture and Implementations

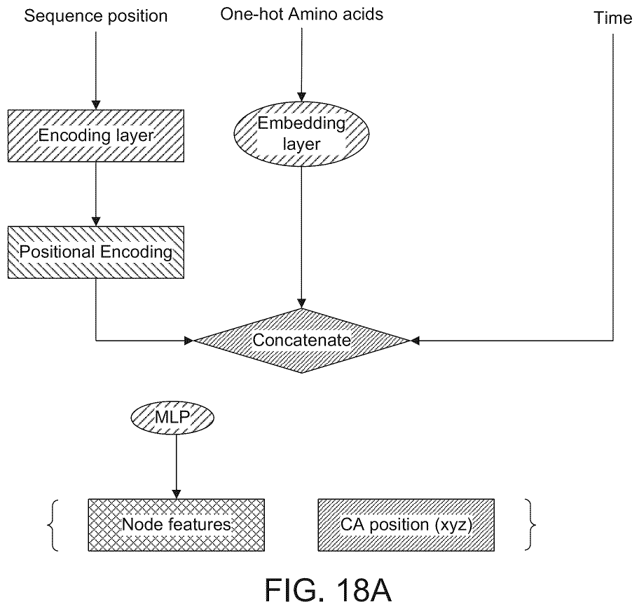

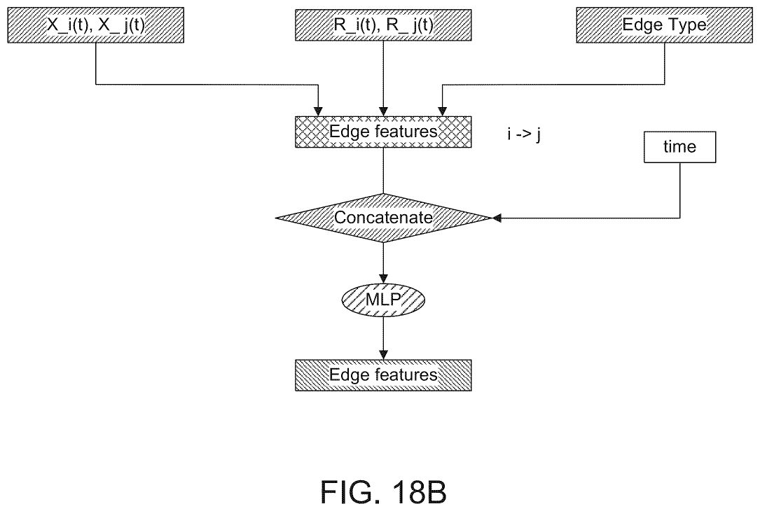

Turning to FIGS. 18A and 18B, additionally or alternatively, a GNN model may be trained and used as a multi-input/multi-task model. FIG. 18A shows a node encoder portion of a GNN architecture that can incorporate both backbone geometry and amino acid sequence data. Architecture portion shown in FIG. 18A utilizes a coupling of sequence position and amino acid side chain type as node features, whereby sequence position can be encoded as previously described herein and amino acid side chain types are input for each node as a one-hot encoding vector (in implementations for which data is generated herein, the model used a 21 element vector, with 20 elements indexing amino acid side chain types and one element indexing an unknown value). Amino acid side chain type encodings were fed into an embedding layer to generate an embedding representation, which was, in turn, combined (e.g., concatenated) with a sequence encoding and provided as input to a multi-layer perceptron (MLP) to generate embedding representations of node features used by the GNN. Time was also provided as input to a node as shown in FIG. 18A.

To account for edges representing orientations that were not to be predicted by a model (e.g., known and/or fixed a priori), time input to those edges was set to 1, while time was input to other edges as described herein, e.g., above. In inpainting tasks, time input for edges that connect two nodes that are not masked for inpainting were provided as a time feature equal to 1; other edges (e.g., edges that connect masked, to be predicted, nodes with each other, and the edges that connect masked nodes with unmasked, known nodes) retained the usual, variable, time input.

Edges were defined via a k nearest neighbor approach, as described herein, using k=8 nearest neighbors in space and k=16 nearest neighbors in sequence, for a total of 24 neighbors per node.

### 1. Four-Task GNN Model

GNN models were trained on four tasks-unconditional backbone generation, folding, and inpainting with amino acid sequence information and inpainting without amino acid sequence information, with 25 percent of the examples used for each training task.

Unconditional backbone generation was performed as described herein, above, but with amino acid side chain types set to unknown values throughout.

For training on folding tasks, models were provided with amino acid side chain types for all nodes as input, and tasked with predicting final positions of backbone sites (e.g., alpha Carbon locations and frame rotations).

Protein inpainting in the context of protein generative models refers to the process of filling in missing or incomplete information within a protein structure. Inpainting techniques in protein generative models can be valuable in various applications, including drug discovery, understanding protein folding, and predicting protein functions when dealing with incomplete or ambiguous protein data. Models used herein can receive amino acid sequence data of unknown, to be predicted portion of the protein structure, or can generate predictions without sequence this (e.g., two different tasks).

Examples for inpainting tasks were created by selecting an example protein structure and, at random, selecting contiguous subregions as hidden, to be inpainted by the model, amounting to about 20-40% of the full protein. Backbone geometry was masked for all nodes within the selected subregions to be hidden, and sequence data was masked for half the examples and provided for the other half.

During training, GNN models were provided with seed values for nodes that were masked, and to be predicted. Initial seed positions of local frames—i.e., alpha Carbon atoms—were selected from Gaussian distributions and seed rotations were selected from a uniform distribution (over SO(3)). To generate Gaussian noise as a seed for portions of proteins to be in-painted, Gaussian noise distributions can be centered between the start and end node (of the masked region) and/or half of a Gaussian can be centered on the start node and the other half centered on the end node. Ground truth/given values were provided as input for known nodes, that represented conditioning input.

For unconditional generation tasks, the neural network do not have access to any extra information such as amino acids sequence (folding), the ground truth orientation and position for some parts of the protein (inpainting), the ground truth orientation and position for the target (binder generation). Folding tasks were performed similarly, but with amino acid sequence data provided.

For inpainting tasks, during generation, positions and orientations for local frame representations of masked nodes were pushed forward according to flow matching methods described herein, from t=0 to t=1, while given/known nodes were held fixed at their input values. In-painting was performed with amino acid sequences masked and/or with amino acid sequence data provided (e.g., via node features).

In certain embodiments, in-painting techniques can be adapted to be used for binder generation, by providing values for the orientation and position of backbone sites, and, optionally, amino acid sequence data for the target. A binder to be designed may be specified in terms of a desired size (e.g., number of nodes) and the additional binder nodes treated as part of a same sequence as the target, but with a gap/jump in position, e.g., separating the binder nodes from the target node by a gap value (e.g., any number larger than a size of the protein, e.g., 100) in sequence position. Gaussian noise can be generated to seed binder design. In certain embodiments, hotspots—i.e., locations on the target where binding is expected to occur—may be known and Gaussian noise can be located on a center of mass (in space) of the hotspots. In certain embodiments, such hotspots may be unknown and/or not defined (e.g., expressly), and Gaussian noise may be placed on a center of mass of the target molecule.

### 2. Example Eight-Task GNN Model

Another example GNN model was created and trained to function on eight tasks, as follows (i) unconditioned monomer generation, (ii) monomer inpainting with amino acid sequence data, (iii) monomer inpainting without amino acid sequence data, (iv) multimer inpainting with amino acid sequence data, (v) multimer inpainting without amino acid sequence data, (vi) folding, (vii) binder generation with known hotspot sites, and (viii) binder generation without known hotspot sites.

Monomer and multimer examples were used. For unconditioned monomer generation tasks, where an example monomer chain was selected, training proceeded as described above, and if a multimer example was selected, one of the chains in the multimer example was retained and the other chains removed before proceeding. A similar approach was used for folding tasks, to retain only one chain in the event a multimer example was selected. All monomer and multimer inpainting tasks were trained as described above. For binder generation tasks, multimer examples were selected and one entire chain of the multimer was masked, with the model tasked to rebuild the masked chain.

The example eight-task GNN model used a same node encoding scheme as the four-task GNN model described above, but used a modified edge encoding scheme. In particular, turning to FIG. 18B, since this GNN model was trained on multimers and aimed to design binders, an additional edge type feature was included to encode information about the amino acid sites it connected as a seven binary element vector. First, for each of the two nodes, i and j, connected by a particular edge, three binary variables describing whether the node (i) is to be generated (e.g., 0 to be generated, 1 to be fixed), (ii) if the node is a surface node (e.g., 0 or 1, set to 0 if the node is an unknown, to be generated node), and (iii) whether the node corresponds to a hotspot site (e.g., 0 or 1, set to 0 if the node is unknown, to be generated). Second, seventh value of the edge type vector was a binary variable identifying whether two nodes were part of a same chain or a different chain (e.g., 0 or 1). Accordingly, 19 total edge features were used-10 sine and cosine values of five dihedral and planar angles, one distance value, seven additional edge type features (three node i type values, three node j type values, and 1 intra/inter chain connection indicator), and time, with as described above, time being set to 1 for known nodes and allowed to vary for nodes to be predicted.

Edges were selected using a k nearest neighbor approach, here considering also hotspots and/or surface nodes, such that if hotspot sites were identified and provided, k=8 nearest neighbors for each hotspot were selected and, if hotspot sites were not provided, k=8 nearest neighbors for surface sites were selected. Eight nearest neighbors in sequence and 3D space were also selected, resulting in 24 neighbors per node.

Training examples were processed and masked as described above for unconditional monomer generation, folding, and inpainting tasks. Binder generation data was prepared by selecting multimers and assigning one chain as the target and masking the other chain as the binder to be generated, while retaining all information for the target. The to-be-generated binder nodes had their sequence information masked by setting the amino acid side chain vector to 1 at its 21^{st} value and setting node type values in the edge type vector to 0.

During training, tasks were selected at random, but weighted to be selected a certain fraction of the time, as shown below:
Monomer tasks (25%)
   Unconditioned monomer generation (25%)
   Inpainting (50%)
Inpainting with known sequence data for masked nodes (50%)
Inpainting with unknown sequence data for masked nodes (50%)
   Folding (25%)
Multimer tasks (75%)
   Inpainting (25%)
Inpainting with known sequence data for masked nodes (50%)
Inpainting with unknown sequence data for masked nodes (50%)
   Binder generation (75%)
Known/identified hotspots (50%)
Unknown hotspots (50%)

Seed values were assigned as described above, with the noise for the binder generation task centered either on a center of mass of hotspot sites, where known, or on a center of mass of the target, where hotspot sites were unknown, and generation proceeded as described herein.

### I.viii Example 8: Generated Results Via a GNN Model Using Local Frame Representation

This example demonstrates and evaluates quality and characteristics of structures generated by machine learning models in accordance with technologies described herein. Results in this example were obtained using a GNN model implemented and trained according to approaches described in Examples 4-7, using a local frame representation whereby each amino acid site was represented using an alpha-Carbon location and rotational component.

### (a) Unconditional Generation

A graph neural network approach was first trained, as described herein, to perform unconditioned generation of peptide backbone structures.

### 1. Loss Scores

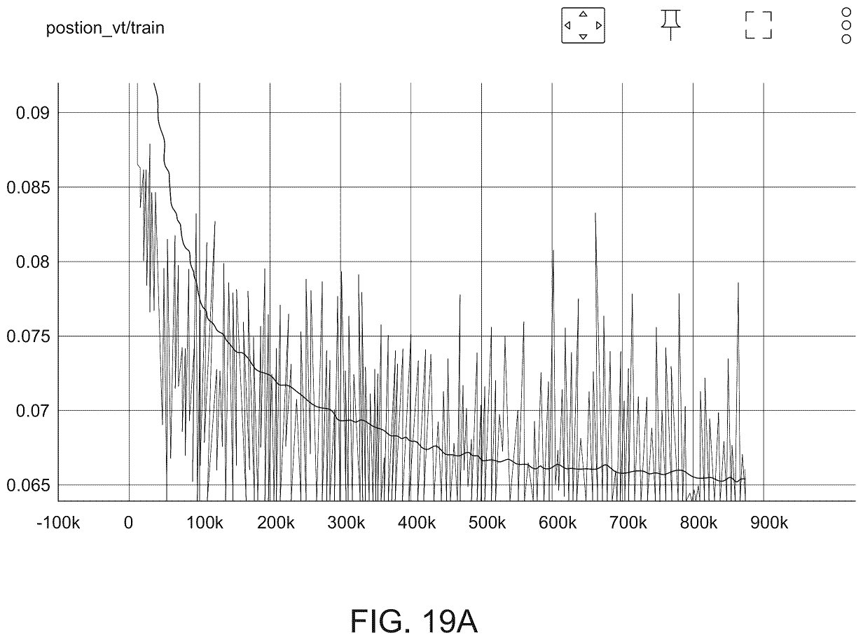

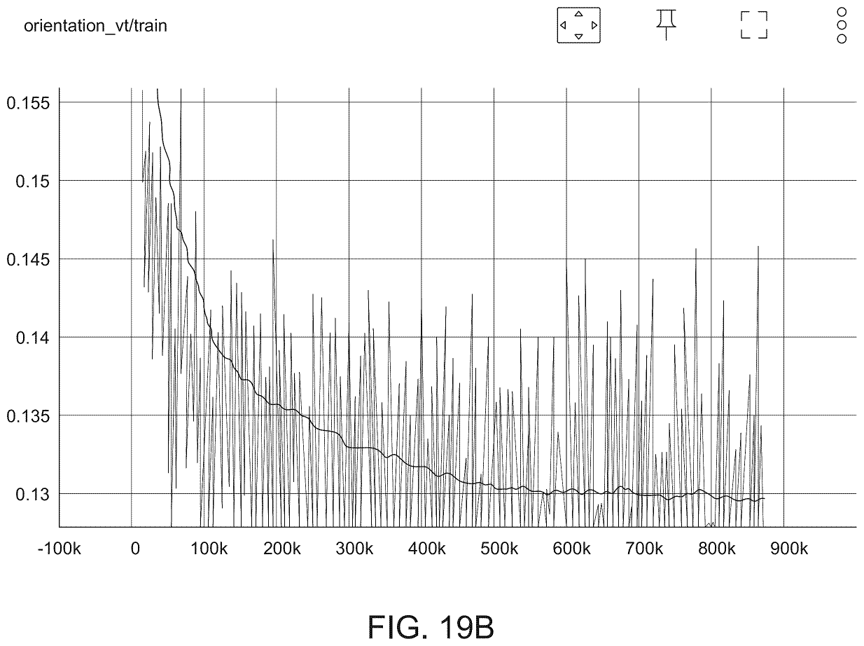

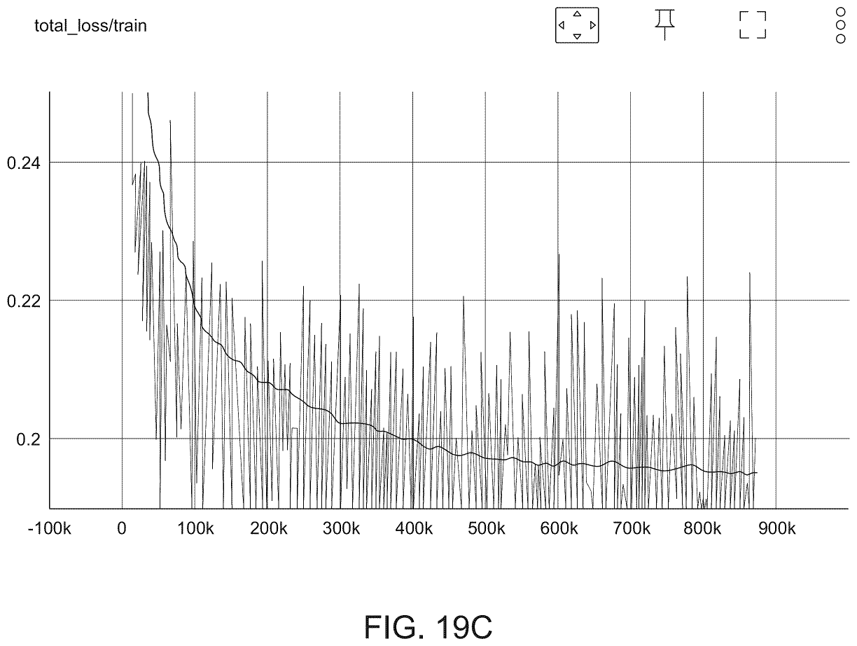

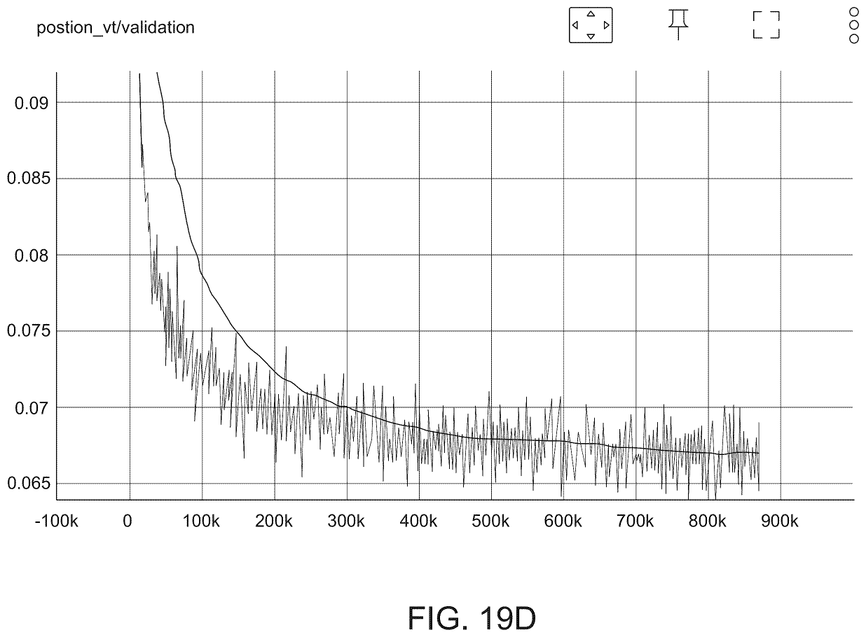

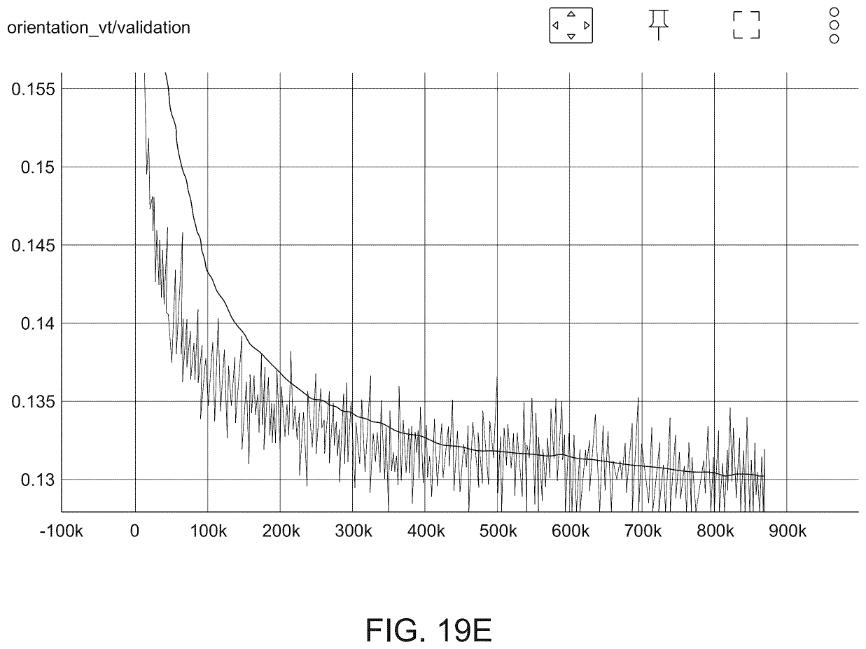

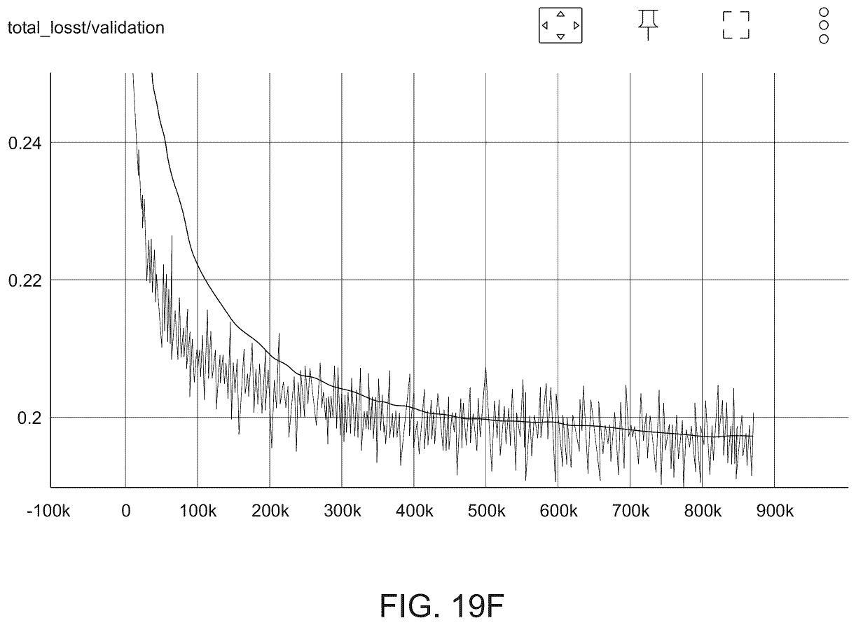

FIGS. 19A-19F show flow matching loss graphs of losses for position, orientation, and total loss for both training and validation sets, with increasing training epoch.

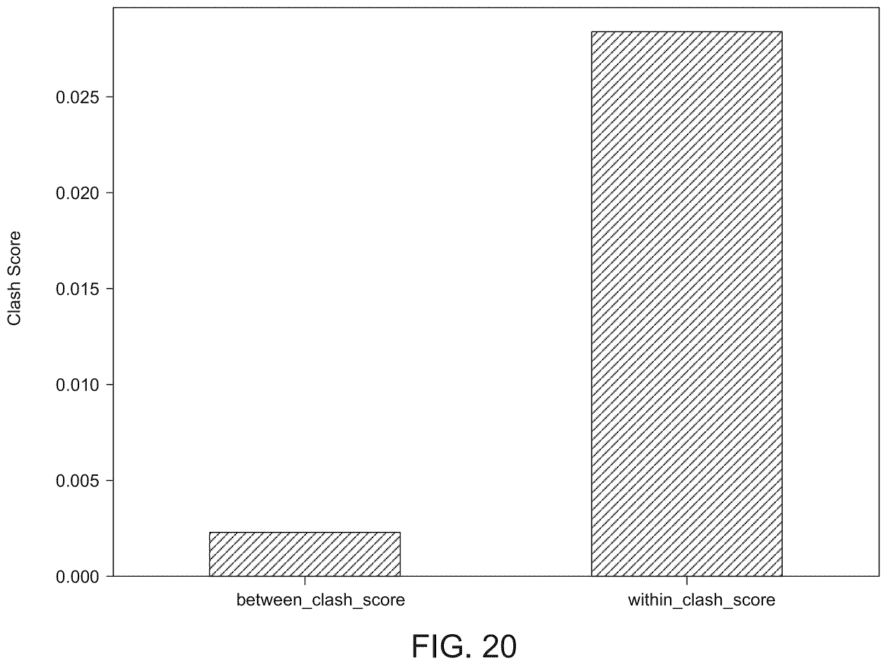

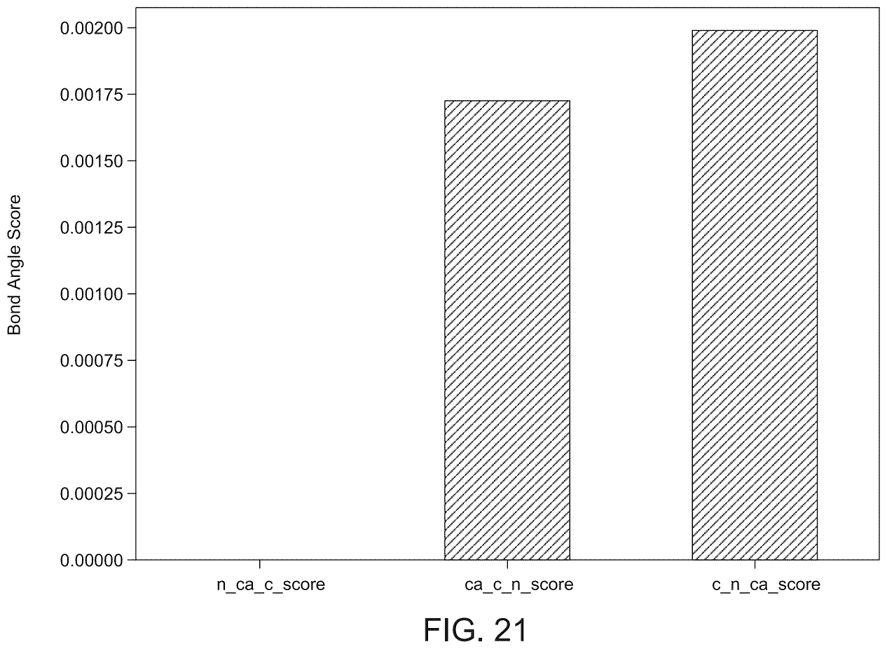

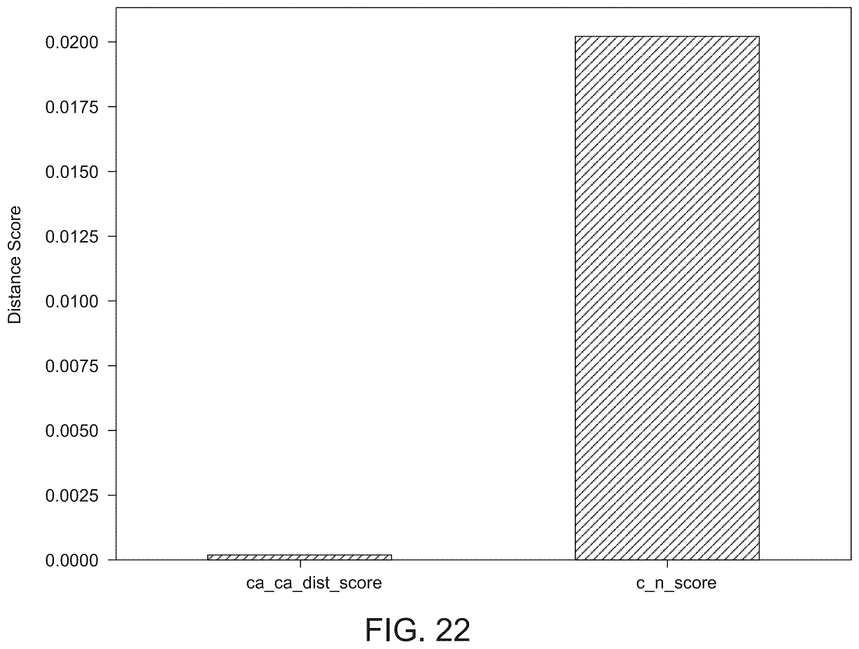

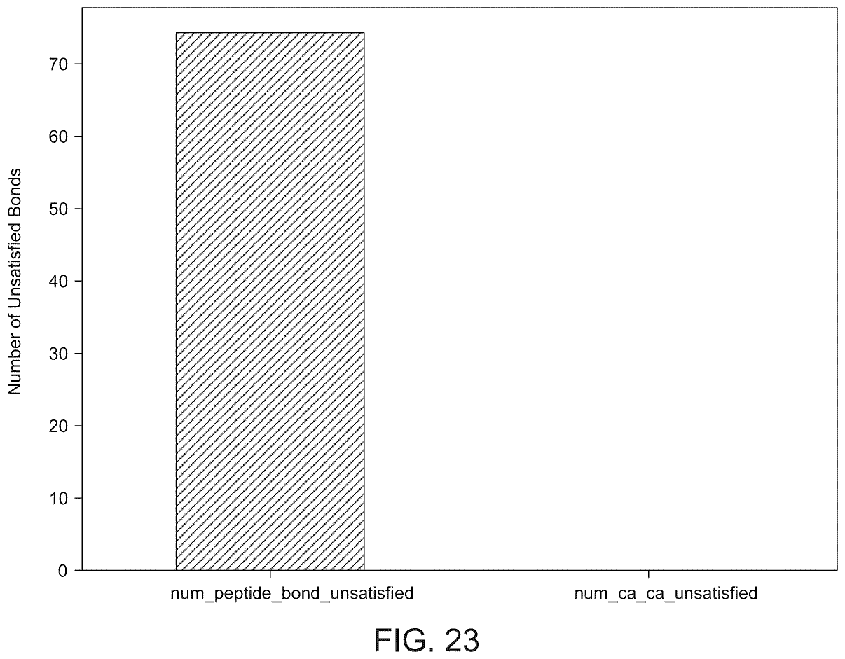

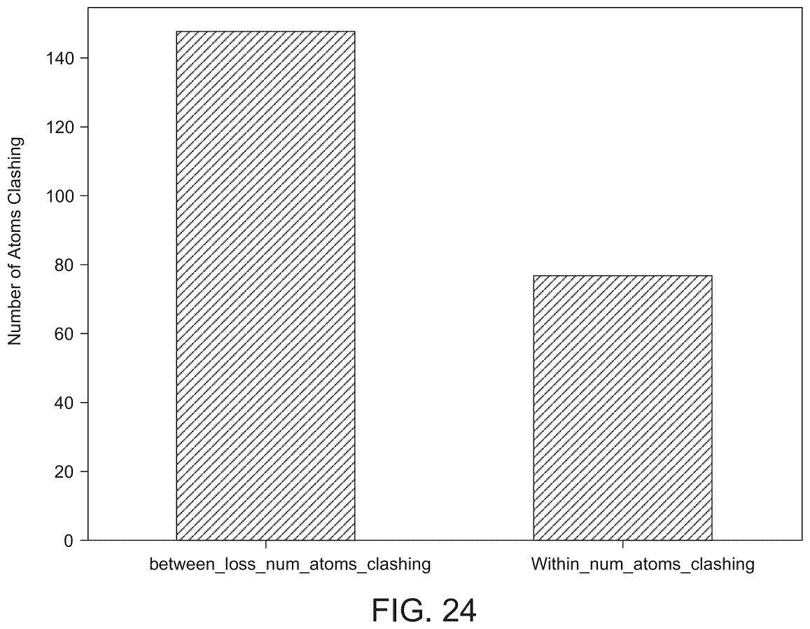

FIGS. 20-24 show graphs of scores for various loss scores described in Example 6, above for various generated structures. FIG. 20 shows values of inter-residue clash scores (“between_clash_score”) and values of intra-residue clash scores (“within_clash_score”). FIG. 21 shows values for bond angle scores determined for N—Cα-C, Cα-C—N, and C—N—Cα bonds. FIG. 22 shows values for distance scores, in particular a consecutive residue Cα distance score (Cα-Cα distance score) and a C—N bond length score, as described herein. FIGS. 23 and 24 provide counts instances various conditions, such as peptide bond lengths, Cα-Cα distances, inter-residue and intra-residue clash restrictions were unsatisfied (e.g., fell outside a tolerance value).

2. Design Benchmark metrics

Generated results were also evaluated according to various design benchmark metrics. Among other things, use of generative machine learning models to create de-novo protein and/or peptide structures aims to extend principles and concepts that govern the folding of protein structures to create structures that are new, diverse, and designable. Various metrics have been devised to evaluate the quality of structures and models that generate them, in accordance with these goals. In this example, generated scaffold models representing new peptide backbones are assessed using a variety of metrics and quality criteria. Metrics were assessed on a set of generated scaffold models representing monomers of different sizes—100, 150, 200, 250, and 300 amino acids—with fifty structures generated for each size.

Designability. Generated structures were evaluated using a designability criterion, which assesses whether a generated scaffold can be assigned a viable protein sequence. This criterion is based on a self-consistency Template Modeling (scTM) approach described in [Trippe et al. (2022)]. Although a computational method, scTM has proven to be an efficient metric at identifying designable folds, focusing on overall topology and geometry. An scTM can be computed for a particular generated structure by predicting a sequence and then using the predicted sequence as an input to a dedicated structure prediction method. In this example, to evaluate designability, a scTM score was determined by using generated structures as input to ProteinMPNN [Dauparas et al., 2022] with a sampling temperature of 0.1, to generate eight sequences per input structure. ESMFold [Lin et al., 2023] was then used to predict a folded structure of each sequence. ESMFold's predicted structures were then compared with the generated structures, with similarity measured via a TM-score [Zhang & Skolnick, 2004], which is a metric of fold agreement ranging from 0 to 1. In conjunction with a TM-score, a RMSD was also calculated to obtain an scRMSD measure. In this manner, 8 scTM and scRMSD scores were generated (one for each sequence from ProteinMPNN) for each de-novo structure, and the sequence with the best score retained (i.e. highest TM-score and lowest RMSD for the scTM and scRMSD scores, respectively). A generated scaffold was considered as designable if it had a scTM is >0.5 or scRMSD is <2 Å.

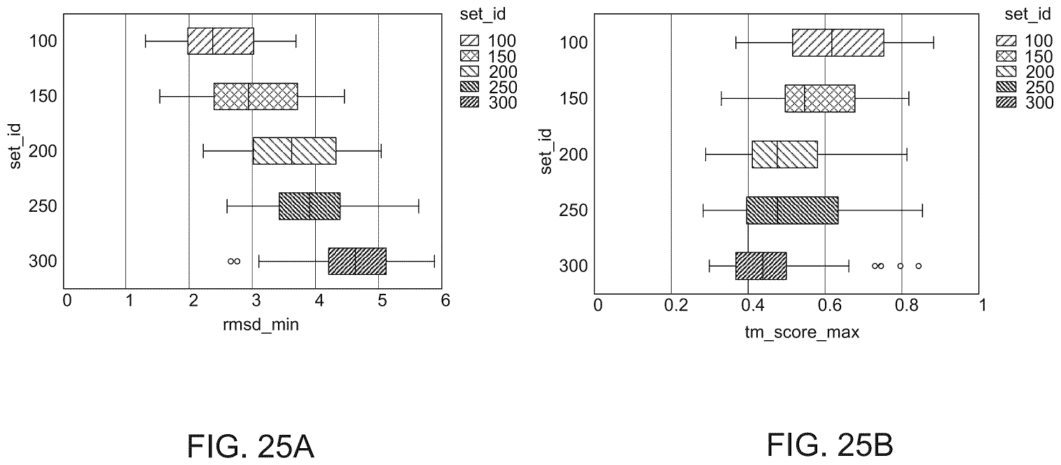

Table 4, below, provides data showing fractions of scaffold determined to be designable for different monomer sizes and FIGS. 25A and 25B show distributions (box and whisker plots) for scRMSD and scTM scores computed for generated structures, respectively.

TABLE 4
Distribution of the designable fraction of
generated structures for each length bin.

| set_id | Designable fraction | Designable fraction |
|---|---|---|
| (size) | (scTM) | (scRMSD) |
| 100 | 0.84 | 0.7 |
| 150 | 0.72 | 0.52 |
| 200 | 0.42 | 0.22 |
| 250 | 0.46 | 0.08 |
| 300 | 0.24 | 0.04 |
| All | 0.536 | 0.312 |

Diversity scores were constructed by running all-to-all Foldseek alignments of designable generated structures against themselves, followed by a pass of TMAlign over resulting alignment hits. Both the TM score and RMSD of alignments are computed to allow comparison with other leading methods. For TM scores, lower mean values indicate that sampled structures are less similar (more diverse), while for RMSD, higher values indicate increased diversity.

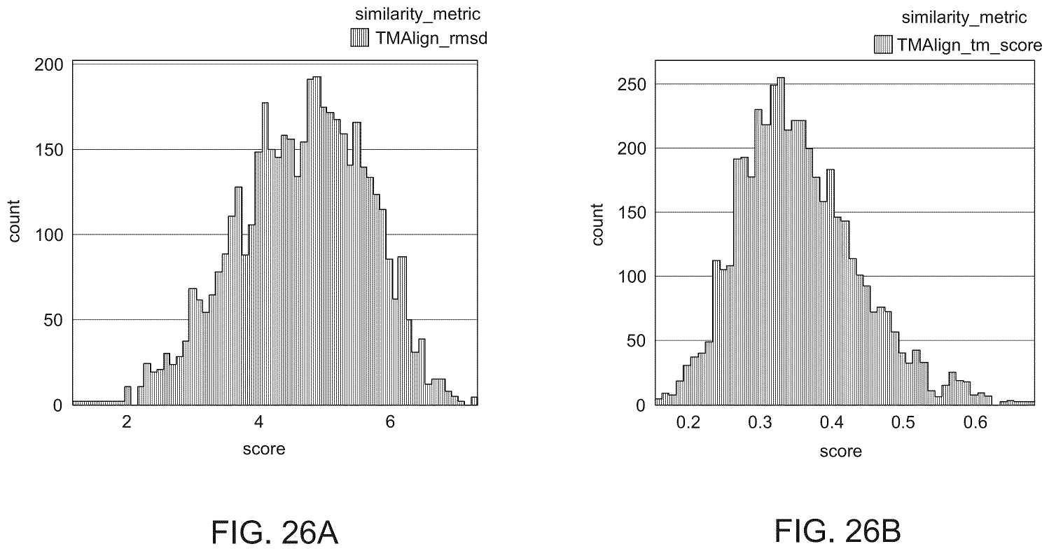

Table 5 provides statistics on diversity scores computed using RMSD and TM scores and FIGS. 26A and 26B plot histograms of determined RMSD and TM scores, respectively.

TABLE 5
Diversity metric statistics.

| | Diversity score | | | | |
|---|---|---|---|---|---|
| Similarity Metric | Mean | Standard Dev. | 25% | 50% | 75% |
| TMAlign RMSD Score | 4.7164 | 0.9923 | 4.06 | 4.8 | 5.45 |
| TMAlign TM Score | 0.3610 | 0.0829 | 0.3024 | 0.3512 | 0.4108 |

Another set of results was produced based on alternative framework as below.

Designability Fraction. The Designability Fraction quantifies the percentage of entries within a collection that can be effectively designed. The assumption of being effectively designed is based on self-consistency between ProteinMPNN [Dauparas et al., 2022] and AlphaFold [Jumper 2021] and/or ESMFold [Lin et al., 2023].

Using MPNN, inverse folding was performed 8 times to generate sequences, which were subsequently folded using a protein folding model (ESMFold or AlphaFold) to predict their structures. The root mean square distance (RMSD) between the original structure and the predicted ones was calculated to assess self-consistency, labeled as ScRMSD. The lowest ScRMSD score from the 8 attempts was selected for each structure. Entries with an ScRMSD score lower than a set threshold (2 Å) were considered designable. The Designability Fraction represents the proportion of entries in the collection deemed designable by these criteria, providing a statistical overview, including the mean and standard error, of designability within a specific collection.

Designability Self-Consistency RMSD. This metric was calculated as the mean ScRMSD for the designable entries in a collection. It represents the average of ScRMSD values of designable entries within a set.

Novelty Fraction. The Novelty Fraction evaluates the proportion of designable entries that are novel within a collection. The designable entries were first filtered (using the same ScRMSD criterion for designability). Then, the fraction of these entries that meet the novelty criterion (max TM score smaller than a threshold) were calculated, along with the mean and standard error. The Novelty criterion was computed between the designs and the training dataset used to train the generative model.

Novelty Average Max TM Score. This metric calculates the average of the maximum TM scores used to compute the Novelty Fraction. This metric is used for discovering unique structures that expand the known protein space, highlighting the innovation potential of the design process. Only entries that meet the designability criteria (based on the ScRMSD score) were considered for calculation. The maximum TM-score between a designable entry and the full set of structures used during training was computed. The average of this max TM score corresponds to the Novelty Average Max TM Score.

Diversity. This metric evaluates the range of structural variations produced by the design process, emphasizing the capability to generate a wide array of distinct structures and highlighting the design process's exploration of the structural space. Only pairs where both entries are designable and belong to the same design set were considered. Diversity was computed as the TM-score pairwise for entries within a collection in the form of the average and standard deviation between those entries.

TABLE 6
Design Benchmark Metrics values for generated structures for each length bin.

| | Designability | Designability | Novelty | Novelty Avg | |
|---|---|---|---|---|---|
| Population | Fraction | ScRMSD | Fraction | Max TM score | Diversity |
| overall | 0.624 ± 0.0031 | 1.899 ± 0.072 | 0.212 ± 0.0033 | 0.579 ± 0.008 | 0.367 ± 0.001 |
| 100 | 0.880 ± 0.046 | 1.256 ± 0.112 | 0.136 ± 0.052 | 0.607 ± 0.021 | 0.371 ± 0.002 |
| 150 | 0.760 ± 0.060 | 1.472 ± 0.126 | 0.053 ± 0.036 | 0.611 ± 0.013 | 0.381 ± 0.002 |
| 200 | 0.560 ± 0.0070 | 1.990 ± 0.157 | 0.286 ± 0.085 | 0.554 ± 0.016 | 0.360 ± 0.002 |
| 250 | 0.600 ± 0.0069 | 1.937 ± 0.129 | 0.300 ± 0.084 | 0.553 ± 0.015 | 0.350 ± 0.002 |
| 300 | 0.320 ± 0.066 | 2.841 ± 0.173 | 0.500 ± 0.125 | 0.520 ± 0.020 | 0.339 ± 0.003 |

### 3. Local Secondary Structure Metrics

Detailed assessment of structure quality was also evaluated by measuring local geometry and properties of secondary structure elements. Local secondary structure metrics were evaluated for the designable fraction that meet the scTM threshold designability criteria described herein.

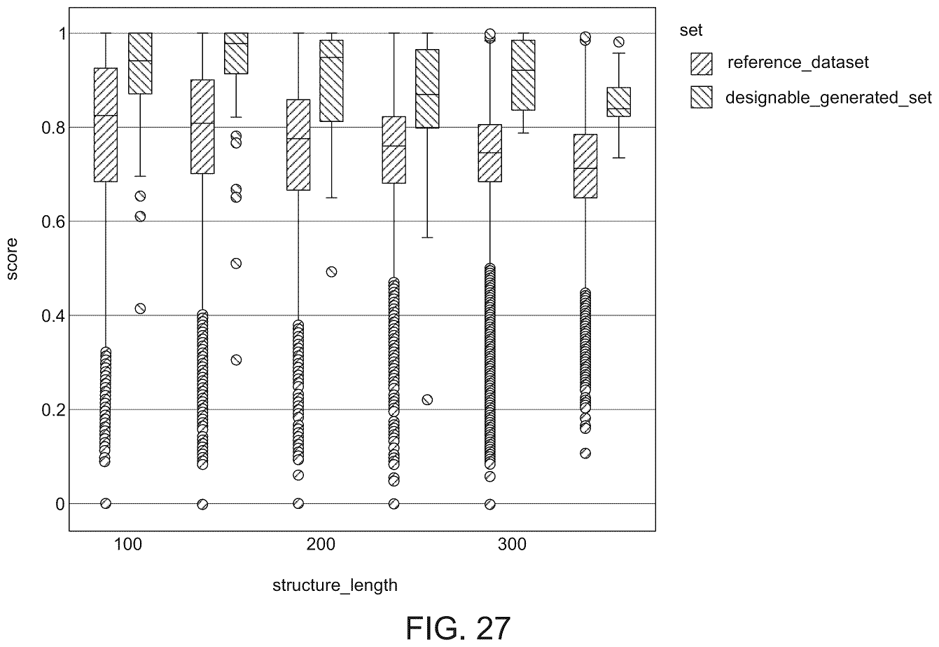

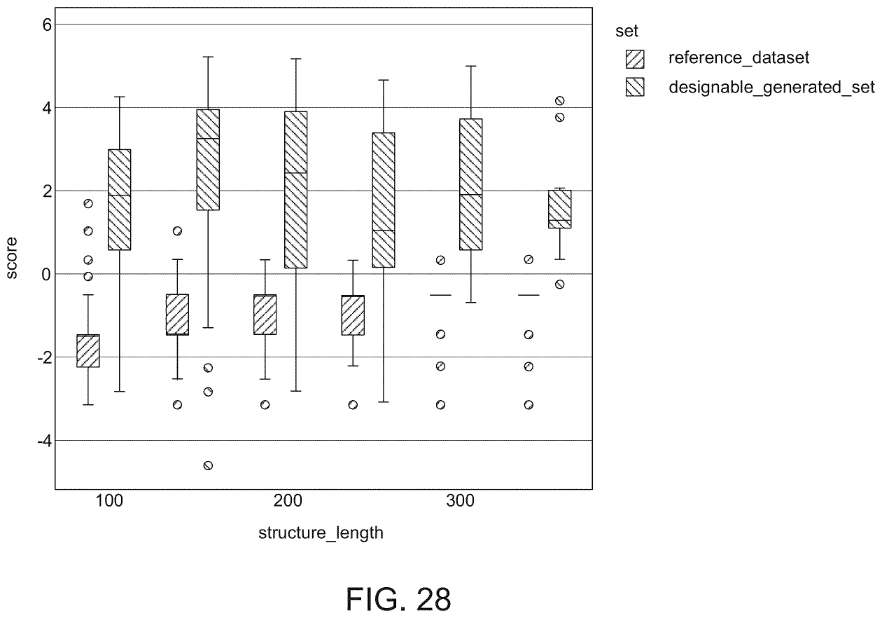

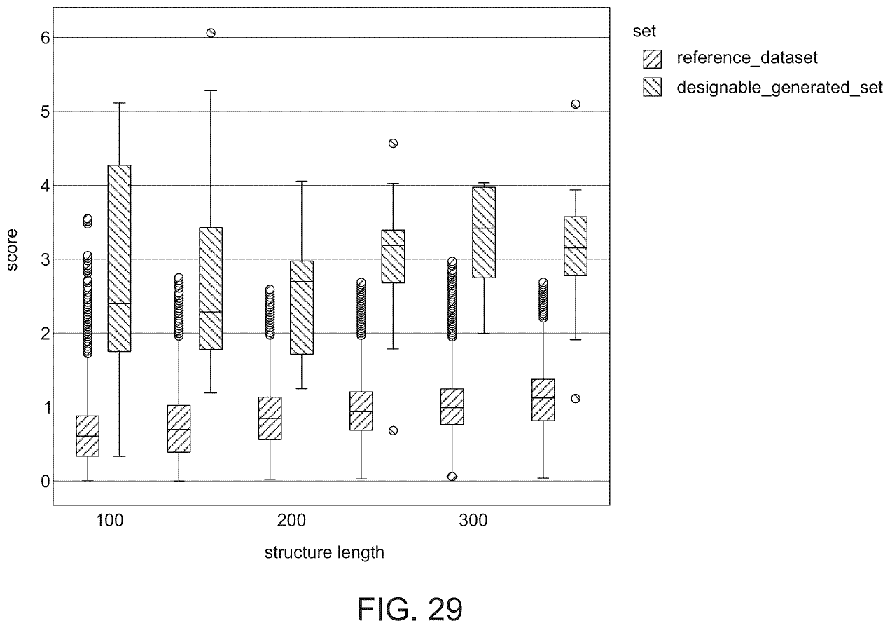

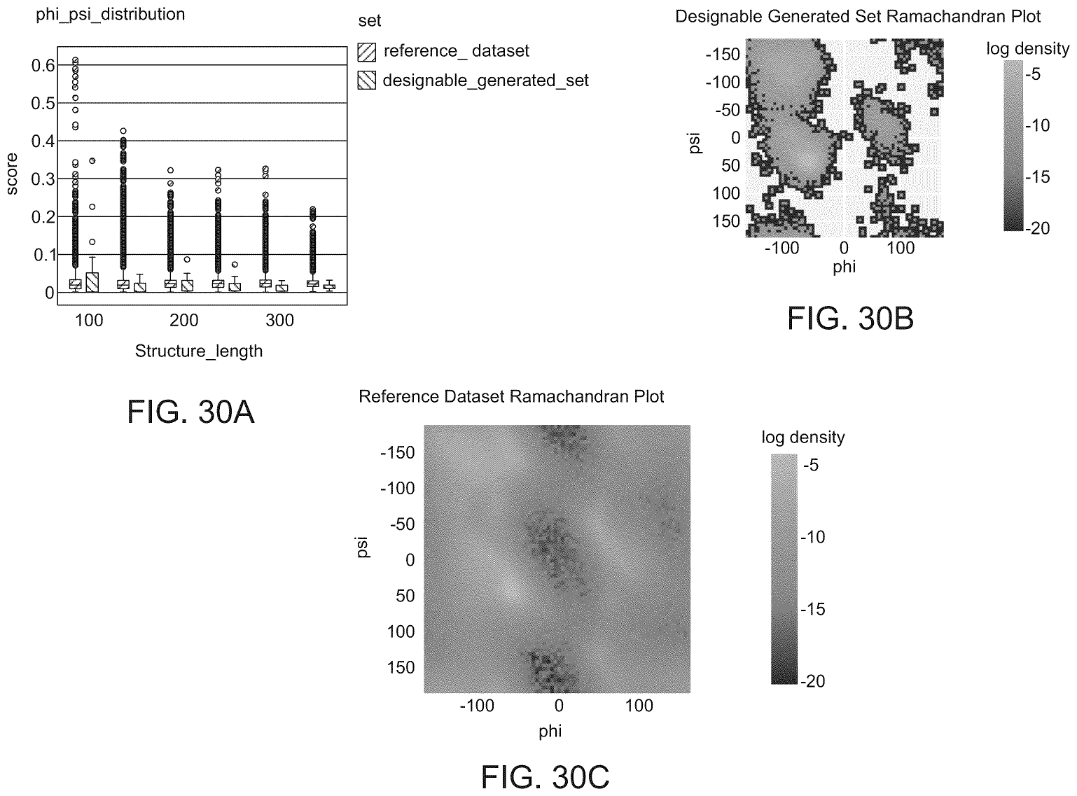

In particular, five metrics were developed and evaluated for both the generated sample and a reference dataset of experimental structures. The reference dataset is the same exact dataset that was used for training the generative models. FIG. 27 shows values of a secondary structure run (“SS run”) metric, which evaluates a percentage of helix/strand residues that are in contiguous secondary structure runs of length greater than a particular cutoff value (here 5 residues). FIG. 28 shows values of a secondary structure composition (“SS composition”) metric, which measures a log likelihood that a structure has a higher fraction of helix and strand content compared to a randomly sampled example (e.g., PDM) structure from a training dataset. FIG. 29 shows values of a secondary structure contact (“SS contact”) metric which measures a mean frequency of helix/strand to helix/strand contact normalized by total helix/strand (evaluated as number of secondary structure runs). FIG. 30A shows plots of backbone dihedral (phi and psi angles) distribution metric, measuring a fraction of phi-psi outliers as determined by computing phi and psi dihedral angles of each residue and scoring based on known dataset phi-psi density grid. Outliers were identified as those residues with scores (log density) below a particular threshold. FIGS. 30B and 30C show Ramachandran plots for aggregate residue phi-psi distributions for generated designable structures as well as a reference dataset, respectively.

These metrics and plots show that the distributions of secondary structure metrics for the generated structures align with those reported for the reference dataset. The scores in the generated set often surpass those in the reference structures, indicating that sampling strategy chosen produces highly structured, compact folds with long stretches.

### 4. Example Generated Structures

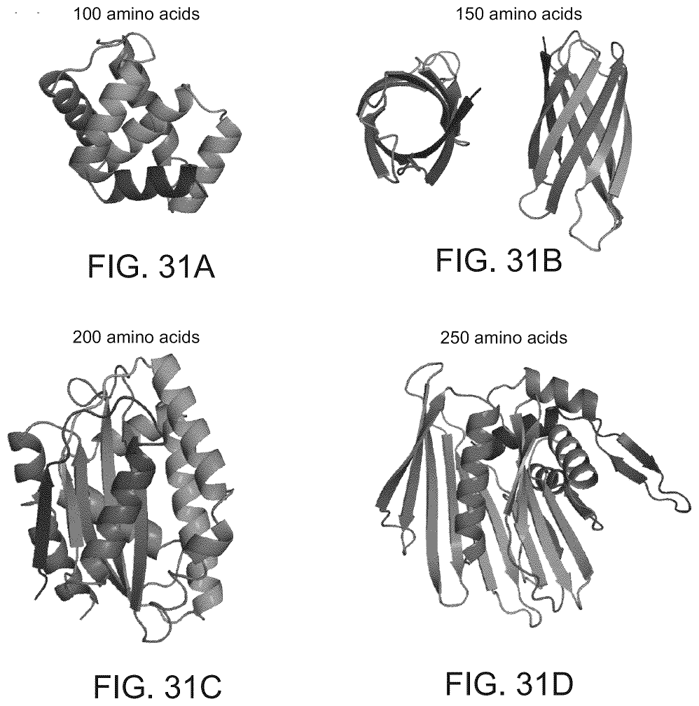

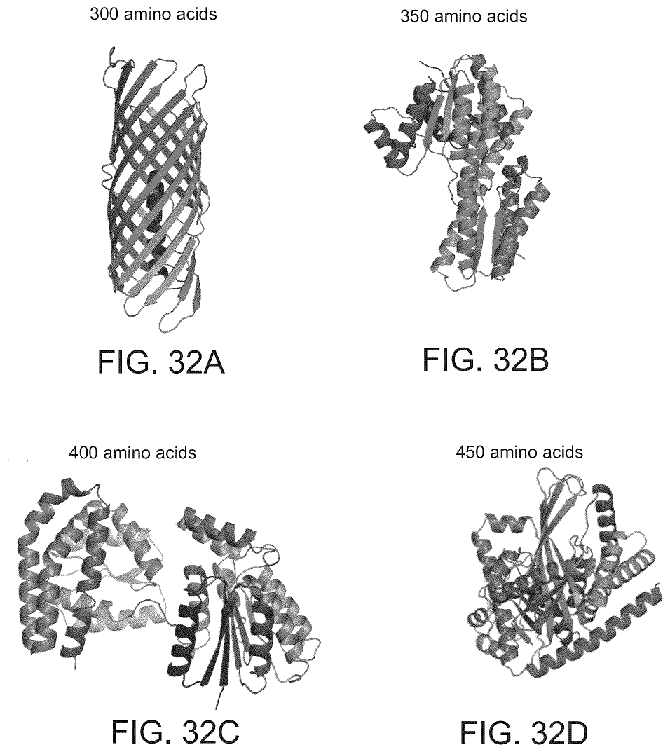

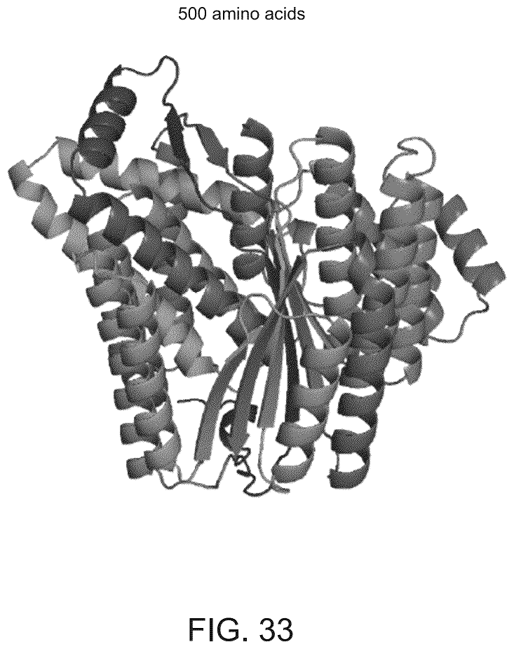

FIGS. 31A-33 show examples of generated protein backbone structures of various sizes, with FIGS. 31A-31D showing example structures generated with 100 amino acids, 150 amino acids, 200 amino acids, and 250 amino acids, respectively, FIGS. 32A-32D showing example structures with 300 amino acids, 350 amino acids, 400 amino acids, and 450 amino acids, respectively, and FIG. 33 showing an example generated structure comprising 500 amino acids.

### (b) Inpainting

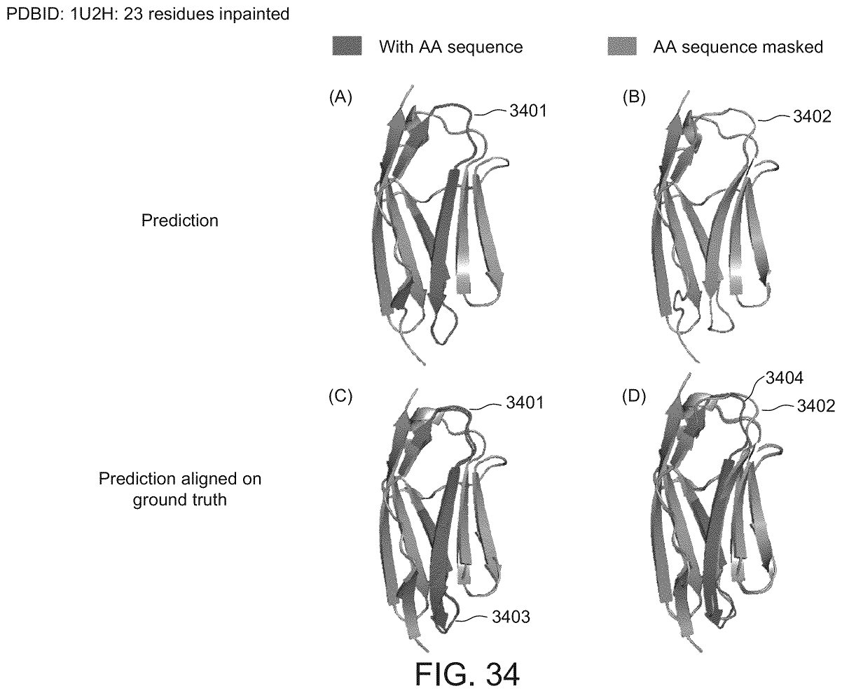

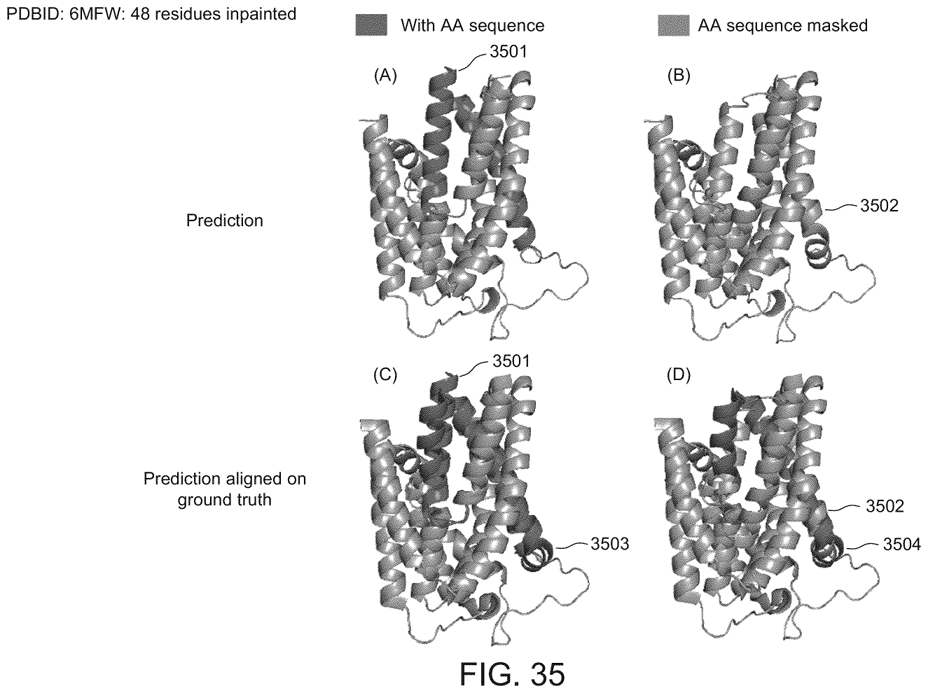

A GNN model was also trained to perform inpainting tasks, e.g., as described in Example 7 herein. Generated predictions for inpainting tasks performed by masking subregions of various PDB structures and using a trained model to recover subregions are shown in FIGS. 34-35.

FIG. 34 shows ribbon diagrams illustrating predictions performed on PDB structure PDB ID 1U2H with 23 residues masked and inpainted by the machine learning model. Predictions 3401 and 3402 are shown alone in the top panes, (A) and (B) and overlaid on ground truth 3403 and 3404 in bottom panels, (C) and (D). Panels (A) and (C) show results obtained where amino acid sequence data was provided, and panels (B) and (D) show results obtained where amino acid sequence data was masked.

FIG. 35 shows ribbon diagrams illustrating predictions performed on PDB structure PDB ID 6MFW with 48 residues masked and inpainted by the machine learning model. Predictions 3501 and 3502 are shown alone in the top panels, (A) and (B) and overlaid on ground truth 3503 and 3504 in bottom panels, (C) and (D). Panels (A) and (C) show results obtained where amino acid sequence data was provided, and panels (B) and (D) show results obtained where amino acid sequence data was masked.

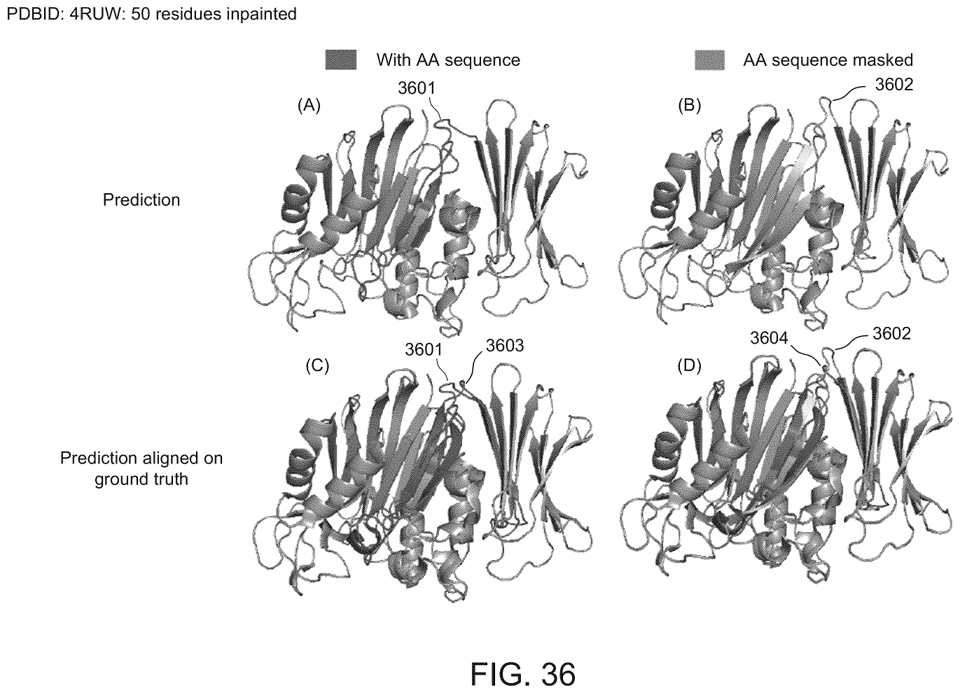

FIG. 36 shows ribbon diagrams illustrating predictions performed on PDB structure PDB ID 4RUW with 50 residues masked and inpainted by the machine learning model. Predictions 3601 and 3602 are shown alone in the top panels, (A) and (B) and overlaid on ground truth 3603 and 3604 in bottom panels, (C) and (D). Panels (A) and (C) show results obtained where amino acid sequence data was provided, and panels (B) and (D) show results obtained where amino acid sequence data was masked.

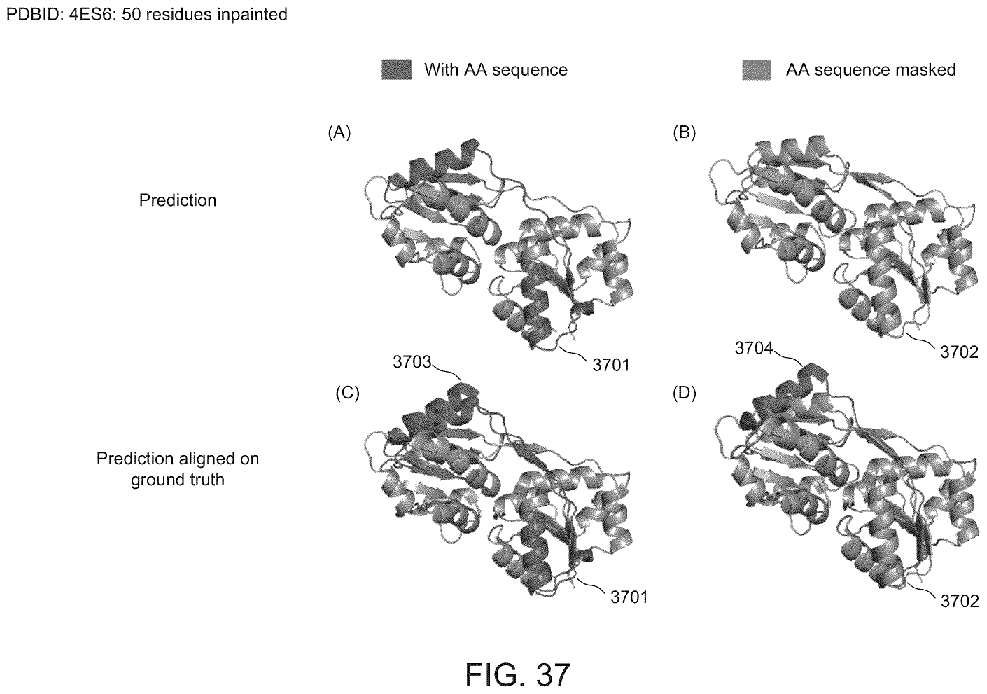

FIG. 37 shows ribbon diagrams illustrating predictions performed on PDB structure PDB ID 4ES6 with 50 residues masked and inpainted by the machine learning model. Predictions 3701 and 3702 are shown alone in the top panels, (A) and (B) and overlaid on ground truth 3703 and 3704 in bottom panels, (C) and (D). Panels (A) and (C) show results obtained where amino acid sequence data was provided, and panels (B) and (D) show results obtained where amino acid sequence data was masked.

### (c) Folding

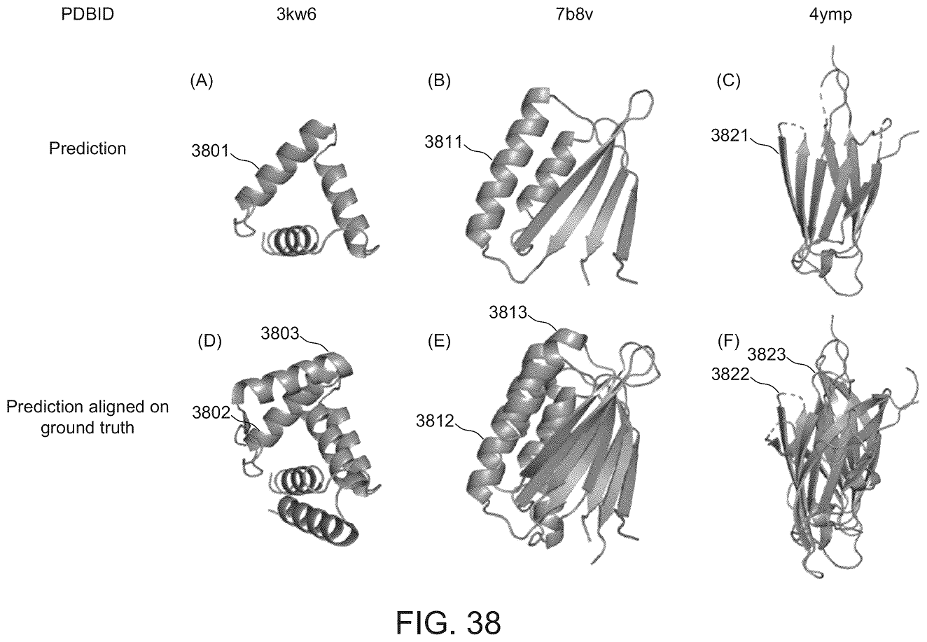

The GNN model was also trained to generate protein folding predictions, e.g., given a sequence, generating a 3D structure (e.g., that it will fold into). FIG. 38 displays ribbon diagrams showing results for several PDB structures (IDs shown along the top), with the prediction shown alone along the top row (elements 3801, 3811, 3821) and the predictions (elements 3802, 3812, 3822) overlaid on ground truth (elements 3803, 3813, 3823) in the bottom row.

### (d) Binder Generation Using Inpainting

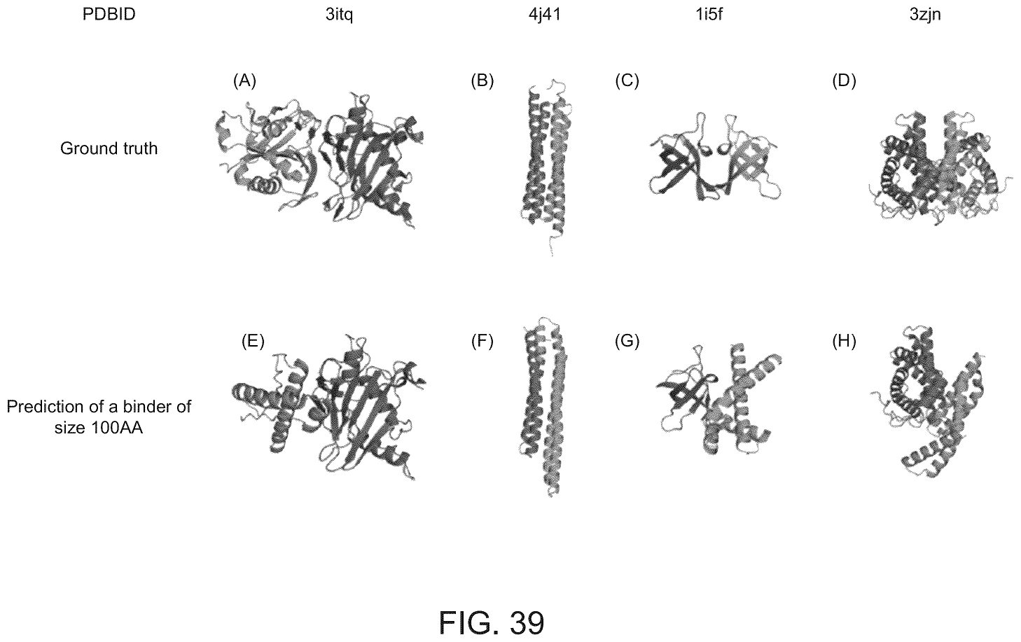

FIG. 39 shows ribbon diagrams of binders generated using inpainting approaches described above, e.g., in Section (c).1 of Example 7 (Example Four-Task GNN Model). The top row shows truth protein binders and while the bottom row shows generated predictions of a binders for example proteins 3itq (A,E), 4j41 (B,F), 1i5f (C,G), and 3zjn (D,H), respectively.

### (e) Binder Generation: Hotspots Unknown

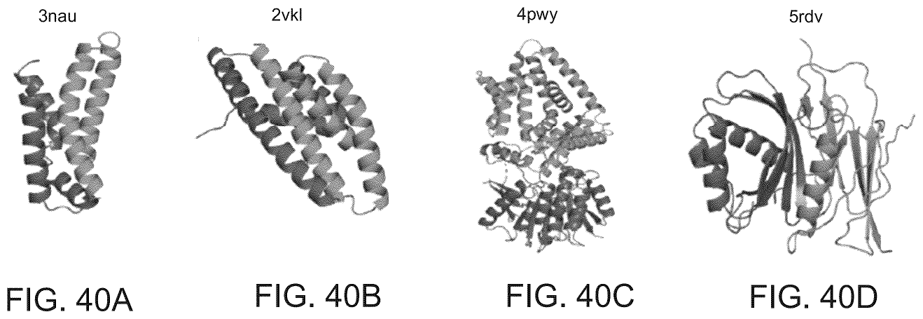

FIGS. 40A-40D show ribbon diagrams of generated binders created using approaches described herein, where hotspot information was unknown, using example proteins with PDB ID 3nau, 2vkl, 4pwy, and 5rdv, respectively.

### (f) Binder Generation: Hotspots Known

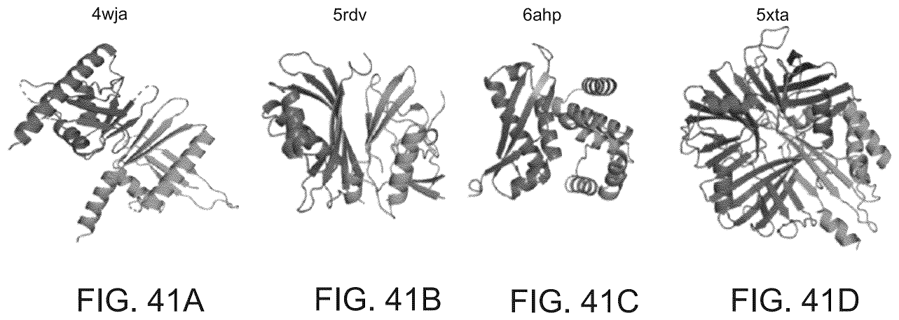

FIGS. 41A-41D show ribbon diagrams of generated binders created using approaches described herein, where hotspot information was known and provided, using example proteins with PDB ID 4wja, 5rdv, 6ahp, and 5xta, respectively.

### I.ix Example 9: Generated Results Via a Transformer Model Using Individual Backbone Atom Representations

This example demonstrates and evaluates performance of a transformer model that used the individual backbone atom representation approach described herein and was trained to produce unconditioned peptide backbone structures.

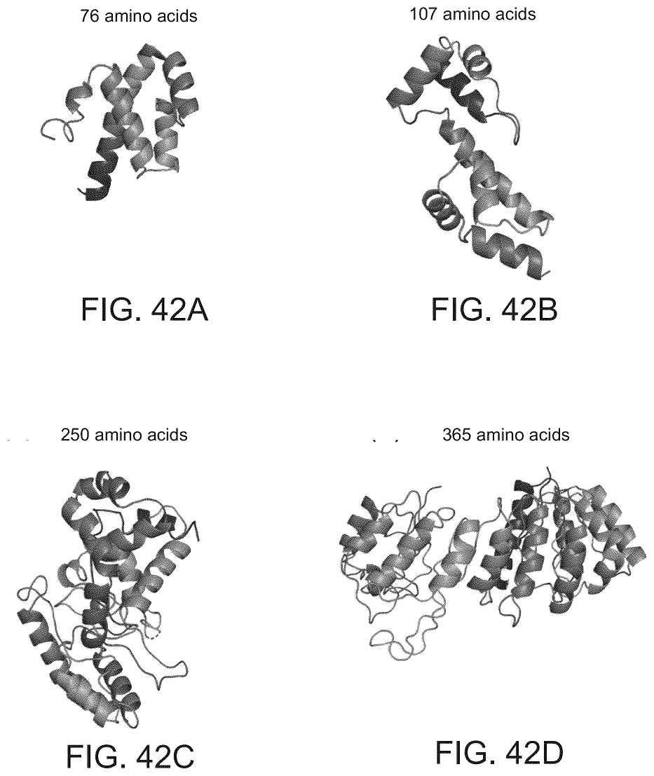

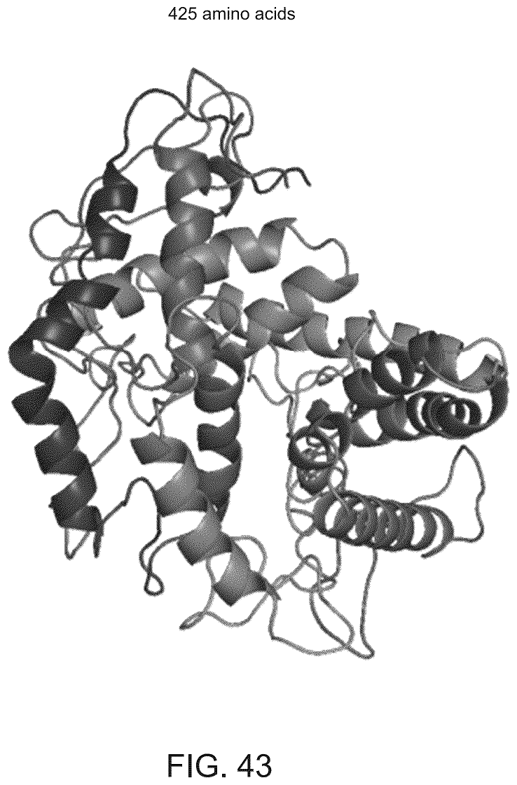

Examples of generated structures are shown in FIGS. 42A-42D and 43, with FIGS. 42A-42D showing images of generated protein structures having 76 amino acids, 107 amino acids, 250 amino acids, and 365 amino acids, respectively and FIG. 43 showing a generated protein structure consisting of 425 amino acids.

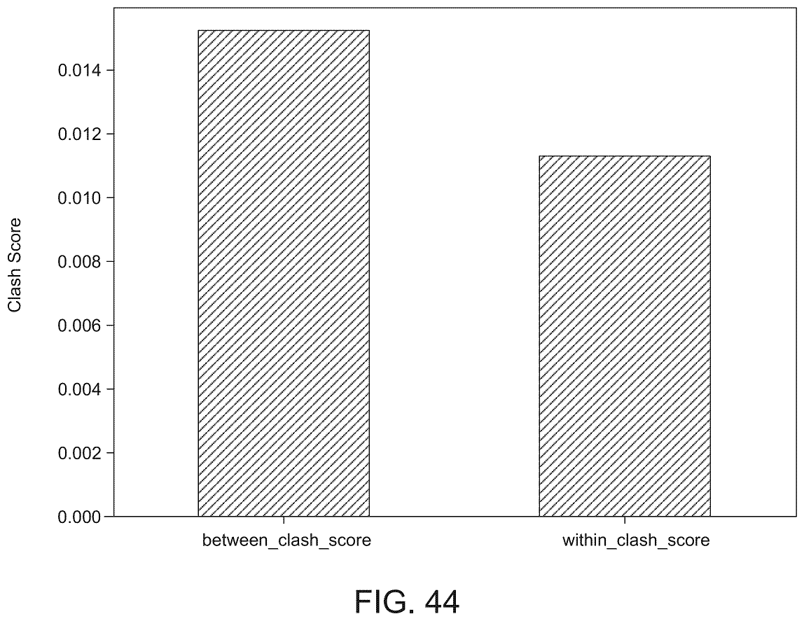

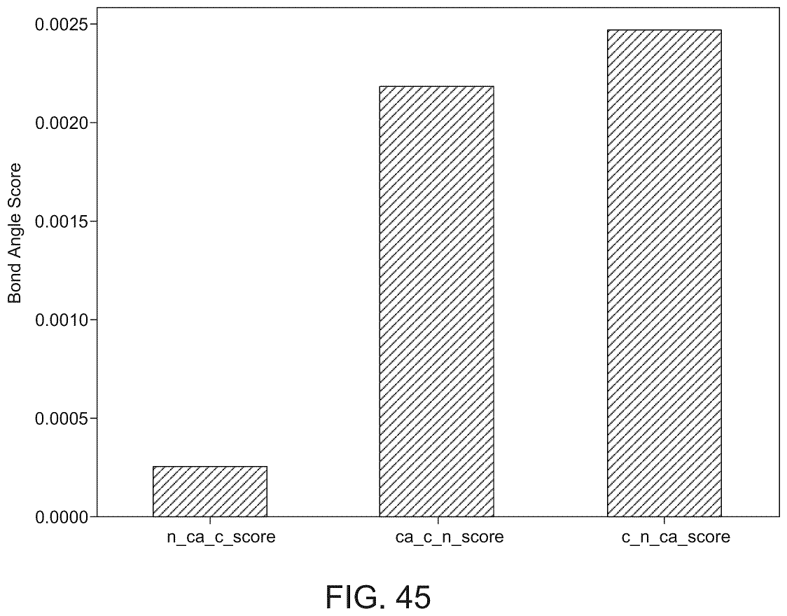

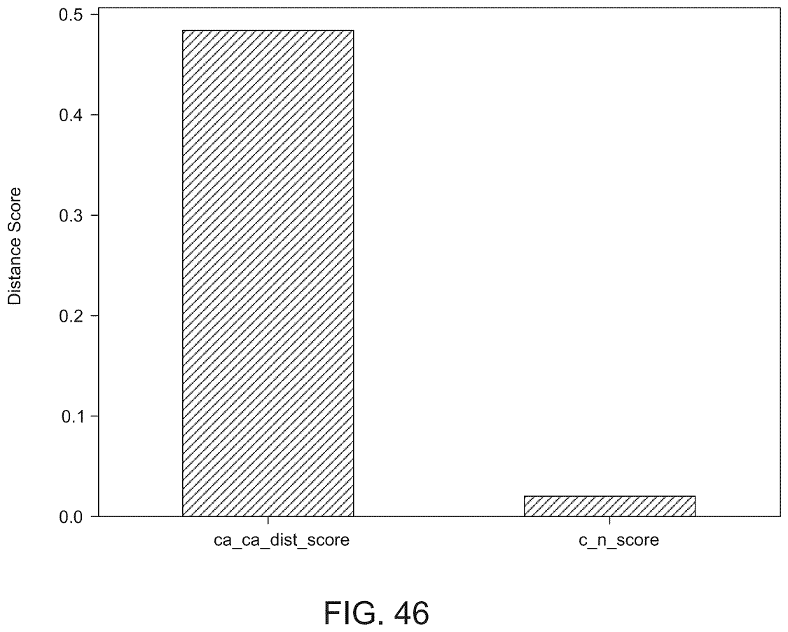

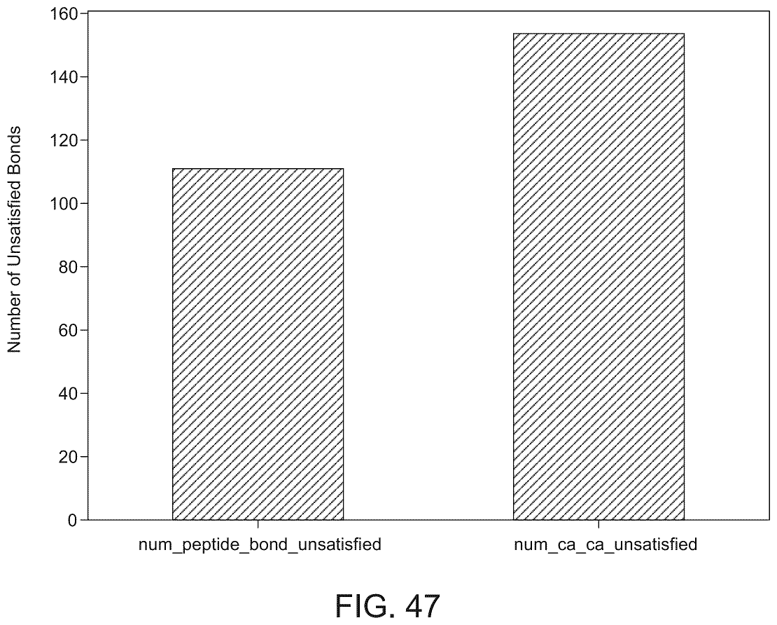

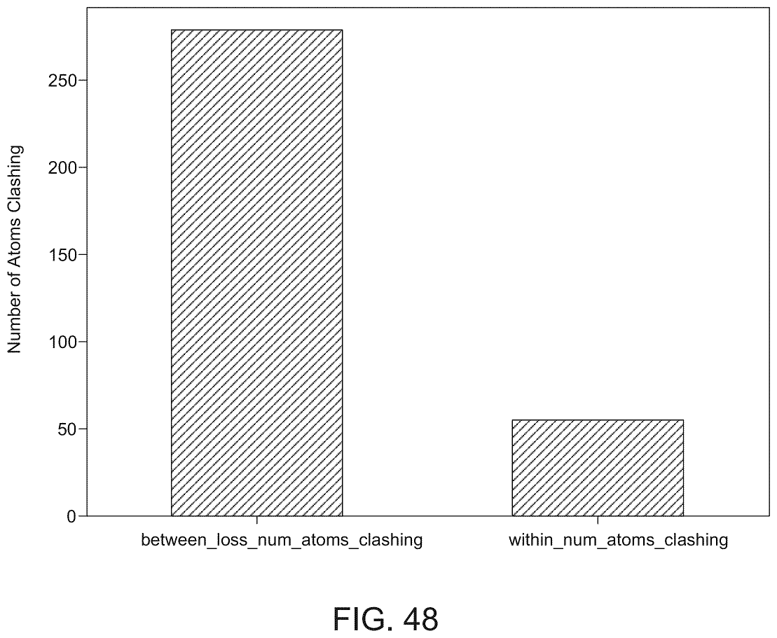

Metrics analogous to those shown in FIGS. 20-24 for the GNN model in Example 8 are shown here for the transformer model in FIGS. 44-48.

### I.x Example 10: Protein Sequence Generation

This example demonstrates training and use of a machine learning model in accordance with certain embodiments of flow matching technologies described herein to generate protein amino acid sequences. The machine learning model of the present example utilized a node encoder and equivariant graph neural network (EGNN) as described in Example 5, section (c), above. An exemplary model with approximately 4.5M parameters consists of 8 Layers Deep Gated Residuals Node Encoder, 4 EGNN Layers, and 8 Layers Deep Gated Residuals Velocity Predictor, using KNN=24 (in Euclidean distance), with encoding dimension 192, message passing dimension 128, and dropout: 0.0.

Protein structures were representing using an augmented version of the local frame and one-hot encoding approach described in the examples above, which was modified to be compatible with the simplex representation approach for sequence design. In particular, polypeptide chains were represented using a combination of the local frame representation to describe peptide backbone structure and a set of twenty-dimensional vector representing individual amino acid residue types. Accordingly, each amino acid site was represented via a set of feature values describing a local frame, namely, an (x, y, z) position of a Ca atom and an orientation (unit quaternions or SO3 or axis angle) and a twenty-dimensional populated with continuous values representing probability of a particular residue type at that amino acid site.

A node decoder was used to generate, as output a vector in R20 that can be used for any objective discussed below.

### (a) Multi-Task Training Process

The machine learning model was trained in a multi-task fashion to perform a variety of sequence generation tasks, including conditional sequence generation.

As described herein, the flow matching framework of the present disclosure facilitates training across a variety of tasks, with distinct tasks only influencing how training data is represented in terms of which, if any, nodes are masked, to be predicted by the machine learning model approach. In this manner, the model is selectively penalized based on the masked nodes, allowing it to train concurrently across the different tasks listed below. The below tasks were carried out for monomers (single chains) as well as multimers (multiple chains), with the exception of the Binder Sequence Design Task, which was only applicable to multimers.

Full Sequence Task. A full sequence generation task trained the model to generate full amino acid sequences, with all nodes captured and represented via simplicial complexes (i.e., a set of simplexes, one for each amino acid site) and conditioned on a protein backbone (e.g., as in an inverse folding task). A categorical variables (amino acid type, polarity, and core/surface) were included and may be randomly masked or unmasked for conditioning purposes.

Segment Inpainting Task. A segment inpainting task trained the model to determine amino acid sequences for a continuous segment within a polypeptide chain, given peptide backbone structure and the amino acid sequence of the remainder of the polypeptide chain. Here, training examples were constructed by selecting a continuous segment amounting to between about 20% to 40% of a sequence for a full chain, and masking amino acid types of the selected segment. Outside the selected segment, amino acid types were retained, and used as input on which to condition generation of the selected segment's sequence. Categorical variables for all nodes were randomly masked or unmasked.

Random Inpainting Task. A random inpainting task trained a model to determine amino acid types at multiple randomly selected amino acid sites within a chain, given knowledge of (e.g., conditioned on) peptide backbone structure and amino acid types at other, known, sites. Training examples were constructed by selecting example chains and randomly selecting sites to mask throughout the chain. Core and surface sites were identified, and approximately 40% of core sites and 20% of surface sits selected to have their amino acid type values masked. Categorical variables for all nodes were randomly masked or unmasked.

Binder Sequence Design Task. A binder sequence design task trained the model to determine an amino acid sequence for a binder, given knowledge of a target (to which the binder binds). Training examples were constructed from example multimers, with one chain identified as a binder (at random) and other chains denoted targets. Amino acid type feature values for the binder chains were masked, and predicted by the machine learning model, while amino acid types for the target chains were known, and allowed to retain their selected, x1, values. Categorical variables for all nodes were randomly masked or unmasked

These task-specific conditions allowed the model to learn from varied data representations effectively, which provided for versatility and depth in the training process.

Since different tasks generate varying numbers of datapoints for training, sampling probabilities for the various tasks were adjusted. This approach ensures that the model achieves optimal performance across all tasks with an equal number of training steps, while preventing overfitting on any single task. This task sampling approach was implemented by randomly selecting a particular task during training, with rates according to the percentages shown below—that is, for example monomers selected 75% of the time, and, where monomers were selected, Full Sequence generation selected to be performed 5% of the time, Segment Inpainting selected 25% of the time, and so on, as listed below.
Monomer (75%)
   Full sequence (5%)
   Segment inpainting: (25%)
   Random inpainting: (70%)
Multimer (25%)
   Full sequence (2.5%)
   Binder design (7.5%)
   Segment inpainting: (25%)
   Random inpainting: (65%)

### (b) Dataset and Training Batch Construction

Training examples were selected from the PDB. Monomers selected were restricted to those having sizes between 32 and 1,100 amino acids. A total of 215,161 PDB structures were, accordingly, selected.

Chains were clustered at 30% or better sequence identity, resulting in 21,096 clusters. A roughly 85%/15% of train/test split resulted in a training dataset of 17,860 clusters (84.66%) and a test dataset of 3,236 clusters (15.34%). The training dataset comprised a total of 191,111 entries and 17,860 clusters of chains. Of these, 9,548 clusters could be used as monomers for a total of 102,895 monomers, and 12,951 clusters comprised multimers, for a total of 88,216 multimers. The test comprised a total of 24,050 entries amounting to 3,236 clusters of chains, with 1,802 monomer clusters, for a total of 13,887 monomers, and 2,157 multimer clusters, for a total of 10,163 multimers (note that an overlap exists in cluster chains for monomers and multimers).

For each protein, only the amino acids with at least 3 atoms (i.e., CA-C—N) were retained. Further filtering was performed on protein size. Additional filtering to further refine the dataset may be performed, for example based on various metrics to allow for improved training, for example to remove chains having backbones that are more than a certain percentage (e.g. 30%) loops, to remove entries with excessive numbers of missing values (e.g., amino acids in the sequence, atom coordinates, etc.), protein size (e.g., diameter), and the like.

During training, batches were created by shuffling cluster indices and randomly selecting one chain from each cluster, as illustrated in FIG. 16.

### (c) Results and Model Performance

Once trained, the machine learning model was used to perform various tasks based on examples selected from the test dataset, and model performance evaluated. Reported results were averaged over five data points (examples) from each cluster (e.g., using a different seed value), thereby ensuring that the evaluation reflects the diversity and complexity inherent in protein sequence design tasks.

For each task, performance was analyzed in terms of both the accuracy and blossom similarity. The following seven tasks were performed and performance evaluated:
1. Multimer—Binder Design. Binder sequences were designed for multiple proteins;
2. Multimer—Random Inpainting. Filling in missing parts (amino acid types) randomly interspersed throughout multiple protein structures;
3. Multimer—Full Sequence Generation. Predicting entire sequences of multiple proteins;
4. Multimer—Segment Inpainting. Completing amino acid sequences for missing segments in multiple protein structures;
5. Monomer—Segment Inpainting. Completing amino acid sequences for missing segments in single protein structures (segment_inpainting_monomer),
6. Monomer—Full Sequence Generation. Predicting entire sequences of single proteins; and
7. Monomer—Random Inpainting. Filling in missing parts (amino acid types) randomly interspersed within single proteins.

Specific performance metrics relevant to each of these challenges, were evaluated, offering a detailed view of the capabilities of sequence generation technologies of the present disclosure in their ability to handle a variety of protein design and prediction tasks.

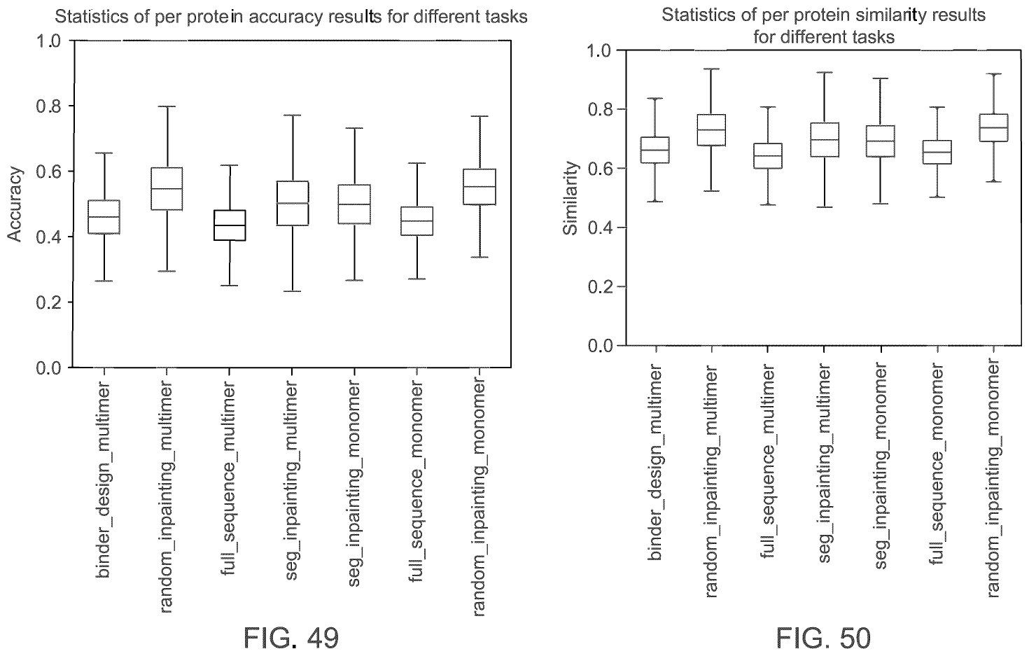

Aggregate statistics describing the model's performance on each of the aforementioned seven tasks in terms of overall accuracy and similarity for each of the aforementioned seven tasks are shown in Table 7, below, highlighting performance for each design challenge. The values can be further improved with subsequence training. FIGS. 49 and 50 provide box-and-whisker plots for accuracy and performance metrics on each task, respectively. For context, historical values for performing similar tasks average slightly above 0.3, whereas state-of-the-art models, such as MPNN AI (e.g., Dauparas et al. “Robust deep learning-based protein sequence design using ProteinMPNN” Science, 2022), demonstrate accuracy slightly above 0.5.

TABLE 7
Averaged values for model performance for various tasks.

| Task names | Accuracy | Blosum_similarity | Support |
|---|---|---|---|
| Binder_design_multimer | 0.4606 | 0.6591 | 441916 |
| Full_sequence_monomer | 0.449 | 0.6519 | 486741 |
| Full_sequence_multimer | 0.4376 | 0.6387 | 1040554 |
| Random_inpainting_monomer | 0.5516 | 0.7347 | 140375 |
| Random_inpainting_multimer | 0.5491 | 0.7304 | 138400 |
| Seg_inpainting_monomer | 0.4981 | 0.6925 | 120995 |
| Seg_inpainting_multimer | 0.5038 | 0.6943 | 119515 |

Model performance was also evaluated with respect to performance depending on the particular type of amino acid, offering insights into the model's performance at the granular level of individual amino acids. This detailed analysis provides a comprehensive view of overall effectiveness of the model, and offers insights for guiding future improvements and tailoring the model to achieve enhanced performance in protein structure prediction tasks. Results of this, individualized, amino acid by amino acid, analysis are provided in Table 8, below, which lists summary statistics (precision, recall, F1 score, and support) for each of the twenty standard amino acid types.

TABLE 8
Model performance by amino acid type.

| Amino Acid | Precision | Recall | F1-score | Support |
|---|---|---|---|---|
| ALA | 0.6353 | 0.4575 | 0.532 | 37272 |
| ARG | 0.4173 | 0.1329 | 0.2016 | 23437 |
| ASN | 0.3631 | 0.3263 | 0.3437 | 17901 |
| ASP | 0.4214 | 0.4627 | 0.4411 | 25781 |
| CYS | 0.466 | 0.3265 | 0.3839 | 5538 |
| GLN | 0.3153 | 0.1091 | 0.162 | 16708 |
| GLU | 0.3061 | 0.3685 | 0.3344 | 30600 |
| GLY | 0.8913 | 0.7508 | 0.815 | 30484 |
| HIS | 0.4292 | 0.1784 | 0.2521 | 10490 |
| ILE | 0.3804 | 0.624 | 0.4727 | 26473 |
| LEU | 0.5212 | 0.6931 | 0.595 | 43477 |
| LYS | 0.2374 | 0.4473 | 0.3102 | 24831 |
| MET | 0.3198 | 0.1447 | 0.1992 | 9954 |
| PHE | 0.4144 | 0.5516 | 0.4733 | 18081 |
| PRO | 0.71 | 0.9173 | 0.8005 | 19617 |
| SER | 0.5039 | 0.2692 | 0.3509 | 26007 |
| THR | 0.4515 | 0.3777 | 0.4113 | 23059 |
| TRP | 0.5422 | 0.1643 | 0.2522 | 5672 |
| TYR | 0.3587 | 0.4099 | 0.3826 | 15172 |
| VAL | 0.4903 | 0.5081 | 0.499 | 31354 |

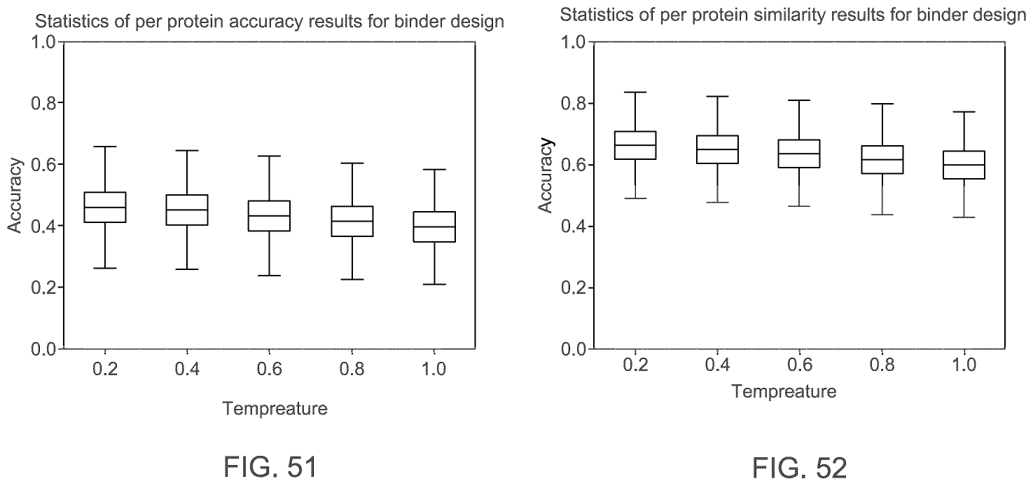

The binder sequence design task was performed multiple times, as temperature was varied, to produce the plots shown in FIGS. 51 and 52. Based on these empirical evaluations, it appears that lower temperature values yielded improved performance for the binder design task, specifically in terms of per-protein accuracy and similarity results.

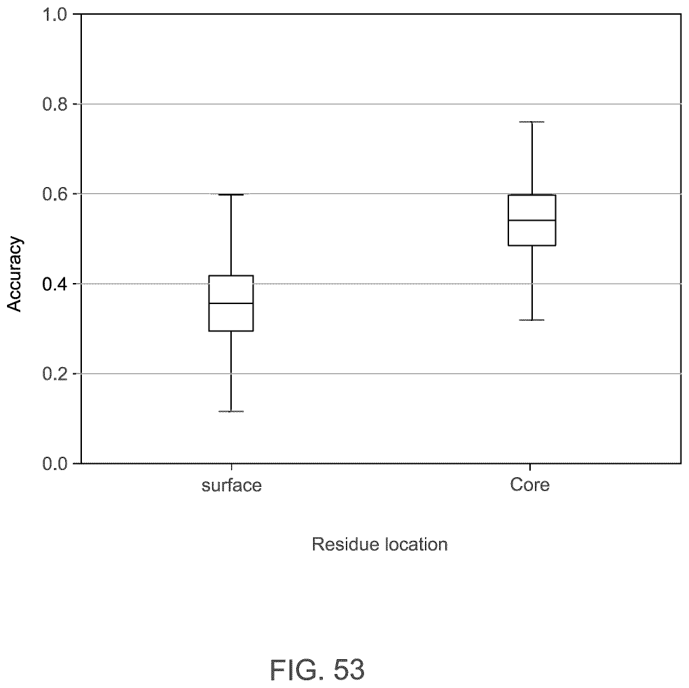

Performance was also analyzed to determine if there was a difference in model performance for core versus surface sites, with results for accuracy shown in FIG. 53. As shown in the figure, accuracy is improved for core sites, where amino acid type is more dependent on a given protein (e.g., peptide backbone) structure, thereby increasing the certainty with which the model predicts sequence information conditioned on backbone structure in those (core) locations. Without wishing to be bound to any particular theory, it is believed that distinction arises because the structural determinants of core residues can be identified with greater certainty as a result of inherent stability and less variable nature of the core regions.

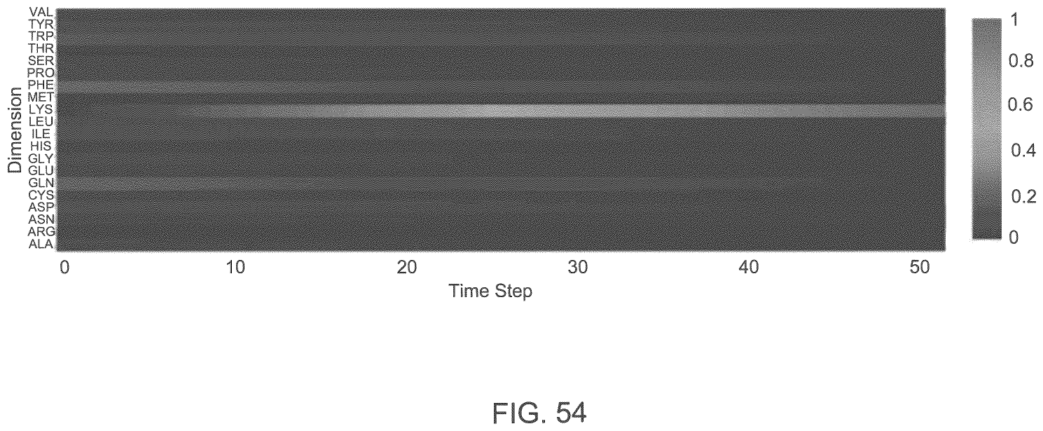

Finally, FIG. 54 shows how amino acid probabilities vary at a particular site over the course of multiple time steps, as the model is used to repeatedly determine sequence velocities and update sequence feature values. In particular, the figure shows a heatmap plotting probabilities for different amino acid types at position 127 in PDB ID 4ib2, chain A, no 1, as they vary over time, ultimately resulting in generation a final value representing a Lysine residue type.

### I.xi Example 11: Side Chain Packing

This example demonstrates and evaluates use of flow matching for generation of 3D side chain geometries, in accordance with various embodiments described herein. Side chain geometries were represented via the complex number approach described herein, and flow matching performed based on Euclidean straight-line paths and constant velocity formulations as described herein.

### (a) Protein Representation

Protein structures were represented as graphs, with node features comprising embeddings of the complex numbers zₜ, embedding of time value t, relative positional encodings of sequence position, amino acid type, a condition mask (described in further detail below), and categorical variables derived from the amino acid type, such as polarity and a core (e.g., of a protein)/surface (e.g., of protein) classification.

The polarity categorical value was a five-value variable identifying an amino acid as belonging to one of five classifications of amino acid type. In particular, the 20 standard amino acids can be classified based on the characteristics of their side chains, which influence their roles and interactions in proteins. Side chains vary in size, shape, charge, and polarity, contributing to the diverse functionality of proteins. Accordingly, side chains may be arranged in categories, as follows

### Nonpolar, Aliphatic

Glycine (Gly, G): No side chain beyond the α-carbon (1 atom).
Alanine (Ala, A): Methyl group (3 atoms).
Valine (Val, V): Isopropyl group (7 atoms).
Leucine (Leu, L): Isobutyl group (9 atoms).
Isoleucine (Ile, I): Sec-butyl group (9 atoms).
Methionine (Met, M): Ethylthioether group (8 atoms, including sulfur).
Proline (Pro, P): Unique cyclic structure (7 atoms).

### Aromatic

Phenylalanine (Phe, F): Benzyl group (11 atoms).
Tyrosine (Tyr, Y): Hydroxyphenyl group (12 atoms).
Tryptophan (Trp, W): Indolyl group (14 atoms, including nitrogen).

### Polar, Uncharged

Serine (Ser, S): Hydroxymethyl group (3 atoms).
Threonine (Thr, T): Hydroxyethyl group (5 atoms).
Cysteine (Cys, C): Thiol group (3 atoms, including sulfur).
Asparagine (Asn, N): Carboxamide group (6 atoms, including 2 nitrogens).
Glutamine (Gln, Q): Longer carboxamide group (8 atoms, including 2 nitrogens).

### Positive (Basic)

Lysine (Lys, K): Alkylammonium group (9 atoms, including nitrogen).
Arginine (Arg, R): Guanidinium group (11 atoms, including 4 nitrogens).
Histidine (His, H): Imidazole group (10 atoms, including 2 nitrogens).

### Negative (Acidic)

Aspartic Acid (Asp, D): Carboxylate group (6 atoms).
Glutamic Acid (Glu, E): Longer carboxylate group (8 atoms).

The number of atoms refers to those directly involved in the side chain, excluding the backbone atoms. These side chains determine the chemical nature and reactivity of amino acids in proteins, influencing their structure, dynamics, and interactions.

The core/surface variable identified residues as either core or surface sites. In proteins, residues can be classified as either “surface” or “core” based on their location within the three-dimensional structure of the protein. Core residues are typically buried inside the protein, away from the solvent, while surface residues are exposed to the solvent, on the exterior of the protein structure.

Core residues are generally hydrophobic (water-fearing) and interact with each other through hydrophobic interactions, helping to stabilize the protein's structure. They are packed tightly together, minimizing any voids within the protein's interior. This tight packing is crucial for the protein's stability, requiring precise spatial arrangement of side chains to optimize van der Waals interactions and minimize any steric clashes.

Surface residues, in contrast, are often polar or charged, allowing them to interact favorably with the aqueous environment. These residues are not as tightly packed as core residues, given the presence of water molecules that can accommodate and even disrupt potential packing arrangements.

Side chain packing is more challenging for surface residues for several reasons:
Flexibility: Surface residues interact with the solvent, making their side chains more flexible and dynamic compared to the tightly packed core residues. This flexibility increases the number of possible conformations, complicating the prediction of their precise arrangement.
Solvent interactions: The presence of water molecules around surface residues introduces additional variables into the packing equation, such as hydrogen bonding and solvent mediated interactions, which are less predictable than the van der Waals forces that primarily influence core residue packing.
Heterogeneity of interactions: Surface residues can participate in a wider range of interactions, including with other proteins, ligands, or solvent molecules, further complicating the prediction of their optimal conformation. For example, Phenylalanine is a hydrophobic amino acid that, when found in the core, contributes to the hydrophobic packing and stability of the protein's interior. Its bulky, aromatic side chain prefers to be tightly packed away from water, minimizing its surface area exposed to the solvent. Glutamic Acid, on the other hand, is a charged, hydrophilic amino acid often found on the protein surface. Its side chain can form salt bridges and hydrogen bonds with the surrounding water molecules or other polar atoms. On the surface, Glutamic Acid's side chain is more exposed to solvent and can adopt multiple conformations, making its packing less predictable and more complex than the more static and hydrophobic environment of the protein core. In summary, the classification of residues into surface and core categories reflects their environmental context within the protein, which in turn influences their behavior, interactions, and the complexity of their side chain packing.

Graph representations of proteins and/or peptides in this example also included edge features. These included Cα-Cα distances, an embedding of time value, five pairs of dihedral angles describing the relative geometry of pairs of residues, and embeddings for mask information derived from hotspots, core/surface, connection type between different chains within a multimer, as well as a condition mask.

A k nearest neighbor approach was used to limit a number of edges for each node, to a local neighborhoods based on Cα-Cα distances. In particular, each node comprised edges only connecting it to its k=16 nearest neighbors.

The neural network model comprised Node Encoder, Equivariant Graph Neural Network, and Velocity Predictor. The model was trained end-to-end (e.g., the model learns everything from the beginning to end, not sequentially). The particular form of the model takes into account invariance and equivariance of 3D data with respect to the action of the special Euclidean group which is the semidirect product

$$SE ( 3 ) = {ℝ}^{3} SO ( 3 ) .$$

The group acts on 3D structures through translations and rotations. Any two protein structures which differ by such an action represent the same protein and should be treated equally by the model.

Within the model, first, the node features are passed to a Node Encoder to obtain an initial latent representation. Since these features are all invariant with respect to SE(3), the choice of architecture is unconstrained. Here, the architecture used was a deep neural network with gated residual connections which allows for expressive encoding of all features, and in particular of the side chain data.

To update the latent representation through repeated application of graph convolution using an equivariant graph neural network (EGNN) architecture similar to the one described above with regard to protein backbone models. The initial equivariant features are given by the pair (ca, q), where ca denotes the centered 3D coordinates of a batch of proteins and q is a tuple of unit quaternions representing the local frames of the residues. At each layer of the network, the equivariant features are updated and used to recalculate the edge features encapsulating the relative geometry, such as Euclidean distances and dihedral angles. Then, invariant features were extracted and the latent representation updated. This process is repeated at each layer.

Third, the final latent representation was decoded with a Velocity Predictor to obtain an estimate of the flow matching vector field. Similar to the encoder, the velocity predictor comprises a deep neural network with gated residual connections.

### (b) Training

Training was performed via a self-supervised learning strategy whereby various portions of example protein chains were masked, and the model tasked with predicting the masked portions. The training dataset comprised protein structures of size 32 up to 1100 (number of residues). For each data point (backbone, sequence, side chain) a number of residues were randomly selected and their side chain torsion angles masked. The precise sampling strategy used here is as follows:
(i) Randomly select a monomer (70%) or a multimer (30%).
(ii) Randomly select a protein cluster.
(iii) Randomly select a protein chain in the cluster.
(iv) Randomly select core residues (40%) and surface residues (20%) in the protein chain.

The set of selected residues is referred to as the condition mask. The neural network is then trained by masking the loss function according to the condition mask.

Accordingly, at each training step, the model received a set of proteins, where for each protein in the set, a condition mask of training residues, an initial seed point z₀˜p₀, and a time value t, is sampled. The set is then collated into a batch. The inputs to the flow matching model \hat{v} accordingly comprise a triple (b, t, zₜ), where the batch data of backbone, sequence, and condition mask is denoted as b. Loss functions were calculated using the description above, including time scheduling, dynamic weighting, and an auxiliary loss:

$${ℒ}_{t} ( b , z ) = \frac{α ( t )}{β ( t )} {ℒ}_{t}^{F M} ( b , z ) + γ ( t ) I [ t > 0.75 ] {ℒ}^{A u x} ( z + ( 1 - t ) \hat{{v}_{t}} ( b , z ) ) ,$$

where the loss function [symbol] is for the model \hat{v}, time value t, featurized batch b, and side chain data z.

### (c) Results

Once trained, performance of the model was evaluated using samples from the test dataset of protein structures (which the model has never seen during training).

The model was evaluated four metrics: 1) MAE, 2) Accuracy, 3) RMSD, 4) Violation, as follows:

Mean Absolute Error (MAE) quantifies the average magnitude of the errors in predictions of torsion angles, without considering their direction. It is defined as:

$$M A E = \frac{1}{N} {Σ}_{i = 1}^{N} \vert {\hat{χ}}_{i} - {χ}_{i} \vert ,$$

where \hat{χ}ᵢ and χᵢ are the predicted and actual torsion angles for the i^{th} side chain, respectively, and N is the total number of side chain predictions made. A lower MAE indicates a higher accuracy in predicting side chain conformations.

Accuracy (e.g., with a threshold of 20 degrees) measures the proportion of predictions where the absolute error in torsion angles does not exceed 20 degrees. This metric reflects the percentage of predictions considered “accurate” within a practical tolerance level for structural biology applications. It is calculated as:

$$Accuracy = \frac{Number of predictions with error \leq 20 °}{Total number of predictions} \times 100$$

The accuracy of prediction models depends significantly on the type and properties of the amino acid. This is because different amino acids have side chains with varying complexity, flexibility, and number of torsion angles. For example, Phenylalanine has a relatively rigid and bulky aromatic side chain. Predicting its torsion angles (especially χ₁ and χ₂ for the ring rotation) can be relatively straightforward due to the limited conformational space arising from its structure. Thus, models may achieve lower MAE and higher accuracy for Phenylalanine. Glutamic Acid, in contrast, has a longer, flexible side chain with multiple torsion angles (χ₁, χ₂, and χ₃) that can adopt a wider range of conformations. This flexibility introduces greater complexity in accurately predicting its side chain conformation, potentially resulting in higher MAE and lower accuracy compared to more rigid amino acids like Phenylalanine.

Overall, the evaluation metrics not only provide a measure of the predictive performance of side chain prediction models, but also underscore the importance of considering amino acid-specific properties in model development and assessment.

Root Mean Square Deviation (RMSD) metric that evaluates the accuracy of protein structure prediction models, particularly for assessing the quality of side chain packing.

It measures the average distance between atoms of predicted and experimentally determined structures after optimal superposition. For side chain packing, RMSD specifically focuses on the differences in the positions of side chain atoms. The RMSD is calculated as follows:

$$R M S D = \sqrt{\frac{1}{N} {\sum }_{i = 1}^{N} {( {d}_{i}^{p r e d} - {d}_{i}^{e x p} )}^{2}} ,$$

where N is the number of atoms considered (typically, only side chain atoms for side chain packing analysis), dᵢ^{pred} is the position of the i^{th} atom in the predicted structure, and dᵢ^{exp} is the position of the i^{th} atom in the reference (experimentally determined) structure.

RMSD provides a quantitative measure of the deviation between the predicted and actual atomic positions, offering insight into the model's accuracy. A lower RMSD value indicates a closer match to the reference structure, suggesting a more accurate prediction.

However, it's important to note that RMSD can be sensitive to local errors and may not fully capture the functional relevance of the predicted structure. In the context of side chain packing, even small deviations in the position of critical side chains can significantly impact the protein's function, despite a low overall RMSD. In summary, RMSD is a crucial metric for evaluating the performance of protein structure prediction models, especially for the precise task of side chain packing, providing a direct measure of the spatial accuracy of the model's predictions.

A violation metric was determined based on presence of/to quantify steric clashes in protein structure prediction, particularly in side chain packing, refer to the physical overlap of atoms within a protein's three-dimensional structure that would result in unrealistic, energetically unfavorable contacts. These clashes occur when the spatial arrangement of atoms violates the principles of allowed atomic distances, leading to configurations that cannot exist due to the hard-sphere nature of electron clouds surrounding atoms. Essentially, atoms cannot occupy the same space due to the repulsive forces that arise when electron clouds overlap, which is a fundamental concept in chemistry known as the Pauli exclusion principle.

In the context of protein structure prediction, accurately modeling the spatial arrangement of amino acid side chains is crucial for predicting a protein's tertiary structure. The side chains of amino acids vary greatly in size, shape, and chemical properties, and their correct positioning is essential for the protein to fold into its functional three-dimensional shape. When predicting these structures, computational models must ensure that side chains are positioned in a way that avoids steric clashes, ensuring realistic and energetically feasible configurations.

Steric clashes are particularly significant for side chain packing for several reasons:
Density of Packing: The interior of a protein is densely packed, with little free space. Correctly positioning side chains to maximize van der Waals interactions while avoiding clashes is crucial for achieving a stable structure.
Flexibility and Conformational Space: Some side chains have a degree of rotational freedom around their bonds (torsion angles), allowing multiple conformations. Identifying the conformation that optimizes interactions without causing steric clashes is a complex problem.
Impact on Function: Improper side chain packing can lead to misfolded proteins, impacting their stability and function. Steric clashes in critical areas may prevent the protein from adopting its functional conformation or interacting correctly with other molecules. For instance, in a protein structure where Phenylalanine and Glutamic Acid are close together, a steric clash could occur if the bulky phenyl ring of Phenylalanine and the side chain carboxyl group of Glutamic Acid are positioned too close without enough space to accommodate their physical volumes. Avoiding such clashes while ensuring the overall structure is stable and functional is a key challenge in protein structure prediction, requiring sophisticated algorithms to explore and optimize the vast conformational space of possible side chain arrangements

The first three metrics are standard and do not depend on adjustable parameters, whereas the violation metric value depends on, e.g., a choice of threshold distance in defining steric clashes. Without wishing to be bound to any particular theory, it is believed that the violation metric provides valuable insights. Performance evaluation provided herein also differentiate between core and surface residues. The accuracy metric is most powerful when applied to core residues, i.e. those amino acids which are buried deep inside the protein structure. This is because the side chains of core residues tend to have low variance. Any inaccuracies in the prediction of their side chains can lead to severe steric clashes. It is therefore paramount for our model to achieve a high accuracy on core residues. The side chains of surface residues on the other hand have higher variance and are much more flexible and dynamic in the way they interact with the environment. Evaluating the predicted side chain conformation of surface residues is therefore an effective tool to assess the generative capabilities of our model. To do so here, steric clashes were measured using the violation metric.

The ability to accurately generate viable 3D side chain packing predictions for monomer structures was evaluated using a test dataset of monomers of size 32 up to 1100. From each cluster, up to four monomers were selected at random (less than four if the cluster does not contain four monomers). The total number of samples obtained in this manner was 4,881. For each sample, the side chains of all residues were generated jointly. Side chains of Alanine (“ALA”) and Glycine (“GLY”) have idealized geometries which are described without any torsion angles. Thus, these residues were omitted from evaluation.

The results are reported by amino acid (Table 9), by torsion angle (Table 10), and by core/surface type (Table 11). The average angle accuracy across all samples, all residues, and all torsion angles is 73.73%.

TABLE 9
Metrics by amino acids (AA).

| AA | MAE | Accuracy | RMSD |
|---|---|---|---|
| ALA | — | — | — |
| ARG | 37.46 | 0.6010 | 1.2744 |
| ASN | 30.65 | 0.7056 | 0.5941 |
| ASP | 20.38 | 0.7222 | 0.4546 |
| CYS | 11.40 | 0.9131 | 0.1495 |
| GLN | 36.28 | 0.6151 | 0.9135 |
| GLU | 33.20 | 0.5666 | 0.8654 |
| GLY | — | — | — |
| HIS | 38.12 | 0.7196 | 0.7679 |
| ILE | 15.51 | 0.8769 | 0.2437 |
| LEU | 15.58 | 0.8608 | 0.3308 |
| LYS | 32.36 | 0.6626 | 0.9381 |
| MET | 33.67 | 0.6846 | 0.6832 |
| PHE | 8.95 | 0.9134 | 0.3252 |
| PRO | 12.07 | 0.8172 | 0.1271 |
| SER | 28.90 | 0.7513 | 0.2865 |
| THR | 16.31 | 0.8734 | 0.2315 |
| TRP | 15.15 | 0.8956 | 0.6126 |
| TYR | 9.12 | 0.9182 | 0.3852 |
| VAL | 12.97 | 0.9079 | 0.1891 |

TABLE 10
Metrics by torsion angle.

| Chi | MAE | Accuracy |
|---|---|---|
| χ1 | 16.50 | 0.8468 |
| χ2 | 25.59 | 0.7316 |
| χ3 | 44.52 | 0.4634 |
| χ4 | 49.31 | 0.4810 |

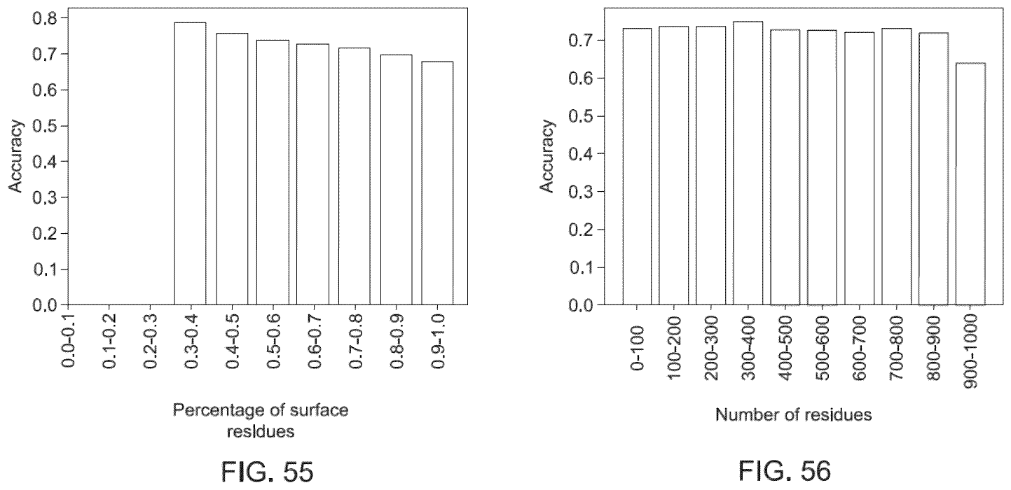

All three metrics MAE, Accuracy, and RMSD differ significantly for core residues vs. surface residues. To further illustrate this point, the accuracy by the percentage of surface residues in the respective monomer is plotted in FIG. 55.

TABLE 11
Metrics by type.

| Type | MAE | Accuracy | RMSD |
|---|---|---|---|
| Core | 15.48 | 0.8617 | 0.2857 |
| Surface | 30.26 | 0.6699 | 0.6604 |
| Total | 25.09 | 0.7373 | 0.5084 |

Finally, the model is able to consistently generate accurate torsion angle predictions for monomers of any size as measured by the number of residues as shown in FIG. 56.

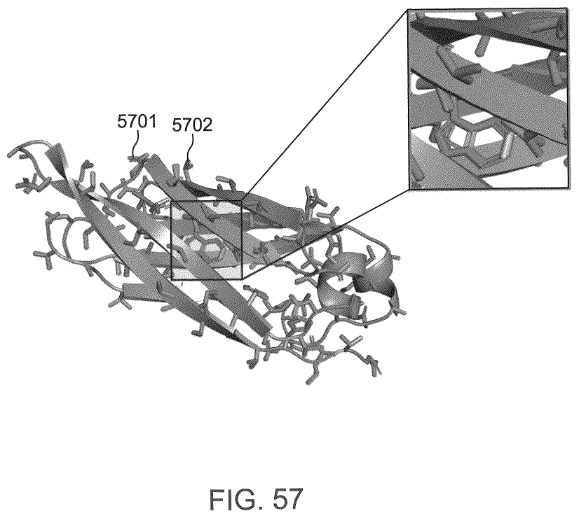

To illustrate the capabilities of the model to generate realistic and highly accurate side chains, the protein structure PDB 4kdw was selected, which is a monomer with a total of 102 residues. The side chains of all of its residues were jointly generated following the ODE dynamics with the model using 100 time steps. As shown FIG. 57, the prediction 5701 is highly accurate as compared to ground truth 5702 and as measured by MAE (13.10), Accuracy (88.29%), and RMSD (21.19).

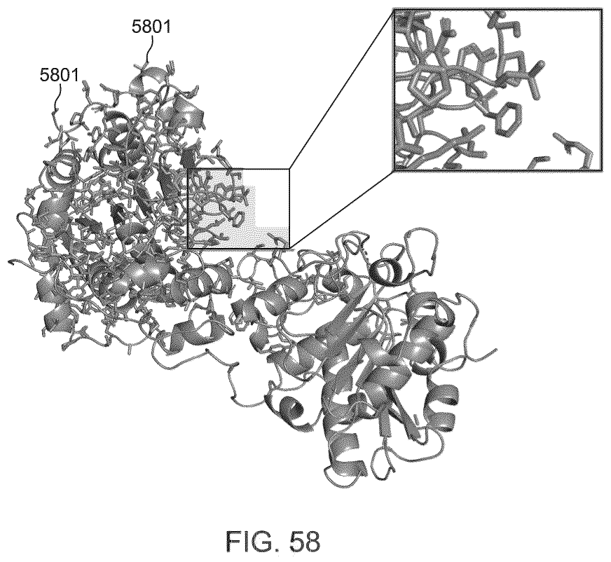

The model was also evaluated on multimer structures. A multimer is an assembly of multiple protein chains. The model can be applied without any constraints to the side chain packing problem for multimers. To illustrate this, the protein structure PDB 3icw was selected, which is a dimer with two chains (A and B) and a total of 582 residues. Chain B (291 residues) was masked and all of its side chains were generated following the ODE dynamics with the model using 100 time steps. As shown in FIG. 58, the prediction 5801 is highly accurate as compared to ground truth 5802 and as measured by MAE (11.92), Accuracy (89.34%), and RMSD (22.40).

The violation metric is a weighted average of 1) clashes between different residues and 2) clashes within a residue. The metric accepts several parameters determining the level of tolerance, i.e. how close two atoms are allowed to be before they are considered as a clash. The structure of a ground truth protein exhibits little to no steric clashes. However, due to aforementioned choice of parameters as well as inaccuracies and errors in experimental data, the violation values of PDB structures are not always zero, but approximately in the range 0.0 to 0.002.

Here, the quality of the side chains generated by the model is assessed using the violation metric. multimers of size 32 up to 1100 were sampled from the test dataset using the following protocol:
(i) From each multimer randomly select a protein chain of size between 10 and 100 if such a chain exists.
(ii) Mask all side chains of the selected protein chain, keep the ground truth side chains of the remainder of the multimer.
(iii) Sample prior for each masked residue.
(iv) Generate all torsion angles of the masked residues using the model.
(v) Compute the violation metric of the masked atoms vs. all atoms.
(vi) Repeat steps (ii)-(v) eight times and select the lowest violation value.

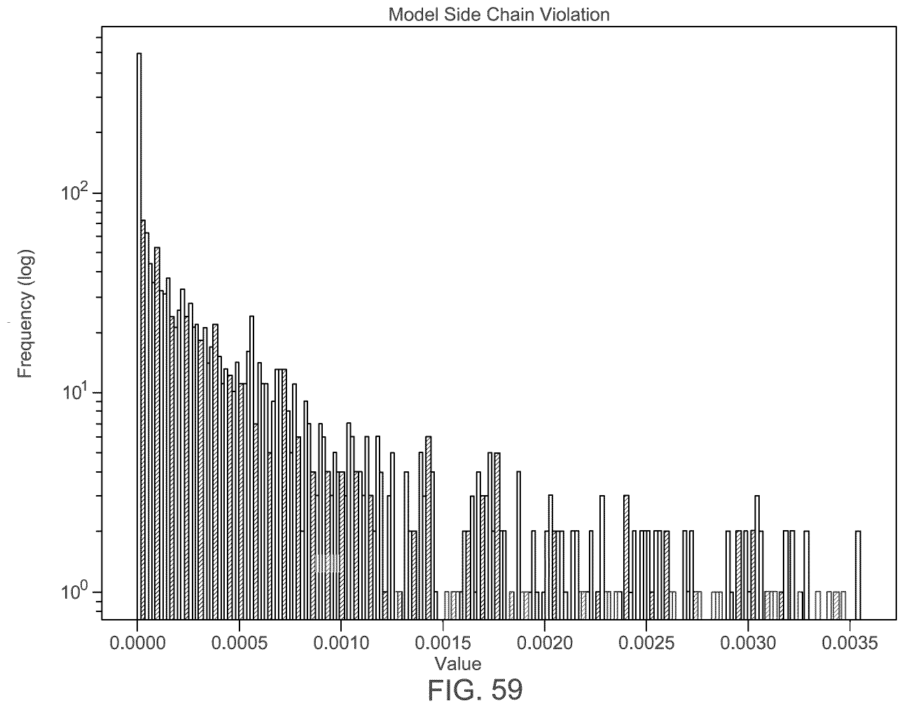

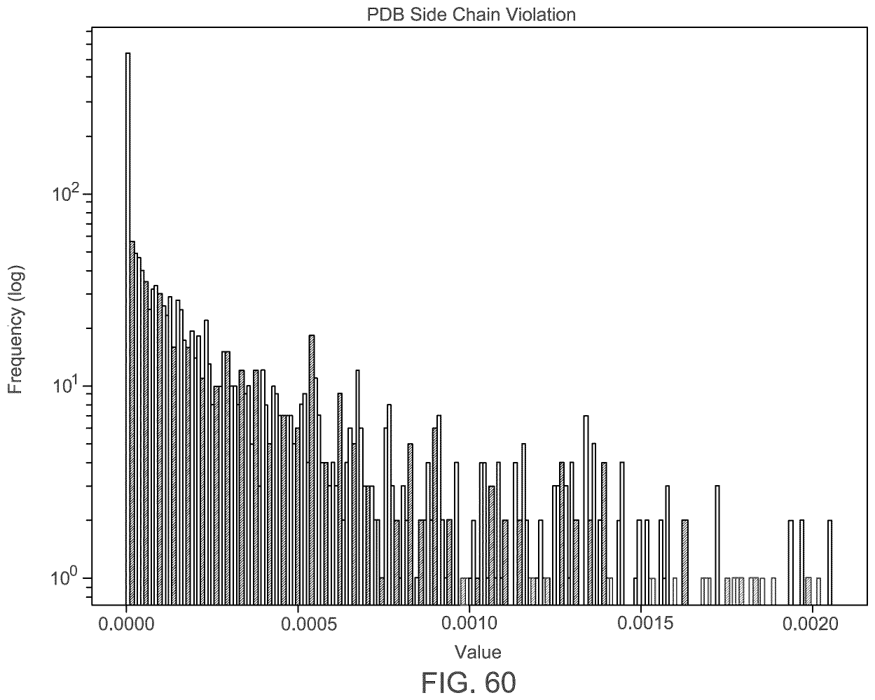

The dataset of multimers satisfying condition (i) consists of 1,792 PDB structures. FIGS. 59 and 60 show the distribution of violation values of the side chain conformations generated by the model vs. PDB structures. For both distributions a cutoff at the 95th percentile was used to remove noisy and erroneous data. The predicted distribution approximates the PDB distribution with remarkable accuracy. The distributions differ only slightly in scale, which can most likely be addressed by minor modifications of model architecture and training optimization.

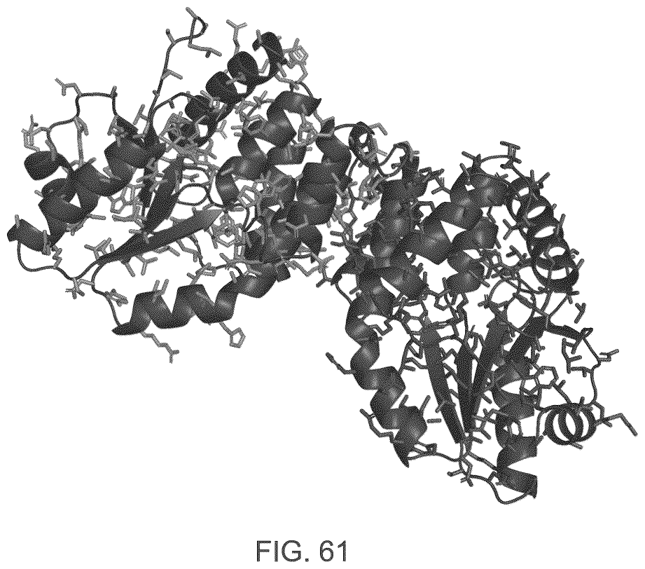

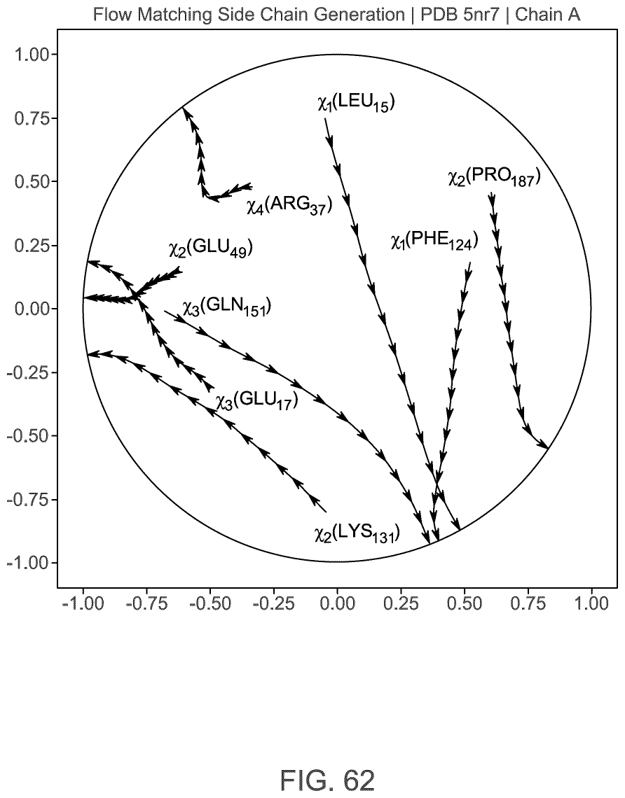

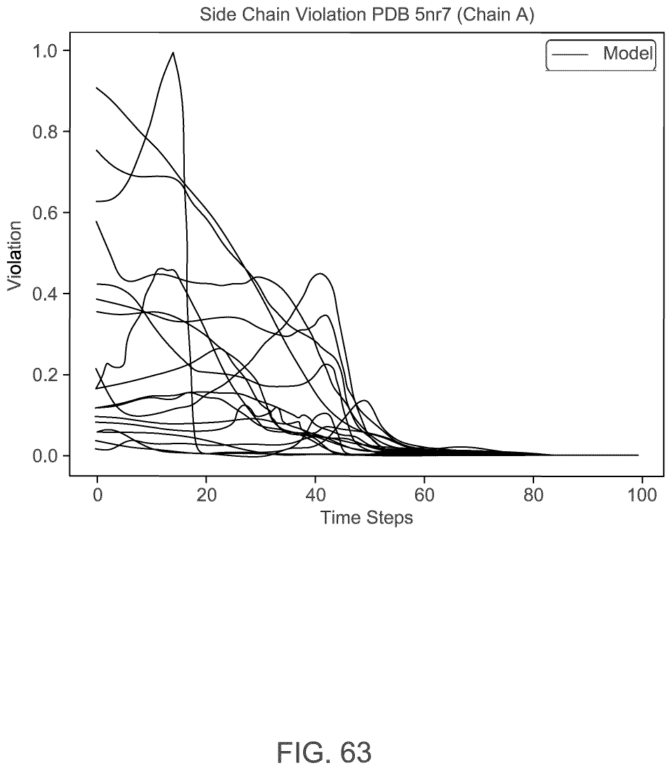

To further assess the model, the protein structure PDB 5nr7 was selected, which is a dimer with two chains (A and B) and a total of 372 residues. Chain A (188 residues) was masked and all of its side chains were generated following the ODE dynamics with the model using 100 time steps. The result is displayed in FIG. 61. The final protein structure prediction, evaluated by the number and severity of steric clashes, shows an accurate result: the violation value is 0.0. FIG. 62 illustrates examples of the generated paths of selected torsion angles represented as complex numbers. The evolution of the violation value per amino acid type throughout generation is depicted in FIG. 63.

The generation of all side chains for an entire protein chain (188 residues) without any steric clashes and violations is remarkable. For comparison, side chains for the same protein PDB 5nr7 (Chain A) was generated by sampling 100,000 times torsion angles uniformly at random. Each generation exhibits substantial clashes, on average 100x more severe than any realistic protein structure. It is virtually impossible to generate realistic side chain conformations by random chance.

### I.xii Example 12: Fold Conditioning

This example demonstrates and evaluates use of flow matching with fold conditioning, in accordance with various embodiments described herein. Two versions of compact constraint used for fold conditioning were used to evaluate flow matching methods as described herein.

### (a) Fold Families for Benchmarking

This section provides an overview of datasets used for selecting fold families used for the model training.

The SCOP (Structural Classification of Proteins) and CATH (Class, Architecture, Topology, and Homologous superfamily) datasets are both comprehensive resources for the classification of protein structures. These databases organize protein domains based on their structural and evolutionary relationships, providing valuable insights into the function and history of proteins. The CATH dataset is a comprehensive resource for the classification of protein domain structures, which groups proteins at four major levels: Class, Architecture, Topology, and Homologous superfamily. At the highest level, the Class level, proteins are grouped based on their secondary structure content (mainly alpha, mainly beta, alpha-beta, special, and few secondary structures). The Architecture level describes the overall shape of the domain structure, while the Topology level provides information about the topological connections between secondary structures. Finally, the Homologous superfamily level groups proteins that are likely to share a common ancestor, indicating a closer evolutionary relationship. The CATH database is widely used in the study of protein structure and function, offering valuable insights into the structural diversity and evolutionary history of proteins.

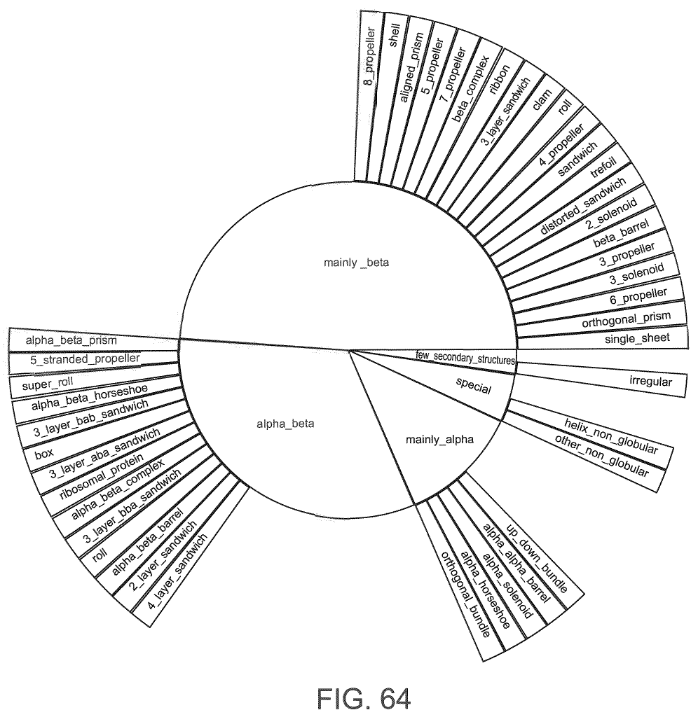

CATH dataset also provides one representative for each fold family at each level. Fold families from the Architecture level of the CATH dataset were selected and the corresponding PDB file provided in the CATH website were downloaded for further use. Sometimes the CATH representative of the fold family is one domain or a segment of a bigger protein structure. In those cases, the PDB were taken and the desired domain/segment was cut. FIG. 64 shows selected hierarchy of CATH. There are 43 folds in the hierarchy, therefore 43 PDBs are selected for subsequent benchmarks.

### (b) Training

For training, the secondary structure elements (e.g., helix, strand, and loop) of a protein were converted into one-hot encoded vectors and added as extra node features to the model. The block adjacency information was also added as an extra edge feature to the model. For inference, the SSE and block adjacency were computed based on a given fold family. Then, multiple samples were generated conditioned on the fold family.

Several metrics were chosen to benchmark the model. These metrics are as follows:
SSE Accuracy: Accuracy measures the percentage of SSEs that the generative models predicted correctly as compared to the ground truth fold:

$$Accuracy = \frac{Number of predictions with error \leq 20 °}{Total number of predictions} \times 100$$

TM Score: The TM score, or Template Modeling score, is a metric used to measure the similarity between two protein structures. It is often used in protein structure prediction and protein structure comparison. The TM score ranges from 0 to 1, where a score of 1 indicates a perfect match between two structures. Generally scores below 0.20 corresponds to randomly chosen unrelated proteins whereas structures with a score higher than 0.5 assume roughly the same fold. The TM score is calculated based on the root-meansquare deviation (RMSD) of the distances between equivalent residues in the two structures, normalized by the length of the protein. Unlike RMSD, which is sensitive to local structural differences, the TM score is more robust and is particularly useful for comparing structures with different global folds but similar overall topology.
   A score near 1 is desirable, but attaining a perfect score would merely result in replicating the input, thereby diminishing the ability to generate diversity.

A typical fold conditioning model included 5M parameters. An example model had 10 deep residual connected layers as an input encoder, 10 EGNN layers, and 10 more deep residual layers as an input decoder.

Values associated with the SSE and/or the block adjacency matrix were masked during the model training. Examples of masking various values during training a multitask model on a variety of tasks are presented in Tables 12-13. A combination of multiple tasks (e.g., inpainting and docking) at the same time may be performed though combination of the existing ones.

TABLE 12
Masking examples during training of a multitask model
on monomer data. BAM (Block Adjacency matrix) and SSE:
0 means fully masked, 1 means not masked, % means randomly
certain portion of the data is masked.

| Category/ | | | | |
|---|---|---|---|---|
| Number of tasks | Task | Seq | BAM | SSE |
| Unconditional: 1 | Unconditional | 0 | 0 | 0 |
| Folding: 1 | Folding | 1 | 0 | 0 |
| Fold | Fold | 0 | % | % |
| conditioning: 3 | Conditioning 1 | | | |
| | Fold | 0 | 1 | 1 |
| | Conditioning 1 | | | |
| | Fold | 0 | 1 | 0 |
| | Conditioning 1 | | | |
| Inpainting: 2 | Inpainting | 1 | % | % |
| | Inpainting | 0 | % | % |

TABLE 13
Masking examples during training of a multitask model
on multimer data. BAM (Block Adjacency matrix) and
SSE: 0 means fully masked, 1 means not masked, % means
randomly certain portion of the data is masked.

| Category/ | | | | |
|---|---|---|---|---|
| Number of tasks | Task | Seq | BAM | SSE |
| Inpainting/2 | Inpainting- | 1 | % | % |
| | multichain | | | |
| | Inpainting- | 0 | % | % |
| | multichain | | | |
| Docking/2 | Pseudo-docking | 1 | 0: binder | 0: binder |
| | | | 1: target | 1: target |
| | Docking | 1 | 1: binder | 1: binder |
| | | | 1: target | 1: target |
| Binder design/2 | Binder design | 0 | 0: binder | 0: binder |
| | | | 1: target | 1: target |
| | Binder design | 0 | 1: binder | 1: binder |
| | | | 1: target | 1: target |
| Folding on | Folding dimer | 1 | 0 | 0 |
| dimer/1 | | | | |
| Unconditional | Unconditional | 0 | 0 | 0 |
| on dimer/1 | dimer | | | |
| Fold | Multichain fold | 0 | % | % |
| conditioning on | conditioning 1 | | | |
| dimer/3 | | | | |
| | Multichain fold | 0 | 1 | 1 |
| | conditioning 2 | | | |
| | Multichain fold | 0 | 1 | 0 |
| | conditioning 3 | | | |

### (c) Results

Two models were trained over roughly 200.000 iterations with distinct configurations:

Model 1: was configured with a cutoff distance of 6.0 Ångströms, utilizing BAM without topology information.

Model 2: was set with an extended cutoff distance of 8.0 Ångströms and incorporated BAM with topology information and with an additional feature: the inclusion of loops in the block adjacency calculations.

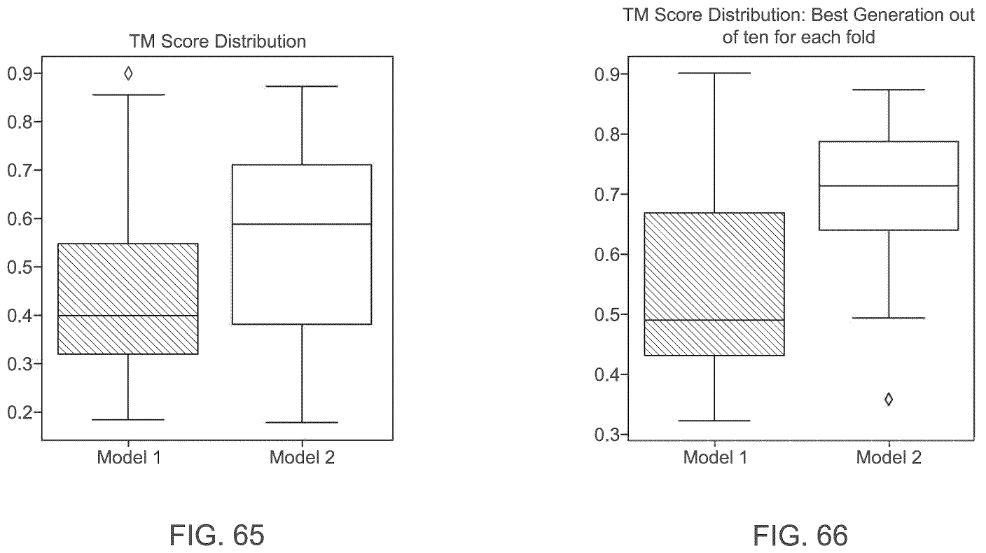

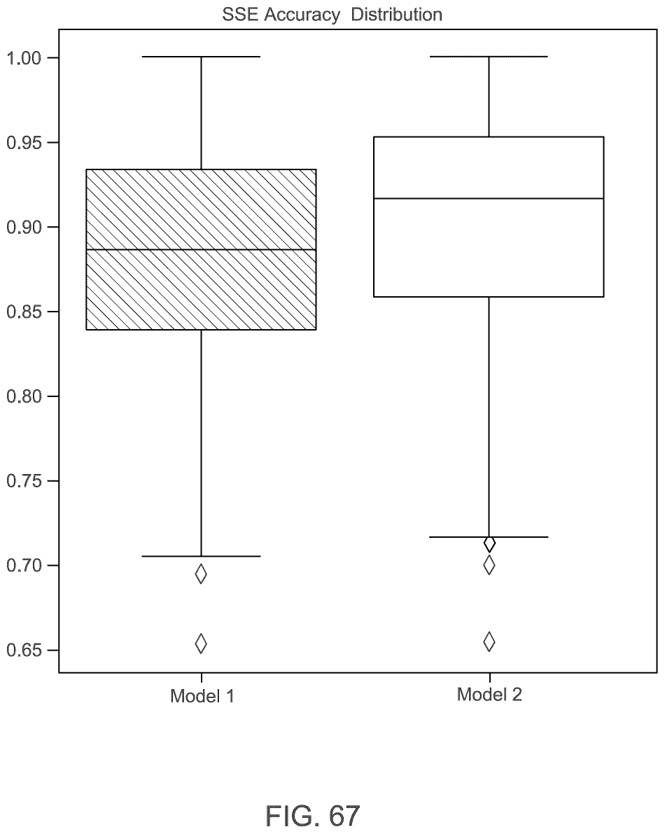

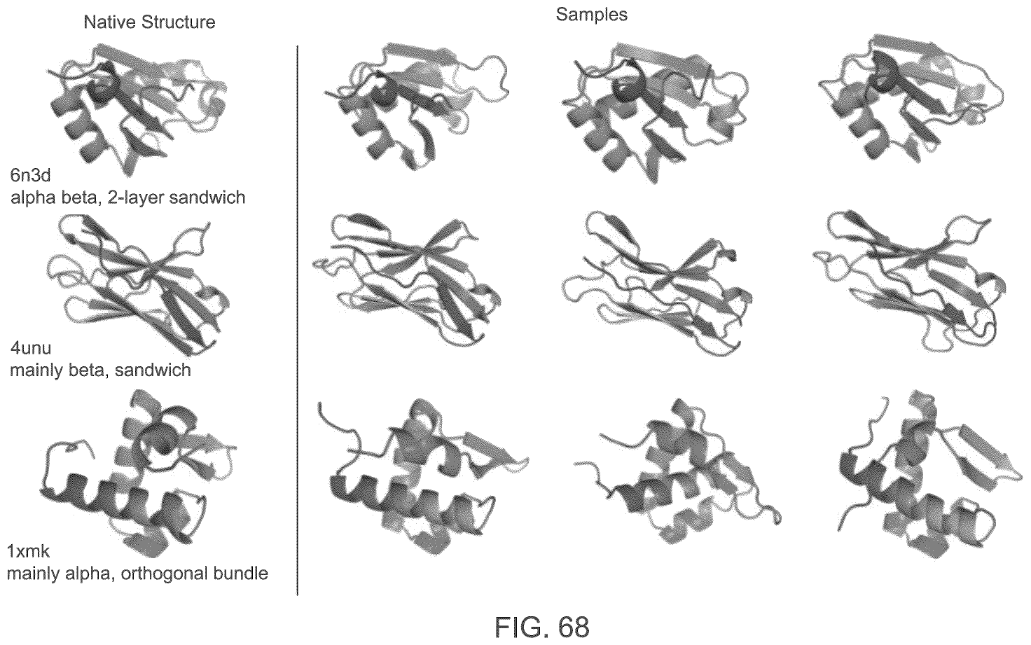

For every fold representative, 10 samples were generated, resulting in a collective 430 generations. Subsequently, the Fold Conditioning Design Benchmarks Metrics were calculated for each generation, in relation to the fold representative. FIGS. 65-67 present the TM scores and SSE accuracy findings. FIG. 65 shows comparative distribution of TM-scores between two models, with Model 1 averaging a TM-score of 0.44 and Model 2 displaying an improved average of 0.55. FIG. 66 shows distribution of the highest TM-scores selected from ten generations for each fold family, with Model 1 averaging a TM-score of 0.54 and Model 2 displaying an improved average of 0.70. FIG. 67 shows SSE Accuracy for the both models: both models show acceptable SSE accuracy, where the Model 2 bypasses Model 1 with an average of 0.90 and a median of 0.92. The ensuing plots demonstrate Model 2's significant enhancements over Model 1, suggesting that integrating relative segment orientations and including loops in the BAM calculation may bolster the model's fold conditioning capabilities. FIG. 68 shows sample generations as compared to CATH representatives on the left. On the right there are three different samples from the model with no post-processing. The samples show a high degree of variability and excellent conditioning on folds.

### CERTAIN REFERENCES

[Lipman 2022] Lipman et al., “Flow Matching for Generative Modeling,” arXiv, 2022.
[Chen and Lipman 2023] Chen and Lipman, “Riemannian Flow Matching on General Geometries,” arXiv, 2023.
[Bose 2023] Bose et al., “SE (3)-Stochastic Flow Matching for Protein Backbone Generation,” arXiv, 2023.
[Yim 2023] Yim et al., “Fast protein backbone generation with SE (3) flow matching,” arXiv 2023.
[Satorras 2021] Satorras, Hoogeboom, and Welling, “E(n) Equivariant Graph Neural Networks,” arXiv, 2021.
[Peebles 2022] Peebles and Xie, “Scalable Diffusion Models with Transformers,” arXiv, 2022.
[Darcet 2023] Darcet et al., “Vision Transformers Need Registers,” arxiv, 2023.
[Su 2021] Su et al., “RoFormer: Enhanced Transformer with Rotary Position Embedding,” arXiv, 2021.
[Vaswani 2017] Vaswani et al., “Attention is all you need,” arXiv, 2017.
[Yang 2019], Yang et al., “Improved protein structure prediction using predicted interresidue orientations,” Proc. Natl. Acad. Sci., 2019.
[Jumper 2021] Jumper et al., “Highly accurate protein structure prediction with AlphaFold,” Nature, (2021) Supplementary Information.
[Trippe 2022] Trippe et al., “Diffusion probabilistic modeling of protein backbones in 3D for the motif-scaffolding problem,” arXiv 2022.
[Daparas 2022] Dauparas et al., “Robust deep learning-based protein sequence design using ProteinMPNN,” Science, 2022.
[Lin 2023] Lin et al., “Evolutionary-scale prediction of atomic-level protein structure with a language model,” Science, 2023.
[Zhang & Skolnick 2004] Zhang and Skolnick, “Scoring function for automated assessment of protein structure template quality,” Proteins 2004.

### EQUIVALENTS

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein.

Throughout the description, where apparatus and systems are described as having, including, or comprising specific components, or where processes and methods are described as having, including, or comprising specific steps, it is contemplated that, additionally, there are apparatus, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

While the invention has been particularly shown and described with reference to specific preferred embodiments, it should be understood by those skilled in the art that various changes in form and detail may be made therein without departing from the spirit and scope of the invention as defined by the appended claims.

1-57. (canceled)

58. A method for in-silico generation and/or prediction of three-dimensional (3D) side chain geometries of a polypeptide chain, the method comprising:
(a) receiving and/or generating, by a processor of a computing device, (i) sequence data representing an amino acid sequence of at least a portion of the polypeptide chain and (ii) a representation of a 3D peptide backbone geometry, and/or folds thereof, for the portion of the polypeptide chain;
(b) receiving and/or generating, by a processor of a computing device, a seed set comprising a plurality of feature vectors, each feature vector comprising side chain geometry components representing a geometry of a side chain at a particular amino acid site;
(c) determining, by the processor, using a machine learning model, one or more velocity fields based at least in part on (i) the sequence data and (ii) the representation of the 3D peptide backbone geometry and/or folds thereof and, beginning with the seed set, updating, by the processor, values of the side chain geometry components of the plurality of feature vectors according to the one or more velocity fields, thereby evolving the values of the side chain geometry components of the plurality of feature vectors from a set of initial starting values into a set of final values representing geometries of each amino acid site of the portion of the polypeptide chain;
(d) creating, by the processor, using, the set of final values, a 3D polypeptide model of the portion of the polypeptide chain representing the 3D geometries of amino acid side chains within the portion of the polypeptide chain; and
(e) storing and/or providing, for display and/or further processing, the 3D polypeptide model.

59-60. (canceled)

61. A method for generating in-silico docking predictions for a plurality of polypeptide chains, the method comprising:
(a) receiving and/or generating, by a processor of a computing device, a first scaffold model representing a first three-dimensional (3D) peptide backbone of at least a portion of a first polypeptide chain;
(b) receiving and/or generating, by the processor, a second scaffold model representing a second 3D peptide backbone of at least a portion of a second polypeptide chain;
(c) receiving and/or generating, by the processor, a seed set comprising a plurality of feature vectors, the plurality of feature vectors comprising (i) a first set of feature vectors corresponding to and representing positions and/or orientations of the first 3D peptide backbone and/or (ii) a second set of feature vectors corresponding to and representing positions and/or orientations of the second 3D peptide backbone;
(d) determining, by the processor, using a machine learning model, based at least in part on the first scaffold model and the second scaffold model, one or more velocity fields, and, beginning with the seed set, updating, by the processor, values of (i) the first set of feature vectors and/or (ii) the second set of feature vectors according to the one or more velocity fields, thereby evolving values of the first and/or second set(s) of feature vector(s) from a set of initial starting values into a set of final values representing final position(s) and/or orientation(s) of the first 3D peptide backbone and/or the second 3D peptide backbone docked with each other;
(e) creating, by the processor, using, the set of final values, a generated polypeptide complex model representing the first and second 3D peptide backbones docked to form a polypeptide complex; and
(f) storing and/or providing the generated polypeptide complex model.

62-67. (canceled)

68. A system for in-silico generation and/or prediction of three-dimensional (3D) side chain geometries of a polypeptide chain, the system comprising:
a processor of a computing device; and
memory having instructions stored thereon, wherein the instructions, when executed by the processor, cause the processor to:
(a) receive and/or generate (i) sequence data representing an amino acid sequence of at least a portion of the polypeptide chain and (ii) a representation of a 3D peptide backbone geometry, and/or folds thereof, for the portion of the polypeptide chain;
(b) receive and/or generate a seed set comprising a plurality of feature vectors, each feature vector comprising side chain geometry components representing a geometry of a side chain at a particular amino acid site;
(c) determine, using a machine learning model, one or more velocity fields based at least in part on (i) the sequence data and (ii) the representation of the 3D peptide backbone geometry and/or folds thereof and, beginning with the seed set, update values of the side chain geometry components of the plurality of feature vectors according to the one or more velocity fields, thereby evolving the values of the side chain geometry components of the plurality of feature vectors from a set of initial starting values into a set of final values representing geometries of each amino acid site of the portion of the polypeptide chain;
(d) create, using, the set of final values, a 3D polypeptide model of the portion of the polypeptide chain representing the 3D geometries of amino acid side chains within the portion of the polypeptide chain; and
(e) store and/or provide the 3D polypeptide model.

69-70. (canceled)

71. A system for generating in-silico docking predictions for a plurality of polypeptide chains, the system comprising:
a processor of a computing device; and
memory having instructions stored thereon, wherein the instructions, when executed by the processor, cause the processor to:
(a) receive and/or generate a first scaffold model representing a first three-dimensional (3D) peptide backbone of at least a portion of a first polypeptide chain;
(b) receive and/or generate a second scaffold model representing a second 3D peptide backbone of at least a portion of a second polypeptide chain;
(c) receive and/or generate a seed set comprising a plurality of feature vectors, the plurality of feature vectors comprising (i) a first set of feature vectors corresponding to and representing positions and/or orientations of the first 3D peptide backbone and/or (ii) a second set of feature vectors corresponding to and representing positions and/or orientations of the second 3D peptide backbone;
(d) determine, using a machine learning model, based at least in part on the first scaffold model and the second scaffold model, one or more velocity fields and, beginning with the seed set, update values of (i) the first set of feature vectors and/or (ii) the second set of feature vectors according to the one or more velocity fields, thereby evolving values of the first and/or second set(s) of feature vector(s) from a set of initial starting values into a set of final values representing final position(s) and/or orientation(s) of the first 3D peptide backbone and/or the second 3D peptide backbone docket with each other;
(e) create, using, the set of final values, a generated polypeptide complex model representing the first and second 3D peptide backbones docked to form a polypeptide complex; and
(f) store and/or provide the generated polypeptide complex model.

72. (canceled)

73. The method of claim 58, wherein step (c) comprises determining the one or more velocity fields and updating the values of the side chain geometry components in an iterative fashion.

74. The method of claim 58, wherein, for each feature vector, the side chain geometry components are or comprise a set of side chain dihedral (x) angles.

75. The method of claim 58, comprising receiving and/or generating, at step (a), a scaffold model representing the 3D peptide backbone geometry.

76. The method of claim 58, comprising receiving and/or generating, at step (a), one or more protein fold representations comprising values encoding (i) secondary structure elements (SSEs), and/or (ii) relative positions and/or orientations of SSEs, for the 3D peptide backbone.

77. The method of claim 61, wherein the first polypeptide chain is at least a portion of an antibody and the second polypeptide chain is at least a portion of an antigen.

78. The method of claim 61, wherein step (d) comprises determining the one or more velocity fields and updating the values of (i) the first set of feature vectors and/or (ii) the second set of feature vectors according to the one or more velocity fields in an iterative fashion.

79. The method of claim 61, comprising conditioning generation of the one or more velocity fields on one or both of (i) an amino acid sequence of at least a portion of the first polypeptide chain and (ii) an amino acid sequence of at least a portion of the second polypeptide chain.

80. The method of claim 61, comprising generating a custom ligand for forming a complex with, and facilitating docking of, the first and second polypeptide chains.

81. The system of claim 68, wherein, at step (c), the instructions cause the processor to determine the one or more velocity fields and update the values of the side chain geometry components in an iterative fashion.

82. The system of claim 68, wherein, for each feature vector, the side chain geometry components are or comprise a set of side chain dihedral (x) angles.

83. The system of claim 68, wherein, at step (a), the instructions cause the processor to receive and/or generate a scaffold model representing the 3D peptide backbone geometry.

84. The system of claim 68, wherein, at step (a), the instructions cause the processor to receive and/or generate one or more protein fold representations comprising values encoding (i) secondary structure elements (SSEs), and/or (ii) relative positions and/or orientations of SSEs, for the 3D peptide backbone.

85. The system of claim 71, wherein the first polypeptide chain is at least a portion of an antibody and the second polypeptide chain is at least a portion of an antigen.

86. The system of claim 71, wherein, at step (d), the instructions cause the processor to determine the one or more velocity fields and update the values of (i) the first set of feature vectors and/or (ii) the second set of feature vectors according to the one or more velocity fields in an iterative fashion.

87. The system of claim 71, wherein the instructions cause the processor to condition generation of the one or more velocity fields on one or both of (i) an amino acid sequence of at least a portion of the first polypeptide chain and (ii) an amino acid sequence of at least a portion of the second polypeptide chain.

88. The system of claim 71, wherein the instructions cause the processor to generate a custom ligand for forming a complex with, and facilitating docking of, the first and second polypeptide chains.
